(19) United States
(12) Patent Application Publication
(10) Pub. No.: US 2015/0368236 A1
(43) Pub. Date: Dec. 24, 2015

(54) 2-(PYRIDIN-3-YL)-5-HETARYL-THIAZOLE COMPOUNDS CARRYING AN IMINE OR IMINE-DERIVED SUBSTITUENT FOR COMBATING INVERTEBRATE PESTS

(71) Applicant: BASF SE, Ludwigshafen (DE)

(72) Inventors: Florian KAISER, Mannheim (DE); Arun NARINE, Mannheim (DE); Karsten KOERBER, Eppelheim (DE); Joachim DICKHAUT, Heidelberg (DE); Jean-Yves WACH, Mannheim (DE); Nina Gertrud BANDUR, Ludwigshafen (DE)

(21) Appl. No.: 14/654,933

(22) PCT Filed: Dec. 23, 2013
(86) PCT No.: PCT/EP2013/077924

§ 371 Date: Jun. 23, 2015

Related U.S. Application Data
(60) Provisional application No. 61/746,139, filed on Dec. 27, 2012.

(51) Int. Cl. C07D 417/14; A01N 43/84; A01N 43/78

(52) U.S. Cl. CPC C07D 417/14; A01N 43/78; A01N 43/84

(57) ABSTRACT

The present invention relates to 2-(pyridin-3-yl)-5-hetaryl-thiazole compounds carrying an imine or imine-derived substituent on the 5-hetaryl ring which are useful for combating or controlling invertebrate pests, in particular arthropod pests and nematodes, and to a method for producing them. The invention also relates to a method for controlling invertebrate pests by using these compounds and to plant propagation material and to an agricultural and a veterinary composition comprising said compounds.

The present invention relates to 2-(pyridin-3-yl)-5-hetaryl-thiazole compounds carrying an imine or imine-derived substituent on the 5-hetaryl ring which are useful for combating or controlling invertebrate pests, in particular arthropod pests and nematodes, and to a method for producing them. The invention also relates to a method for controlling invertebrate pests by using these compounds and to plant propagation material and to an agricultural and a veterinary composition comprising said compounds.

Invertebrate pests and in particular arthropods and nematodes destroy growing and harvested crops and attack wooden dwelling and commercial structures, causing large economic loss to the food supply and to property. While a large number of pesticidal agents are known, due to the ability of target pests to develop resistance to said agents, there is an ongoing need for new agents for combating invertebrate pests, in particular insects, arachnids and nematodes.

Certain diaryl-thiazoles and substituted pyridyl thiazole heterocycles are disclosed in Bioorganic & Medicinal Chemistry Letters 2012, 22(9), 3083-3088, in WO 2012/021696, in WO 2011/133733, in EP-A-117082, in EP-A-149884 and EP-A1205478 as enzyme inhibitors and pharmaceutical agents. Amidine-substituted diaryl-thiazoles are disclosed in Bioorganic & Medicinal Chemistry 2010, 18(10), 3551-3558 as antiprotozoal agents.

WO 2010/006713 describes pyridyl thiazole-substituted heterocycle derivatives and their use as pesticides. Other pyridyl thiazole-substituted heterocycle pesticidal compounds are likewise disclosed in WO 2011/134964, WO 2011/138285 and WO 2012/000896. WO 2010/129497 describes pyridyl thiazole amines and their applications as pesticides. Similar pesticidal compounds are likewise disclosed in WO 2011/128304 and WO 2012/030681. Pesticidal 3-pyridyl thiazole carboxamides have been described in U.S. Pat. No. 4,260,765. WO 2009/149858 describes pyridyl thiazole carboxamide derivatives and their applications as pesticide. Similar pesticidal carboxamide compounds are likewise disclosed in WO 2011/128304. 4-Haloalkyl-3-heterocyclylpyridines as pesticides are disclosed in WO 98/57969. Similar compounds are likewise disclosed in WO 2000/035285 and US 2003/0162812. Heterocyclyl-substituted thiazole derivatives and their use as fungicides have been described in WO 2007/033780. Substituted haloalkyl thiazole derivatives and their use as insecticides are disclosed in WO 2004/056177.

However, these documents do not describe compounds having the characteristic substituents as claimed in the present invention.

It is an object of the present invention to provide compounds that have a good pesticidal activity, in particular insecticidal activity, and show a broad activity spectrum against a large number of different invertebrate pests, especially against difficult to control arthropod pests and/or nematodes.

It has been found that these objectives can be achieved by 2-(pyridin-3-yl)-5-hetaryl-thiazole compounds of the formula I below, by their stereoisomers and by their salts, in particular their agriculturally or veterinarily acceptable salts.

Therefore, in a first aspect, the invention relates to 2-(pyridin-3-yl)-5-hetaryl-thiazole compounds of formula I

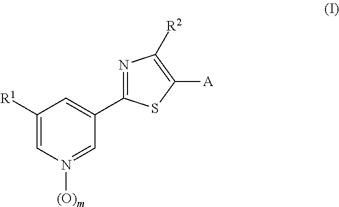

wherein
R¹ is selected from the group consisting of hydrogen, cyano and halogen;
R² is selected from the group consisting of hydrogen, halogen, cyano, C₁-C₆-alkyl and C₃-C₆-cycloalkyl, where the two last-mentioned radicals may be partially or fully halogenated and/or may be substituted by one or more, e.g. 1, 2 or 3, in particular 1, radicals R³;
A is a heteroaromatic radical selected from the radicals of formulae A-1 to A-29:

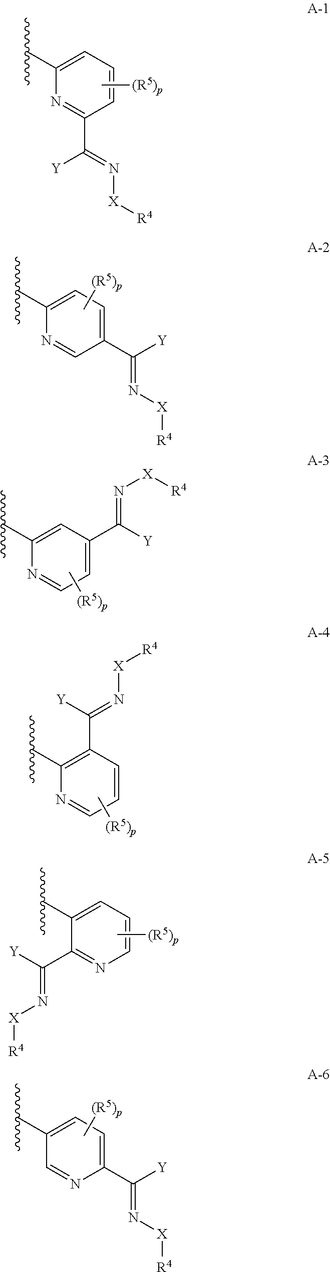

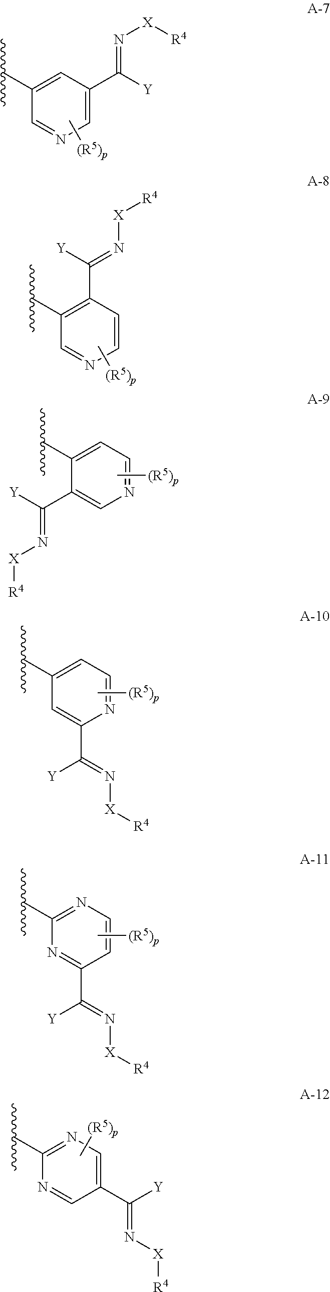

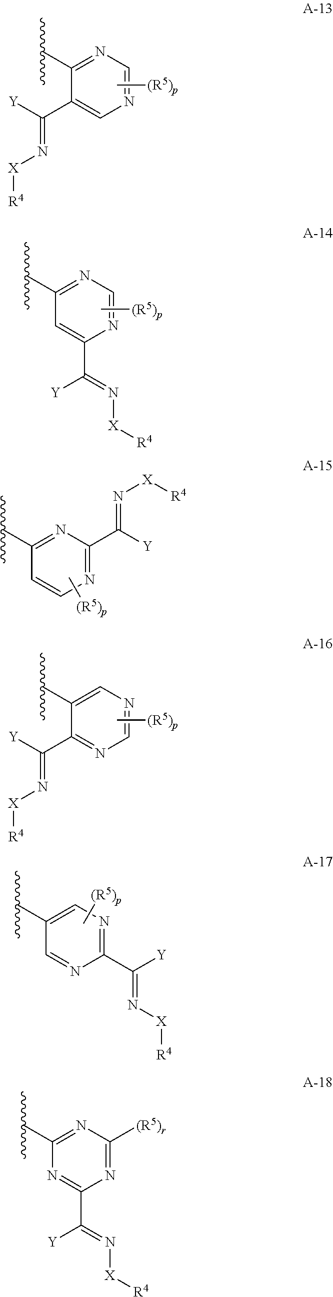

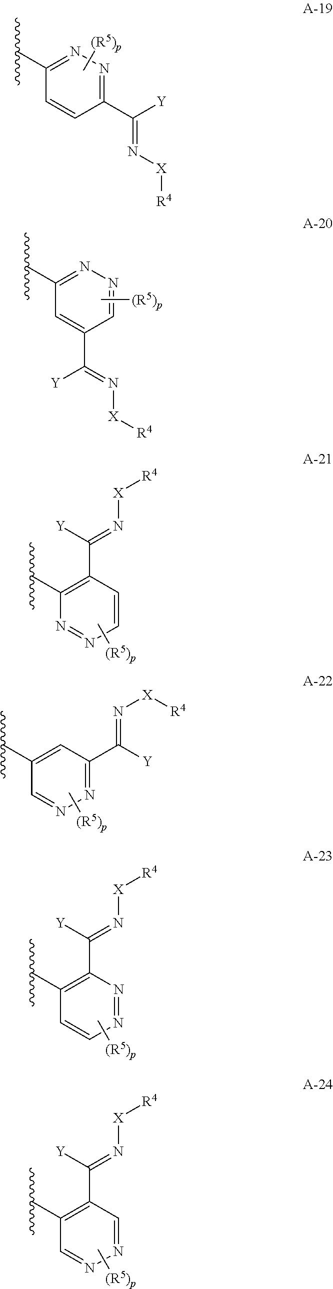

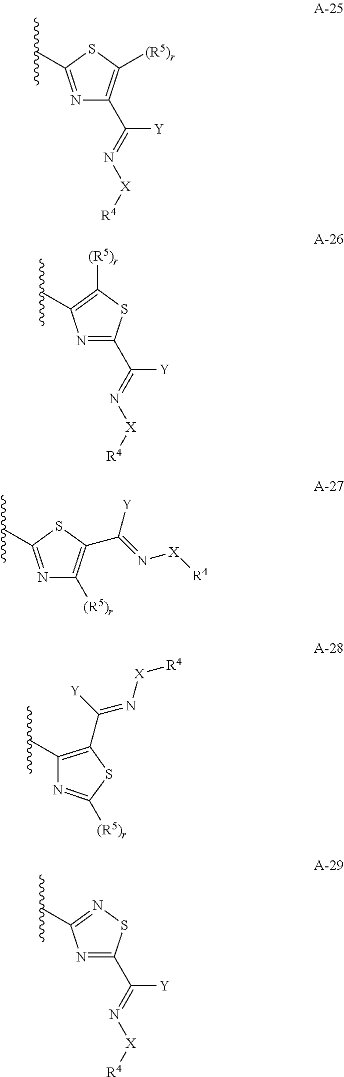

wherein
the zigzag line denotes the bond to the thiazole ring;
X is O, N—R^{4a}, S(O)ₙ or a chemical bond;
Y is selected from hydrogen, C₁-C₆-alkyl, C₃-C₆-cycloalkyl, where the two last-mentioned radicals may be partially or fully halogenated and/or may be substituted by one or more radicals R⁷;
   C(═O)NR^{9a}R^{9b} and C(═NR^{9a})R⁷;
R³ is each independently from one another selected from the group consisting of cyano, —SCN, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkyl, C₃-C₈-halocycloalkyl, Si(R¹¹)₂R¹², OR¹⁶, OSO₂R¹⁶, S(O)ₙR¹⁶, S(O)ₙNR^{17a}R^{17b}, NR^{17a}R^{17b}, C(═O)NR^{17a}R^{17b}, C(═S)NR^{17a}R^{17b}, C(═O)OR¹⁶,
   phenyl, optionally substituted with 1, 2, 3, 4 or 5, preferably 1, 2 or 3, in particular 1, substituents R¹⁵; and
   a 3-, 4-, 5-, or 6-membered saturated, partly unsaturated or maximally unsaturated heterocyclic ring comprising 1, 2 or 3 heteroatoms or heteroatom groups selected from N, O, S, NO, SO and SO₂ as ring members, wherein the heterocyclic ring is optionally substituted with one or more, e.g. 1, 2 or 3, preferably 1 or 2, in particular 1, substituents R¹⁵; and
   R³ as a substituent on a cycloalkyl ring is additionally selected from C₁-C₆-alkyl, C₁-C₆-haloalkyl, C₂-C₆-alkenyl, C₂-C₆-haloalkenyl, C₂-C₆-alkynyl and C₂-C₆-haloalkynyl;
R⁴ is selected from the group consisting of hydrogen, cyano, C₁-C₁₀-alkyl, C₃-C₈-cycloalkyl, C₂-C₁₀-alkenyl, C₂-C₁₀-alkynyl, wherein the four last-mentioned radicals may be partially or fully halogenated and/or may be substituted with one or more, e.g. 1, 2 or 3, in particular 1, radicals R⁷;
   OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
   phenyl, optionally substituted with 1, 2, 3, 4 or 5, preferably 1, 2 or 3, in particular 1, substituents R¹⁰; and
   a 3-, 4-, 5-, 6- or 7-membered saturated, partly unsaturated or maximally unsaturated heterocyclic ring comprising 1, 2, 3 or 4 heteroatoms or heteroatom groups selected from N, O, S, NO, SO and SO₂ as ring members, where the heterocyclic ring is optionally substituted with one or more, e.g. 1, 2 or 3, preferably 1 or 2, in particular 1, substituents R¹⁰;
R^{4a} is selected from the group consisting of hydrogen, C₁-C₆-alkyl, C₁-C₆-haloalkyl, C₃-C₈-cycloalkyl, C₃-C₈-halocycloalkyl, C₁-C₆-alkoxy, C₁-C₆-haloalkoxy, phenyl and benzyl, where the phenyl ring in the two last-mentioned radicals may carry 1, 2, 3, 4 or 5, preferably 1, 2 or 3, in particular 1, substituents selected from halogen, C₁-C₄-alkyl, C₁-C₄-haloalkyl, C₁-C₄-alkoxy and C₁-C₄-haloalkoxy;
R⁵ is each independently from one another selected from the group consisting of halogen, cyano, azido, nitro, SCN, SF₅, C₁-C₁₀-alkyl, C₃-C₈-cycloalkyl, C₂-C₁₀-alkenyl, C₂-C₁₀-alkynyl, wherein the four last-mentioned radicals may be partially or fully halogenated and/or may be substituted with one or more, e.g. 1, 2 or 3, in particular 1, radicals R⁷;
   OR¹⁶, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
   phenyl, optionally substituted with 1, 2, 3, 4, or 5, preferably 1, 2 or 3, in particular 1, substituents R⁶;
   and
   a 3-, 4-, 5-, 6- or 7-membered saturated, partly unsaturated or maximally unsaturated heterocyclic ring comprising 1, 2, 3 or 4 heteroatoms or heteroatom groups selected from N, O, S, NO, SO and SO₂ as ring members, where the heterocyclic ring is optionally substituted with one or more, e.g. 1, 2 or 3, preferably 1 or 2, in particular 1, substituents R⁶;
   or two R⁵, together with the ring atoms they are bonded to, form a 3-, 4-, 5-, 6-, 7- or 8-membered saturated, partially unsaturated or maximally unsaturated carbocyclic or heterocyclic ring, where the heterocyclic ring contains 1 or 2 heteroatoms or heteroatom groups selected from N, O, S, NO, SO and SO₂ as ring members, where the carbocyclic or heterocyclic ring may contain 1 or 2 groups selected from C═O, C═S and C═NR^{17a} as ring members; and where the carbocyclic or heterocyclic ring may be substituted by one or more, e.g. 1, 2 or 3, preferably 1 or 2, in particular 1, radicals selected from the group consisting of halogen, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₁-C₆-alkylthio, C₁-C₆-haloalkylthio, phenyl which may be substituted by 1, 2, 3, 4 or 5 radicals R¹⁵, and a 3-, 4-, 5-, 6- or 7-membered saturated, partly unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more, e.g. 1, 2 or 3, preferably 1 or 2, in particular 1, radicals R¹⁵;
R⁶ is each independently from one another selected from the group consisting of halogen, cyano, nitro, SCN, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted with one or more, e.g. 1, 2 or 3, in particular 1, radicals R⁷;
   OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
   phenyl, optionally substituted with 1, 2, 3, 4, or 5, preferably 1, 2 or 3, in particular 1, substituents R¹⁰; and
   a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more, e.g. 1, 2 or 3, preferably 1 or 2, in particular 1, substituents selected independently from one another from halogen, cyano, NO₂, C₁-C₆-alkyl, C₁-C₆-haloalkyl, C₁-C₆-alkoxy and C₁-C₆-haloalkoxy;
   or
   two R⁶ present together on one carbon atom of a partly saturated heterocyclic ring may form together a group ═O, ═CR¹³R¹⁴; ═S, ═NR^{17a}, ═NOR¹⁶ or ═NNR^{17a};
   or
   two R⁶ bound on adjacent carbon atoms may together form a bridge selected from CH₂CH₂CH₂CH₂, CH═CH—CH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR^{17a}, CH₂CH═N, CH═CH—NR^{17a}, OCH═N and SCH═N, thus forming together with the carbon atoms to which they are bonded to a 5-membered or 6-membered saturated, partly unsaturated or aromatic carbocyclic or heterocyclic ring, wherein the ring may optionally be substituted with one or two substituents selected from ═O, OH, CH₃, OCH₃, halogen, cyano, halomethyl and halomethoxy;
R⁷ is each independently from one another selected from the group consisting of cyano, azido, nitro, —SCN, SF₅, C₃-C₈-cycloalkyl, C₃-C₈-halocycloalkyl, Si(R¹¹)₂R¹², OR¹⁶, OSO₂R¹⁶, S(O)ₙR¹⁶, S(O)ₙNR^{17a}R^{17b}, NR^{17a}R^{17b}, C(═O)NR^{17a}R^{17b}, C(═S)NR^{17a}R^{17b}, C(═O)OR¹⁶,
   phenyl, optionally substituted with 1, 2, 3, 4 or 5, preferably 1, 2 or 3, in particular 1, substituents R¹⁵, and
   a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more, e.g. 1, 2 or 3, preferably 1 or 2, in particular 1, substituents R¹⁵,
   or
   two R⁷ present on the same carbon atom of an alkyl, alkenyl, alkynyl or cycloalkyl group may together form a group ═O, ═CR¹³R¹⁴; ═S, ═NR^{17a}, ═NOR¹⁶ or ═NNR^{17a};
   or
   two R⁷, together with the carbon atom(s) of an alkyl, alkenyl, alkynyl or cycloalkyl group to which they are bonded, may form a 3-, 4-, 5-, 6-, 7- or 8-membered saturated or partly unsaturated carbocyclic or heterocyclic ring, where the heterocyclic ring comprises 1, 2, 3 or 4 heteroatoms or heteroatom groups selected from N, O, S, NO, SO and SO₂ as ring members, and where the carbocyclic or heterocyclic ring is optionally substituted with one or more, e.g. 1, 2 or 3, preferably 1 or 2, in particular 1, substituents R¹⁵; and
   R⁷ as a substituent on a cycloalkyl ring is additionally selected from the group consisting of C₁-C₆-alkyl, C₁-C₆-haloalkyl, C₂-C₆-alkenyl, C₂-C₆-haloalkenyl, C₂-C₆-alkynyl and C₂-C₆-haloalkynyl, where the aliphatic moieties in these six radicals may be substituted by one or more, e.g. 1, 2 or 3, in particular 1, radicals R¹⁹; and
   R⁷ in the groups —C(═NR^{9a})R⁷, —C(═O)R⁷ and —C(═S)R⁷ is additionally selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl and C₂-C₆-haloalkynyl, where the aliphatic moieties in the six last-mentioned radicals may be substituted by one or more, e.g. 1, 2 or 3, in particular 1, radicals R¹⁹;
R⁸ is each independently from one another selected from the group consisting of hydrogen, cyano, C₁-C₆-alkyl, C₁-C₆-haloalkyl, C₃-C₈-cycloalkyl, C₃-C₈-cycloalkyl-C₁-C₄-alkyl-, C₃-C₈-halocycloalkyl, C₂-C₆-alkenyl, C₂-C₆-haloalkenyl, C₂-C₆-alkynyl, C₂-C₆ haloalkynyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —Si(R¹¹)₂R¹², S(O)ₙR¹⁶, S(O)ₙNR^{17a}R^{17b}, NR^{17a}R^{17b}, —N═CR¹³R¹⁴, —C(═O)R¹⁸, C(═O)NR^{17a}R^{17b}, C(═S)NR^{17a}R^{17b}, C(═O)OR¹⁶, phenyl, optionally substituted with 1, 2, 3, 4 or 5, preferably 1, 2 or 3, in particular 1, substituents R¹⁵; and
   a 3-, 4-, 5-, 6- or 7-membered saturated, partly unsaturated or maximally unsaturated heterocyclic ring comprising 1, 2 or 3 heteroatoms selected from N, O, S, NO, SO and SO₂ as ring members, where the heterocyclic ring is optionally substituted with one or more, e.g. 1, 2 or 3, preferably 1 or 2, in particular 1, substituents R₁₅;
R^{9a}, R^{9b} are each independently from one another selected from the group consisting of hydrogen, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl, C₁-C₆-alkoxy, C₁-C₆-haloalkoxy, C₁-C₆-alkylthio, C₁-C₆-haloalkylthio, S(O)ₙR¹⁶, —S(O)ₙNR^{17a}R^{17b}, C(═O)R¹⁸, C(═O)OR¹⁶, C(═O)NR^{17a}R^{17b}, C(═S)R¹⁸, C(═S)SR¹⁶, C(═S)NR^{17a}R^{17b}, C(═NR^{17a})R¹⁸;
   phenyl, optionally substituted with 1, 2, 3, 4 or 5, preferably 1, 2 or 3, in particular 1, substituents R¹⁵; and
   a 3-, 4-, 5-, 6- or 7-membered saturated, partly unsaturated or maximally unsaturated heterocyclic ring comprising 1, 2, 3 or 4 heteroatoms selected from N, O, S, NO, SO and SO₂ as ring members, where the heterocyclic ring is optionally substituted with one or more, e.g. 1, 2 or 3, preferably 1 or 2, in particular 1, substituents R¹⁵;
   or,
   R^{9a} and R^{9b}, together with the nitrogen atom they are bonded to, form a 3-, 4-, 5-, 6-, 7- or 8-membered saturated, partly unsaturated or maximally unsaturated ring, wherein the heterocyclic ring may additionally contain one or two heteroatoms or heteroatom groups selected from N, O, S, NO, SO and SO₂ as ring members, wherein the heterocyclic ring may optionally be substituted with one or more, e.g. 1, 2 or 3, preferably 1 or 2, in particular 1, radicals selected from halogen, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl, C₁-C₆-alkoxy, C₁-C₆-haloalkoxy, C₁-C₆-alkylthio, C₁-C₆-haloalkylthio, phenyl, optionally substituted with 1, 2, 3, 4 or 5 substituents R¹⁵; and a 3-, 4-, 5-, -6, or 7-membered saturated, partly unsaturated or maximally unsaturated heterocyclic ring comprising 1, 2 or 3 heteroatoms selected from N, O, S, NO, SO and SO₂ as ring members, where the heterocyclic ring is optionally substituted with one or more, e.g. 1, 2 or 3, preferably 1 or 2, in particular 1, substituents R¹⁵;
   or
   R^{9a} and R^{9b} together form a ═CR¹³R¹⁴, ═NR^{17a} or ═NOR¹⁶ radical;
R¹⁰ is each independently from one another selected from the group consisting of halogen, cyano, azido, nitro, SCN, SF₅, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may partially or fully halogenated and/or may be substituted with one or more, e.g. 1, 2 or 3, in particular 1, radicals R¹⁹;
   Si(R¹¹)₂R¹², OR¹⁶, OS(O)ₙR¹⁶, —S(O)ₙR¹⁶, S(O)ₙNR^{17a}R^{17b}, NR^{17a}R^{17b}, C(═O)R¹⁸, C(═O)OR¹⁶, —C(═NR^{17a})R¹⁸, C(═O)NR^{17a}R^{17b}, C(═S)NR^{17a}R^{17b},
   phenyl, optionally substituted with 1, 2, 3, 4 or 5, preferably 1, 2 or 3, in particular 1, radicals selected from halogen, cyano, nitro, C₁-C₆-alkyl, C₁-C₆-haloalkyl, C₁-C₆-alkoxy and C₁-C₆-haloalkoxy; and
   a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more, e.g. 1, 2 or 3, preferably 1 or 2, in particular 1, substituents selected independently from one another from halogen, cyano, NO₂, C₁-C₆-alkyl, C₁-C₆-haloalkyl, C₁-C₆-alkoxy and C₁-C₆-haloalkoxy;
   or
   two R¹⁰ present together on the same carbon atom of a heterocyclic may together form a group ═O, ═CR¹³R¹⁴; ═S, ═NR^{17a}, ═NOR¹⁶ or ═NNR^{17a};
   or
   two R¹⁰ on adjacent carbon atoms may together form a bridge selected from CH₂CH₂CH₂CH₂, CH═CH—CH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR^{17a}, CH₂CH═N, CH═CH—NR^{17a} and OCH═N, SCH═N, thus forming together with the carbon atoms to which they are bonded a 5-membered or 6-membered partly unsaturated or aromatic carbocyclic or heterocyclic ring, wherein the ring may be substituted with one or two substituents selected from ═O, OH, CH₃, OCH₃, halogen, cyano, halomethyl and halomethoxy;
R¹¹, R¹² are each independently from one another selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy, C₁-C₆-haloalkoxy, C₁-C₆-alkoxy-C₁-C₄-alkyl, C₁-C₆-haloalkoxy-C₁-C₄-alkyl and
   phenyl, optionally substituted with 1, 2, 3, 4 or 5, preferably 1, 2 or 3, in particular 1, substituents R¹⁵;
R¹³, R¹⁴ are each independently from one another selected from the group consisting of hydrogen, C₁-C₄-alkyl, C₃-C₆-cycloalkyl, C₁-C₄-alkoxy-C₁-C₄-alkyl, phenyl and benzyl;
R¹⁵ is each independently from one another selected from the group consisting of halogen, cyano, nitro, OH, SH, SCN, SF₅, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl,
   C₁-C₆-alkyl, C₂-C₆-alkenyl, C₂-C₆-alkynyl, C₃-C₈-cycloalkyl, wherein the four last-mentioned aliphatic and cycloaliphatic radicals may be unsubstituted, partially or fully halogenated and/or may carry 1 or 2 radicals selected from hydroxyl and C₁-C₄-alkoxy;
   phenyl, benzyl, pyridyl and phenoxy, wherein the four last-mentioned radicals may be unsubstituted or may carry 1, 2 or 3, in particular 1, substituents selected from halogen, C₁-C₆-alkyl, C₁-C₆-haloalkyl, C₁-C₆-alkoxy, C₁-C₆-haloalkoxy, (C₁-C₆-alkoxy)carbonyl, (C₁-C₆-alkyl)amino and di-(C₁-C₆-alkyl)amino;
   or
   two R¹⁵ present on the same carbon atom of a heterocyclic ring may together form a group ═O, ═CH(C₁-C₄), ═C(C₁-C₄-alkyl)C₁-C₄-alkyl, ═N(C₁-C₆-alkyl) or ═NO(C₁-C₆-alkyl);
R¹⁶ is each independently from one another selected from the group consisting of hydrogen, C₁-C₆-alkyl, C₂-C₆-alkenyl, C₂-C₆-alkynyl, C₃-C₈-cycloalkyl, wherein the four last-mentioned radicals may be unsubstituted, partially or fully halogenated and/or may carry 1 or 2 radicals selected from hydroxyl and C₁-C₄-alkoxy, phenyl, benzyl, pyridyl and phenoxy, wherein the four last-mentioned radicals may be unsubstituted or carry 1, 2 or 3, in particular 1, substituents selected from halogen, C₁-C₆-alkyl, C₁-C₆-haloalkyl, C₁-C₆-alkoxy, C₁-C₆-haloalkoxy and (C₁-C₆-alkoxy)carbonyl;
R^{17a}, R^{17b} are each independently from one another selected from the group consisting of hydrogen, cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl, C₁-C₆-alkyl, C₂-C₆-alkenyl, C₂-C₆-alkynyl, C₃-C₈-cycloalkyl, wherein the four last-mentioned aliphatic and cycloaliphatic radicals may be unsubstituted, partially or fully halogenated and/or may carry 1 or 2 radicals selected from hydroxyl and C₁-C₄-alkoxy; C₃-C₈-cycloalkyl-C₁-C₄-alkyl,
   phenyl, benzyl, pyridyl and phenoxy, wherein the four last-mentioned radicals may be unsubstituted or carry 1, 2 or 3, in particular 1, substituents selected from halogen, C₁-C₆-alkyl, C₁-C₆-haloalkyl, C₁-C₆-alkoxy, C₁-C₆-haloalkoxy and (C₁-C₆-alkoxy)carbonyl,
   or
   R^{17a} and R^{17b}, together with the nitrogen atom they are bonded to, form a 3-, 4-, 5-, 6- or 7-membered saturated, partly unsaturated or maximally unsaturated ring, wherein the heterocyclic ring may additionally contain one or two heteroatoms or heteroatom groups selected from N, O, S, NO, SO and SO₂ as ring members, wherein the heterocyclic ring may be substituted with one or more, e.g. 1, 2 or 3, preferably 1 or 2, in particular 1, substituents selected from halogen, cyano, C₁-C₄-alkyl, C₁-C₄-haloalkyl, C₁-C₄-alkoxy and C₁-C₄-haloalkoxy;
R¹⁸ is each independently from one another selected from the group consisting hydrogen, halogen, OH, SH, SCN, SF₅, C₁-C₆-alkyl, C₂-C₆-alkenyl, C₂-C₆-alkynyl, C₃-C₈-cycloalkyl, wherein the four last-mentioned aliphatic and cycloaliphatic radicals may be unsubstituted, partially or fully halogenated and/or may carry 1 or 2 radicals selected from hydroxyl and C₁-C₄-alkoxy;
   C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, phenyl, benzyl, pyridyl and phenoxy, wherein the four last-mentioned radicals may be unsubstituted or may carry 1, 2 or 3, in particular 1, substituents selected from halogen, C₁-C₆-alkyl, C₁-C₆-haloalkyl, C₁-C₆-alkoxy, C₁-C₆-haloalkoxy, (C₁-C₆-alkoxy)carbonyl, (C₁-C₆-alkyl)amino and di-(C₁-C₆-alkyl)amino,
R¹⁹ is each independently from one another selected from the group consisting of cyano, nitro, OH, SH, SCN, SF₅, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl,
   C₃-C₈-cycloalkyl, wherein the last-mentioned radical may be unsubstituted, partially or fully halogenated and/or may carry 1 or 2 radicals selected from hydroxyl and C₁-C₄-alkoxy;
   phenyl, benzyl, pyridyl and phenoxy, wherein the four last-mentioned radicals may be unsubstituted or may carry 1, 2 or 3, in particular 1, substituents selected from halogen, C₁-C₆-alkyl, C₁-C₆-haloalkyl, C₁-C₆-alkoxy, C₁-C₆-haloalkoxy, (C₁-C₆-alkoxy)carbonyl, (C₁-C₆-alkyl)amino and di-(C₁-C₆-alkyl)amino,
   or
   two R¹⁹ present on the same carbon atom may together form a group ═O, ═CH(C₁-C₄), ═C(C₁-C₄-alkyl)C₁-C₄-alkyl, ═N(C₁-C₆-alkyl) or ═NO(C₁-C₆-alkyl); and R¹⁹ as a substituent on a cycloalkyl ring is additionally selected from the group consisting of C₁-C₆-alkyl, C₁-C₆-haloalkyl, C₂-C₆-alkenyl, C₂-C₆-haloalkenyl, C₂-C₆-alkynyl and C₂-C₆-haloalkynyl;
m is 0 or 1;
n is 0, 1 or 2;
p is 0, 1 or 2; and
r is 0 or 1;
and/or an enantiomer, diastereomer, E/Z-isomer or agriculturally or veterinarily acceptable salts thereof.

It has to be noted that the structures shown above for ring A do not limit the compounds to a specific steroisomerism at the nitrogen atom of the imine (derived) group C(Y)═N—X—R⁴; i.e. the group Y and the group X—R⁴ can be both cis or trans to each other. Preferably, however, Y and the group X—R⁴ are cis to each other.

The present invention also provides an agricultural composition comprising at least one compound of the formula I as defined herein and/or an agriculturally acceptable salt thereof and at least one liquid or solid carrier.

The present invention also provides a veterinary composition comprising at least one compound of the formula I as defined herein and/or a veterinarily acceptable salt thereof and at least one liquid or solid carrier.

The present invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of formula I or a salt thereof as defined herein.

The present invention also relates to plant propagation material, in particular seed, comprising at least one compound of formula I and/or an agriculturally acceptable salt thereof as defined herein.

The present invention further relates to a method for treating or protecting an animal from infestation or infection by parasites which comprises bringing the animal in contact with a parasiticidally effective amount of a compound of the formula I or a veterinarily acceptable salt thereof as defined herein. Bringing the animal in contact with the compound I, its salt or the veterinary composition of the invention means applying or administering it to the animal. The invention also relates to a compound I or a veterinarily acceptable salt thereof as defined herein for use as a medicament and especially for treating or protecting an animal from infestation or infection by parasites; and also to the use of a compound I or a veterinarily acceptable salt thereof as defined herein for preparing a medicament for treating an animal from infestation or infection by parasites.

The term “steroisomers” encompasses both optical isomers, such as enantiomers or diastereomers, the latter existing due to more than one center of chirality in the molecule, as well as geometrical isomers (cis/trans isomers).

Depending on the substitution pattern, the compounds of the formula I may have one or more centers of chirality, in which case they are present as mixtures of enantiomers or diastereomers. The invention provides both the pure enantiomers or diastereomers and their mixtures and the use according to the invention of the pure enantiomers or diastereomers of the compound I or its mixtures. Suitable compounds of the formula I also include all possible geometrical stereoisomers (cis/trans isomers) and mixtures thereof.

The term N-oxides relates to a form of compounds I in which at least one nitrogen atom is present in oxidized form (as NO) and especially to compounds I, wherein m is 1.

The compounds of the present invention may be amorphous or may exist in one or more different crystalline states (polymorphs) which may have a different macroscopic properties such as stability or show different biological properties such as activities. The present invention includes both amorphous and crystalline compounds of the formula I, mixtures of different crystalline states of the respective compound I, as well as amorphous or crystalline salts thereof.

Salts of the compounds of the formula I are preferably agriculturally and veterinarily acceptable salts. They can be formed in a customary method, e.g. by reacting the compound with an acid of the anion in question if the compound of formula I has a basic functionality or by reacting an acidic compound of formula I with a suitable base.

Suitable agriculturally acceptable salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH⁴⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyethoxy)ethylammonium, bis(2-hydroxyethyl)ammonium, benzyltrimethylammonium and benzyl-triethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting a compound of formulae I with an acid of the corresponding anion, preferably of hydrochloric acid, hydrobromic acid, sulfuric acid, phosphoric acid or nitric acid.

By the term “veterinarily acceptable salts” is meant salts of those cations or anions which are known and accepted in the art for the formation of salts for veterinary use. Suitable acid addition salts, e.g. formed by compounds of formula I containing a basic nitrogen atom, e.g. an amino group, include salts with inorganic acids, for example hydrochlorides, sulphates, phosphates, and nitrates and salts of organic acids for example acetic acid, maleic acid, dimaleic acid, fumaric acid, difumaric acid, methane sulfenic acid, methane sulfonic acid, and succinic acid.

The term “invertebrate pest” as used herein encompasses animal populations, such as insects, arachnids and nematodes, which may attack plants, thereby causing substantial damage to the plants attacked, as well as ectoparasites which may infest animals, in particular warm blooded animals such as e.g. mammals or birds, or other higher animals such as reptiles, amphibians or fish, thereby causing substantial damage to the animals infested.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. The plant propagation materials may be treated prophylactically with a plant protection compound either at or before planting or transplanting. Said young plants may also be protected before transplantation by a total or partial treatment by immersion or pouring.

The term “plants” comprises any types of plants including “non-cultivated plants” and in particular “cultivated plants”.

The term “non-cultivated plants” refers to any wild type species or related species or related genera of a cultivated plant.

The term “cultivated plants” is to be understood as including plants which have been modified by breeding, mutagenesis or genetic engineering including but not limiting to agricultural biotech products on the market or in development (cf. http://www.bio.org/speeches/pubs/er/agri_products.asp). Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-translational modification of protein(s), oligo- or polypeptides e. g. by glycosylation or polymer additions such as prenylated, acetylated or farnesylated moieties or PEG moieties.

Plants that have been modified by breeding, mutagenesis or genetic engineering, e. g. have been rendered tolerant to applications of specific classes of herbicides, such as auxin herbicides such as dicamba or 2,4-D; bleacher herbicides such as hydroxylphenylpyruvate dioxygenase (HPPD) inhibitors or phytoene desaturase (PDS) inhibitors; acetolactate synthase (ALS) inhibitors such as sulfonyl ureas or imidazolinones; enolpyruvylshikimate-3-phosphate synthase (EPSPS) inhibitors, such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i. e. bromoxynil or ioxynil) herbicides as a result of conventional methods of breeding or genetic engineering. Furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are e. g. described in Pest Managem. Sci. 61, 2005, 246; 61, 2005, 258; 61, 2005, 277; 61, 2005, 269; 61, 2005, 286; 64, 2008, 326; 64, 2008, 332; Weed Sci. 57, 2009, 108; Austral. J. Agricult. Res. 58, 2007, 708; Science 316, 2007, 1185; and references quoted therein. Several cultivated plants have been rendered tolerant to herbicides by conventional methods of breeding (mutagenesis), e. g. Clearfield® summer rape (Canola, BASF SE, Germany) being tolerant to imidazolinones, e. g. imazamox, or ExpressSun® sunflowers (DuPont, USA) being tolerant to sulfonyl ureas, e. g. tribenuron. Genetic engineering methods have been used to render cultivated plants such as soybean, cotton, corn, beets and rape, tolerant to herbicides such as glyphosate and glufosinate, some of which are commercially available under the trade names RoundupReady® (glyphosate-tolerant, Monsanto, U.S.A.), Cultivance® (imidazolinone tolerant, BASF SE, Germany) and LibertyLink® (glufosinate-tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as δ-endotoxins, e. g. CryIA(b), CryIA(c), CryIF, CryIF(a2), CryIIA(b), CryIIIA, CryIIIB(b1) or Cry9c; vegetative insecticidal proteins (VIP), e. g. VIP1, VIP2, VIP3 or VIP3A; insecticidal proteins of bacteria colonizing nematodes, e. g. Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, e. g. WO 02/015701). Further examples of such toxins or genetically modified plants capable of synthesizing such toxins are disclosed, e. g., in EP-A 374 753, WO 93/007278, WO 95/34656, EP-A 427 529, EP-A 451 878, WO 03/18810 und WO 03/52073. The methods for producing such genetically modified plants are generally known to the person skilled in the art and are described, e. g. in the publications mentioned above. These insecticidal proteins contained in the genetically modified plants impart to the plants producing these proteins tolerance to harmful pests from all taxonomic groups of athropods, especially to beetles (Coeloptera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme Phosphinothricin-N-Acetyltransferase [PAT]); NuCOTN® 33B (cotton cultivars producing the Cry1Ac toxin), Bollgard® I (cotton cultivars producing the Cry1Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt11 (e. g. Agrisure® CB) and Bt176 from Syngenta Seeds SAS, France, (corn cultivars producing the Cry1Ab toxin and PAT enzyme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e. g. EP-A 392 225), plant disease resistance genes (e. g. potato cultivars, which express resistance genes acting against Phytophthora infestans derived from the mexican wild potato Solanum bulbocastanum) or T4-lysozym (e. g. potato cultivars capable of synthesizing these proteins with increased resistance against bacteria such as Erwinia amylvora). The methods for producing such genetically modified plants are generally known to the person skilled in the art and are described, e. g. in the publications mentioned above.

Furthermore, plants are also covered that are by the use of recombinant DNA techniques capable to synthesize one or more proteins to increase the productivity (e. g. bio mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of substances of content or new substances of content, specifically to improve human or animal nutrition, e. g. oil crops that produce health-promoting long-chain omega-3 fatty acids or unsaturated omega-9 fatty acids (e. g. Nexera® rape, DOW Agro Sciences, Canada).

Furthermore, plants are also covered that contain by the use of recombinant DNA techniques a modified amount of substances of content or new substances of content, specifically to improve raw material production, e. g. potatoes that produce increased amounts of amylopectin (e. g. Amflora® potato, BASF SE, Germany).

The organic moieties mentioned in the above definitions of the variables are—like the term halogen—collective terms for individual listings of the individual group members. The prefix Cₙ-Cₘ indicates in each case the possible number of carbon atoms in the group.

The term halogen denotes in each case fluorine, bromine, chlorine or iodine, in particular fluorine, chlorine or bromine.

The term “alkyl” as used herein and in the alkyl moieties of alkoxy, alkylthio, alkylsulfinyl, alkylsulfonyl, alkylcarbonyl, alkoxycarbonyl and the like refers to saturated straight-chain or branched hydrocarbon radicals having 1 to 2 (“C₁-C₂-alkyl”), 1 to 3 (“C₁-C₃-alkyl”), 1 to 4 (“C₁-C₄-alkyl”), 1 to 6 (“C₁-C₆-alkyl”), 1 to 8 (“C₁-C₈-alkyl”) or 1 to 10 (“C₁-C₁₀-alkyl”) carbon atoms. C₁-C₂-Alkyl is methyl or ethyl. C₁-C₃-Alkyl is additionally propyl and isopropyl. C₁-C₄-Alkyl is additionally butyl, 1-methylpropyl (sec-butyl), 2-methylpropyl (isobutyl) or 1,1-dimethylethyl (tert-butyl). C₁-C₆-Alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl. C₁-C₈-Alkyl is additionally also, for example, heptyl, octyl, 2-ethylhexyl and positional isomers thereof. C₁-C₁₀-Alkyl is additionally also, for example, nonyl, decyl and positional isomers thereof.

The term “haloalkyl” as used herein, which is also expressed as “alkyl which is partially or fully halogenated”, refers to straight-chain or branched alkyl groups having 1 to 2 (“C₁-C₂-haloalkyl”), 1 to 3 (“C₁-C₃-haloalkyl”), 1 to 4 (“C₁-C₄-haloalkyl”), 1 to 6 (“C₁-C₆-haloalkyl”), 1 to 8 (“C₁-C₈-haloalkyl”) or 1 to 10 (“C₁-C₁₀-haloalkyl”) carbon atoms (as mentioned above), where some or all of the hydrogen atoms in these groups are replaced by halogen atoms as mentioned above. C₁-C₂-Haloalkyl is, for example, chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl or pentafluoroethyl. C₁-C₃-Haloalkyl is additionally, for example, 1-fluoropropyl, 2-fluoropropyl, 3-fluoropropyl, 1,1-difluoropropyl, 2,2-difluoropropyl, 1,2-difluoropropyl, 3,3-difluoropropyl, 3,3,3-trifluoropropyl, heptafluoropropyl, 1,1,1-trifluoroprop-2-yl, 3-chloropropyl and the like. Examples for C₁-C₄-haloalkyl are, apart those mentioned for C₁-C₃-haloalkyl, 4-chlorobutyl and the like. Examples for C₁-C₆-haloalkyl are, apart those mentioned for C₁-C₄-haloalkyl, 5-chloropentyl, 6-chlorohexyl and the like. Examples for C₁-C₁₀-haloalkyl are, apart those mentioned for C₁-C₆-haloalkyl, 7-chloroheptyl and the like.

“Halomethyl” is methyl in which 1, 2 or 3 of the hydrogen atoms are replaced by halogen atoms. Examples are bromomethyl, chloromethyl, fluoromethyl, dichloromethyl, trichloromethyl, difluoromethyl, trifluoromethyl, chlorofluoromethyl, dichlorofluoromethyl, chlorodifluoromethyl and the like.

The term “alkenyl” as used herein refers to monounsaturated straight-chain or branched hydrocarbon radicals having 2 to 3 (“C₂-C₃-alkenyl”), 2 to 4 (“C₂-C₄-alkenyl”), 2 to 6 (“C₂-C₆-alkenyl”), 2 to 8 (“C₂-C₈-alkenyl”) or 2 to 10 (“C₂-C₁₀-alkenyl”) carbon atoms and a double bond in any position, for example C₂-C₃-alkenyl, such as ethenyl, 1-propenyl, 2-propenyl or 1-methylethenyl; C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl; C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like, or C₂-C₁₀-alkenyl, such as the radicals mentioned for C₂-C₆-alkenyl and additionally 1-heptenyl, 2-heptenyl, 3-heptenyl, 1-octenyl, 2-octenyl, 3-octenyl, 4-octenyl, 1-nonenyl, 2-nonenyl, 3-nonenyl, 4-nonenyl, 1-decenyl, 2-decenyl, 3-decenyl, 4-decenyl, 5-decenyl and the positional isomers thereof.

The term “haloalkenyl” as used herein, which is also expressed as “alkenyl which is partially or fully halogenated”, refers to unsaturated straight-chain or branched hydrocarbon radicals having 2 to 4 (“C₂-C₄-haloalkenyl”), 2 to 6 (“C₂-C₆-haloalkenyl”), 2 to 8 (“C₂-C₆-haloalkenyl”) or 2 to 10 (“C₂-C₁₀-haloalkenyl”) carbon atoms and a double bond in any position (as mentioned above), where some or all of the hydrogen atoms in these groups are replaced by halogen atoms as mentioned above, in particular fluorine, chlorine and bromine, for example chlorovinyl, chloroallyl and the like.

The term “alkynyl” as used herein refers to straight-chain or branched hydrocarbon groups having 2 to 3 (“C₂-C₃-alkynyl”), 2 to 4 (“C₂-C₄-alkynyl”), 2 to 6 (“C₂-C₆-alkynyl”), 2 to 8 (“C₂-C₈-alkynyl”), or 2 to 10 (“C₂-C₁₀-alkynyl”) carbon atoms and one or two triple bonds in any position, for example C₂-C₃-alkynyl, such as ethynyl, 1-propynyl or 2-propynyl; C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;

The term “haloalkynyl” as used herein, which is also expressed as “alkynyl which is partially or fully halogenated”, refers to unsaturated straight-chain or branched hydrocarbon radicals having 2 to 4 (“C₂-C₄-haloalkynyl”), 3 to 4 (“C₃-C₄-haloalkynyl”), 2 to 6 (“C₂-C₆-haloalkynyl”), 2 to 8 (“C₂-C₈-haloalkynyl”) or 2 to 10 (“C₂-C₁₀-haloalkynyl”) carbon atoms and one or two triple bonds in any position (as mentioned above), where some or all of the hydrogen atoms in these groups are replaced by halogen atoms as mentioned above, in particular fluorine, chlorine and bromine;

The term “cycloalkyl” as used herein refers to mono- or bi- or polycyclic saturated hydrocarbon radicals having 3 to 8 (“C₃-C₈-cycloalkyl”), in particular 3 to 6 carbon atoms (“C₃-C₆-cycloalkyl”) or 3 or 4 carbon atoms (“C₃-C₄-cycloalkyl”). Preferably, C₃-C₄-cycloalkyl and C₃-C₄-cycloalkyl are monocyclic. Examples for C₃-C₄-cycloalkyl are cyclopropyl and cyclobutyl. Examples of monocyclic radicals having 3 to 6 carbon atoms comprise cyclopropyl, cyclobutyl, cyclopentyl and cyclohexyl. Examples of monocyclic radicals having 3 to 8 carbon atoms comprise cyclopropyl, cyclobutyl, cyclopentyl, cyclohexyl, cycloheptyl and cyclooctyl. Examples of bicyclic radicals having 7 or 8 carbon atoms comprise bicyclo[2.2.1]heptyl, bicyclo[3.1.1]heptyl, bicyclo[2.2.2]octyl and bicyclo[3.2.1]octyl. Preferably, the term cycloalkyl denotes a monocyclic saturated hydrocarbon radical.

The term “halocycloalkyl” as used herein, which is also expressed as “cycloalkyl which is partially or fully halogenated”, refers to mono- or bi- or polycyclic saturated hydrocarbon groups having 3 to 8 (“C₃-C₈-halocycloalkyl”) or preferably 3 to 6 (“C₃-C₆-halocycloalkyl”) carbon ring members (as mentioned above) in which some or all of the hydrogen atoms are replaced by halogen atoms as mentioned above, in particular fluorine, chlorine and bromine.

The term “cycloalkyl-C₁-C₄-alkyl” refers to a C₃-C₈-cycloalkyl group (“C₃-C₈-cycloalkyl-C₁-C₄-alkyl”), preferably a C₃-C₆-cycloalkyl group (“C₃-C₆-cycloalkyl-C₁-C₄-alkyl”), more preferably a C₃-C₄-cycloalkyl group (“C₃-C₄-cycloalkyl-C₁-C₄-alkyl”) as defined above (preferably a monocyclic cycloalkyl group) which is bound to the remainder of the molecule via a C₁-C₄-alkyl group, as defined above. Examples for C₃-C₄-cycloalkyl-C₁-C₄-alkyl are cyclopropylmethyl, cyclopropylethyl, cyclopropylpropyl, cyclobutylmethyl, cyclobutylethyl and cyclobutylpropyl, Examples for C₃-C₆-cycloalkyl-C₁-C₄-alkyl, apart those mentioned for C₃-C₄-cycloalkyl-C₁-C₄-alkyl, are cyclopentylmethyl, cyclopentylethyl, cyclopentylpropyl, cyclohexylmethyl, cyclohexylethyl and cyclohexylpropyl. Examples for C₃-C₈-cycloalkyl-C₁-C₄-alkyl, apart those mentioned for C₃-C₆-cycloalkyl-C₁-C₄-alkyl, are cycloheptylmethyl, cycloheptylethyl, cyclooctylmethyl and the like. The term “C₃-C₈-cycloalkyl-C₁-C₆-alkyl” refers to a C₃-C₈-cycloalkyl group as defined above (preferably a monocyclic cycloalkyl group) which is bound to the remainder of the molecule via a C₁-C₆-alkyl group, as defined above. Examples for C₃-C₈-cycloalkyl-C₁-C₆-alkyl, apart those mentioned for C₃-C₈-cycloalkyl-C₁-C₄-alkyl, are cyclopropylpentyl, cyclopropylhexyl, cyclobutylpentyl, cyclobutylhexyl, cyclopentylpenty, cyclopentylhexyl and the like.

The term “C₃-C₈-halocycloalkyl-C₁-C₄-alkyl” refers to a C₃-C₈-halocycloalkyl group as defined above which is bound to the remainder of the molecule via a C₁-C₄-alkyl group, as defined above.

The term “C₁-C₂-alkoxy” is a C₁-C₂-alkyl group, as defined above, attached via an oxygen atom. The term “C₁-C₃-alkoxy” is a C₁-C₃-alkyl group, as defined above, attached via an oxygen atom. The term “C₁-C₄-alkoxy” is a C₁-C₄-alkyl group, as defined above, attached via an oxygen atom. The term “C₁-C₆-alkoxy” is a C₁-C₆-alkyl group, as defined above, attached via an oxygen atom. The term “C₁-C₁₀-alkoxy” is a C₁-C₁₀-alkyl group, as defined above, attached via an oxygen atom. C₁-C₂-Alkoxy is methoxy or ethoxy. C₁-C₃-Alkoxy is additionally, for example, n-propoxy and 1-methylethoxy (isopropoxy). C₁-C₄-Alkoxy is additionally, for example, butoxy, 1-methylpropoxy (secbutoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof.

The term “C₁-C₂-haloalkoxy” is a C₁-C₂-haloalkyl group, as defined above, attached via an oxygen atom. The term “C₁-C₃-haloalkoxy” is a C₁-C₃-haloalkyl group, as defined above, attached via an oxygen atom. The term “C₁-C₄-haloalkoxy” is a C₁-C₄-haloalkyl group, as defined above, attached via an oxygen atom. The term “C₁-C₆-haloalkoxy” is a C₁-C₆-haloalkyl group, as defined above, attached via an oxygen atom. The term “C₁-C₁₀-haloalkoxy” is a C₁-C₁₀-haloalkyl group, as defined above, attached via an oxygen atom. C₁-C₂-Halomethoxy is, for example, OCH₂F, OCHF₂, OCF₃, OCH₂Cl, OCHCl₂, OCCl₃, chlorofluoromethoxy, dichlorofluoromethoxy or chlorodifluoromethoxy. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₃-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy or 1-(CH₂Br)-2-bromoethoxy. C₁-C₄-Haloalkoxy is additionally, for example, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy or dodecafluorohexoxy.

The term “C₁-C₄-alkoxy-C₁-C₄-alkyl” as used herein, refers to a straight-chain or branched alkyl group having 1 to 4 carbon atoms, as defined above, where one hydrogen atom is replaced by a C₁-C₄-alkoxy group, as defined above. The term “C₁-C₆-alkoxy-C₁-C₄-alkyl” as used herein, refers to a straight-chain or branched alkyl group having 1 to 4 carbon atoms, as defined above, where one hydrogen atom is replaced by a C₁-C₆-alkoxy group, as defined above. The term “C₁-C₆-alkoxy-C₁-C₆-alkyl” as used herein, refers to a straight-chain or branched alkyl group having 1 to 6 carbon atoms, as defined above, where one hydrogen atom is replaced by a C₁-C₆-alkoxy group, as defined above. Examples are methoxymethyl, ethoxymethyl, propoxymethyl, isopropoxymethyl, n-butoxymethyl, sec-butoxymethyl, isobutoxymethyl, tert-butoxymethyl, 1-methoxyethyl, 1-ethoxyethyl, 1-propoxyethyl, 1-isopropoxyethyl, 1-n-butoxyethyl, 1-sec-butoxyethyl, 1-isobutoxyethyl, 1-tert-butoxyethyl, 2-methoxyethyl, 2-ethoxyethyl, 2-propoxyethyl, 2-isopropoxyethyl, 2-n-butoxyethyl, 2-sec-butoxyethyl, 2-isobutoxyethyl, 2-tert-butoxyethyl, 1-methoxypropyl, 1-ethoxypropyl, 1-propoxypropyl, 1-isopropoxypropyl, 1-n-butoxypropyl, 1-sec-butoxypropyl, 1-isobutoxypropyl, 1-tert-butoxypropyl, 2-methoxypropyl, 2-ethoxypropyl, 2-propoxypropyl, 2-isopropoxypropyl, 2-n-butoxypropyl, 2-sec-butoxypropyl, 2-isobutoxypropyl, 2-tert-butoxypropyl, 3-methoxypropyl, 3-ethoxypropyl, 3-propoxypropyl, 3-isopropoxypropyl, 3-n-butoxypropyl, 3-sec-butoxypropyl, 3-isobutoxypropyl, 3-tert-butoxypropyl and the like.

The term “C₁-C₆-alkoxy-methyl” as used herein, refers to methyl in which one hydrogen atom is replaced by a C₁-C₆-alkoxy group, as defined above. Examples are methoxymethyl, ethoxymethyl, propoxymethyl, isopropoxymethyl, n-butoxymethyl, sec-butoxymethyl, isobutoxymethyl, tert-butoxymethyl, pentyloxymethyl, hexyloxymethyl and the like.

C₁-C₆-Haloalkoxy-C₁-C₆-alkyl is a straight-chain or branched alkyl group having from 1 to 6, especially 1 to 4 carbon atoms (═C₁-C₆-haloalkoxy-C₁-C₄-alkyl), wherein one of the hydrogen atoms is replaced by a C₁-C₆-alkoxy group and wherein at least one, e.g. 1, 2, 3, 4 or all of the remaining hydrogen atoms (either in the alkoxy moiety or in the alkyl moiety or in both) are replaced by halogen atoms. C₁-C₄-Haloalkoxy-C₁-C₄-alkyl is a straight-chain or branched alkyl group having from 1 to 4 carbon atoms, wherein one of the hydrogen atoms is replaced by a C₁-C₄-alkoxy group and wherein at least one, e.g. 1, 2, 3, 4 or all of the remaining hydrogen atoms (either in the alkoxy moiety or in the alkyl moiety or in both) are replaced by halogen atoms. Examples are difluoromethoxymethyl (CHF₂OCH₂), trifluoromethoxymethyl, 1-difluoromethoxyethyl, 1-trifluoromethoxyethyl, 2-difluoromethoxyethyl, 2-trifluoromethoxyethyl, difluoromethoxy-methyl (CH₃OCF₂), 1,1-difluoro-2-methoxyethyl, 2,2-difluoro-2-methoxyethyl and the like.

The term “C₁-C₂-alkylthio” is a C₁-C₂-alkyl group, as defined above, attached via a sulfur atom. The term “C₁-C₃-alkylthio” is a C₁-C₃-alkyl group, as defined above, attached via a sulfur atom. The term “C₁-C₄-alkylthio” is a C₁-C₄-alkyl group, as defined above, attached via a sulfur atom. The term “C₁-C₆-alkylthio” is a C₁-C₆-alkyl group, as defined above, attached via a sulfur atom. The term “C₁-C₁₀-alkylthio” is a C₁-C₁₀-alkyl group, as defined above, attached via a sulfur atom. C₁-C₂-Alkylthio is methylthio or ethylthio. C₁-C₃-Alkylthio is additionally, for example, n-propylthio or 1-methylethylthio (isopropylthio). C₁-C₄-Alkylthio is additionally, for example, butylthio, 1-methylpropylthio (sec-butylthio), 2-methylpropylthio (isobutylthio) or 1,1-dimethylethylthio (tert-butylthio). C₁-C₆-Alkylthio is additionally, for example, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio. C₁-C₈-Alkylthio is additionally, for example, heptylthio, octylthio, 2-ethylhexylthio and positional isomers thereof. C₁-C₁₀-Alkylthio is additionally, for example, nonylthio, decylthio and positional isomers thereof.

The term “C₁-C₂-haloalkylthio” is a C₁-C₂-haloalkyl group, as defined above, attached via a sulfur atom. The term “C₁-C₃-haloalkylthio” is a C₁-C₃-haloalkyl group, as defined above, attached via a sulfur atom. The term “C₁-C₄-haloalkylthio” is a C₁-C₄-haloalkyl group, as defined above, attached via a sulfur atom. The term “C₁-C₆-haloalkylthio” is a C₁-C₆-haloalkyl group, as defined above, attached via a sulfur atom. The term “C₁-C₁₀-haloalkylthio” is a C₁-C₁₀-haloalkyl group, as defined above, attached via a sulfur atom. C₁-C₂-Haloalkylthio is, for example, SCH₂F, SCHF₂, SCF₃, SCH₂Cl, SCHC₂, SCCl₃, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio or SC₂F₅. C₁-C₃-Haloalkylthio is additionally, for example, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, SCH₂—C₂F₅, SCF₂—C₂F₅, 1-(CH₂F)-2-fluoroethylthio, 1-(CH₂Cl)-2-chloroethylthio or 1-(CH₂Br)-2-bromoethylthio. C₁-C₄-Haloalkylthio is additionally, for example, 4-fluorobutylthio, 4-chlorobutylthio, 4-bromobutylthio or nonafluorobutylthio. C₁-C₆-Haloalkylthio is additionally, for example, 5-fluoropentylthio, 5-chloropentylthio, 5-brompentylthio, 5-iodopentylthio, undecafluoropentylthio, 6-fluorohexylthio, 6-chlorohexylthio, 6-bromohexylthio, 6-iodohexylthio or dodecafluorohexylthio.

The term “C₁-C₂-alkylsulfinyl” is a C₁-C₂-alkyl group, as defined above, attached via a sulfinyl [S(O)] group. The term “C₁-C₄-alkylsulfinyl” is a C₁-C₄-alkyl group, as defined above, attached via a sulfinyl [S(O)] group. The term “C₁-C₆-alkylsulfinyl” is a C₁-C₆-alkyl group, as defined above, attached via a sulfinyl [S(O)] group. The term “C₁-C₁₀-alkylsulfinyl” is a C₁-C₁₀-alkyl group, as defined above, attached via a sulfinyl [S(O)] group. C₁-C₂-Alkylsulfinyl is methylsulfinyl or ethylsulfinyl. C₁-C₄-Alkylsulfinyl is additionally, for example, n-propylsulfinyl, 1-methylethylsulfinyl (isopropylsulfinyl), butylsulfinyl, 1-methylpropylsulfinyl (sec-butylsulfinyl), 2-methylpropylsulfinyl (isobutylsulfinyl) or 1,1-dimethylethylsulfinyl (tert-butylsulfinyl). C₁-C₆-Alkylsulfinyl is additionally, for example, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl. C₁-C₈-Alkylsulfinyl is additionally, for example, heptylsulfinyl, octylsulfinyl, 2-ethylhexylsulfinyl and positional isomers thereof. C₁-C₁₀-Alkylsulfinyl is additionally, for example, nonylsulfinyl, decylsulfinyl and positional isomers thereof.

The term “C₁-C₂-haloalkylsulfinyl” is a C₁-C₂-haloalkyl group, as defined above, attached via a sulfinyl [S(O)] group. The term “C₁-C₄-haloalkylsulfinyl” is a C₁-C₄-haloalkyl group, as defined above, attached via a sulfinyl [S(O)] group. The term “C₁-C₆-haloalkylsulfinyl” is a C₁-C₆-haloalkyl group, as defined above, attached via a sulfinyl [S(O)] group. The term “C₁-C₁₀-haloalkylsulfinyl” is a C₁-C₁₀-haloalkyl group, as defined above, attached via a sulfinyl [S(O)] group. C₁-C₂-Haloalkylsulfinyl is, for example, S(O)CH₂F, S(O)CHF₂, S(O)CF₃, S(O)CH₂Cl, S(O)CHCl₂, S(O)CCl₃, chlorofluoromethylsulfinyl, dichlorofluoromethylsulfinyl, chlorodifluoromethylsulfinyl, 2-fluoroethylsulfinyl, 2-chloroethylsulfinyl, 2-bromoethylsulfinyl, 2-iodoethylsulfinyl, 2,2-difluoroethylsulfinyl, 2,2,2-trifluoroethylsulfinyl, 2-chloro-2-fluoroethylsulfinyl, 2-chloro-2,2-difluoroethylsulfinyl, 2,2-dichloro-2-fluoroethylsulfinyl, 2,2,2-trichloroethylsulfinyl or S(O)C₂F₅. C₁-C₄-Haloalkylsulfinyl is additionally, for example, 2-fluoropropylsulfinyl, 3-fluoropropylsulfinyl, 2,2-difluoropropylsulfinyl, 2,3-difluoropropylsulfinyl, 2-chloropropylsulfinyl, 3-chloropropylsulfinyl, 2,3-dichloropropylsulfinyl, 2-bromopropylsulfinyl, 3-bromopropylsulfinyl, 3,3,3-trifluoropropylsulfinyl, 3,3,3-trichloropropylsulfinyl, S(O)CH₂—C₂F₅, S(O)CF₂—C₂F₅, 1-(CH₂F)-2-fluoroethylsulfinyl, 1-(CH₂Cl)-2-chloroethylsulfinyl, 1-(CH₂Br)-2-bromoethylsulfinyl, 4-fluorobutylsulfinyl, 4-chlorobutylsulfinyl, 4-bromobutylsulfinyl or nonafluorobutylsulfinyl. C₁-C₆-Haloalkylsulfinyl is additionally, for example, 5-fluoropentylsulfinyl, 5-chloropentylsulfinyl, 5-brompentylsulfinyl, 5-iodopentylsulfinyl, undecafluoropentylsulfinyl, 6-fluorohexylsulfinyl, 6-chlorohexylsulfinyl, 6-bromohexylsulfinyl, 6-iodohexylsulfinyl or dodecafluorohexylsulfinyl.

The term “C₁-C₂-alkylsulfonyl” is a C₁-C₂-alkyl group, as defined above, attached via a sulfonyl [S(O)₂] group. The term “C₁-C₃-alkylsulfonyl” is a C₁-C₃-alkyl group, as defined above, attached via a sulfonyl [S(O)₂] group. The term “C₁-C₄-alkylsulfonyl” is a C₁-C₄-alkyl group, as defined above, attached via a sulfonyl [S(O)₂] group. The term “C₁-C₆-alkylsulfonyl” is a C₁-C₆-alkyl group, as defined above, attached via a sulfonyl [S(O)₂] group. The term “C₁-C₁₀-alkylsulfonyl” is a C₁-C₁₀-alkyl group, as defined above, attached via a sulfonyl [S(O)₂] group. C₁-C₂-Alkylsulfonyl is methylsulfonyl or ethylsulfonyl. C₁-C₃-Alkylsulfonyl is additionally, for example, n-propylsulfonyl or 1-methylethylsulfonyl (isopropylsulfonyl). C₁-C₄-Alkylsulfonyl is additionally, for example, butylsulfonyl, 1-methylpropylsulfonyl (sec-butylsulfonyl), 2-methylpropylsulfonyl (isobutylsulfonyl) or 1,1-dimethylethylsulfonyl (tert-butylsulfonyl). C₁-C₆-Alkylsulfonyl is additionally, for example, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl. C₁-C₈-Alkylsulfonyl is additionally, for example, heptylsulfonyl, octylsulfonyl, 2-ethylhexylsulfonyl and positional isomers thereof. C₁-C₁₀-Alkylsulfonyl is additionally, for example, nonylsulfonyl, decylsulfonyl and positional isomers thereof.

The term “C₁-C₂-haloalkylsulfonyl” is a C₁-C₂-haloalkyl group, as defined above, attached via a sulfonyl [S(O)₂] group. The term “C₁-C₃-haloalkylsulfonyl” is a C₁-C₃-haloalkyl group, as defined above, attached via a sulfonyl [S(O)₂] group. The term “C₁-C₄-haloalkylsulfonyl” is a C₁-C₄-haloalkyl group, as defined above, attached via a sulfonyl [S(O)₂] group. The term “C₁-C₆-haloalkylsulfonyl” is a C₁-C₆-haloalkyl group, as defined above, attached via a sulfonyl [S(O)₂] group. The term “C₁-C₁₀-haloalkylsulfonyl” is a C₁-C₁₀-haloalkyl group, as defined above, attached via a sulfonyl [S(O)₂] group. C₁-C₂-Haloalkylsulfonyl is, for example, S(O)₂CH₂F, S(O)₂CHF₂, S(O)₂CF₃, S(O)₂CH₂Cl, S(O)₂CHCl₂, S(O)₂CCl₃, chlorofluoromethylsulfonyl, dichlorofluoromethylsulfonyl, chlorodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl or S(O)₂C₂F₅. C₁-C₃-Haloalkylsulfonyl is additionally, for example, 2-fluoropropylsulfonyl, 3-fluoropropylsulfonyl, 2,2-difluoropropylsulfonyl, 2,3-difluoropropylsulfonyl, 2-chloropropylsulfonyl, 3-chloropropylsulfonyl, 2,3-dichloropropylsulfonyl, 2-bromopropylsulfonyl, 3-bromopropylsulfonyl, 3,3,3-trifluoropropylsulfonyl, 3,3,3-trichloropropylsulfonyl, S(O)₂CH₂—C₂F₅, S(O)₂CF₂—C₂F₅, 1-(CH₂F)-2-fluoroethylsulfonyl, 1-(CH₂Cl)-2-chloroethylsulfonyl or 1-(CH₂Br)-2-bromoethylsulfonyl. C₁-C₄-Haloalkylsulfonyl is additionally, for example, 4-fluorobutylsulfonyl, 4-chlorobutylsulfonyl, 4-bromobutylsulfonyl or nonafluorobutylsulfonyl. C₁-C₆-Haloalkylsulfonyl is additionally, for example, 5-fluoropentylsulfonyl, 5-chloropentylsulfonyl, 5-brompentylsulfonyl, 5-iodopentylsulfonyl, undecafluoropentylsulfonyl, 6-fluorohexylsulfonyl, 6-chlorohexylsulfonyl, 6-bromohexylsulfonyl, 6-iodohexylsulfonyl or dodecafluorohexylsulfonyl.

The substituent “oxo” replaces a CH₂ group by a C(═O) group.

The term “alkylcarbonyl” is a C₁-C₆-alkyl (“C₁-C₆-alkylcarbonyl”), preferably a C₁-C₄-alkyl (“C₁-C₄-alkylcarbonyl”) group, as defined above, attached via a carbonyl [C(═O)] group. Examples are acetyl (methylcarbonyl), propionyl (ethylcarbonyl), propylcarbonyl, isopropylcarbonyl, n-butylcarbonyl and the like.

The term “haloalkylcarbonyl” is a C₁-C₆-haloalkyl (“C₁-C₆-haloalkylcarbonyl”), preferably a C₁-C₄-haloalkyl (“C₁-C₄-haloalkylcarbonyl”) group, as defined above, attached via a carbonyl [C(═O)] group. Examples are trifluoromethylcarbonyl, 2,2,2-trifluoroethylcarbonyl and the like.

The term “alkoxycarbonyl” is a C₁-C₆-alkoxy (“C₁-C₆-alkoxycarbonyl”), preferably a C₁-C₄-alkoxy (“C₁-C₄-alkoxycarbonyl”) group, as defined above, attached via a carbonyl [C(═O)] group. Examples are methoxycarbonyl), ethoxycarbonyl, propoxycarbonyl, isopropoxycarbonyl, n-butoxycarbonyl and the like.

The term “haloalkoxycarbonyl” is a C₁-C₆-haloalkoxy (“C₁-C₆-haloalkoxycarbonyl”), preferably a C₁-C₄-haloalkoxy (“C₁-C₄-haloalkoxycarbonyl”) group, as defined above, attached via a carbonyl [C(═O)] group. Examples are trifluoromethoxycarbonyl, 2,2,2-trifluoroethoxycarbonyl and the like.

The term “C₁-C₆-alkylamino” is a group —N(H)C₁-C₆-alkyl. Examples are methylamino, ethylamino, propylamino, isopropylamino, butylamino and the like.

The term “di-(C₁-C₆-alkyl)amino” is a group —N(C₁-C₆-alkyl)₂. Examples are dimethylamino, diethylamino, ethylmethylamino, dipropylamino, diisopropylamino, methylpropylamino, methylisopropylamino, ethylpropylamino, ethylisopropylamino, dibutylamino and the like.

The term “3-, 4-, 5- or 6-membered saturated, partly unsaturated or maximum unsaturated heterocyclic ring containing 1, 2 or 3 (or 4) heteroatoms or heteroatom groups selected from N, O, S, NO, SO and SO₂, as ring members” [wherein “maximum unsaturated” includes also “aromatic”] as used herein denotes monocyclic radicals, the monocyclic radicals being saturated, partially unsaturated or maximum unsaturated (including ing aromatic). The term “3-, 4-, 5-, 6- or 7-membered saturated, partly unsaturated or maximum unsaturated heterocyclic ring containing 1, 2 or 3 (or 4) heteroatoms or heteroatom groups selected from N, O, S, NO, SO and SO₂, as ring members” [wherein “maximum unsaturated” includes also “aromatic”] as used herein further also encompasses 7-membered heteromonocyclic radicals containing 1, 2 or 3 (or 4) heteroatoms or heteroatom groups selected from N, O, S, NO, SO and SO₂, as ring members, the monocyclic radicals being saturated, partially unsaturated or maximum unsaturated (including aromatic. The term “3-, 4-, 5-, 6-, 7- or 8-membered saturated, partially unsaturated or maximum unsaturated heterocyclic ring containing 1, 2 or 3 (or 4) heteroatoms or heteroatom groups selected from N, O, S, NO, SO and SO₂, as ring members” [wherein “maximum unsaturated” includes also “aromatic”] as used herein further also encompasses 8-membered heteromonocyclic radicals containing 1, 2 or 3 (or 4) heteroatoms or heteroatom groups selected from N, O, S, NO, SO and SO₂, as ring members, the monocyclic radicals being saturated, partially unsaturated or maximum unsaturated (including aromatic). Unsaturated rings contain at least one C—C and/or C—N and/or N—N double bond(s). Partially unsaturated rings contain less than the maximum number of C—C and/or C—N and/or N—N double bond(s) allowed by the ring size. Maximum unsaturated rings contain as many conjugated C—C and/or C—N and/or N—N double bonds as allowed by the ring size. Maximum unsaturated 5- or 6-membered heterocyclic rings are aromatic. 7- and 8-membered rings cannot be aromatic. They are homoaromatic (7-membered ring, 3 double bonds) or have 4 double bonds (8-membered ring). The heterocyclic ring may be attached to the remainder of the molecule via a carbon ring member or via a nitrogen ring member. As a matter of course, the heterocyclic ring contains at least one carbon ring atom. If the ring contains more than one O ring atom, these are not adjacent.

Examples of a 3-, 4-, 5-, 6- or 7-membered saturated heterocyclic ring include: Oxiranyl, thiiranyl, aziridinyl, oxetanyl, thietanyl, azetidinyl, tetrahydrofuran-2-yl, tetrahydrofuran-3-yl, tetrahydrothien-2-yl, tetrahydrothien-3-yl, pyrrolidin-1-yl, pyrrolidin-2-yl, pyrrolidin-3-yl, pyrazolidin-1-yl, pyrazolidin-3-yl, pyrazolidin-4-yl, pyrazolidin-5-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolidin-5-yl, isothiazolidin-2-yl, isothiazolidin-3-yl, isothiazolidin-4-yl, isothiazolidin-5-yl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-1-yl, 1,3,4-triazolidin-2-yl, 2-tetrahydropyranyl, 4-tetrahydropyranyl, 1,3-dioxan-5-yl, 1,4-dioxan-2-yl, piperidin-1-yl, piperidin-2-yl, piperidin-3-yl, piperidin-4-yl, hexahydropyridazin-3-yl, hexahydropyridazin-4-yl, hexahydropyrimidin-2-yl, hexahydropyrimidin-4-yl, hexahydropyrimidin-5-yl, piperazin-1-yl, piperazin-2-yl, 1,3,5-hexahydrotriazin-1-yl, 1,3,5-hexahydrotriazin-2-yl and 1,2,4-hexahydrotriazin-3-yl, morpholin-2-yl, morpholin-3-yl, morpholin-4-yl, thiomorpholin-2-yl, thiomorpholin-3-yl, thiomorpholin-4-yl, 1-oxothiomorpholin-2-yl, 1-oxothiomorpholin-3-yl, 1-oxothiomorpholin-4-yl, 1,1-dioxothiomorpholin-2-yl, 1,1-dioxothiomorpholin-3-yl, 1,1-dioxothiomorpholin-4-yl, azepan-1-, -2-, -3- or -4-yl, oxepan-2-, -3-, -4- or -5-yl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl and the like.

Examples of a 3-, 4-, 5-, 6- or 7-membered partially unsaturated heterocyclic ring include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, 4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl.

Examples for a 3-, 4-, 5-, 6- or 7-membered maximally unsaturated (including aromatic) heterocyclic ring are 5- or 6-membered heteroaromatic rings, such as 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 1-imidazolyl, 2-imidazolyl, 4-imidazolyl, 1,3,4-triazol-1-yl, 1,3,4-triazol-2-yl, 2-pyridinyl, 3-pyridinyl, 4-pyridinyl, 1-oxopyridin-2-yl, 1-oxopyridin-3-yl, 1-oxopyridin-4-yl, 3-pyridazinyl, 4-pyridazinyl, 2-pyrimidinyl, 4-pyrimidinyl, 5-pyrimidinyl and 2-pyrazinyl, and also homoaromatic radicals, such as 1H-azepine, 1H-[1,3]-diazepine and 1H-[1,4]-diazepine.

A saturated 3-, 4-, 5-, 6-, 7- or 8-membered ring, wherein the ring may contain 1 or 2 heteroatoms or heteroatom groups selected from N, O, S, NO, SO and SO₂ and/or 1 or 2 groups selected from C═O, C═S and C═NR^{17a} as ring members is either carbocyclic or heterocyclic. Examples are, in addition to the saturated heteromonocyclic rings mentioned above, carbocyclic rings, such as cyclopropyl, cyclopropanonyl, cyclobutyl, cyclobutanonyl, cyclopentyl, cyclopentanonyl, cyclohexyl, cyclohexanonyl, cyclohexadienonyl, cycloheptyl, cycloheptanonyl, cyclooctyl, cyclooctanonyl, furan-2-onyl, pyrrolidine-2-onyl, pyrrolidine-2,5-dionyl, piperidine-2-only, piperidine-2,6-dionyl and the like.

The remarks made below concerning preferred embodiments of the variables of the compounds of formula I, especially with respect to their substituents A, X, Y, R¹, R², R³, R⁴, R^{4a}, R⁵, R⁶, R⁷, R⁸, R^{9a}, R^{9b}, R¹⁰, R¹¹, R¹², R¹³, R¹⁴, R¹⁵, R¹⁶, R^{17a}, R^{17b}, R¹⁸, R¹⁹, m, n, p and r, the features of the use and method according to the invention and of the composition of the invention are valid both on their own and, in particular, in every possible combination with each other.

Preferably, R¹ is selected from hydrogen and halogen, more preferably from hydrogen and fluorine and is specifically hydrogen.

In a preferred embodiment, R² is selected from hydrogen, cyano, C₁-C₆-alkyl, C₃-C₆-cycloalkyl and C₃-C₆-halocycloalkyl, where the two last-mentioned radicals may be substituted by one or more, e.g. 1, 2 or 3, preferably 1 or 2, in particular 1, radicals R³, where R³ has one of the above general or, in particular, one of the below preferred meanings. More preferably, R² is selected from hydrogen and C₁-C₆-alkyl which may be substituted by one or more radicals R³, where R³ has one of the above general or, in particular, one of the below preferred meanings; and in particular from hydrogen and C₁-C₆-hydroxyalkyl. In an alternative embodiment, R² is selected from hydrogen, halogen and C₁-C₄-alkyl. In particular, R² is selected from hydrogen and C₁-C₄-alkyl. Specifically, R² is hydrogen or methyl and very specifically hydrogen. In another alternative embodiment R² is selected from C₁-C₄-haloalkyl; and in particular from fluoromethyl, difluoromethyl and trifluoromethyl.

R³ as an optional substituent on a C₁-C₆-(halo)alkyl or C₃-C₆-(halo)cycloalkyl group R² is preferably selected from cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkyl, C₃-C₈-halocycloalkyl, amino, C₁-C₆-alkylamino, di(C₁-C₆-alkyl)amino and phenyl; and as a substituent on a C₃-C₆-cycloalkyl group is additionally preferably selected from C₁-C₆-alkyl and C₁-C₆-haloalkyl. In an alternatively preferred embodiment, R³ as an optional substituent on a C₁-C₆-(halo)alkyl or C₃-C₆-(halo)cycloalkyl group R² is OR¹⁶, where OR¹⁶ has one of the above general or, in particular, one of the below preferred meanings and is in particular H (so that R² is in particular hydroxyl). If the C₁-C₆-(halo)alkyl or C₃-C₆-(halo)cycloalkyl group R² carries one or more radicals R³, it carries preferably only one radical R³.

A is preferably selected from the radicals of formulae A-1 to A-10; i.e. from A-1, A-2, A3, A-4, A-5, A-6, A-7, A.8, A-9 and A-10. More preferably, A is selected from the radicals of formulae A-1 to A-4; i.e. from A-1, A-2, A-3 and A-4, and is specifically a radical of formula A-1.

X is preferably selected from O, NR^{4a}, SO₂ and a chemical bond; and more preferably from O, NR^{4a} and a chemical bond, where R^{4a} has one of the above general or, in particular, one of the below preferred meanings.

X is in particular selected from O and NR^{4a}, where R^{4a} has one of the above general or, in particular, one of the below preferred meanings.

R^{4a} is preferably selected from hydrogen, C₁-C₄-alkyl, C₁-C₄-haloalkyl, C₃-C₆-cycloalkyl, C₃-C₆-halocycloalkyl and phenyl which may carry 1, 2 or 3, in particular 1, substituents selected from halogen, C₁-C₄-alkyl, C₁-C₄-haloalkyl, C₁-C₄-alkoxy and C₁-C₄-haloalkoxy, more preferably from hydrogen, C₁-C₄-alkyl and phenyl; and in particular from hydrogen and C₁-C₄-alkyl. Specifically, R^{4a} is hydrogen or methyl.

Y is preferably selected from hydrogen, C₁-C₄-alkyl, C₁-C₄-haloalkyl, C₃-C₆-cycloalkyl and C₃-C₆-halocycloalkyl; more preferably from hydrogen, C₁-C₄-alkyl and C₁-C₄-haloalkyl; and in particular from hydrogen and C₁-C₄-alkyl. Specifically, Y is hydrogen.

R⁴ is preferably selected from hydrogen, C₁-C₁₀-alkyl, C₃-C₈-cycloalkyl, C₂-C₁₀-alkenyl, C₂-C₁₀-alkynyl, wherein the four last-mentioned radicals may be partially or fully halogenated and/or may be substituted with one or more radicals R⁷; C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, S(O)ₙR⁸, phenyl, optionally substituted with 1, 2, 3, 4 or 5 substituents R¹⁰; and a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more substituents R¹⁰;

where R⁷, R⁸, R^{9a}, R^{9b} and R¹⁰ have one of the above general or, in particular, one of the below preferred meanings.

R⁴ is more preferably selected from hydrogen, C₁-C₁₀-alkyl, C₃-C₈-cycloalkyl, C₂-C₁₀-alkenyl, C₂-C₁₀-alkynyl, wherein the four last-mentioned radicals may be partially or fully halogenated and/or may be substituted with one or more, e.g. 1, 2 or 3, in particular 1, radicals R⁷; C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, phenyl, optionally substituted with 1, 2 or 3, in particular 1, substituents R¹⁰; and a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more, e.g. 1, 2 or 3, preferably 1 or 2, in particular 1, substituents R¹⁰, where R⁷, R⁸, R^{9a}, R^{9b} and R¹⁰ have one of the above general or, in particular, one of the below preferred meanings.

Even more preferably, R⁴ is selected from hydrogen, C₁-C₆-alkyl, C₁-C₆-haloalkyl, C₁-C₆-alkyl substituted by one radical R⁷, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl, C(═O)R⁷, C(═O)NR^{9a}R^{9b}, C(═O)OR⁸, phenyl, optionally substituted with 1, 2 or 3, in particular 1, substituents selected from halogen, cyano, C₁-C₄-alkyl, C₁-C₄-haloalkyl, C₁-C₄-alkoxy and C₁-C₄-haloalkoxy; and a 5- or 6-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with 1 or 2, in particular 1, substituents selected from halogen, cyano, C₁-C₄-alkyl, C₁-C₄-haloalkyl, C₁-C₄-alkoxy and C₁-C₄-haloalkoxy, where R⁷, R⁸, R^{9a} and R^{9b} have one of the above general or, in particular, one of the below preferred meanings.

In particular, R⁴ is selected from hydrogen, C₁-C₆-alkyl, C₁-C₆-haloalkyl, C₁-C₆-alkyl substituted by one radical R⁷, C₃-C₆-cycloalkyl, C₃-C₆-halocycloalkyl, C₂-C₆-alkenyl, C₂-C₆-alkynyl, C(═O)R⁷, C(═O)NR^{9a}R^{9b}, C(═O)OR⁸ and phenyl, optionally substituted with 1, 2 or 3, in particular 1, substituents selected from halogen, where R⁷, R⁸, R^{9a} and R^{9b} have one of the above general or, in particular, one of the below preferred meanings.

In case that X is O, R⁴ is in particular selected from hydrogen, C₁-C₆-alkyl, C₁-C₆-haloalkyl, C₁-C₆-alkyl substituted by one radical R⁷, C₃-C₆-cycloalkyl, C₃-C₆-halocycloalkyl, C₂-C₆-alkenyl, C₂-C₆-alkynyl and phenyl, optionally substituted with 1, 2 or 3, in particular 1, substituents selected from halogen.

In case that X is NR^{4a}, R⁴ is in particular selected from C₁-C₆-alkyl, C₁-C₆-haloalkyl, C₁-C₆-alkyl substituted by one radical R⁷, C₃-C₆-cycloalkyl, C₃-C₆-halocycloalkyl, C₂-C₆-alkenyl, C₂-C₆-alkynyl, C(═O)R⁷, C(═O)NR^{9a}R^{9b}, C(═O)OR⁸ and phenyl, optionally substituted with 1, 2 or 3, in particular 1, substituents selected from halogen; and more particularly from C₁-C₆-alkyl, C₁-C₆-haloalkyl, C₁-C₆-alkyl substituted by one radical R⁷, C(═O)R⁷, C(═O)NR^{9a}R^{9b}, C(═O)OR⁸ and phenyl, optionally substituted with 1, 2 or 3, in particular 1, substituents selected from halogen, where R⁷, R⁸, R^{9a} and R^{9b} have one of the above general or, in particular, one of the below preferred meanings.

R⁷ as a substituent on an alkyl, cycloalkyl, alkenyl or alkynyl group R⁴ is preferably selected from cyano, C₃-C₆-cycloalkyl, C₃-C₆-halocycloalkyl, C₁-C₆-alkoxy, C₁-C₆-haloalkoxy, C₁-C₆-alkylthio, C₁-C₆-haloalkylthio, NR^{17a}R^{17b}, C(═O)NR^{17a}R^{17b}, C(═S)NR^{17a}R^{17b}, C(═O)OR¹⁶, phenyl, optionally substituted with 1, 2 or 3, in particular 1, substituents selected from halogen, cyano, C₁-C₄-alkyl, C₁-C₄-haloalkyl, C₁-C₄-alkoxy and C₁-C₄-haloalkoxy; and a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more, e.g. 1, 2 or 3, preferably 1 or 2, in particular 1, substituents selected from halogen, cyano, C₁-C₄-alkyl, C₁-C₄-haloalkyl, C₁-C₄-alkoxy and C₁-C₄-haloalkoxy, where R¹⁶, R^{17a} and R^{17b} have one of the above general or, in particular, one of the below preferred meanings.

More preferably, R⁷ as a substituent on an alkyl, cycloalkyl, alkenyl or alkynyl group R⁴ is selected from C₃-C₆-cycloalkyl, C₃-C₆-halocycloalkyl and phenyl, optionally substituted with 1, 2 or 3, in particular 1, substituents selected from halogen, C₁-C₄-alkyl, C₁-C₄-haloalkyl, C₁-C₄-alkoxy and C₁-C₄-haloalkoxy and in particular from halogen. Specifically, R⁷ as a substituent on an alkyl, cycloalkyl, alkenyl or alkynyl group R⁴ is selected from cyclopropyl and phenyl.

R⁷ as a substituent in a C(═O)R⁷ or C(═S)R⁷ group R⁴ is preferably selected from C₁-C₄-alkyl, C₁-C₄-haloalkyl, C₃-C₆-cycloalkyl, C₃-C₆-cycloalkyl-C₁-C₄-alkyl, C₃-C₆-halocycloalkyl, C₁-C₆-alkoxy, C₁-C₆-haloalkoxy, C₁-C₆-alkylthio, C₁-C₆-haloalkylthio, NR^{17a}R^{17b}, phenyl, benzyl, where the phenyl ring in the two last-mentioned radicals is optionally substituted with 1, 2 or 3, in particular 1, substituents selected from halogen, cyano, C₁-C₄-alkyl, C₁-C₄-haloalkyl, C₁-C₄-alkoxy and C₁-C₄-haloalkoxy; and a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more, e.g. 1, 2, or 3, preferably or 2, in particular 1, substituents selected from halogen, cyano, C₁-C₄-alkyl, C₁-C₄-haloalkyl, C₁-C₄-alkoxy and C₁-C₄-haloalkoxy.

More preferably, R⁷ as a substituent in a C(═O)R⁷ or C(═S)R⁷ group R⁴ is selected from C₁-C₄-alkyl, C₁-C₄-haloalkyl, C₃-C₆-cycloalkyl, C₃-C₆-cycloalkyl-C₁-C₄-alkyl, C₃-C₆-halocycloalkyl, C₁-C₆-alkoxy, C₁-C₆-haloalkoxy, NR^{17a}R^{17b}, phenyl and benzyl, where the phenyl ring in the two last-mentioned radicals is optionally substituted with 1, 2 or 3, in particular 1, substituents selected from halogen, C₁-C₄-alkyl, C₁-C₄-haloalkyl, C₁-C₄-alkoxy and C₁-C₄-haloalkoxy. Specifically, R⁷ as a substituent in a C(═O)R⁷ or C(═S)R⁷ group R⁴ is selected from C₁-C₄-alkyl, C₃-C₆-cycloalkyl, C₁-C₆-alkoxy, NR^{17a}R^{17b}, phenyl and benzyl.

R⁸ as a substituent in a C(═O)OR⁸ group R⁴ is preferably selected from hydrogen, C₁-C₄-alkyl, C₁-C₄-haloalkyl, C₃-C₆-cycloalkyl, C₃-C₆-cycloalkyl-C₁-C₄-alkyl, C₃-C₆-halocycloalkyl, phenyl, benzyl, where the phenyl ring in the two last-mentioned radicals is optionally substituted with 1, 2 or 3, in particular 1, substituents selected from halogen, cyano, C₁-C₄-alkyl, C₁-C₄-haloalkyl, C₁-C₄-alkoxy and C₁-C₄-haloalkoxy; and a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more, e.g. 1, 2, or 3, preferably or 2, in particular 1, substituents selected from halogen, cyano, C₁-C₄-alkyl, C₁-C₄-haloalkyl, C₁-C₄-alkoxy and C₁-C₄-haloalkoxy. More preferably, R⁸ as a substituent in a C(═O)OR⁸ group R⁴ is preferably selected from hydrogen, C₁-C₄-alkyl, C₁-C₄-haloalkyl, C₃-C₆-cycloalkyl, C₃-C₆-halocycloalkyl, phenyl and benzyl, and is specifically C₁-C₄-alkyl.

In the above definitions of R⁴, R^{9a}, R^{9b}, R^{17a} and R^{17b}, independently of each other, are preferably selected from hydrogen, C₁-C₄-alkyl, C₁-C₄-haloalkyl, C₃-C₆-cycloalkyl, C₃-C₆-cycloalkyl-C₁-C₄-alkyl, C₃-C₆-halocycloalkyl, phenyl, benzyl, where the phenyl ring in the two last-mentioned radicals is optionally substituted with 1, 2 or 3, in particular 1, substituents selected from halogen, cyano, C₁-C₄-alkyl, C₁-C₄-haloalkyl, C₁-C₄-alkoxy and C₁-C₄-haloalkoxy; and a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more, e.g. 1, 2, or 3, preferably or 2, in particular 1, substituents selected from halogen, cyano, C₁-C₄-alkyl, C₁-C₄-haloalkyl, C₁-C₄-alkoxy and C₁-C₄-haloalkoxy. More preferably, they are, independently of each other, selected from hydrogen, C₁-C₄-alkyl, C₁-C₄-haloalkyl, C₃-C₆-cycloalkyl and C₃-C₆-cycloalkyl-C₁-C₄-alkyl, and are specifically selected from hydrogen and C₁-C₄-alkyl.

In an alternative more preferred embodiment R⁴ is selected from hydrogen, C₁-C₆-alkyl, C₁-C₆-haloalkyl, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl, C₁-C₆-alkyl carrying one radical R⁷; C(═O)R⁷, C(═O)NR^{9a}R^{9b}, S(O)ₙR⁸, phenyl, optionally substituted with 1, 2, 3, 4 or 5 substituents R¹⁰; and a 5- or 6-membered saturated, partly unsaturated or maximally unsaturated (i.e. aromatic) heterocyclic ring comprising 1, 2 or 3 heteroatoms or heteroatom groups selected from N, O, S, NO, SO and SO₂ as ring members, where the heterocyclic ring is optionally substituted with one or more, preferably 1, 2 or 3, substituents R¹⁰;

where R⁷, R⁸, R^{9a}, R^{9b}, R¹⁰ and n have one of the above general or, in particular, one of the below preferred meanings.

In particular, R⁴ is selected from hydrogen, C₁-C₆-alkyl, C₁-C₆-haloalkyl, C₂-C₆-alkenyl, C₂-C₆-alkynyl, C₁-C₆-alkyl carrying one radical R⁷; C(═O)R⁷, C(═O)NR^{9a}R^{9b}, S(O)ₙR⁸, phenyl, optionally substituted with 1, 2, 3, 4 or 5 substituents R¹⁰; and a 5- or 6-membered saturated, partly unsaturated or maximally unsaturated (i.e. aromatic) heterocyclic ring comprising 1, 2 or 3 heteroatoms selected from N and O as ring members, where the heterocyclic ring is optionally substituted with one or more, preferably 1, 2 or 3, substituents R¹⁰;

where R⁷, R⁸, R^{9a}, R^{9b}, R¹⁰ and n have one of the above general or, in particular, one of the below preferred meanings.

In these alternative more preferred or particular embodiments, preferably
R⁷ as a substituent on C₁-C₆-alkyl is selected from C₃-C₆-cycloalkyl, C₃-C₆-halocycloalkyl, OR¹⁶ (where R¹⁶ is preferably C₁-C₆-alkyl or C₁-C₆-haloalkyl) and phenyl, optionally substituted with 1, 2 or 3 substituents selected from halogen, cyano, C₁-C₄-alkyl, C₁-C₄-haloalkyl, C₁-C₄-alkoxy and C₁-C₄-haloalkoxy; and
R⁷ as a substituent in a C(═O)R⁷ group is selected from C₁-C₄-alkyl, C₁-C₄-haloalkyl, NR^{17a}R^{17b} and phenyl which is optionally substituted with 1, 2 or 3 substituents selected from halogen, cyano, C₁-C₄-alkyl, C₁-C₄-haloalkyl, C₁-C₄-alkoxy and C₁-C₄-haloalkoxy;
R⁸ is selected from C₁-C₆-alkyl, C₁-C₆-haloalkyl and phenyl which is optionally substituted with 1, 2 or 3 substituents selected from halogen, cyano, C₁-C₄-alkyl, C₁-C₄-haloalkyl, C₁-C₄-alkoxy and C₁-C₄-haloalkoxy;
R^{9a} is selected from hydrogen and C₁-C₆-alkyl;
R^{9b} is selected from hydrogen, C₁-C₆-alkyl and C₁-C₆-haloalkyl;
R¹⁰ is selected from halogen, cyano, C₁-C₆-alkyl, C₁-C₆-haloalkyl, C₁-C₆-alkoxy, and C₁-C₆-haloalkoxy,
   or
   two R¹⁰ present together on the same carbon atom of a heterocyclic may together form a group ═O;
   or
   two R¹⁰ on adjacent carbon atoms may together form a bridge selected from CH₂CH₂CH₂CH₂, CH═CH—CH═CH, OCH₂CH₂CH₂, CH₂OCH₂CH₂, OCH₂CH₂O, OCH₂OCH₂, CH₂CH₂CH₂, CH═CHCH₂, CH₂CH₂O, CH₂OCH₂, and O(CH₂)O; in particular selected from CH═CH—CH═CH, OCH₂CH₂O, and O(CH₂)O; thus forming together with the carbon atoms to which they are bonded a 5-membered or 6-membered carbocyclic or heterocyclic ring; and
n is 2;
where R¹⁶, R^{17a} and R^{17b} have one of the above general or, in particular, one of the below preferred meanings.

In these alternative more preferred or particular embodiments, more preferably
R⁷ as a substituent on C₁-C₆-alkyl is selected from C₃-C₆-cycloalkyl, C₁-C₆-alkoxy and phenyl, optionally substituted with 1, 2 or 3 substituents selected from halogen, cyano, C₁-C₄-alkyl, C₁-C₄-haloalkyl, C₁-C₄-alkoxy and C₁-C₄-haloalkoxy; and
R⁷ as a substituent in a C(═O)R⁷ group is selected from C₁-C₄-alkyl, NR^{17a}R^{17b} and phenyl which is optionally substituted with 1, 2 or 3 substituents selected from halogen, cyano, C₁-C₄-alkyl, C₁-C₄-haloalkyl, C₁-C₄-alkoxy and C₁-C₄-haloalkoxy;
R⁸ is selected from C₁-C₆-alkyl;
R^{9a} is selected from hydrogen and C₁-C₆-alkyl;
R^{9b} is selected from hydrogen, C₁-C₆-alkyl and C₁-C₆-haloalkyl;
R¹⁰ is selected from halogen, cyano, C₁-C₆-alkyl, C₁-C₆-haloalkyl, C₁-C₆-alkoxy, and C₁-C₆-haloalkoxy,
   or
   two R¹⁰ present together on the same carbon atom of a heterocyclic may together form a group ═O;
   or
   two R¹⁰ on adjacent carbon atoms may together form a bridge selected from CH═CH—CH═CH, OCH₂CH₂O, and O(CH₂)O; thus forming together with the carbon atoms to which they are bonded a 5-membered or 6-membered carbocyclic or heterocyclic ring; and
n is 2.

In these preferred and more preferred definitions of R⁷, preferably
R^{17a} is selected from hydrogen and C₁-C₆-alkyl; and
R^{17a} is selected from hydrogen, C₁-C₆-alkyl, C₁-C₆-haloalkyl, C₃-C₆-cycloalkyl, C₃-C₆-halocycloalkyl, C₃-C₆-cycloalkyl-C₁-C₄-alkyl, phenyl and benzyl; and in particular from hydrogen, C₁-C₆-alkyl, C₁-C₆-haloalkyl and C₃-C₆-cycloalkyl-C₁-C₄-alkyl.

Preferably, R⁵ is selected from halogen, cyano, C₁-C₆-alkyl, C₁-C₆-haloalkyl, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl, C₁-C₄-alkoxy, C₁-C₄-haloalkoxy, C₁-C₄-alkylthio, C₁-C₄-haloalkylthio, phenyl, optionally substituted with 1, 2 or 3, in particular 1, substituents R⁶; and a 3-, 4-, 5-, 6- or 7-membered saturated, partly unsaturated or maximally unsaturated heterocyclic ring comprising 1, 2, 3 or 4 heteroatoms or heteroatom groups selected from O, S, N, NO, SO and SO₂ as ring members, where the heterocyclic ring is optionally substituted with one or more, e.g. 1, 2, or 3, preferably or 2, in particular 1, substituents R⁶, where R⁶ has one of the above general or, in particular, one of the below preferred meanings.

More preferably, R⁵ is selected from halogen, C₁-C₆-alkyl, C₁-C₆-haloalkyl, C₁-C₄-alkoxy, C₁-C₄-haloalkoxy, C₁-C₄-alkylthio, C₁-C₄-haloalkylthio, phenyl, optionally substituted with 1, 2 or 3, in particular 1, substituents R⁶; and a 3-, 4-, 5-, 6- or 7-membered saturated, partly unsaturated or maximally unsaturated heterocyclic ring comprising 1, 2, 3 or 4 heteroatoms or heteroatom groups selected from O, S, N, NO, SO and SO₂ as ring members, where the heterocyclic ring is optionally substituted with one or more, e.g. 1, 2, or 3, preferably or 2, in particular 1, substituents R⁶, where R⁶ has one of the above general or, in particular, one of the below preferred meanings.

Preferably, the 3-, 4-, 5-, 6- or 7-membered saturated, partly unsaturated or maximally unsaturated heterocyclic ring R⁵ is selected from rings of formulae B-1 to B-135:

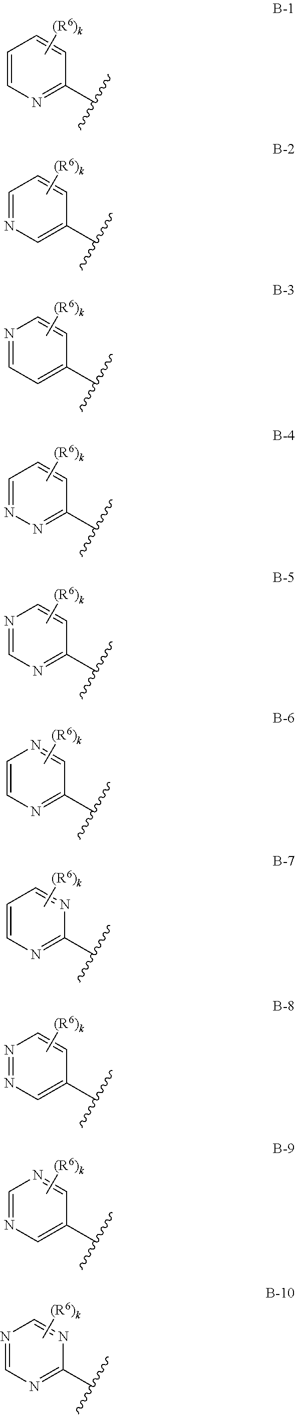

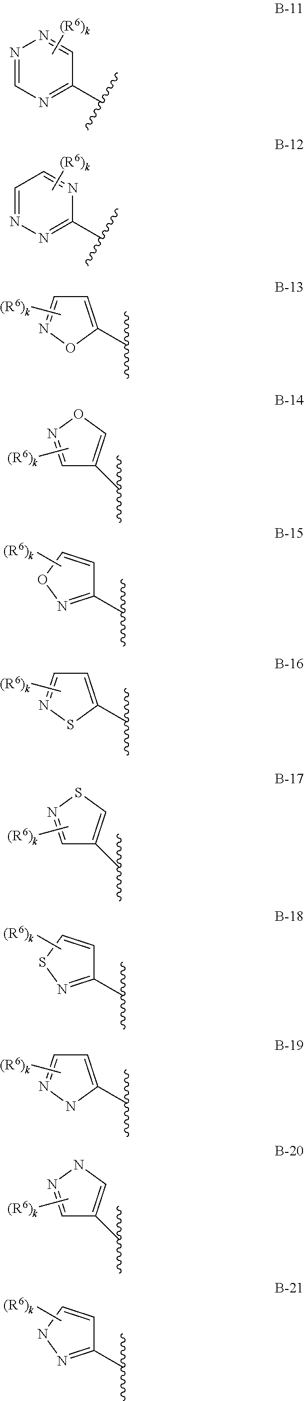

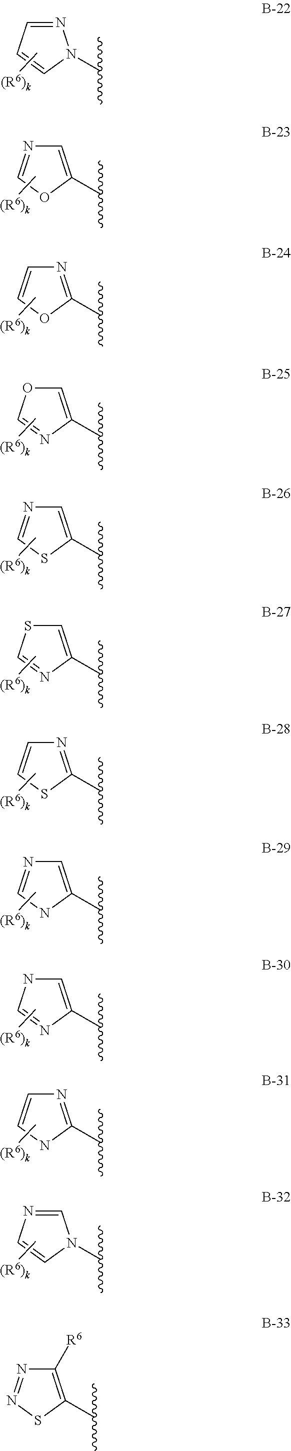

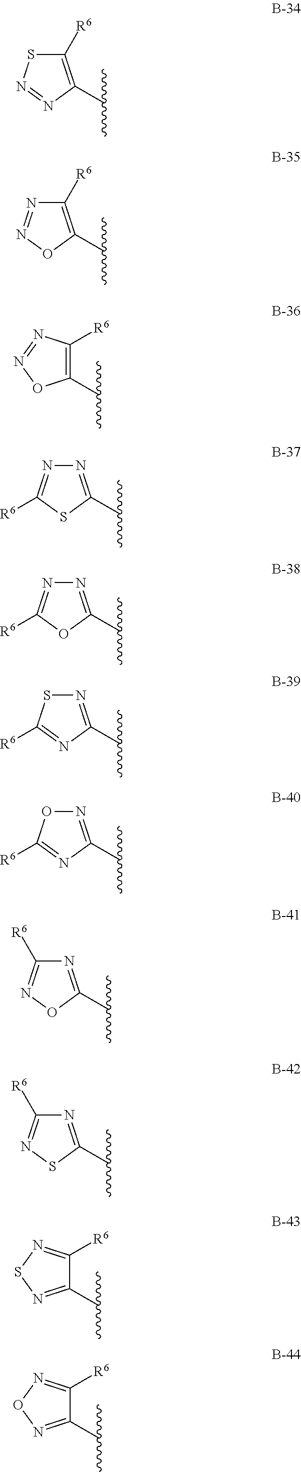

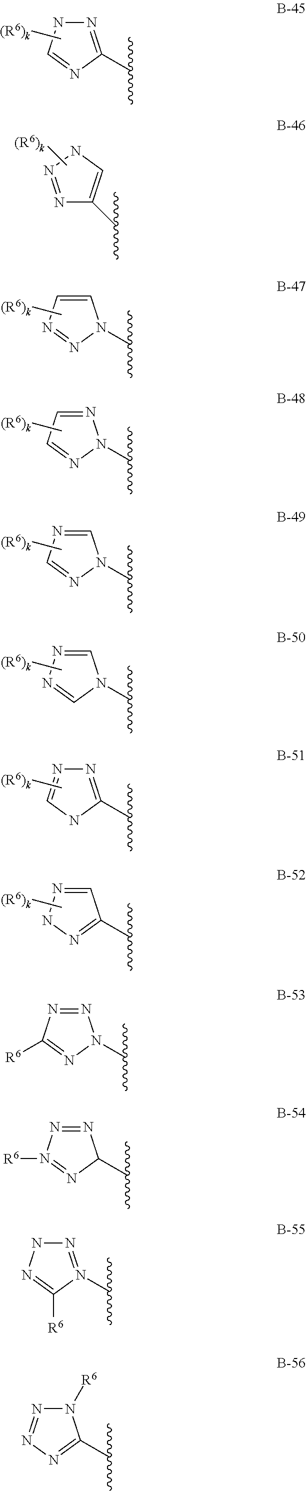

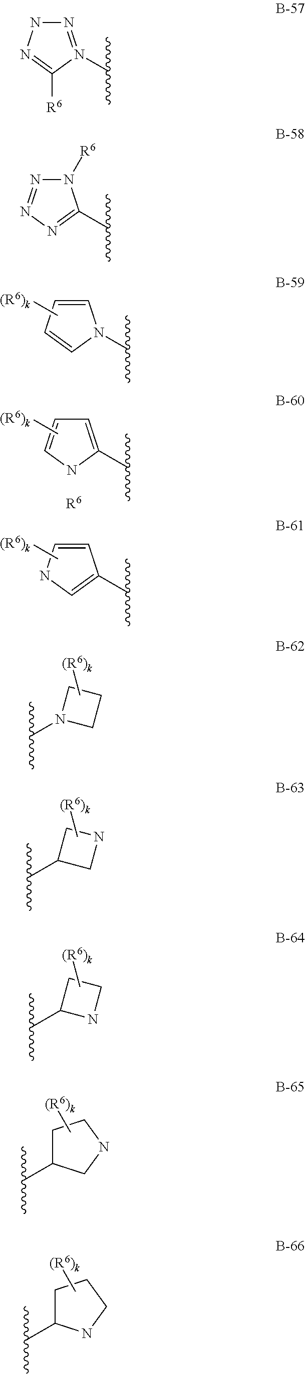

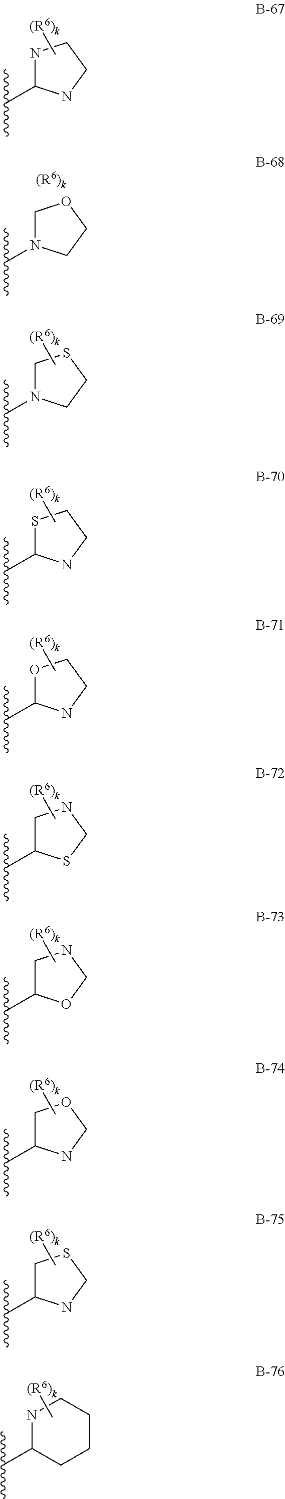

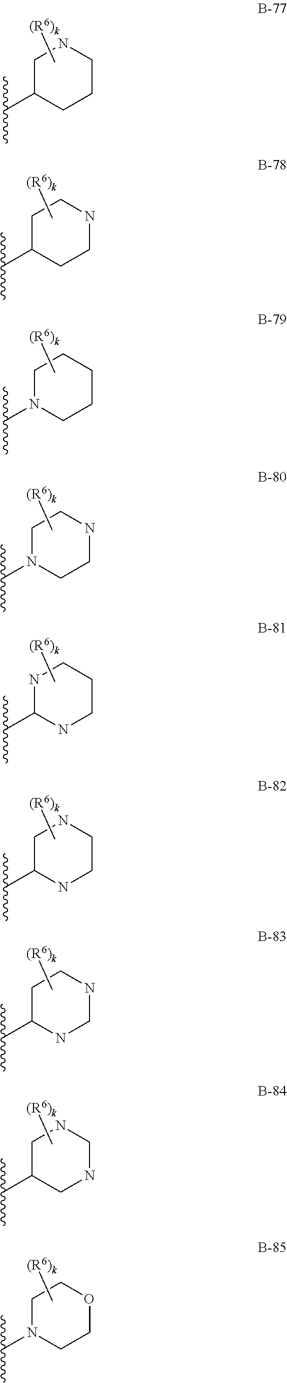

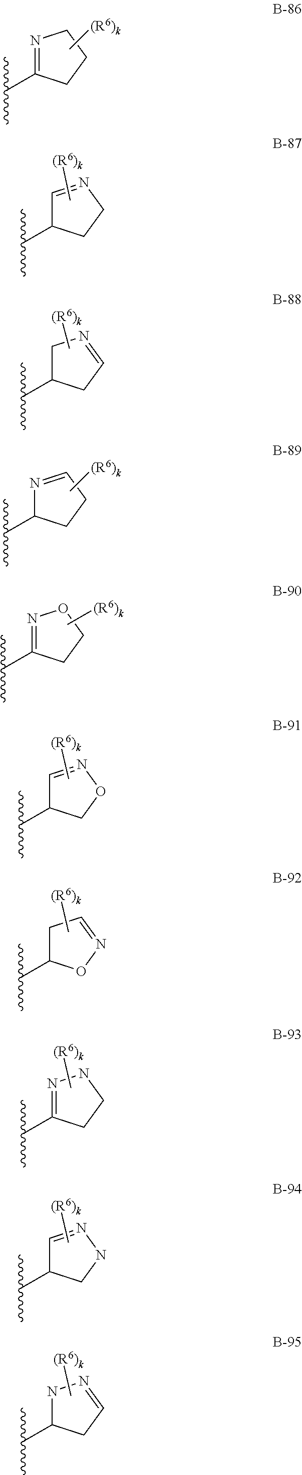

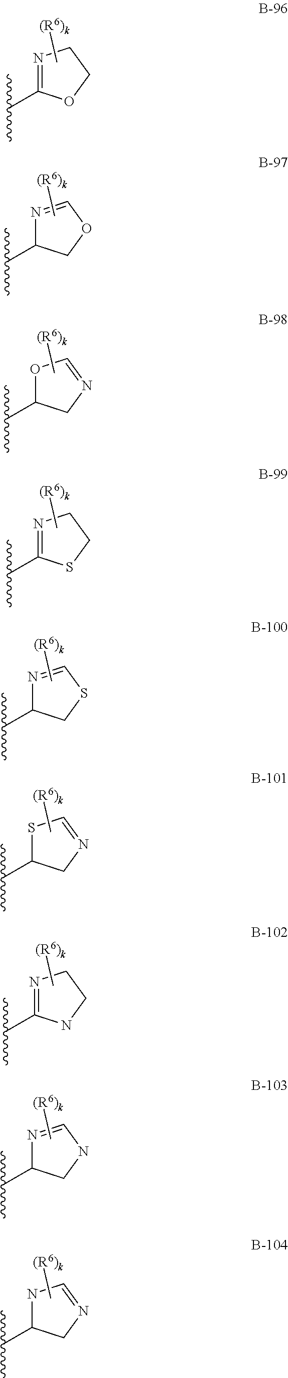

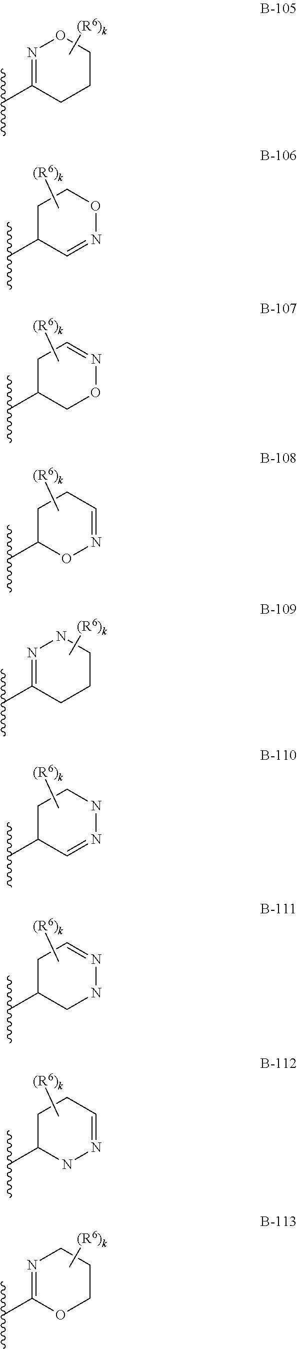

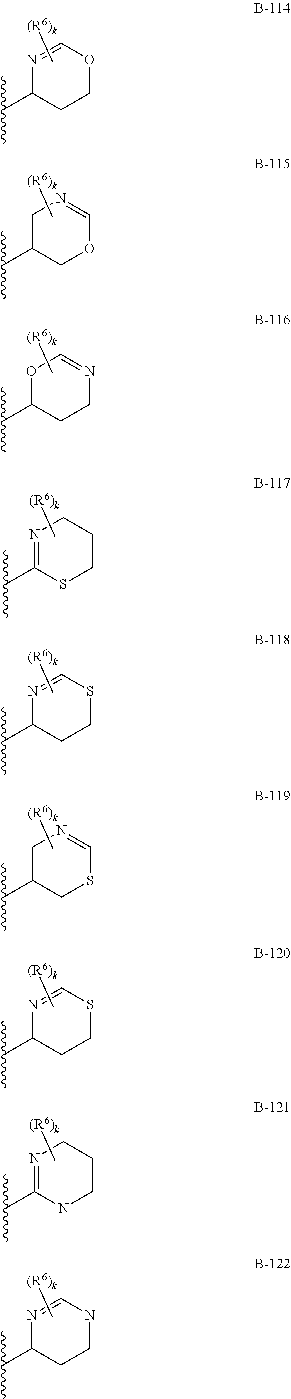

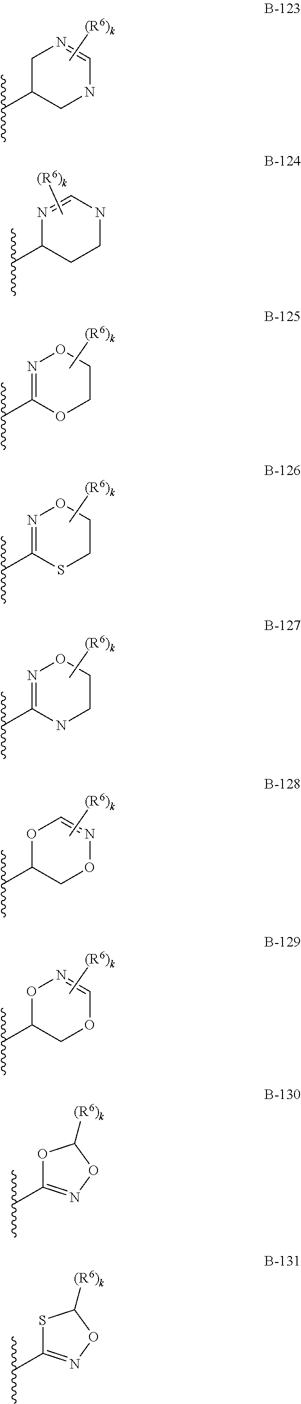

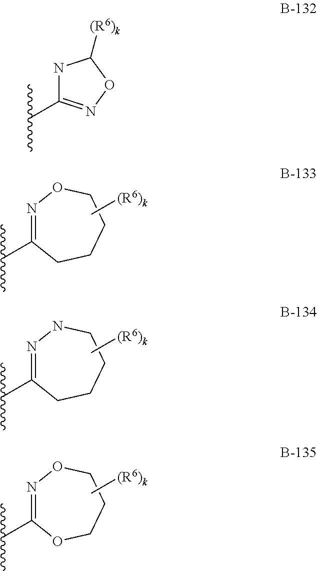

wherein the zigzag line denotes the bond to the remainder of the molecule, k is 0, 1, 2, 3, 4, 5 or 6 and R⁶ has one of the above general or, in particular, one of the below preferred meanings.

More preferably, the 3-, 4-, 5-, 6- or 7-membered saturated, partly unsaturated or maximally unsaturated heterocyclic ring R⁵ is selected from rings of formulae B-1 to B-32 and even more preferably from rings B-1 to B-3, B-5, B-7, B-9 and B-26 to B-28. In particular, the 3-, 4-, 5-, 6- or 7-membered saturated, partly unsaturated or maximally unsaturated heterocyclic ring R⁵ is selected from rings of formulae B-1 to B-3, B-5, B-7 and B-9 and specifically from rings of formulae B-1 and B-7.

Thus, even more preferably, R⁵ is selected from halogen, C₁-C₆-alkyl, C₁-C₆-haloalkyl, C₁-C₄-alkoxy, C₁-C₄-haloalkoxy, C₁-C₄-alkylthio, C₁-C₄-haloalkylthio, phenyl, optionally substituted with 1, 2 or 3, in particular 1, substituents R⁶; and a heteroaromatic ring selected from pyridyl, pyrimidyl and thiazolyl, where the heterocyclic ring is optionally substituted with one or more, e.g. 1, 2, or 3, preferably or 2, in particular 1, substituents R⁶, where R⁶ has one of the above general or, in particular, one of the below preferred meanings. In particular, R⁵ is selected from halogen, C₁-C₆-alkyl, C₁-C₆-haloalkyl, C₁-C₄-alkoxy, C₁-C₄-haloalkoxy, C₁-C₄-alkylthio, C₁-C₄-haloalkylthio, phenyl, optionally substituted with 1, 2 or 3, in particular 1, substituents R⁶; and a heteroaromatic ring selected from pyridyl and pyrimidyl (and specifically from pyridin-2-yl and 2-pyrimidin-2-yl), where the heteroaromatic ring is optionally substituted with one or more, e.g. 1, 2, or 3, preferably or 2, in particular 1, substituents R⁶, where R⁶ has one of the above general or, in particular, one of the below preferred meanings.

Preferably, each R⁶ is independently selected from the group consisting of halogen, cyano, nitro, C₁-C₆-alkyl, C₃-C₈-cycloalkyl, wherein the two last-mentioned aliphatic and cycloaliphatic radicals may be partially or fully halogenated and/or may be substituted with one or more radicals R⁷; OR⁸, NR^{9a}R^{9b}, S(O)ₙR⁸, S(O)ₙNR^{9a}R^{9b}, C(═O)R⁷, C(═O)NR^{9a}R^{9b} and C(═O)OR⁸, or

two R⁶ present together on one carbon atom of a partly saturated heterocyclic ring may be ═O, ═CR¹³R¹⁴; ═S, ═NR^{17a}, ═NOR¹⁶ or ═NNR^{17a},
where R⁷, R⁸, R^{9a}, R^{9b}, R¹³, R¹⁴, R¹⁶ and R^{17a} have one of the above general or, in particular, one of the below preferred meanings.

Preferably, k is 0 or 1.

In all rings A-1 to A-17 and A-19 to A-24 p is preferably 0 or 1 and specifically 0.

Preferably, r is 0.

If not specified otherwise above, R⁷, R⁸, R^{9a}, R^{9b}, R¹⁰, R¹¹, R¹², R¹³, R¹⁴, R¹⁵, R¹⁶, R^{17a}, R^{17b}, R¹⁸, R¹⁹ have following preferred meanings:

In case R⁷ is a substituent on an alkyl, alkenyl or alkynyl group, it is preferably selected from the group consisting of cyano, C₃-C₈-cycloalkyl, C₃-C₈-halocycloalkyl, —OR¹⁶, —SR¹⁶, —C(═O)N(R^{17a})R^{17b}, —C(═S)N(R^{17a})R^{17b}, —C(═O)OR¹⁶, phenyl which may be substituted by 1, 2, 3, 4 or 5 radicals R¹⁵, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹⁵; where R¹⁵, R¹⁶, R^{17a} and R^{17b} have one of the meanings given above or in particular one of the preferred meanings given below.

In case R⁷ is a substituent on an alkyl, alkenyl or alkynyl group, it is even more preferably selected from the group consisting of cyano, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, —C(═O)N(R^{17a})R^{17b}, —C(═S)N(R^{17a})R^{17b}, —C(═O)OR¹⁶, phenyl which may be substituted by 1, 2, 3, 4 or 5 radicals R¹⁵, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹⁵; where R¹⁵, R¹⁶, R^{17a} and R^{17b} have one of the meanings given above or in particular one of the preferred meanings given below. In particular it is selected from the group consisting of cyano, C₃-C₆-cycloalkyl, C₃-C₆-halocycloalkyl, —C(═O)N(R^{17a})R^{17b}, —C(═S)N(R^{17a})R^{17b}, —C(═O)OR¹⁶, phenyl which may be substituted by 1, 2, 3, 4 or 5 radicals R¹⁵, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹⁵; where R¹⁵, R¹⁶, R^{17a} and R^{17b} have one of the meanings given above or in particular one of the preferred meanings given below.

In case R⁷ is a substituent on a cycloalkyl group, it is preferably selected from the group consisting of cyano, C₁-C₆-alkyl, C₁-C₆-haloalkyl, C₁-C₆-alkoxy-C₁-C₆-alkyl, —OR¹⁶, —OSO₂R¹⁶, —SR¹⁶, —N(R^{17a})R^{17b}, —C(═O)N(R^{17a})R^{17b}, —C(═S)N(R^{17a})R^{17b}, —C(═O)OR¹⁶, phenyl which may be substituted by 1, 2, 3, 4 or 5 radicals R¹⁵, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹⁵; where R¹⁵, R¹⁶, R^{17a} and R^{17b} have one of the meanings given above or in particular one of the preferred meanings given below.

In case R⁷ is a substituent on a cycloalkyl group, it is even more preferably selected from the group consisting of halogen, C₁-C₄-alkyl, C₁-C₃-haloalkyl, C₁-C₄-alkoxy and C₁-C₃-haloalkoxy. In particular, R⁷ as a substituent on a cycloalkyl group is selected from halogen, C₁-C₄-alkyl and C₁-C₃-haloalkyl.

In case of R⁷ in a group —C(═O)R⁷, —C(═S)R⁷, or —C(═NR^{9a})R⁷, R⁷ is preferably selected from the group consisting of hydrogen, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, —OR¹⁶, —SR¹⁶, —N(R^{17a})R^{17b}, phenyl which may be substituted by 1, 2, 3, 4 or 5 radicals R¹⁵, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹⁵; where R¹⁵, R¹⁶, R^{17a} and R^{17b} have one of the meanings given above or in particular one of the preferred meanings given below.

In case of R⁷ in a group —C(═O)R⁸, —C(═S)R⁷, or —C(═NR^{9a})R⁷, R⁷ is more preferably selected from the group consisting of C₁-C₆-alkyl, C₁-C₆-haloalkyl, C₃-C₈-cycloalkyl, C₃-C₈-halocycloalkyl, C₁-C₆-alkoxy, C₁-C₆-haloalkoxy, —N(R^{17a})R^{17b}, phenyl which may be substituted by 1, 2, 3, 4 or 5 radicals R¹⁵, and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹⁵; where R¹⁵, R^{17a} and R^{17b} have has one of the meanings given above or in particular one of the preferred meanings given below.

Preferably, each R⁸ is independently selected from the group consisting of hydrogen, C₁-C₆-alkyl, C₁-C₆-haloalkyl, C₃-C₈-cycloalkyl, C₃-C₈-halocycloalkyl, C₃-C₈-cycloalkyl-C₁-C₄-alkyl, phenyl which may be substituted by 1, 2, 3, 4 or 5 radicals R¹⁵; and a 3-, 4-, 5-, 6- or 7-membered saturated, partially unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁵, where R¹⁵ has one of the meanings given above or in particular one of the preferred meanings given below.

More preferably, each R⁸ is independently selected from the group consisting of hydrogen, C₁-C₆-alkyl, C₁-C₆-haloalkyl, phenyl which may be substituted by 1, 2, 3, 4 or 5 radicals R¹⁵; and a 5- or 6-membered heteroaromatic ring containing 1, 2 or 3 heteroatoms selected from N, O and S, as ring members, where the heteroaromatic ring may be substituted by one or more radicals R¹⁵; where R¹⁵ has one of the meanings given above or in particular one of the preferred meanings given below.

R^{9a} and R^{9b} are, independently of each other, preferably selected from hydrogen, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-cycloalkyl-C₁-C₄-alkyl, C₃-C₆-halocycloalkyl, C₁-C₄-alkylcarbonyl, C₁-C₄-haloalkylcarbonyl, C₁-C₄-alkylaminocarbonyl, di(C₁-C₄-alkyl)aminocarbonyl, C₁-C₄-haloalkylaminocarbonyl, C₃-C₆-cycloalkylaminocarbonyl, C₃-C₆-halocycloalkylaminocarbonyl, phenyl, benzyl, where the phenyl ring in the two last-mentioned radicals is optionally substituted with 1, 2 or 3, in particular 1, substituents selected from halogen, cyano, C₁-C₄-alkyl, C₁-C₄-haloalkyl, C₁-C₄-alkoxy and C₁-C₄-haloalkoxy; and a 3-, 4-, 5-, 6- or 7-membered saturated, partially unsaturated or maximally unsaturated heterocyclic ring comprising 1, 2 or 3 heteroatoms or heteroatom groups selected from N, O, S, NO, SO and SO₂, as ring members, where the heterocyclic ring is optionally substituted with one or more, preferably 1, 2 or 3, in particular 1, substituents selected from halogen, CN, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₁-C₄-haloalkoxy, C₁-C₄-alkylthio and C₁-C₄-haloalkylthio; or

R^{9a} and R^{9b}, together with the nitrogen atom to which they are bound, form a 5- or 6-membered saturated, partially unsaturated or aromatic heterocyclic ring, which additionally may contain 1 or 2 further heteroatoms or heteroatom groups selected from N, O, S, NO, SO and SO₂, as ring members, where the heterocyclic ring may carry 1 or 2, in particular 1, substituents selected from halogen, CN, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₁-C₄-haloalkoxy, C₁-C₄-alkylthio and C₁-C₄-haloalkylthio.

More preferably, R^{9a} and R^{9b} are, independently of each other, selected from hydrogen, C₁-C₄-alkyl, C₁-C₄-haloalkyl, C₃-C₆-cycloalkyl, C₃-C₆-cycloalkyl-C₁-C₄-alkyl, C₃-C₆-halocycloalkyl, phenyl, benzyl, where the phenyl ring in the two last-mentioned radicals is optionally substituted with 1, 2 or 3, in particular 1, substituents selected from halogen, cyano, C₁-C₄-alkyl, C₁-C₄-haloalkyl, C₁-C₄-alkoxy and C₁-C₄-haloalkoxy; and a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more, e.g. 1, 2, or 3, preferably or 2, in particular 1, substituents selected from halogen, cyano, C₁-C₄-alkyl, C₁-C₄-haloalkyl, C₁-C₄-alkoxy and C₁-C₄-haloalkoxy. In particular, they are, independently of each other, selected from hydrogen, C₁-C₄-alkyl, C₁-C₄-haloalkyl and C₃-C₆-cycloalkyl, and are specifically selected from hydrogen and C₁-C₄-alkyl.

Each R¹⁰ and each R¹⁵ are independently of each occurrence and independently of each other preferably selected from halogen, CN, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-halocycloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl and C₁-C₄-haloalkylsulfonyl, more preferably from halogen, CN, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl and C₁-C₄-haloalkylsulfonyl, and in particular from halogen, CN, C₁-C₄-alkyl, C₁-C₄-haloalkyl, C₁-C₄-alkoxy and C₁-C₄-haloalkoxy.

Each R¹¹ and each R¹² are independently of each occurrence and independently of each other preferably selected from C₁-C₄-alkyl and are in particular methyl.

Each R¹³ and each R¹⁴ are independently of each occurrence and independently of each other preferably selected from the group consisting of hydrogen and C₁-C₄-alkyl.

Each R¹⁶ is independently preferably selected from hydrogen, C₁-C₆-alkyl, C₁-C₆-haloalkyl, phenyl and benzyl, wherein the two last-mentioned radicals may be unsubstituted or carry 1, 2 or 3, in particular 1, substituents selected from halogen, C₁-C₆-alkyl, C₁-C₆-haloalkyl, C₁-C₆-alkoxy and C₁-C₆-haloalkoxy. More preferably, each R¹⁶ is independently selected from hydrogen and C₁-C₆-alkyl.

R^{17a} and R^{17b} are, independently of each other, preferably selected from hydrogen, C₁-C₄-alkyl, C₁-C₄-haloalkyl, C₂-C₄-alkenyl, C₂-C₄-haloalkenyl, C₂-C₄-alkynyl, C₂-C₄-haloalkynyl, C₃-C₆-cycloalkyl, C₃-C₆-cycloalkyl-C₁-C₄-alkyl, C₃-C₆-halocycloalkyl, phenyl and benzyl, where the phenyl ring in the two last-mentioned radicals is optionally substituted with 1, 2 or 3, in particular 1, substituents selected from halogen, cyano, C₁-C₄-alkyl, C₁-C₄-haloalkyl, C₁-C₄-alkoxy and C₁-C₄-haloalkoxy;

or
R^{17a} and R^{17b}, together with the nitrogen atom to which they are bound, form a 5- or 6-membered saturated, partially unsaturated or aromatic heterocyclic ring, which additionally may contain 1 or 2 further heteroatoms or heteroatom groups selected from N, O, S, NO, SO and SO₂, as ring members, where the heterocyclic ring may carry 1 or 2, in particular 1, substituents selected from halogen, CN, C₁-C₄-alkyl, C₁-C₄-haloalkyl, C₁-C₄-alkoxy and C₁-C₄-haloalkoxy. In particular, R^{17a} and R^{17b} are, independently of each other, selected from hydrogen, C₁-C₄-alkyl, C₁-C₄-haloalkyl and C₃-C₆-cycloalkyl, and are specifically selected from hydrogen and C₁-C₄-alkyl.

R¹⁸ is preferably selected from the group consisting of hydrogen, C₁-C₆-alkyl, C₁-C₆-haloalkyl, C₃-C₈-cycloalkyl, C₃-C₈-halocycloalkyl, —OH, —SH, C₁-C₆-alkoxy, C₁-C₆-haloalkoxy and phenyl which may be substituted by 1, 2 or 3 radicals selected from halogen, C₁-C₄-alkyl, C₁-C₄-haloalkyl, C₁-C₄-alkoxy and C₁-C₄-haloalkoxy.

In case R¹⁹ is a substituent on an alkyl, alkenyl or alkynyl group, it is preferably selected from the group consisting of cyano, —OH, —SH, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₃-C₈-cycloalkyl, C₃-C₈-halocycloalkyl and phenyl which may be substituted by 1, 2 or 3 radicals selected from halogen, C₁-C₄-alkyl, C₁-C₄-haloalkyl, C₁-C₄-alkoxy and C₁-C₄-haloalkoxy.

In case R¹⁹ is a substituent on a cycloalkyl group, it is preferably selected from the group consisting of cyano, —OH, —SH, C₁-C₄-alkoxy, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₁-C₆-alkyl, C₁-C₆-haloalkyl, C₃-C₈-cycloalkyl, C₃-C₈-halocycloalkyl and phenyl which may be substituted by 1, 2 or 3 radicals selected from C₁-C₄-alkyl, C₁-C₄-haloalkyl, C₁-C₄-alkoxy and C₁-C₄-haloalkoxy.

In case R¹⁹ is a substituent on a cycloalkyl group, it is even more preferably selected from the group consisting of C₁-C₄-alkyl, C₁-C₃-haloalkyl, C₁-C₄-alkoxy and C₁-C₃-haloalkoxy. In particular, R¹⁹ as a substituent on a cycloalkyl group is selected from C₁-C₄-alkyl and C₁-C₃-haloalkyl.

m is specifically 0.

In a specific embodiment, the compound I is a compound of formula I.1

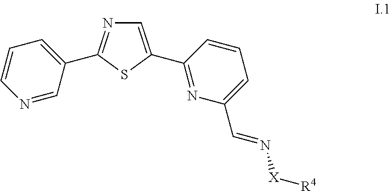

wherein X and R⁴ have one of the above general or, in particular, one of the above preferred meanings.

The zigzag line is intended to show that compounds I.1 are not limited to a specific steroisomerism at the nitrogen atom of the imine (derived) group C(H)═N—X—R⁴; i.e. the pyridyl ring to which this group is bonded and the group X—R⁴ can be both cis or trans to each other. Preferably, however, they are trans to each other.

In another specific embodiment, the compound I is a compound of formula I.2

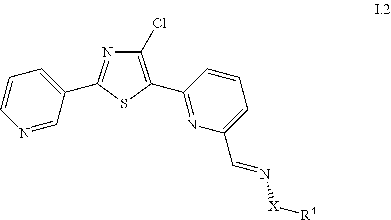

wherein X and R⁴ have one of the above general or, in particular, one of the above preferred meanings.

The zigzag line is intended to show that compounds I.2 are not limited to a specific steroisomerism at the nitrogen atom of the imine (derived) group C(H)═N—X—R⁴; i.e. the pyridyl ring to which this group is bonded and the group X—R⁴ can be both cis or trans to each other. Preferably, however, they are trans to each other.

In another specific embodiment, the compound I is a compound of formula I.3

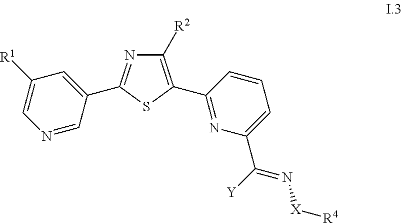

wherein X, Y, R¹, R² and R⁴ have one of the above general or, in particular, one of the above preferred meanings.

The zigzag line is intended to show that compounds I.3 are not limited to a specific steroisomerism at the nitrogen atom of the imine (derived) group C(Y)═N—X—R⁴; i.e. the pyridyl ring to which this group is bonded and the group X—R⁴ can be both cis or trans to each other. Preferably, however, they are trans to each other.

In another specific embodiment, the compound I is a compound of formula I.4

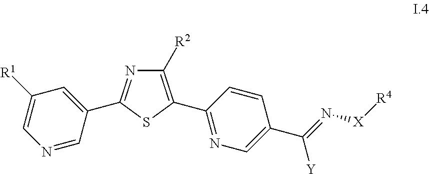

wherein X, Y, R¹, R² and R⁴ have one of the above general or, in particular, one of the above preferred meanings.

The zigzag line is intended to show that compounds I.4 are not limited to a specific steroisomerism at the nitrogen atom of the imine (derived) group C(Y)═N—X—R⁴; i.e. the pyridyl ring to which this group is bonded and the group X—R⁴ can be both cis or trans to each other. Preferably, however, they are trans to each other.

In another specific embodiment, the compound I is a compound of formula I.5

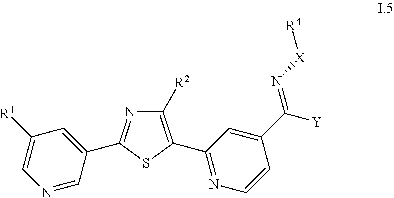

wherein X, Y, R¹, R² and R⁴ have one of the above general or, in particular, one of the above preferred meanings.

The zigzag line is intended to show that compounds I.5 are not limited to a specific steroisomerism at the nitrogen atom of the imine (derived) group C(Y)═N—X—R⁴; i.e. the pyridyl ring to which this group is bonded and the group X—R⁴ can be both cis or trans to each other. Preferably, however, they are trans to each other.

In another specific embodiment, the compound I is a compound of formula I.6

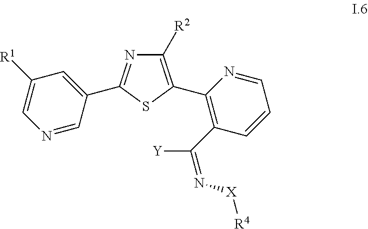

wherein X, Y, R¹, R² and R⁴ have one of the above general or, in particular, one of the above preferred meanings.

The zigzag line is intended to show that compounds I.6 are not limited to a specific steroisomerism at the nitrogen atom of the imine (derived) group C(Y)═N—X—R⁴; i.e. the pyridyl ring to which this group is bonded and the group X—R⁴ can be both cis or trans to each other. Preferably, however, they are trans to each other.

Examples of preferred compounds are compounds of the following formulae Ia.1 to Ia.216, where X, Y and R⁴ have one of the general or preferred meanings given above and R^{5a} is hydrogen or has one of the general or preferred meanings given above for R⁵. Examples of preferred compounds are the individual compounds compiled in the tables 1 to 12960 below. Moreover, the meanings mentioned below for the individual variables in the tables are per se, independently of the combination in which they are mentioned, a particularly preferred embodiment of the substituents in question. It has to be noted that the structures shown do not limit the compounds to a specific steroisomerism at the nitrogen atom of the imine (derived) group C(Y)═N—X—R⁴.

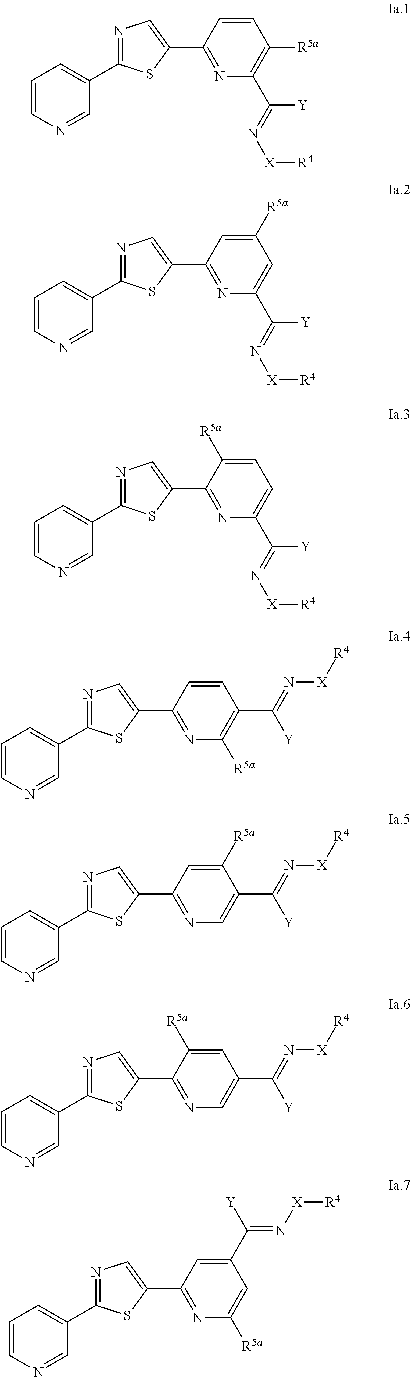

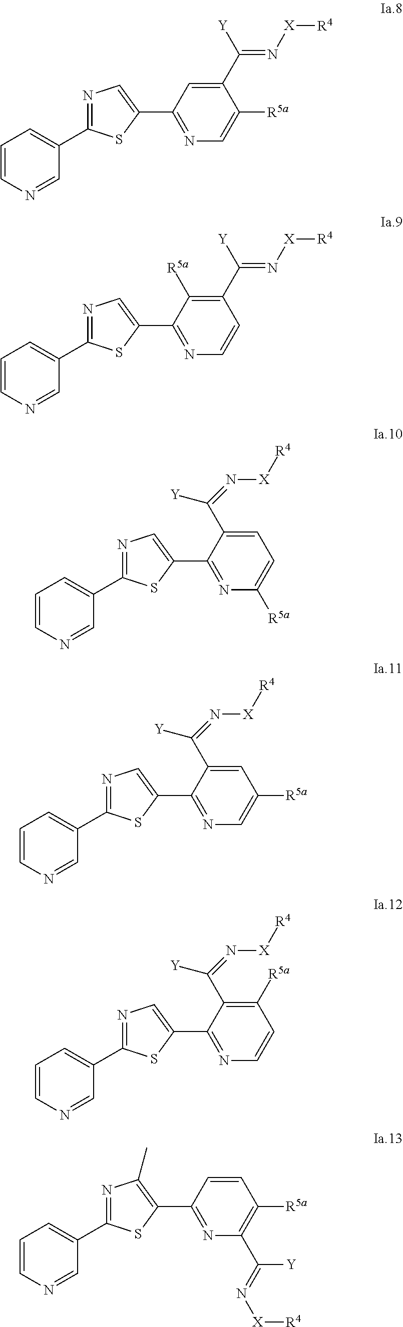

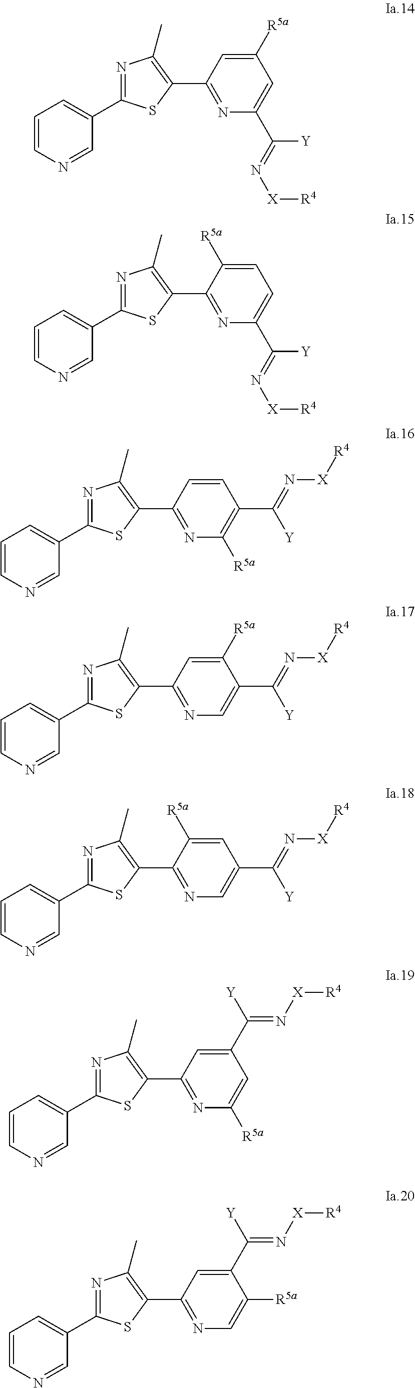

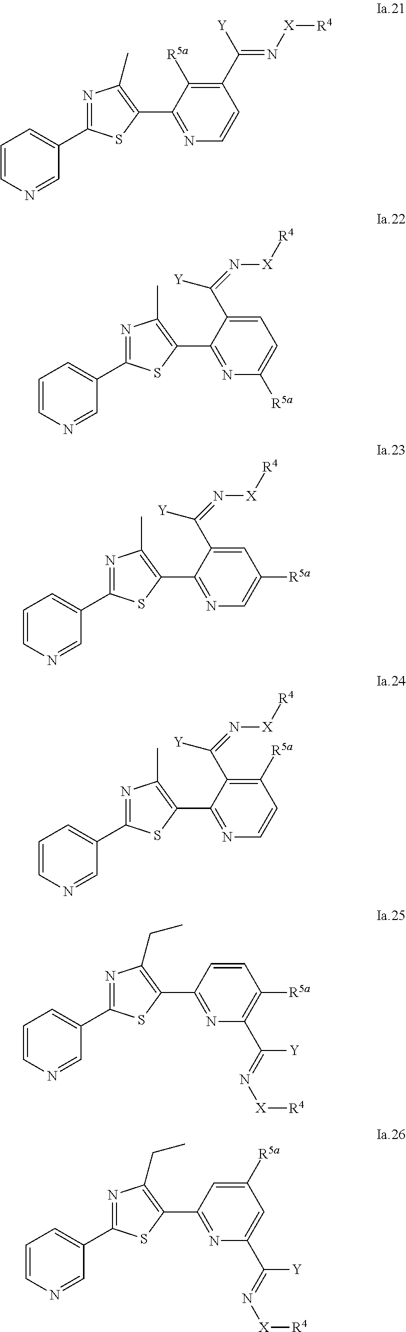

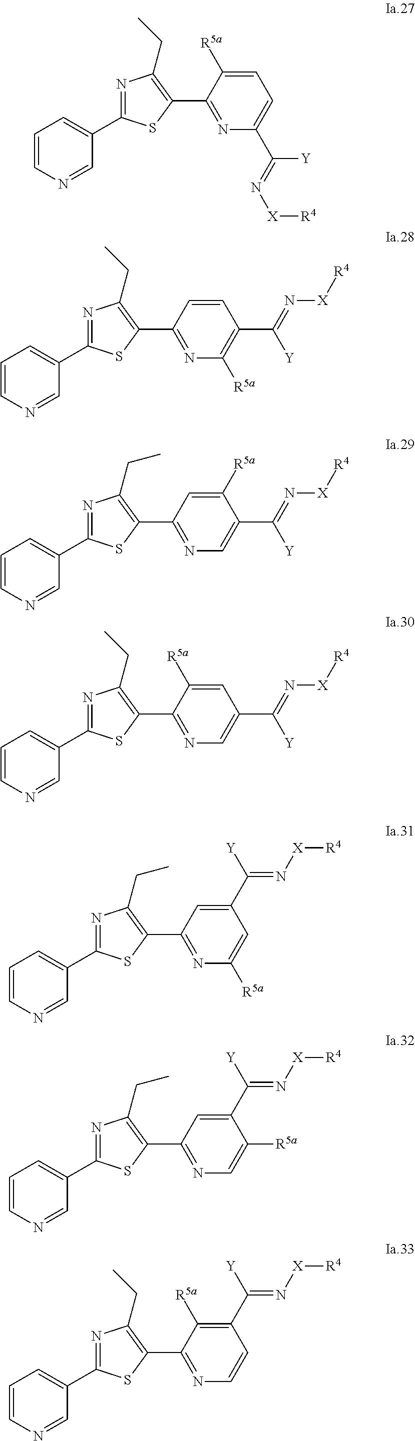

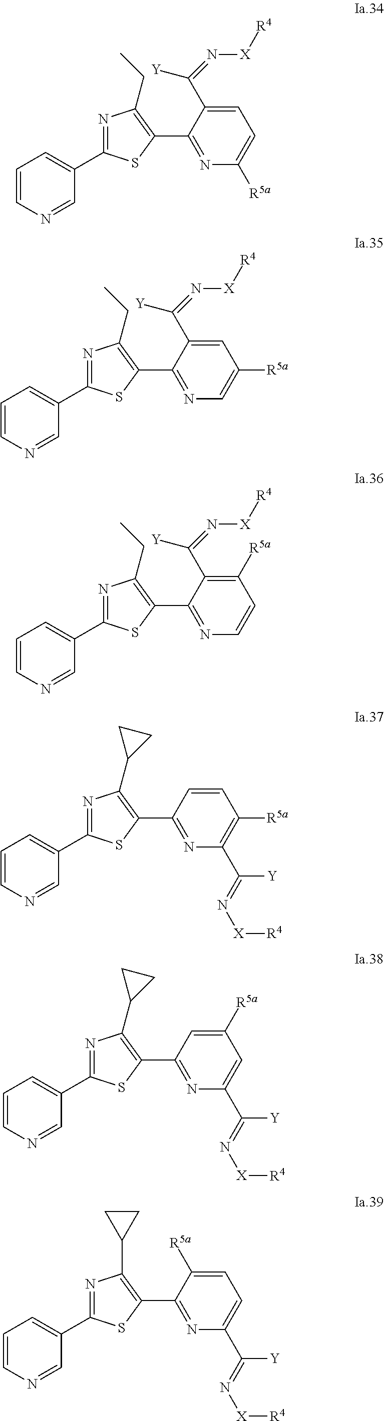

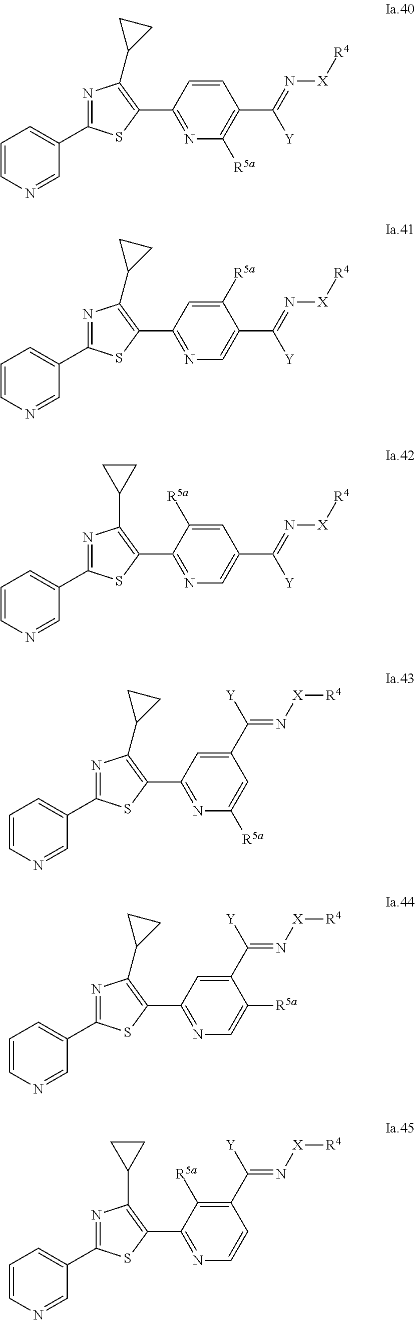

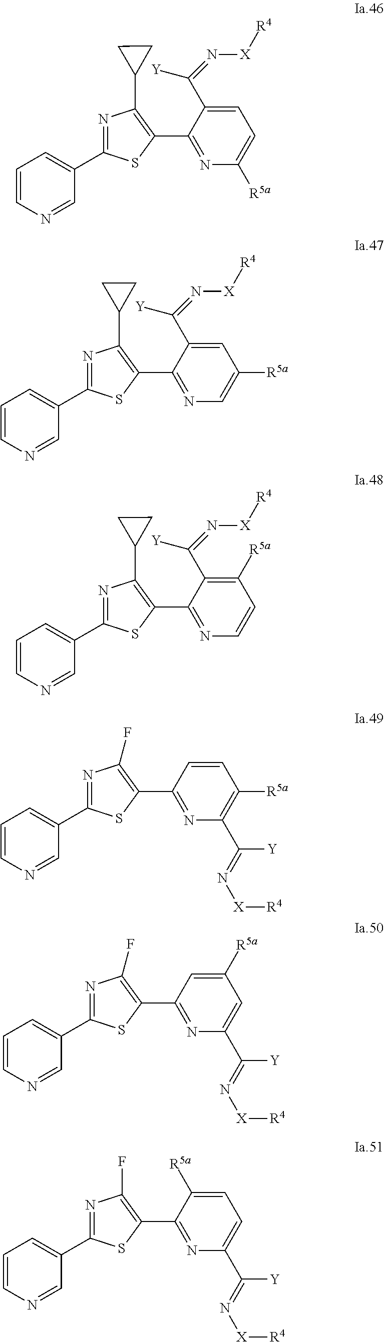

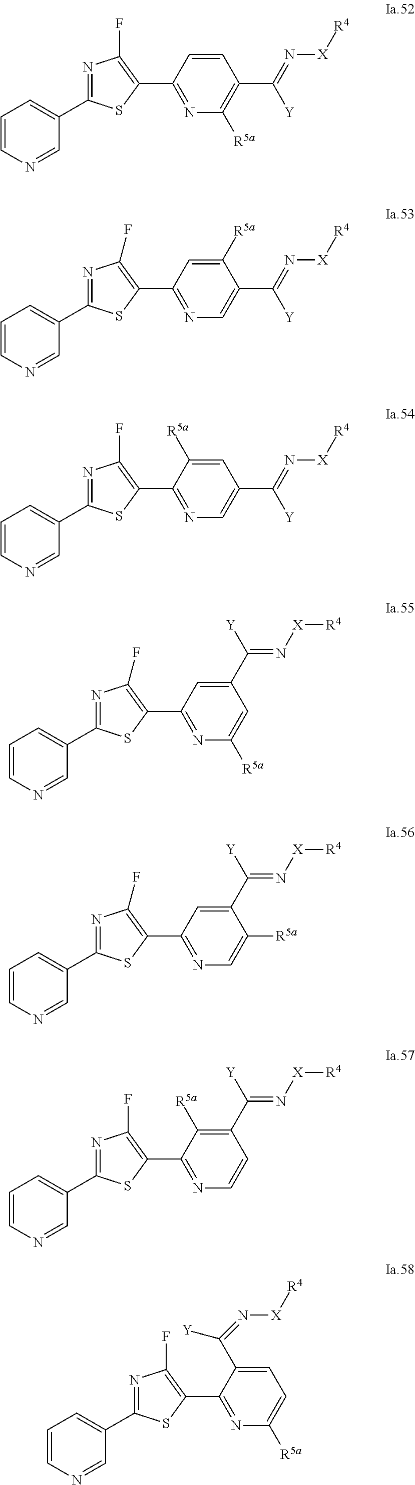

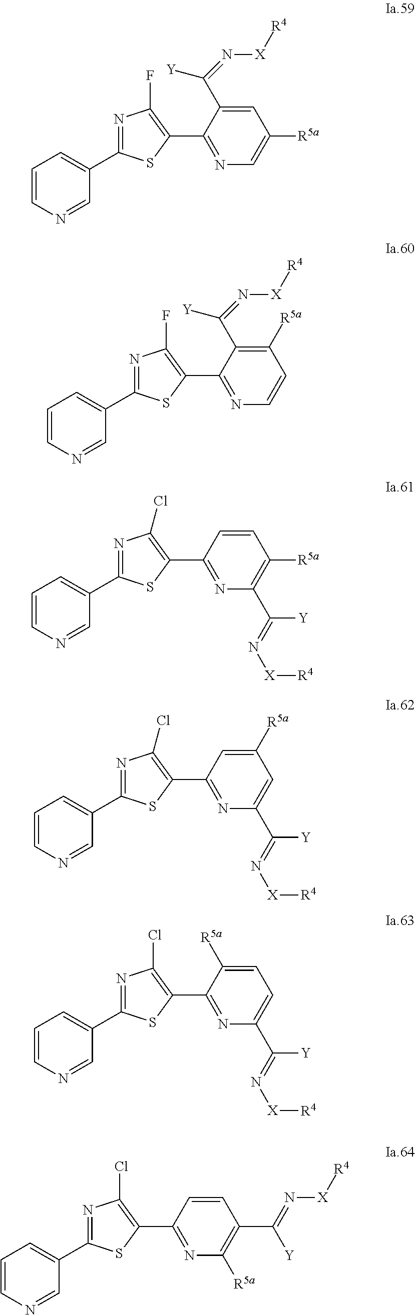

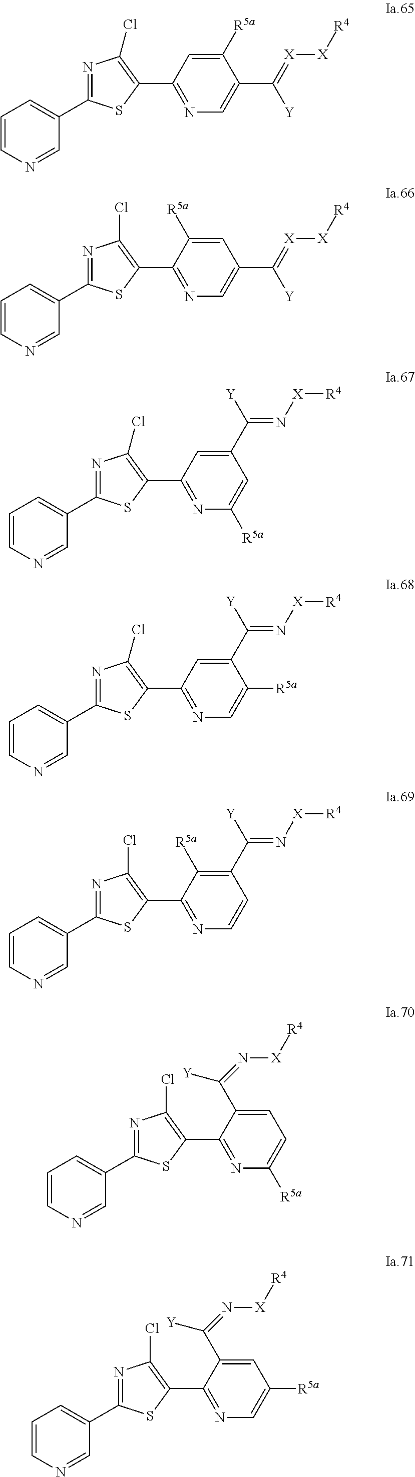

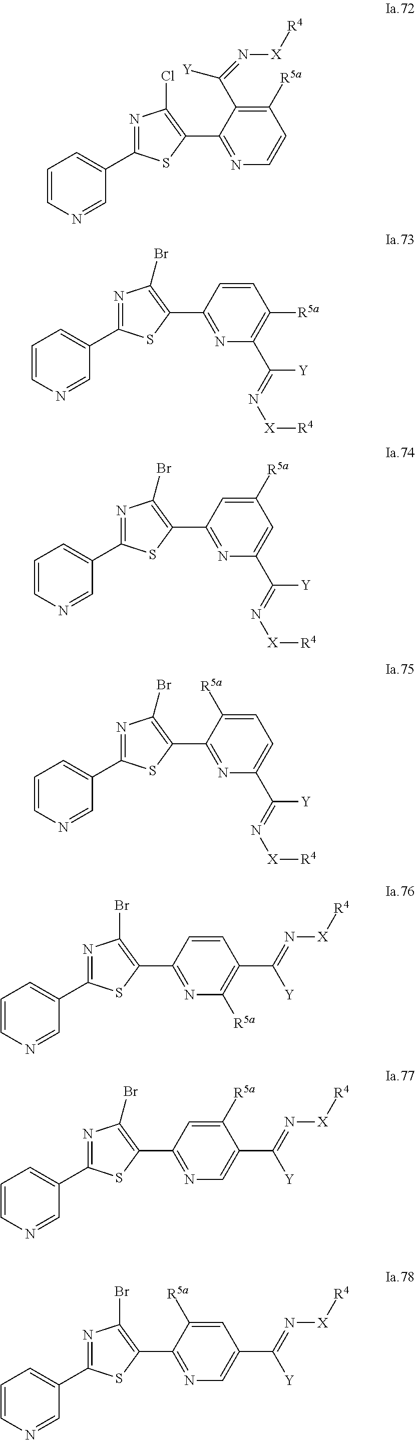

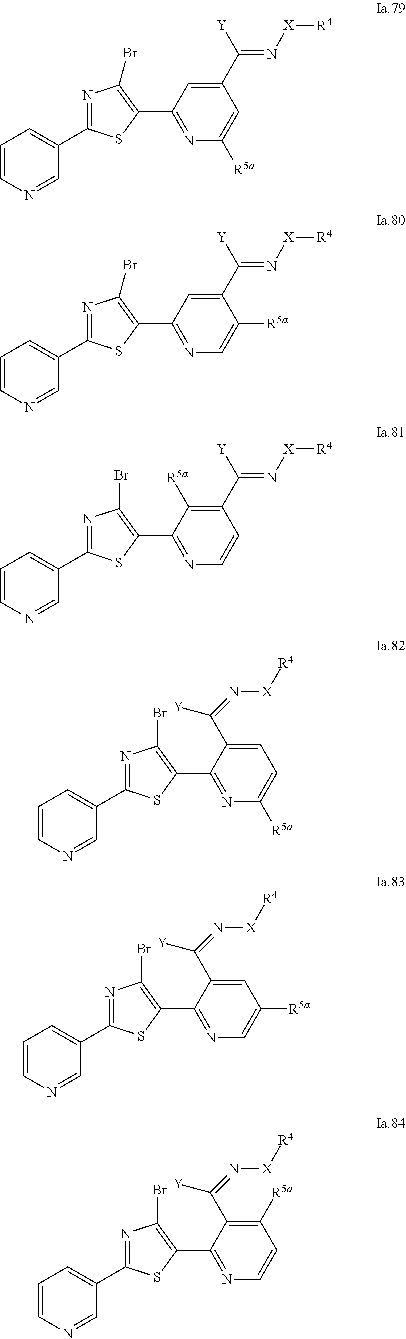

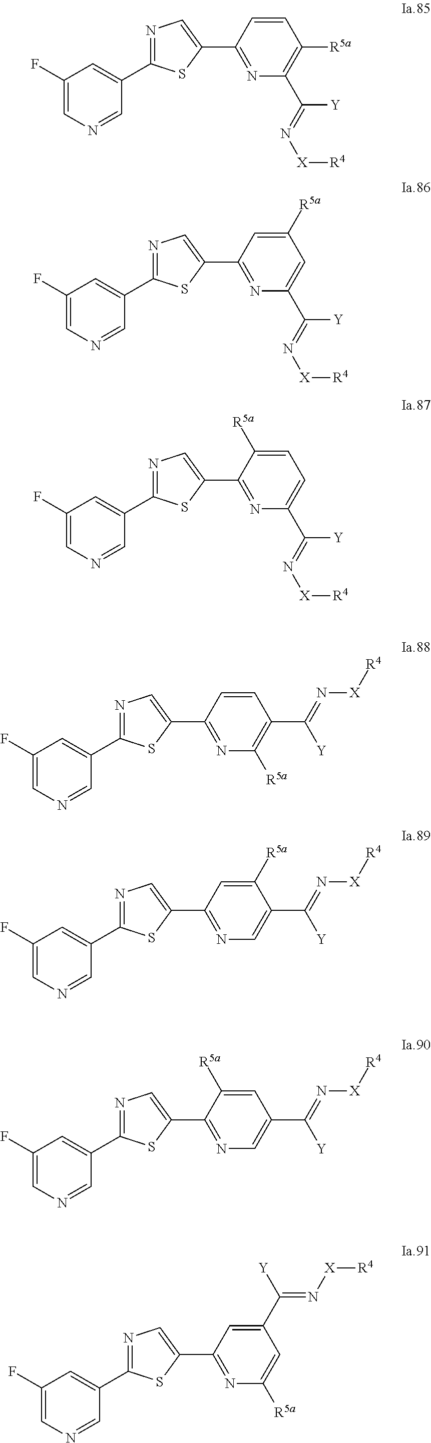

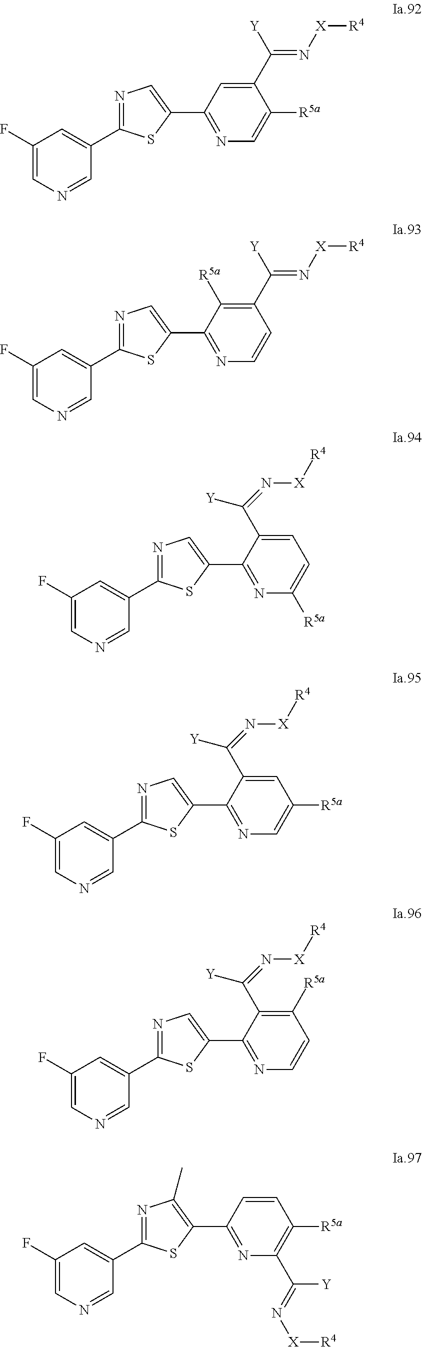

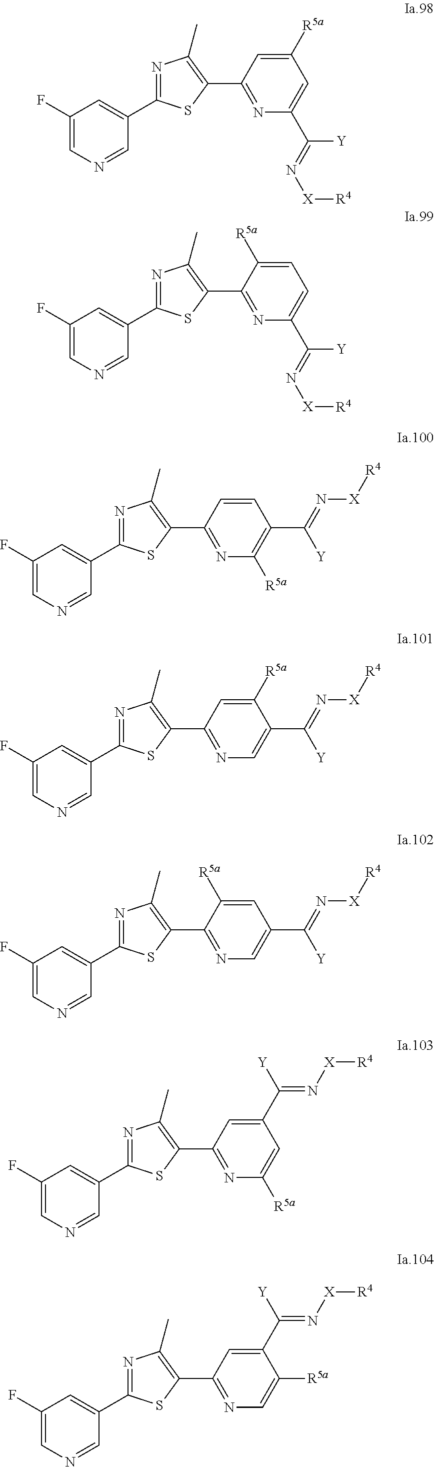

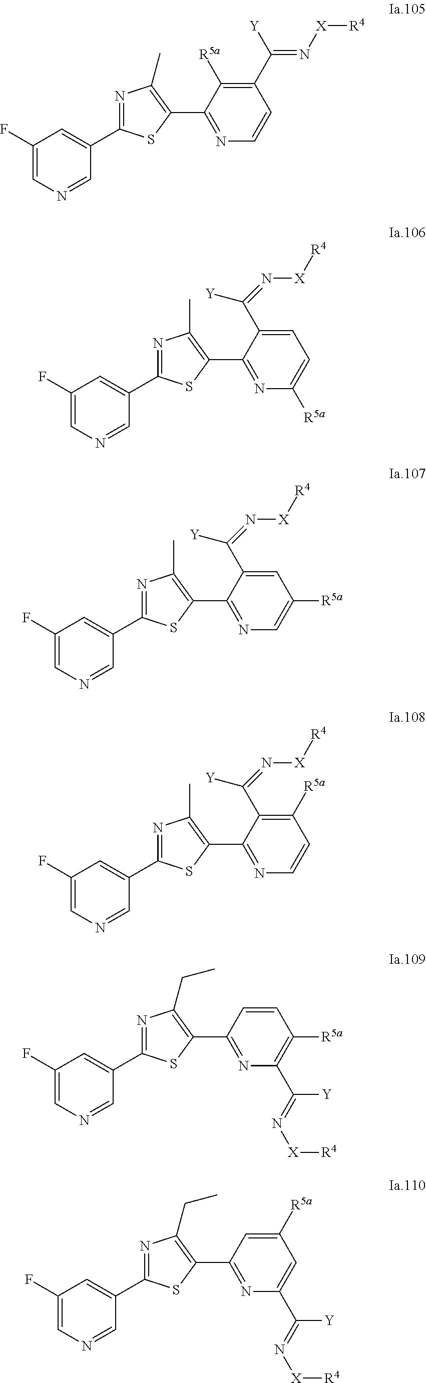

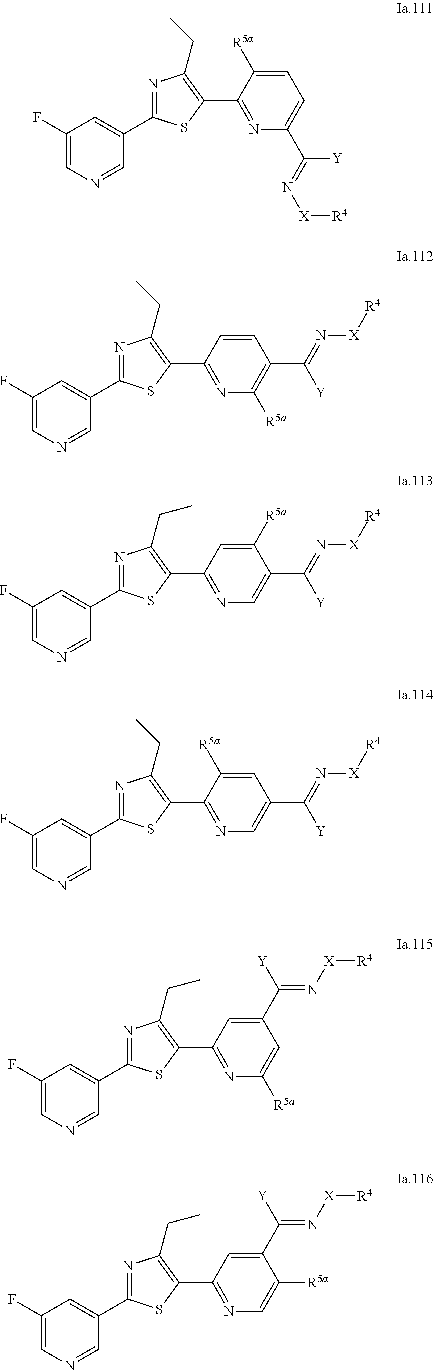

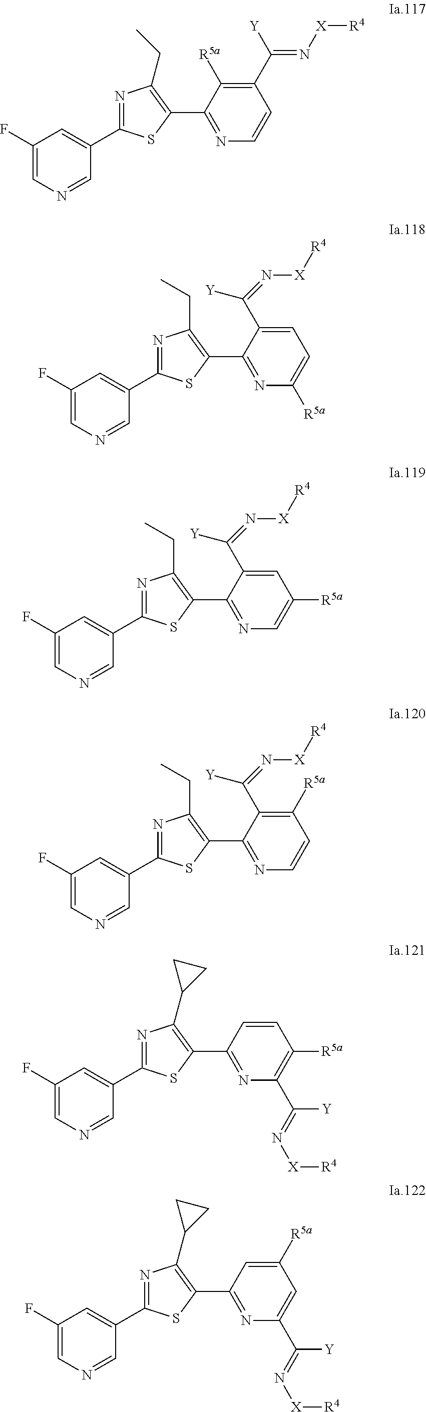

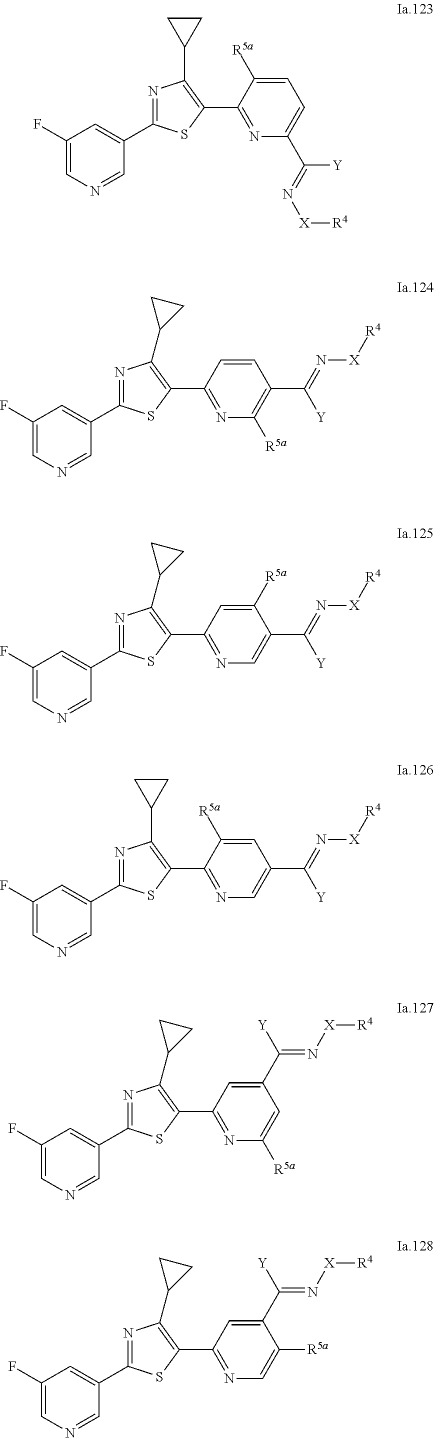

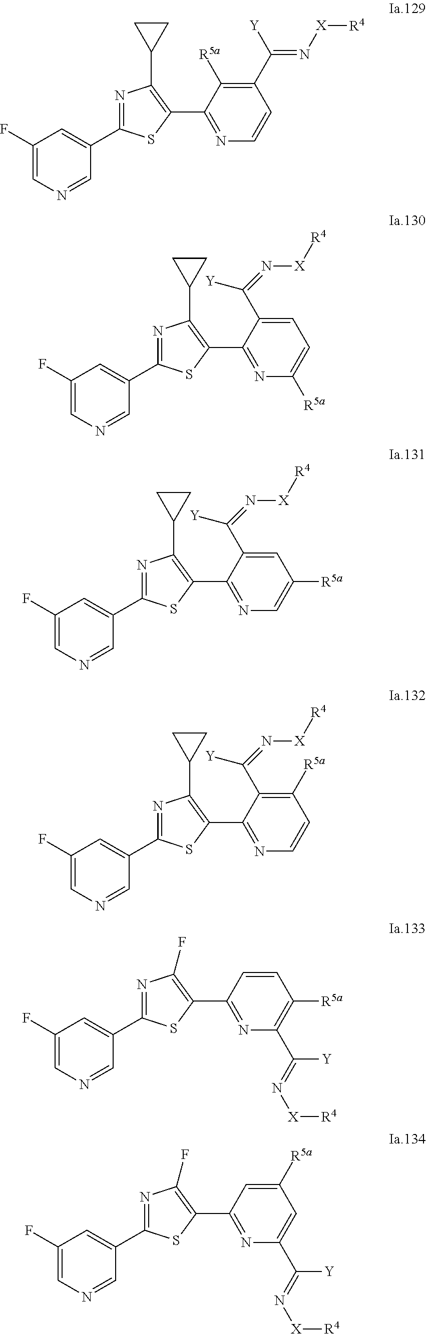

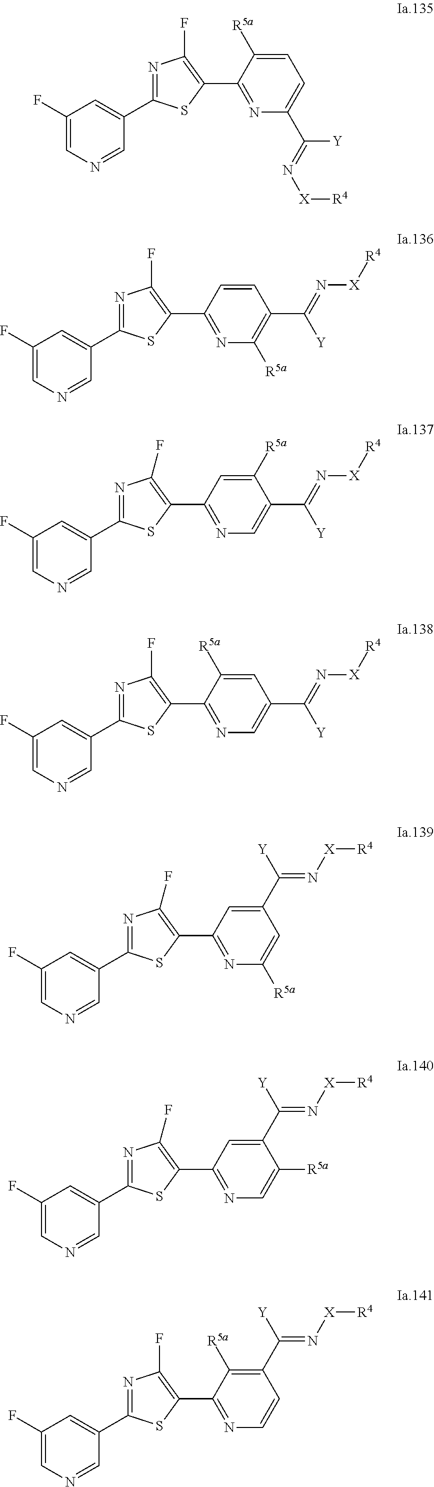

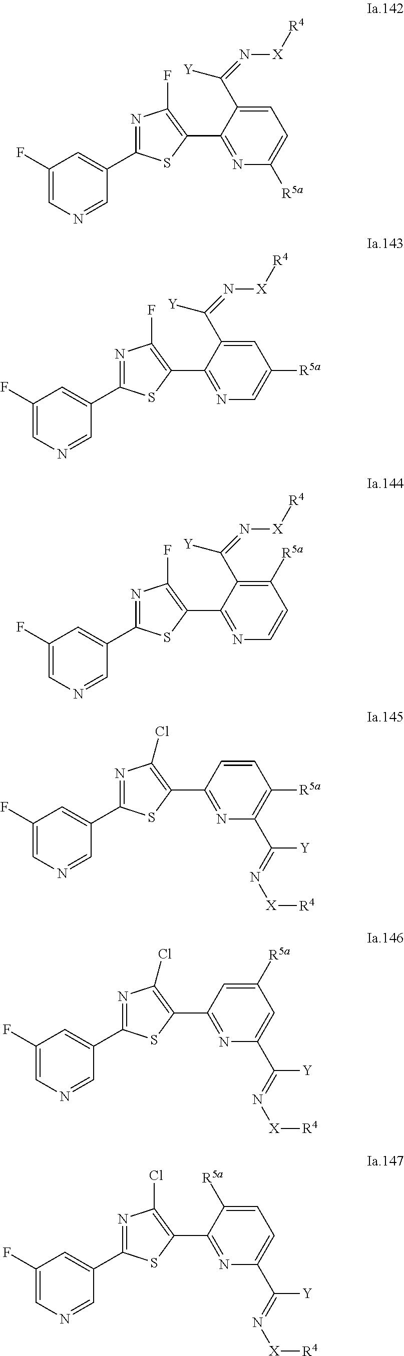

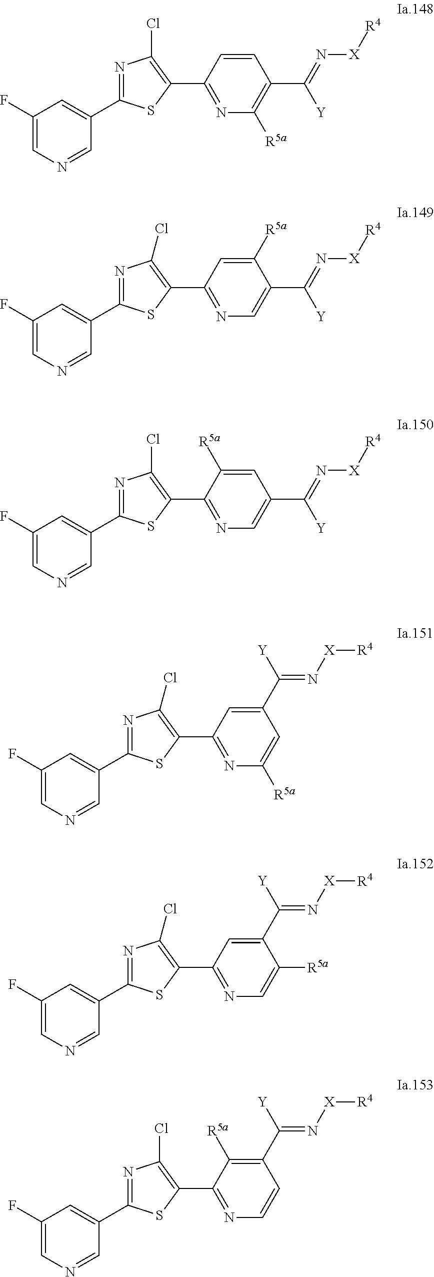

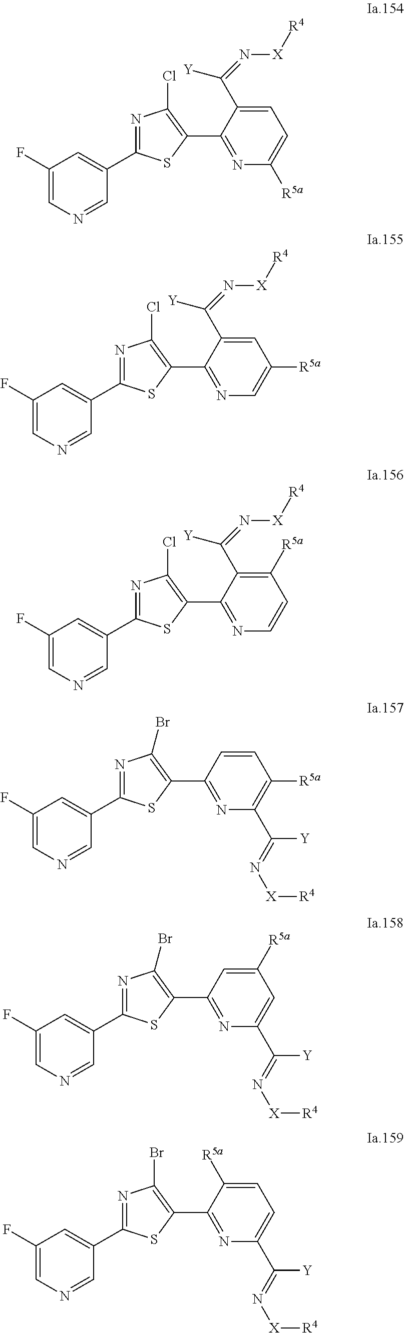

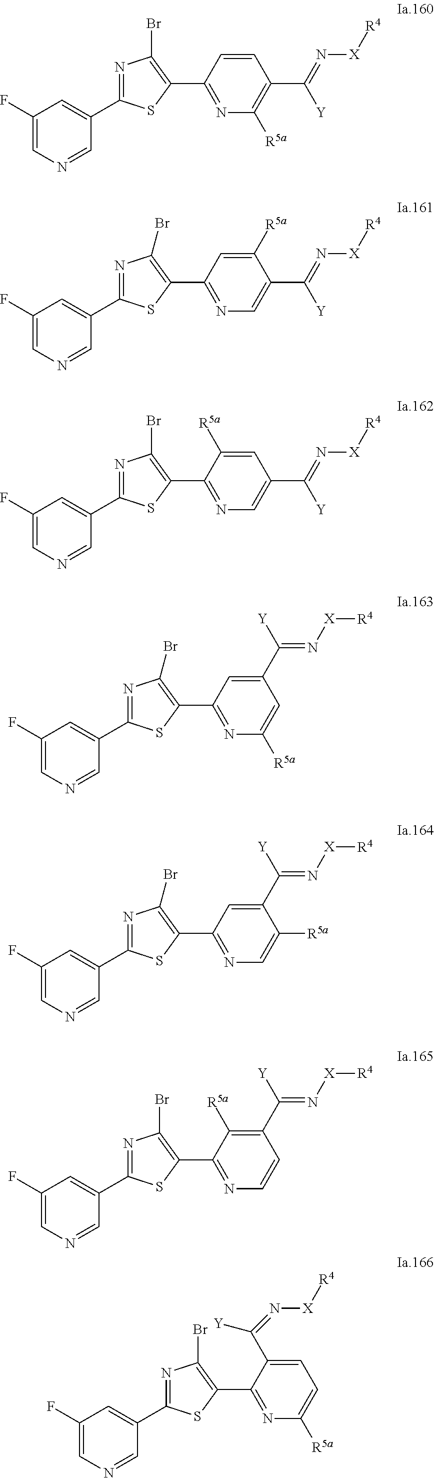

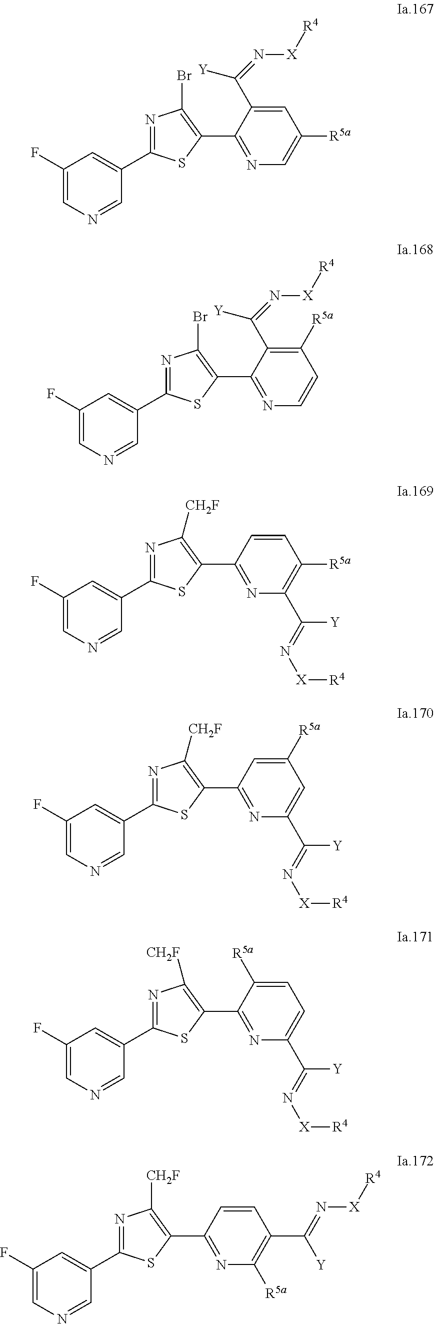

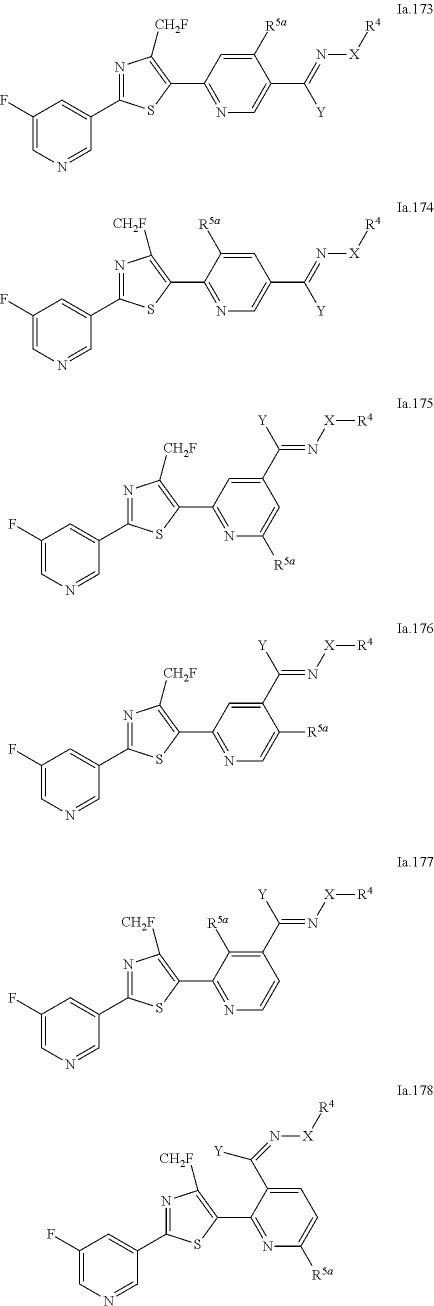

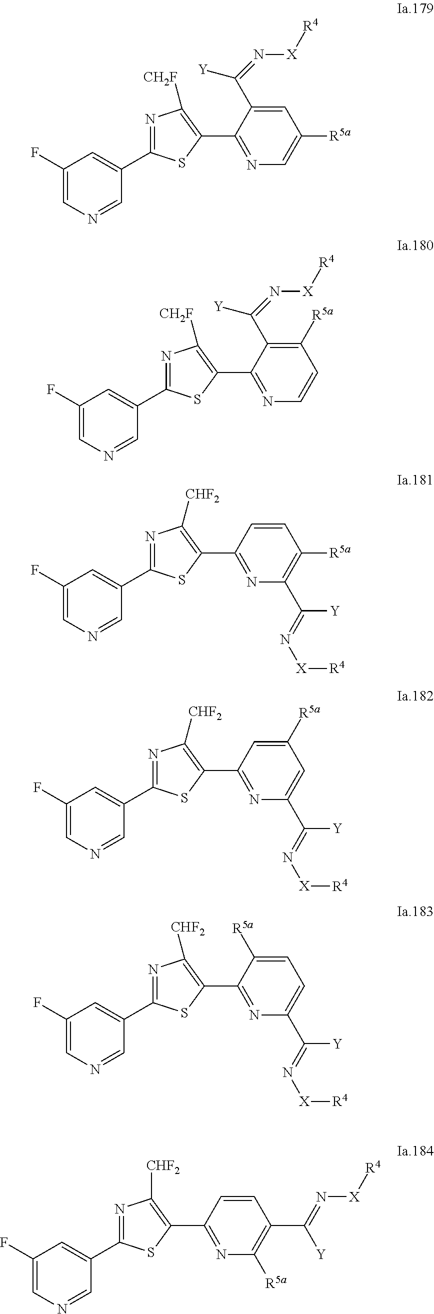

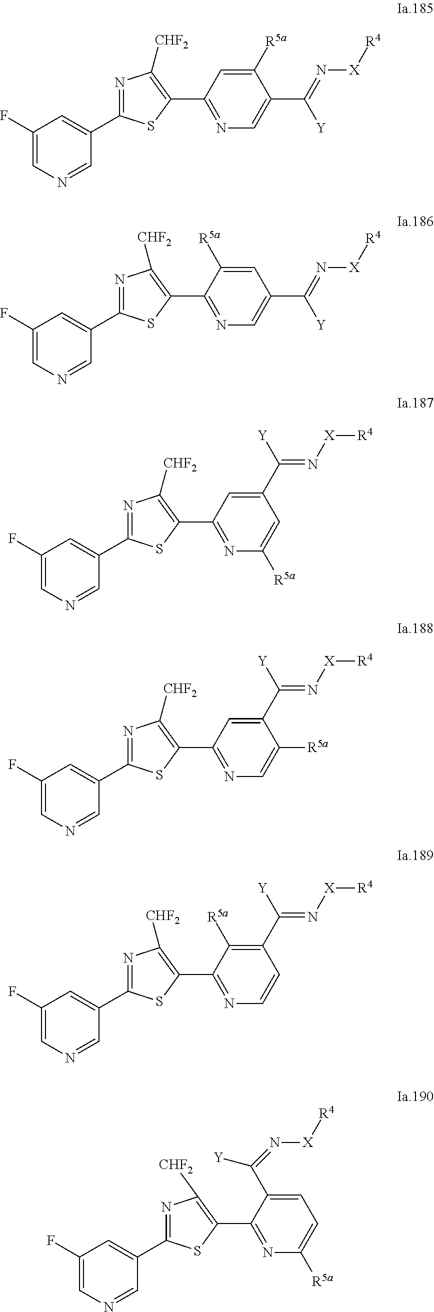

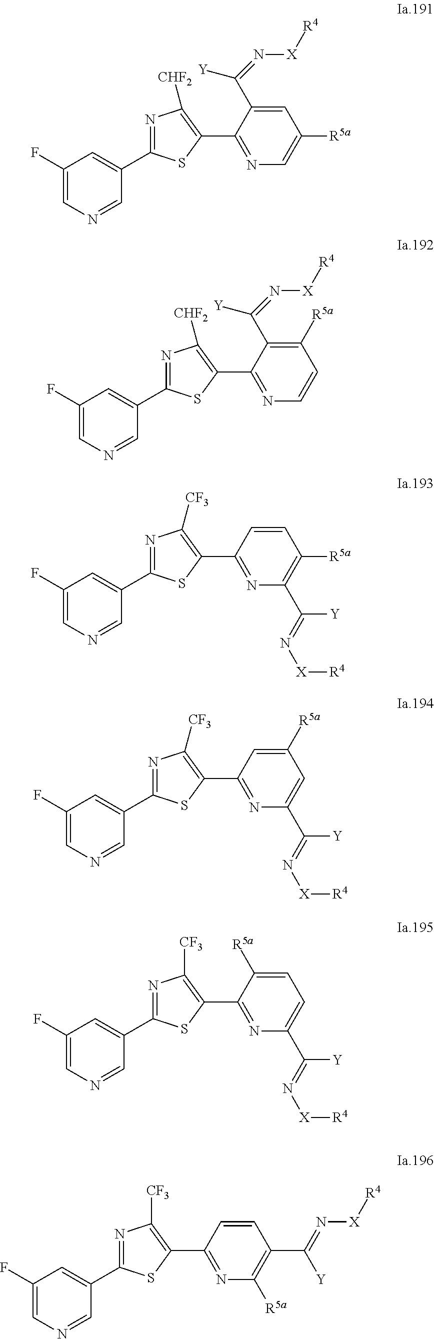

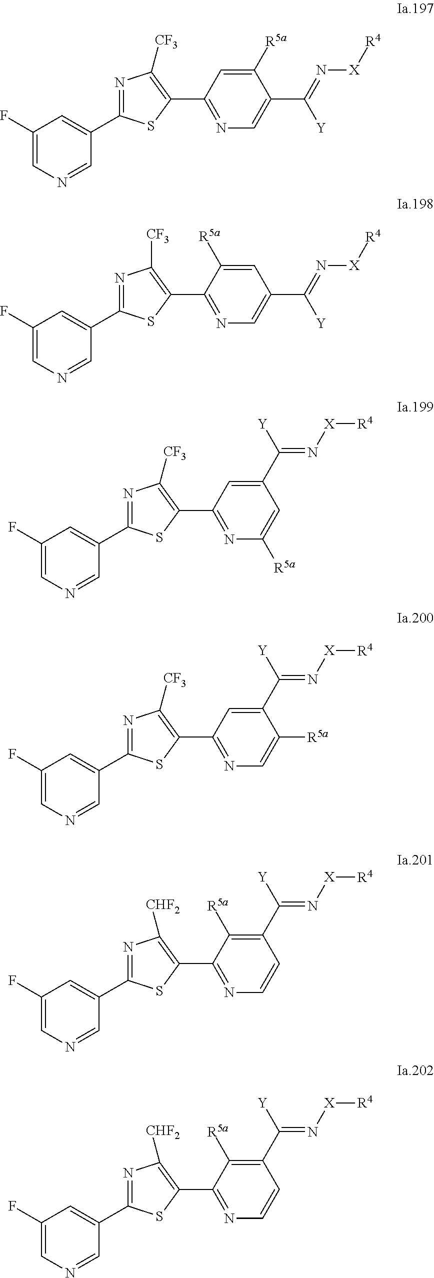

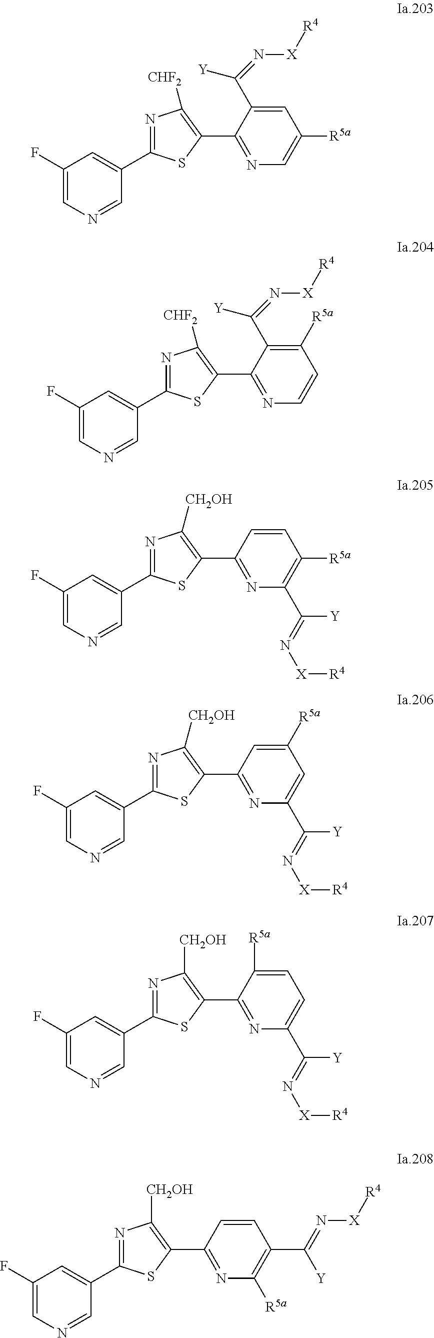

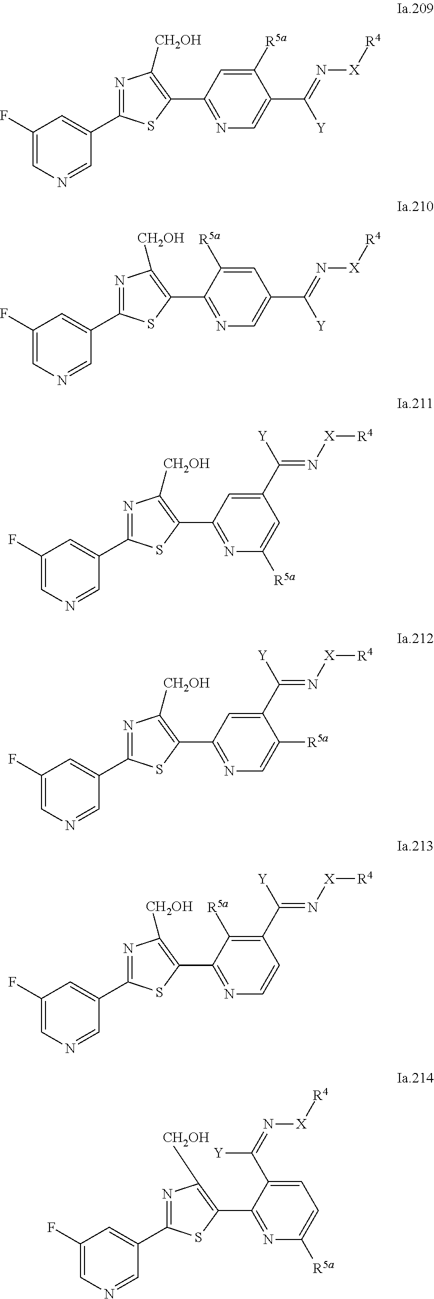

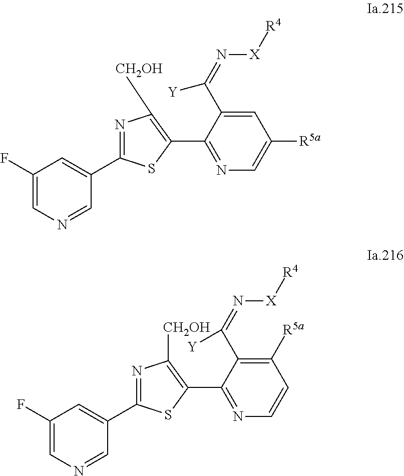

### Table 1

Compounds of the formula Ia.1 in which Y is hydrogen, R^{5a} is hydrogen and the combination of X and R⁴ for a compound corresponds in each case to one row of Table A

### Table 2

Compounds of the formula Ia.1 in which Y is hydrogen, R^{5a} is methyl and the combination of X and R⁴ for a compound corresponds in each case to one row of Table A

### Table 3

Compounds of the formula Ia.1 in which Y is hydrogen, R^{5a} is ethyl and the combination of X and R⁴ for a compound corresponds in each case to one row of Table A

### Table 4

Compounds of the formula Ia.1 in which Y is hydrogen, R^{5a} is F and the combination of X and R⁴ for a compound corresponds in each case to one row of Table A

### Table 5

Compounds of the formula Ia.1 in which Y is hydrogen, R^{5a} is Cl and the combination of X and R⁴ for a compound corresponds in each case to one row of Table A

### Table 6

Compounds of the formula Ia.1 in which Y is hydrogen, R^{5a} is CF₃ and the combination of X and R⁴ for a compound corresponds in each case to one row of Table A

### Table 7

Compounds of the formula Ia.1 in which Y is hydrogen, R^{5a} is OCF₃ and the combination of X and R⁴ for a compound corresponds in each case to one row of Table A

### Table 8

Compounds of the formula Ia.1 in which Y is hydrogen, R^{5a} is SCF₃ and the combination of X and R⁴ for a compound corresponds in each case to one row of Table A

### Table 9

Compounds of the formula Ia.1 in which Y is hydrogen, R^{5a} is pyridin-2-yl and the combination of X and R⁴ for a compound corresponds in each case to one row of Table A

### Table 10

Compounds of the formula Ia.1 in which Y is hydrogen, R^{5a} is pyrimidin-2-yl and the combination of X and R⁴ for a compound corresponds in each case to one row of Table A

### Tables 11 to 20

Compounds of the formula Ia.1 in which R^{5a} is as defined in any of tables 1 to 10, Y is methyl and the combination of X and R⁴ for a compound corresponds in each case to one row of Table A

### Tables 21 to 30

Compounds of the formula Ia.1 in which R^{5a} is as defined in any of tables 1 to 10, Y is ethyl and the combination of X and R⁴ for a compound corresponds in each case to one row of Table A

### Tables 31 to 40

Compounds of the formula Ia.1 in which R^{5a} is as defined in any of tables 1 to 10, Y is isopropyl and the combination of X and R⁴ for a compound corresponds in each case to one row of Table A

### Tables 41 to 50

Compounds of the formula Ia.1 in which R^{5a} is as defined in any of tables 1 to 10, Y is cyclopropyl and the combination of X and R⁴ for a compound corresponds in each case to one row of Table A

### Tables 51 to 60

Compounds of the formula Ia.1 in which R^{5a} is as defined in any of tables 1 to 10, Y is CF₃ and the combination of X and R⁴ for a compound corresponds in each case to one row of Table A

### Tables 61 to 120

Compounds of the formula Ia.2 in which the combination of Y and R^{5a} is as defined in tables 1 to 60, and the combination of X and R⁴ for a compound corresponds in each case to one row of Table A

### Tables 121 to 180

Compounds of the formula Ia.3 in which the combination of Y and R^{5a} is as defined in tables 1 to 60, and the combination of X and R⁴ for a compound corresponds in each case to one row of Table A

### Tables 181 to 240

Compounds of the formula Ia.4 in which the combination of Y and R^{5a} is as defined in tables 1 to 60, and the combination of X and R⁴ for a compound corresponds in each case to one row of Table A

### Tables 241 to 300

Compounds of the formula Ia.5 in which the combination of Y and R^{5a} is as defined in tables 1 to 60, and the combination of X and R⁴ for a compound corresponds in each case to one row of Table A

### Tables 301 to 360

Compounds of the formula Ia.6 in which the combination of Y and R^{5a} is as defined in tables 1 to 60, and the combination of X and R⁴ for a compound corresponds in each case to one row of Table A

### Tables 361 to 420

Compounds of the formula Ia.7 in which the combination of Y and R^{5a} is as defined in tables 1 to 60, and the combination of X and R⁴ for a compound corresponds in each case to one row of Table A

### Tables 421 to 480

Compounds of the formula Ia.8 in which the combination of Y and R^{5a} is as defined in tables 1 to 60, and the combination of X and R⁴ for a compound corresponds in each case to one row of Table A

### Tables 481 to 540

Compounds of the formula Ia.9 in which the combination of Y and R^{5a} is as defined in tables 1 to 60, and the combination of X and R⁴ for a compound corresponds in each case to one row of Table A

### Tables 541 to 600

Compounds of the formula Ia.10 in which the combination of Y and R^{5a} is as defined in tables 1 to 60, and the combination of X and R⁴ for a compound corresponds in each case to one row of Table A

### Tables 601 to 660

Compounds of the formula Ia.11 in which the combination of Y and R^{5a} is as defined in tables 1 to 60, and the combination of X and R⁴ for a compound corresponds in each case to one row of Table A

### Tables 661 to 720

Compounds of the formula Ia.12 in which the combination of Y and R^{5a} is as defined in tables 1 to 60, and the combination of X and R⁴ for a compound corresponds in each case to one row of Table A

### Tables 721 to 780

Compounds of the formula Ia.13 in which the combination of Y and R^{5a} is as defined in tables 1 to 60, and the combination of X and R⁴ for a compound corresponds in each case to one row of Table A

### Tables 781 to 840

Compounds of the formula Ia.14 in which the combination of Y and R^{5a} is as defined in tables 1 to 60, and the combination of X and R⁴ for a compound corresponds in each case to one row of Table A

### Tables 841 to 900

Compounds of the formula Ia.15 in which the combination of Y and R^{5a} is as defined in tables 1 to 60, and the combination of X and R⁴ for a compound corresponds in each case to one row of Table A

### Tables 901 to 960

Compounds of the formula Ia.16 in which the combination of Y and R^{5a} is as defined in tables 1 to 60, and the combination of X and R⁴ for a compound corresponds in each case to one row of Table A

### Tables 961 to 1020

Compounds of the formula Ia.17 in which the combination of Y and R^{5a} is as defined in tables 1 to 60, and the combination of X and R⁴ for a compound corresponds in each case to one row of Table A

### Tables 1021 to 1080

Compounds of the formula Ia.18 in which the combination of Y and R^{5a} is as defined in tables 1 to 60, and the combination of X and R⁴ for a compound corresponds in each case to one row of Table A

### Tables 1081 to 1140

Compounds of the formula Ia.19 in which the combination of Y and R^{5a} is as defined in tables 1 to 60, and the combination of X and R⁴ for a compound corresponds in each case to one row of Table A

### Tables 1141 to 1200

Compounds of the formula Ia.20 in which the combination of Y and R^{5a} is as defined in tables 1 to 60, and the combination of X and R⁴ for a compound corresponds in each case to one row of Table A

### Tables 1201 to 1260

Compounds of the formula Ia.21 in which the combination of Y and R^{5a} is as defined in tables 1 to 60, and the combination of X and R⁴ for a compound corresponds in each case to one row of Table A

### Tables 1261 to 1320

Compounds of the formula Ia.22 in which the combination of Y and R^{5a} is as defined in tables 1 to 60, and the combination of X and R⁴ for a compound corresponds in each case to one row of Table A

### Tables 1321 to 1380

Compounds of the formula Ia.23 in which the combination of Y and R^{5a} is as defined in tables 1 to 60, and the combination of X and R⁴ for a compound corresponds in each case to one row of Table A

### Tables 1381 to 1440

Compounds of the formula Ia.24 in which the combination of Y and R^{5a} is as defined in tables 1 to 60, and the combination of X and R⁴ for a compound corresponds in each case to one row of Table A

### Tables 1441 to 1500

Compounds of the formula Ia.25 in which the combination of Y and R^{5a} is as defined in tables 1 to 60, and the combination of X and R⁴ for a compound corresponds in each case to one row of Table A

### Tables 1501 to 1560

Compounds of the formula Ia.26 in which the combination of Y and R^{5a} is as defined in tables 1 to 60, and the combination of X and R⁴ for a compound corresponds in each case to one row of Table A

### Tables 1561 to 1620

Compounds of the formula Ia.27 in which the combination of Y and R^{5a} is as defined in tables 1 to 60, and the combination of X and R⁴ for a compound corresponds in each case to one row of Table A

### Tables 1621 to 1680

Compounds of the formula Ia.28 in which the combination of Y and R^{5a} is as defined in tables 1 to 60, and the combination of X and R⁴ for a compound corresponds in each case to one row of Table A

### Tables 1681 to 1740

Compounds of the formula Ia.29 in which the combination of Y and R^{5a} is as defined in tables 1 to 60, and the combination of X and R⁴ for a compound corresponds in each case to one row of Table A

### Tables 1741 to 1800

Compounds of the formula Ia.30 in which the combination of Y and R^{5a} is as defined in tables 1 to 60, and the combination of X and R⁴ for a compound corresponds in each case to one row of Table A

### Tables 1801 to 1860

Compounds of the formula Ia.31 in which the combination of Y and R^{5a} is as defined in tables 1 to 60, and the combination of X and R⁴ for a compound corresponds in each case to one row of Table A

### Tables 1861 to 1920

Compounds of the formula Ia.32 in which the combination of Y and R^{5a} is as defined in tables 1 to 60, and the combination of X and R⁴ for a compound corresponds in each case to one row of Table A

### Tables 1921 to 1980

Compounds of the formula Ia.33 in which the combination of Y and R^{5a} is as defined in tables 1 to 60, and the combination of X and R⁴ for a compound corresponds in each case to one row of Table A

### Tables 1981 to 2040

Compounds of the formula Ia.34 in which the combination of Y and R^{5a} is as defined in tables 1 to 60, and the combination of X and R⁴ for a compound corresponds in each case to one row of Table A

### Tables 2041 to 2100

Compounds of the formula Ia.35 in which the combination of Y and R^{5a} is as defined in tables 1 to 60, and the combination of X and R⁴ for a compound corresponds in each case to one row of Table A

### Tables 2101 to 2160

Compounds of the formula Ia.36 in which the combination of Y and R^{5a} is as defined in tables 1 to 60, and the combination of X and R⁴ for a compound corresponds in each case to one row of Table A

### Tables 2161 to 2220

Compounds of the formula Ia.37 in which the combination of Y and R^{5a} is as defined in tables 1 to 60, and the combination of X and R⁴ for a compound corresponds in each case to one row of Table A

### Tables 2221 to 2280

Compounds of the formula Ia.38 in which the combination of Y and R^{5a} is as defined in tables 1 to 60, and the combination of X and R⁴ for a compound corresponds in each case to one row of Table A

### Tables 2281 to 2340

Compounds of the formula Ia.39 in which the combination of Y and R^{5a} is as defined in tables 1 to 60, and the combination of X and R⁴ for a compound corresponds in each case to one row of Table A

### Tables 2341 to 2400

Compounds of the formula Ia.40 in which the combination of Y and R^{5a} is as defined in tables 1 to 60, and the combination of X and R⁴ for a compound corresponds in each case to one row of Table A

### Tables 2401 to 2460

Compounds of the formula Ia.41 in which the combination of Y and R^{5a} is as defined in tables 1 to 60, and the combination of X and R⁴ for a compound corresponds in each case to one row of Table A

### Tables 2461 to 2520

Compounds of the formula Ia.42 in which the combination of Y and R^{5a} is as defined in tables 1 to 60, and the combination of X and R⁴ for a compound corresponds in each case to one row of Table A

### Tables 2521 to 2580

Compounds of the formula Ia.43 in which the combination of Y and R^{5a} is as defined in tables 1 to 60, and the combination of X and R⁴ for a compound corresponds in each case to one row of Table A

### Tables 2581 to 2640

Compounds of the formula Ia.44 in which the combination of Y and R^{5a} is as defined in tables 1 to 60, and the combination of X and R⁴ for a compound corresponds in each case to one row of Table A

### Tables 2641 to 2700

Compounds of the formula Ia.45 in which the combination of Y and R^{5a} is as defined in tables 1 to 60, and the combination of X and R⁴ for a compound corresponds in each case to one row of Table A

### Tables 2701 to 2760

Compounds of the formula Ia.46 in which the combination of Y and R^{5a} is as defined in tables 1 to 60, and the combination of X and R⁴ for a compound corresponds in each case to one row of Table A

### Tables 2761 to 2820

Compounds of the formula Ia.47 in which the combination of Y and R^{5a} is as defined in tables 1 to 60, and the combination of X and R⁴ for a compound corresponds in each case to one row of Table A

### Tables 2821 to 2880

Compounds of the formula Ia.48 in which the combination of Y and R^{5a} is as defined in tables 1 to 60, and the combination of X and R⁴ for a compound corresponds in each case to one row of Table A

### Tables 2881 to 2940

Compounds of the formula Ia.49 in which the combination of Y and R^{5a} is as defined in tables 1 to 60, and the combination of X and R⁴ for a compound corresponds in each case to one row of Table A

### Tables 2941 to 3000

Compounds of the formula Ia.50 in which the combination of Y and R^{5a} is as defined in tables 1 to 60, and the combination of X and R⁴ for a compound corresponds in each case to one row of Table A

### Tables 3001 to 3060

Compounds of the formula Ia.51 in which the combination of Y and R^{5a} is as defined in tables 1 to 60, and the combination of X and R⁴ for a compound corresponds in each case to one row of Table A

### Tables 3061 to 3120

Compounds of the formula Ia.52 in which the combination of Y and R^{5a} is as defined in tables 1 to 60, and the combination of X and R⁴ for a compound corresponds in each case to one row of Table A

### Tables 3121 to 3180

Compounds of the formula Ia.53 in which the combination of Y and R^{5a} is as defined in tables 1 to 60, and the combination of X and R⁴ for a compound corresponds in each case to one row of Table A

### Tables 3181 to 3240

Compounds of the formula Ia.54 in which the combination of Y and R^{5a} is as defined in tables 1 to 60, and the combination of X and R⁴ for a compound corresponds in each case to one row of Table A

### Tables 3241 to 3300

Compounds of the formula Ia.55 in which the combination of Y and R^{5a} is as defined in tables 1 to 60, and the combination of X and R⁴ for a compound corresponds in each case to one row of Table A

### Tables 3301 to 3360

Compounds of the formula Ia.56 in which the combination of Y and R^{5a} is as defined in tables 1 to 60, and the combination of X and R⁴ for a compound corresponds in each case to one row of Table A

### Tables 3361 to 3420

Compounds of the formula Ia.57 in which the combination of Y and R^{5a} is as defined in tables 1 to 60, and the combination of X and R⁴ for a compound corresponds in each case to one row of Table A

### Tables 3421 to 3480

Compounds of the formula Ia.58 in which the combination of Y and R^{5a} is as defined in tables 1 to 60, and the combination of X and R⁴ for a compound corresponds in each case to one row of Table A

### Tables 3481 to 3540

Compounds of the formula Ia.59 in which the combination of Y and R^{5a} is as defined in tables 1 to 60, and the combination of X and R⁴ for a compound corresponds in each case to one row of Table A

### Tables 3541 to 3600

Compounds of the formula Ia.60 in which the combination of Y and R^{5a} is as defined in tables 1 to 60, and the combination of X and R⁴ for a compound corresponds in each case to one row of Table A

### Tables 3601 to 3660

Compounds of the formula Ia.61 in which the combination of Y and R^{5a} is as defined in tables 1 to 60, and the combination of X and R⁴ for a compound corresponds in each case to one row of Table A

### Tables 3661 to 3720

Compounds of the formula Ia.62 in which the combination of Y and R^{5a} is as defined in tables 1 to 60, and the combination of X and R⁴ for a compound corresponds in each case to one row of Table A

### Tables 3721 to 3780

Compounds of the formula Ia.63 in which the combination of Y and R^{5a} is as defined in tables 1 to 60, and the combination of X and R⁴ for a compound corresponds in each case to one row of Table A

### Tables 3781 to 3840

Compounds of the formula Ia.64 in which the combination of Y and R^{5a} is as defined in tables 1 to 60, and the combination of X and R⁴ for a compound corresponds in each case to one row of Table A

### Tables 3841 to 3900

Compounds of the formula Ia.65 in which the combination of Y and R^{5a} is as defined in tables 1 to 60, and the combination of X and R⁴ for a compound corresponds in each case to one row of Table A

### Tables 3901 to 3960

Compounds of the formula Ia.66 in which the combination of Y and R^{5a} is as defined in tables 1 to 60, and the combination of X and R⁴ for a compound corresponds in each case to one row of Table A

### Tables 3961 to 4020

Compounds of the formula Ia.67 in which the combination of Y and R^{5a} is as defined in tables 1 to 60, and the combination of X and R⁴ for a compound corresponds in each case to one row of Table A

### Tables 4021 to 4080

Compounds of the formula Ia.68 in which the combination of Y and R^{5a} is as defined in tables 1 to 60, and the combination of X and R⁴ for a compound corresponds in each case to one row of Table A

### Tables 4081 to 4140

Compounds of the formula Ia.69 in which the combination of Y and R^{5a} is as defined in tables 1 to 60, and the combination of X and R⁴ for a compound corresponds in each case to one row of Table A

### Tables 4141 to 4200

Compounds of the formula Ia.70 in which the combination of Y and R^{5a} is as defined in tables 1 to 60, and the combination of X and R⁴ for a compound corresponds in each case to one row of Table A

### Tables 4201 to 4260

Compounds of the formula Ia.71 in which the combination of Y and R^{5a} is as defined in tables 1 to 60, and the combination of X and R⁴ for a compound corresponds in each case to one row of Table A

### Tables 4261 to 4320

Compounds of the formula Ia.72 in which the combination of Y and R^{5a} is as defined in tables 1 to 60, and the combination of X and R⁴ for a compound corresponds in each case to one row of Table A

### Tables 4321 to 4380

Compounds of the formula Ia.73 in which the combination of Y and R^{5a} is as defined in tables 1 to 60, and the combination of X and R⁴ for a compound corresponds in each case to one row of Table A

### Tables 4381 to 4440

Compounds of the formula Ia.74 in which the combination of Y and R^{5a} is as defined in tables 1 to 60, and the combination of X and R⁴ for a compound corresponds in each case to one row of Table A

### Tables 4441 to 4500

Compounds of the formula Ia.75 in which the combination of Y and R^{5a} is as defined in tables 1 to 60, and the combination of X and R⁴ for a compound corresponds in each case to one row of Table A

### Tables 4501 to 4560

Compounds of the formula Ia.76 in which the combination of Y and R^{5a} is as defined in tables 1 to 60, and the combination of X and R⁴ for a compound corresponds in each case to one row of Table A

### Tables 4561 to 4620

Compounds of the formula Ia.77 in which the combination of Y and R^{5a} is as defined in tables 1 to 60, and the combination of X and R⁴ for a compound corresponds in each case to one row of Table A

### Tables 4621 to 4680

Compounds of the formula Ia.78 in which the combination of Y and R^{5a} is as defined in tables 1 to 60, and the combination of X and R⁴ for a compound corresponds in each case to one row of Table A

### Tables 4681 to 4740

Compounds of the formula Ia.79 in which the combination of Y and R^{5a} is as defined in tables 1 to 60, and the combination of X and R⁴ for a compound corresponds in each case to one row of Table A

### Tables 4741 to 4800

Compounds of the formula Ia.80 in which the combination of Y and R^{5a} is as defined in tables 1 to 60, and the combination of X and R⁴ for a compound corresponds in each case to one row of Table A

### Tables 4801 to 4860

Compounds of the formula Ia.81 in which the combination of Y and R^{5a} is as defined in tables 1 to 60, and the combination of X and R⁴ for a compound corresponds in each case to one row of Table A

### Tables 4861 to 4920

Compounds of the formula Ia.82 in which the combination of Y and R^{5a} is as defined in tables 1 to 60, and the combination of X and R⁴ for a compound corresponds in each case to one row of Table A

### Tables 4921 to 4980

Compounds of the formula Ia.83 in which the combination of Y and R^{5a} is as defined in tables 1 to 60, and the combination of X and R⁴ for a compound corresponds in each case to one row of Table A

### Tables 4981 to 5040

Compounds of the formula Ia.84 in which the combination of Y and R^{5a} is as defined in tables 1 to 60, and the combination of X and R⁴ for a compound corresponds in each case to one row of Table A

### Tables 5041 to 5100

Compounds of the formula Ia.85 in which the combination of Y and R^{5a} is as defined in tables 1 to 60, and the combination of X and R⁴ for a compound corresponds in each case to one row of Table A

### Tables 5101 to 5160

Compounds of the formula Ia.86 in which the combination of Y and R^{5a} is as defined in tables 1 to 60, and the combination of X and R⁴ for a compound corresponds in each case to one row of Table A

### Tables 5161 to 5220

Compounds of the formula Ia.87 in which the combination of Y and R^{5a} is as defined in tables 1 to 60, and the combination of X and R⁴ for a compound corresponds in each case to one row of Table A

### Tables 5221 to 5280

Compounds of the formula Ia.88 in which the combination of Y and R^{5a} is as defined in tables 1 to 60, and the combination of X and R⁴ for a compound corresponds in each case to one row of Table A

### Tables 5280 to 5340

Compounds of the formula Ia.89 in which the combination of Y and R^{5a} is as defined in tables 1 to 60, and the combination of X and R⁴ for a compound corresponds in each case to one row of Table A

### Tables 5341 to 5400

Compounds of the formula Ia.90 in which the combination of Y and R^{5a} is as defined in tables 1 to 60, and the combination of X and R⁴ for a compound corresponds in each case to one row of Table A

### Tables 5401 to 5460

Compounds of the formula Ia.91 in which the combination of Y and R^{5a} is as defined in tables 1 to 60, and the combination of X and R⁴ for a compound corresponds in each case to one row of Table A

### Tables 5461 to 5520

Compounds of the formula Ia.92 in which the combination of Y and R^{5a} is as defined in tables 1 to 60, and the combination of X and R⁴ for a compound corresponds in each case to one row of Table A

### Tables 5521 to 5580

Compounds of the formula Ia.93 in which the combination of Y and R^{5a} is as defined in tables 1 to 60, and the combination of X and R⁴ for a compound corresponds in each case to one row of Table A

### Tables 5581 to 5640

Compounds of the formula Ia.94 in which the combination of Y and R^{5a} is as defined in tables 1 to 60, and the combination of X and R⁴ for a compound corresponds in each case to one row of Table A

### Tables 5641 to 5700

Compounds of the formula Ia.95 in which the combination of Y and R^{5a} is as defined in tables 1 to 60, and the combination of X and R⁴ for a compound corresponds in each case to one row of Table A

### Tables 5701 to 5760

Compounds of the formula Ia.96 in which the combination of Y and R^{5a} is as defined in tables 1 to 60, and the combination of X and R⁴ for a compound corresponds in each case to one row of Table A

### Tables 5761 to 5820

Compounds of the formula Ia.97 in which the combination of Y and R^{5a} is as defined in tables 1 to 60, and the combination of X and R⁴ for a compound corresponds in each case to one row of Table A

### Tables 5821 to 5880

Compounds of the formula Ia.98 in which the combination of Y and R^{5a} is as defined in tables 1 to 60, and the combination of X and R⁴ for a compound corresponds in each case to one row of Table A

### Tables 5881 to 5940

Compounds of the formula Ia.99 in which the combination of Y and R^{5a} is as defined in tables 1 to 60, and the combination of X and R⁴ for a compound corresponds in each case to one row of Table A

### Tables 5941 to 6000

Compounds of the formula Ia.100 in which the combination of Y and R^{5a} is as defined in tables 1 to 60, and the combination of X and R⁴ for a compound corresponds in each case to one row of Table A

### Tables 6001 to 6060

Compounds of the formula Ia.101 in which the combination of Y and R^{5a} is as defined in tables 1 to 60, and the combination of X and R⁴ for a compound corresponds in each case to one row of Table A

### Tables 6061 to 6120

Compounds of the formula Ia.102 in which the combination of Y and R^{5a} is as defined in tables 1 to 60, and the combination of X and R⁴ for a compound corresponds in each case to one row of Table A

### Tables 6121 to 6180

Compounds of the formula Ia.103 in which the combination of Y and R^{5a} is as defined in tables 1 to 60, and the combination of X and R⁴ for a compound corresponds in each case to one row of Table A

### Tables 6181 to 6240

Compounds of the formula Ia.104 in which the combination of Y and R^{5a} is as defined in tables 1 to 60, and the combination of X and R⁴ for a compound corresponds in each case to one row of Table A

### Tables 6241 to 6300

Compounds of the formula Ia.105 in which the combination of Y and R^{5a} is as defined in tables 1 to 60, and the combination of X and R⁴ for a compound corresponds in each case to one row of Table A

### Tables 6301 to 6360

Compounds of the formula Ia.106 in which the combination of Y and R^{5a} is as defined in tables 1 to 60, and the combination of X and R⁴ for a compound corresponds in each case to one row of Table A

### Tables 6361 to 6420

Compounds of the formula Ia.107 in which the combination of Y and R^{5a} is as defined in tables 1 to 60, and the combination of X and R⁴ for a compound corresponds in each case to one row of Table A

### Tables 6421 to 6480

Compounds of the formula Ia.108 in which the combination of Y and R^{5a} is as defined in tables 1 to 60, and the combination of X and R⁴ for a compound corresponds in each case to one row of Table A

### Tables 6481 to 6540

Compounds of the formula Ia.109 in which the combination of Y and R^{5a} is as defined in tables 1 to 60, and the combination of X and R⁴ for a compound corresponds in each case to one row of Table A

### Tables 6541 to 6600

Compounds of the formula Ia.110 in which the combination of Y and R^{5a} is as defined in tables 1 to 60, and the combination of X and R⁴ for a compound corresponds in each case to one row of Table A

### Tables 6601 to 6660

Compounds of the formula Ia.111 in which the combination of Y and R^{5a} is as defined in tables 1 to 60, and the combination of X and R⁴ for a compound corresponds in each case to one row of Table A

### Tables 6661 to 6720

Compounds of the formula Ia.112 in which the combination of Y and R^{5a} is as defined in tables 1 to 60, and the combination of X and R⁴ for a compound corresponds in each case to one row of Table A

### Tables 6721 to 6780

Compounds of the formula Ia.113 in which the combination of Y and R^{5a} is as defined in tables 1 to 60, and the combination of X and R⁴ for a compound corresponds in each case to one row of Table A

### Tables 6781 to 6840

Compounds of the formula Ia.114 in which the combination of Y and R^{5a} is as defined in tables 1 to 60, and the combination of X and R⁴ for a compound corresponds in each case to one row of Table A

### Tables 6841 to 6900

Compounds of the formula Ia.115 in which the combination of Y and R^{5a} is as defined in tables 1 to 60, and the combination of X and R⁴ for a compound corresponds in each case to one row of Table A

### Tables 6901 to 6960

Compounds of the formula Ia.116 in which the combination of Y and R^{5a} is as defined in tables 1 to 60, and the combination of X and R⁴ for a compound corresponds in each case to one row of Table A

### Tables 6961 to 7020

Compounds of the formula Ia.117 in which the combination of Y and R^{5a} is as defined in tables 1 to 60, and the combination of X and R⁴ for a compound corresponds in each case to one row of Table A

### Tables 7021 to 7080

Compounds of the formula Ia.118 in which the combination of Y and R^{5a} is as defined in tables 1 to 60, and the combination of X and R⁴ for a compound corresponds in each case to one row of Table A

### Tables 7081 to 7140

Compounds of the formula Ia.119 in which the combination of Y and R^{5a} is as defined in tables 1 to 60, and the combination of X and R⁴ for a compound corresponds in each case to one row of Table A

### Tables 7141 to 7200

Compounds of the formula Ia.120 in which the combination of Y and R^{5a} is as defined in tables 1 to 60, and the combination of X and R⁴ for a compound corresponds in each case to one row of Table A

### Tables 7201 to 7260

Compounds of the formula Ia.121 in which the combination of Y and R^{5a} is as defined in tables 1 to 60, and the combination of X and R⁴ for a compound corresponds in each case to one row of Table A

### Tables 7261 to 7320

Compounds of the formula Ia.122 in which the combination of Y and R^{5a} is as defined in tables 1 to 60, and the combination of X and R⁴ for a compound corresponds in each case to one row of Table A

### Tables 7321 to 7380

Compounds of the formula Ia.123 in which the combination of Y and R^{5a} is as defined in tables 1 to 60, and the combination of X and R⁴ for a compound corresponds in each case to one row of Table A

### Tables 7381 to 7440

Compounds of the formula Ia.124 in which the combination of Y and R^{5a} is as defined in tables 1 to 60, and the combination of X and R⁴ for a compound corresponds in each case to one row of Table A

### Tables 7441 to 7500

Compounds of the formula Ia.125 in which the combination of Y and R^{5a} is as defined in tables 1 to 60, and the combination of X and R⁴ for a compound corresponds in each case to one row of Table A

### Tables 7501 to 7560

Compounds of the formula Ia.126 in which the combination of Y and R^{5a} is as defined in tables 1 to 60, and the combination of X and R⁴ for a compound corresponds in each case to one row of Table A

### Tables 7561 to 7620

Compounds of the formula Ia.127 in which the combination of Y and R^{5a} is as defined in tables 1 to 60, and the combination of X and R⁴ for a compound corresponds in each case to one row of Table A

### Tables 7621 to 7680

Compounds of the formula Ia.128 in which the combination of Y and R^{5a} is as defined in tables 1 to 60, and the combination of X and R⁴ for a compound corresponds in each case to one row of Table A

### Tables 7681 to 7740

Compounds of the formula Ia.129 in which the combination of Y and R^{5a} is as defined in tables 1 to 60, and the combination of X and R⁴ for a compound corresponds in each case to one row of Table A

### Tables 7741 to 7800

Compounds of the formula Ia.130 in which the combination of Y and R^{5a} is as defined in tables 1 to 60, and the combination of X and R⁴ for a compound corresponds in each case to one row of Table A

### Tables 7801 to 7860

Compounds of the formula Ia.131 in which the combination of Y and R^{5a} is as defined in tables 1 to 60, and the combination of X and R⁴ for a compound corresponds in each case to one row of Table A

### Tables 7861 to 7920

Compounds of the formula Ia.132 in which the combination of Y and R^{5a} is as defined in tables 1 to 60, and the combination of X and R⁴ for a compound corresponds in each case to one row of Table A

### Tables 7921 to 7980

Compounds of the formula Ia.133 in which the combination of Y and R^{5a} is as defined in tables 1 to 60, and the combination of X and R⁴ for a compound corresponds in each case to one row of Table A

### Tables 7981 to 8040

Compounds of the formula Ia.134 in which the combination of Y and R^{5a} is as defined in tables 1 to 60, and the combination of X and R⁴ for a compound corresponds in each case to one row of Table A

### Tables 8041 to 8100

Compounds of the formula Ia.135 in which the combination of Y and R^{5a} is as defined in tables 1 to 60, and the combination of X and R⁴ for a compound corresponds in each case to one row of Table A

### Tables 8101 to 8160

Compounds of the formula Ia.136 in which the combination of Y and R^{5a} is as defined in tables 1 to 60, and the combination of X and R⁴ for a compound corresponds in each case to one row of Table A

### Tables 8161 to 8220

Compounds of the formula Ia.137 in which the combination of Y and R^{5a} is as defined in tables 1 to 60, and the combination of X and R⁴ for a compound corresponds in each case to one row of Table A

### Tables 8221 to 8280

Compounds of the formula Ia.138 in which the combination of Y and R^{5a} is as defined in tables 1 to 60, and the combination of X and R⁴ for a compound corresponds in each case to one row of Table A

### Tables 8281 to 8340

Compounds of the formula Ia.139 in which the combination of Y and R^{5a} is as defined in tables 1 to 60, and the combination of X and R⁴ for a compound corresponds in each case to one row of Table A

### Tables 8341 to 8400

Compounds of the formula Ia.140 in which the combination of Y and R^{5a} is as defined in tables 1 to 60, and the combination of X and R⁴ for a compound corresponds in each case to one row of Table A

### Tables 8401 to 8460

Compounds of the formula Ia.141 in which the combination of Y and R^{5a} is as defined in tables 1 to 60, and the combination of X and R⁴ for a compound corresponds in each case to one row of Table A

### Tables 8461 to 8520

Compounds of the formula Ia.142 in which the combination of Y and R^{5a} is as defined in tables 1 to 60, and the combination of X and R⁴ for a compound corresponds in each case to one row of Table A

### Tables 8521 to 8580

Compounds of the formula Ia.143 in which the combination of Y and R^{5a} is as defined in tables 1 to 60, and the combination of X and R⁴ for a compound corresponds in each case to one row of Table A

### Tables 8581 to 8640

Compounds of the formula Ia.144 in which the combination of Y and R^{5a} is as defined in tables 1 to 60, and the combination of X and R⁴ for a compound corresponds in each case to one row of Table A

### Tables 8641 to 8700

Compounds of the formula Ia.145 in which the combination of Y and R^{5a} is as defined in tables 1 to 60, and the combination of X and R⁴ for a compound corresponds in each case to one row of Table A

### Tables 8701 to 8760

Compounds of the formula Ia.146 in which the combination of Y and R^{5a} is as defined in tables 1 to 60, and the combination of X and R⁴ for a compound corresponds in each case to one row of Table A

### Tables 8761 to 8820

Compounds of the formula Ia.147 in which the combination of Y and R^{5a} is as defined in tables 1 to 60, and the combination of X and R⁴ for a compound corresponds in each case to one row of Table A

### Tables 8821 to 8880

Compounds of the formula Ia.148 in which the combination of Y and R^{5a} is as defined in tables 1 to 60, and the combination of X and R⁴ for a compound corresponds in each case to one row of Table A

### Tables 8881 to 8940

Compounds of the formula Ia.149 in which the combination of Y and R^{5a} is as defined in tables 1 to 60, and the combination of X and R⁴ for a compound corresponds in each case to one row of Table A

### Tables 8941 to 9000

Compounds of the formula Ia.150 in which the combination of Y and R^{5a} is as defined in tables 1 to 60, and the combination of X and R⁴ for a compound corresponds in each case to one row of Table A

### Tables 9001 to 9060

Compounds of the formula Ia.151 in which the combination of Y and R^{5a} is as defined in tables 1 to 60, and the combination of X and R⁴ for a compound corresponds in each case to one row of Table A

### Tables 9061 to 9120

Compounds of the formula Ia.152 in which the combination of Y and R^{5a} is as defined in tables 1 to 60, and the combination of X and R⁴ for a compound corresponds in each case to one row of Table A

### Tables 9121 to 9180

Compounds of the formula Ia.153 in which the combination of Y and R^{5a} is as defined in tables 1 to 60, and the combination of X and R⁴ for a compound corresponds in each case to one row of Table A

### Tables 9181 to 9240

Compounds of the formula Ia.154 in which the combination of Y and R^{5a} is as defined in tables 1 to 60, and the combination of X and R⁴ for a compound corresponds in each case to one row of Table A

### Tables 9241 to 9300

Compounds of the formula Ia.155 in which the combination of Y and R^{5a} is as defined in tables 1 to 60, and the combination of X and R⁴ for a compound corresponds in each case to one row of Table A

### Tables 9301 to 9360

Compounds of the formula Ia.156 in which the combination of Y and R^{5a} is as defined in tables 1 to 60, and the combination of X and R⁴ for a compound corresponds in each case to one row of Table A

### Tables 9361 to 9420

Compounds of the formula Ia.157 in which the combination of Y and R^{5a} is as defined in tables 1 to 60, and the combination of X and R⁴ for a compound corresponds in each case to one row of Table A

### Tables 9421 to 9480

Compounds of the formula Ia.158 in which the combination of Y and R^{5a} is as defined in tables 1 to 60, and the combination of X and R⁴ for a compound corresponds in each case to one row of Table A

### Tables 9481 to 9540

Compounds of the formula Ia.159 in which the combination of Y and R^{5a} is as defined in tables 1 to 60, and the combination of X and R⁴ for a compound corresponds in each case to one row of Table A

### Tables 9541 to 9600

Compounds of the formula Ia.160 in which the combination of Y and R^{5a} is as defined in tables 1 to 60, and the combination of X and R⁴ for a compound corresponds in each case to one row of Table A

### Tables 9601 to 9660

Compounds of the formula Ia.161 in which the combination of Y and R^{5a} is as defined in tables 1 to 60, and the combination of X and R⁴ for a compound corresponds in each case to one row of Table A

### Tables 9661 to 9720

Compounds of the formula Ia.162 in which the combination of Y and R^{5a} is as defined in tables 1 to 60, and the combination of X and R⁴ for a compound corresponds in each case to one row of Table A

### Tables 9721 to 9780

Compounds of the formula Ia.163 in which the combination of Y and R^{5a} is as defined in tables 1 to 60, and the combination of X and R⁴ for a compound corresponds in each case to one row of Table A

### Tables 9781 to 9840

Compounds of the formula Ia.164 in which the combination of Y and R^{5a} is as defined in tables 1 to 60, and the combination of X and R⁴ for a compound corresponds in each case to one row of Table A

### Tables 9841 to 9900

Compounds of the formula Ia.165 in which the combination of Y and R^{5a} is as defined in tables 1 to 60, and the combination of X and R⁴ for a compound corresponds in each case to one row of Table A

### Tables 9901 to 9960

Compounds of the formula Ia.166 in which the combination of Y and R^{5a} is as defined in tables 1 to 60, and the combination of X and R⁴ for a compound corresponds in each case to one row of Table A

### Tables 9961 to 10020

Compounds of the formula Ia.167 in which the combination of Y and R^{5a} is as defined in tables 1 to 60, and the combination of X and R⁴ for a compound corresponds in each case to one row of Table A

### Tables 10021 to 10080

Compounds of the formula Ia.168 in which the combination of Y and R^{5a} is as defined in tables 1 to 60, and the combination of X and R⁴ for a compound corresponds in each case to one row of Table A

### Tables 10081 to 10140

Compounds of the formula Ia.169 in which the combination of Y and R^{5a} is as defined in tables 1 to 60, and the combination of X and R⁴ for a compound corresponds in each case to one row of Table A

### Tables 10141 to 10200

Compounds of the formula Ia.170 in which the combination of Y and R^{5a} is as defined in tables 1 to 60, and the combination of X and R⁴ for a compound corresponds in each case to one row of Table A

### Tables 10201 to 10260

Compounds of the formula Ia.171 in which the combination of Y and R^{5a} is as defined in tables 1 to 60, and the combination of X and R⁴ for a compound corresponds in each case to one row of Table A

### Tables 10261 to 10320

Compounds of the formula Ia.172 in which the combination of Y and R^{5a} is as defined in tables 1 to 60, and the combination of X and R⁴ for a compound corresponds in each case to one row of Table A

### Tables 10321 to 10380

Compounds of the formula Ia.173 in which the combination of Y and R^{5a} is as defined in tables 1 to 60, and the combination of X and R⁴ for a compound corresponds in each case to one row of Table A

### Tables 10381 to 10440

Compounds of the formula Ia.174 in which the combination of Y and R^{5a} is as defined in tables 1 to 60, and the combination of X and R⁴ for a compound corresponds in each case to one row of Table A

### Tables 10441 to 10500

Compounds of the formula Ia.175 in which the combination of Y and R^{5a} is as defined in tables 1 to 60, and the combination of X and R⁴ for a compound corresponds in each case to one row of Table A

### Tables 10501 to 10560

Compounds of the formula Ia.176 in which the combination of Y and R^{5a} is as defined in tables 1 to 60, and the combination of X and R⁴ for a compound corresponds in each case to one row of Table A

### Tables 10561 to 10620

Compounds of the formula Ia.177 in which the combination of Y and R^{5a} is as defined in tables 1 to 60, and the combination of X and R⁴ for a compound corresponds in each case to one row of Table A

### Tables 10621 to 10680

Compounds of the formula Ia.178 in which the combination of Y and R^{5a} is as defined in tables 1 to 60, and the combination of X and R⁴ for a compound corresponds in each case to one row of Table A

### Tables 10681 to 10740

Compounds of the formula Ia.179 in which the combination of Y and R^{5a} is as defined in tables 1 to 60, and the combination of X and R⁴ for a compound corresponds in each case to one row of Table A

### Tables 10741 to 10800

Compounds of the formula Ia.180 in which the combination of Y and R^{5a} is as defined in tables 1 to 60, and the combination of X and R⁴ for a compound corresponds in each case to one row of Table A

### Tables 10801 to 10860

Compounds of the formula Ia.181 in which the combination of Y and R^{5a} is as defined in tables 1 to 60, and the combination of X and R⁴ for a compound corresponds in each case to one row of Table A

### Tables 10861 to 10920

Compounds of the formula Ia.182 in which the combination of Y and R^{5a} is as defined in tables 1 to 60, and the combination of X and R⁴ for a compound corresponds in each case to one row of Table A

### Tables 10921 to 10980

Compounds of the formula Ia.183 in which the combination of Y and R^{5a} is as defined in tables 1 to 60, and the combination of X and R⁴ for a compound corresponds in each case to one row of Table A

### Tables 10981 to 11040

Compounds of the formula Ia.184 in which the combination of Y and R^{5a} is as defined in tables 1 to 60, and the combination of X and R⁴ for a compound corresponds in each case to one row of Table A

### Tables 11041 to 11100

Compounds of the formula Ia.185 in which the combination of Y and R^{5a} is as defined in tables 1 to 60, and the combination of X and R⁴ for a compound corresponds in each case to one row of Table A

### Tables 11101 to 11160

Compounds of the formula Ia.186 in which the combination of Y and R^{5a} is as defined in tables 1 to 60, and the combination of X and R⁴ for a compound corresponds in each case to one row of Table A

### Tables 11161 to 11220

Compounds of the formula Ia.187 in which the combination of Y and R^{5a} is as defined in tables 1 to 60, and the combination of X and R⁴ for a compound corresponds in each case to one row of Table A

### Tables 11221 to 11280

Compounds of the formula Ia.188 in which the combination of Y and R^{5a} is as defined in tables 1 to 60, and the combination of X and R⁴ for a compound corresponds in each case to one row of Table A

### Tables 11281 to 11340

Compounds of the formula Ia.189 in which the combination of Y and R^{5a} is as defined in tables 1 to 60, and the combination of X and R⁴ for a compound corresponds in each case to one row of Table A

### Tables 11341 to 11400

Compounds of the formula Ia.190 in which the combination of Y and R^{5a} is as defined in tables 1 to 60, and the combination of X and R⁴ for a compound corresponds in each case to one row of Table A

### Tables 11401 to 11460

Compounds of the formula Ia.191 in which the combination of Y and R^{5a} is as defined in tables 1 to 60, and the combination of X and R⁴ for a compound corresponds in each case to one row of Table A

### Tables 11461 to 11520

Compounds of the formula Ia.192 in which the combination of Y and R^{5a} is as defined in tables 1 to 60, and the combination of X and R⁴ for a compound corresponds in each case to one row of Table A

### Tables 11521 to 11580

Compounds of the formula Ia.193 in which the combination of Y and R^{5a} is as defined in tables 1 to 60, and the combination of X and R⁴ for a compound corresponds in each case to one row of Table A

### Tables 11581 to 11640

Compounds of the formula Ia.194 in which the combination of Y and R^{5a} is as defined in tables 1 to 60, and the combination of X and R⁴ for a compound corresponds in each case to one row of Table A

### Tables 11641 to 11700

Compounds of the formula Ia.195 in which the combination of Y and R^{5a} is as defined in tables 1 to 60, and the combination of X and R⁴ for a compound corresponds in each case to one row of Table A

### Tables 11701 to 11760

Compounds of the formula Ia.196 in which the combination of Y and R^{5a} is as defined in tables 1 to 60, and the combination of X and R⁴ for a compound corresponds in each case to one row of Table A

### Tables 11761 to 11820

Compounds of the formula Ia.197 in which the combination of Y and R^{5a} is as defined in tables 1 to 60, and the combination of X and R⁴ for a compound corresponds in each case to one row of Table A

### Tables 11821 to 11880

Compounds of the formula Ia.198 in which the combination of Y and R^{5a} is as defined in tables 1 to 60, and the combination of X and R⁴ for a compound corresponds in each case to one row of Table A

### Tables 11881 to 11940

Compounds of the formula Ia.199 in which the combination of Y and R^{5a} is as defined in tables 1 to 60, and the combination of X and R⁴ for a compound corresponds in each case to one row of Table A

### Tables 11941 to 12000

Compounds of the formula Ia.200 in which the combination of Y and R^{5a} is as defined in tables 1 to 60, and the combination of X and R⁴ for a compound corresponds in each case to one row of Table A

### Tables 12001 to 12060

Compounds of the formula Ia.201 in which the combination of Y and R^{5a} is as defined in tables 1 to 60, and the combination of X and R⁴ for a compound corresponds in each case to one row of Table A

### Tables 12061 to 12120

Compounds of the formula Ia.202 in which the combination of Y and R^{5a} is as defined in tables 1 to 60, and the combination of X and R⁴ for a compound corresponds in each case to one row of Table A

### Tables 12121 to 12180

Compounds of the formula Ia.203 in which the combination of Y and R^{5a} is as defined in tables 1 to 60, and the combination of X and R⁴ for a compound corresponds in each case to one row of Table A

### Tables 12181 to 12240

Compounds of the formula Ia.204 in which the combination of Y and R^{5a} is as defined in tables 1 to 60, and the combination of X and R⁴ for a compound corresponds in each case to one row of Table A

### Tables 12241 to 12300

Compounds of the formula Ia.205 in which the combination of Y and R^{5a} is as defined in tables 1 to 60, and the combination of X and R⁴ for a compound corresponds in each case to one row of Table A

### Tables 12301 to 12360

Compounds of the formula Ia.206 in which the combination of Y and R^{5a} is as defined in tables 1 to 60, and the combination of X and R⁴ for a compound corresponds in each case to one row of Table A

### Tables 12361 to 12420

Compounds of the formula Ia.207 in which the combination of Y and R^{5a} is as defined in tables 1 to 60, and the combination of X and R⁴ for a compound corresponds in each case to one row of Table A

### Tables 12421 to 12480

Compounds of the formula Ia.208 in which the combination of Y and R^{5a} is as defined in tables 1 to 60, and the combination of X and R⁴ for a compound corresponds in each case to one row of Table A

### Tables 12481 to 12540

Compounds of the formula Ia.209 in which the combination of Y and R^{5a} is as defined in tables 1 to 60, and the combination of X and R⁴ for a compound corresponds in each case to one row of Table A

### Tables 12541 to 12600

Compounds of the formula Ia.210 in which the combination of Y and R^{5a} is as defined in tables 1 to 60, and the combination of X and R⁴ for a compound corresponds in each case to one row of Table A

### Tables 12601 to 12660

Compounds of the formula Ia.211 in which the combination of Y and R^{5a} is as defined in tables 1 to 60, and the combination of X and R⁴ for a compound corresponds in each case to one row of Table A

### Tables 12661 to 12720

Compounds of the formula Ia.212 in which the combination of Y and R^{5a} is as defined in tables 1 to 60, and the combination of X and R⁴ for a compound corresponds in each case to one row of Table A

### Tables 12721 to 12780

Compounds of the formula Ia.213 in which the combination of Y and R^{5a} is as defined in tables 1 to 60, and the combination of X and R⁴ for a compound corresponds in each case to one row of Table A

### Tables 12781 to 12840

Compounds of the formula Ia.214 in which the combination of Y and R^{5a} is as defined in tables 1 to 60, and the combination of X and R⁴ for a compound corresponds in each case to one row of Table A

### Tables 12841 to 12900

Compounds of the formula Ia.215 in which the combination of Y and R^{5a} is as defined in tables 1 to 60, and the combination of X and R⁴ for a compound corresponds in each case to one row of Table A

### Tables 12901 to 12960

Compounds of the formula Ia.216 in which the combination of Y and R^{5a} is as defined in tables 1 to 60, and the combination of X and R⁴ for a compound corresponds in each case to one row of Table A

TABLE A

| No. | X | R⁴ |
|---|---|---|
| A-1 | O | H |
| A-2 | O | methyl |
| A-3 | O | ethyl |
| A-4 | O | isopropyl |
| A-5 | O | CH₂CF₃ |
| A-6 | O | (CH₂)₂CF₃ |
| A-7 | O | CH₂-c-propyl |
| A-8 | O | allyl |
| A-9 | O | propargyl |
| A-10 | O | phenyl |
| A-11 | O | benzyl |
| A-12 | O | (CH₂)₂OC₂H₅ |
| A-13 | NH | H |
| A-14 | NH | methyl |
| A-15 | NH | ethyl |
| A-16 | NH | isopropyl |
| A-17 | NH | CH₂CF₃ |
| A-18 | NH | (CH₂)₂CF₃ |
| A-19 | NH | CH₂-c-propyl |
| A-20 | NH | allyl |
| A-21 | NH | propargyl |
| A-22 | NH | phenyl |
| A-23 | NH | 2-fluorophenyl |
| A-24 | NH | 3-fluorophenyl |
| A-25 | NH | 4-fluorophenyl |
| A-26 | NH | 2,3-difluorophenyl |
| A-27 | NH | 2,4-difluorophenyl |
| A-28 | NH | 2,5-difluorophenyl |
| A-29 | NH | 2,6-difluorophenyl |
| A-30 | NH | 3,4-difluorophenyl |
| A-31 | NH | 3,5-difluorophenyl |
| A-32 | NH | 3,4,5-trifluorophenyl |
| A-33 | NH | 2,4,6-trifluorophenyl |
| A-34 | NH | 2,3,5,6-tetrafluorophenyl |
| A-35 | NH | 2-chlorophenyl |
| A-36 | NH | 3-chlorophenyl |
| A-37 | NH | 4-chlorophenyl |
| A-38 | NH | 3,4-dichlorophenyl |
| A-39 | NH | 3,5-dichlorophenyl |
| A-40 | NH | 3,4,5-trichlorophenyl |
| A-41 | NH | 2,4,6-trichlorophenyl |
| A-42 | NH | 3-chloro-4-fluorophenyl |
| A-43 | NH | 4-chloro-3-fluorophenyl |
| A-44 | NH | 2-cyanophenyl |
| A-45 | NH | 3-cyanophenyl |
| A-46 | NH | 4-cyanophenyl |
| A-47 | NH | 2-methylphenyl |
| A-48 | NH | 3-methylphenyl |
| A-49 | NH | 4-methylphenyl |
| A-50 | NH | 2,3-dimethylphenyl |
| A-51 | NH | 2,4-dimethylphenyl |
| A-52 | NH | 2,5-dimethylphenyl |
| A-53 | NH | 2,6-dimethylphenyl |
| A-54 | NH | 3,4-dimethylphenyl |
| A-55 | NH | 3,5-dimethylphenyl |
| A-56 | NH | 3,4,5-trimethylphenyl |
| A-57 | NH | 2,4,6-trimethylphenyl |
| A-58 | NH | 2-ethylphenyl |
| A-59 | NH | 3-ethylphenyl |
| A-60 | NH | 4-ethylphenyl |
| A-61 | NH | 2-(trifluoro)methylphenyl |
| A-62 | NH | 3-(trifluoro)methylphenyl |
| A-63 | NH | 4-(trifluoro)methylphenyl |
| A-64 | NH | 2-methoxyphenyl |
| A-65 | NH | 3-methoxyphenyl |
| A-66 | NH | 4-methoxyphenyl |
| A-67 | NH | 2-(trifluoromethoxy)phenyl |
| A-68 | NH | 3-(trifluoromethoxy)phenyl |
| A-69 | NH | 4-(trifluoromethoxy)phenyl |
| A-70 | NH | 2,6-difluoro-4-(trifluoromethyl)phenyl |
| A-71 | NH | 2,6-dichloro-4-(trifluoromethyl)phenyl |
| A-72 | NH | 2-pyridyl |
| A-73 | NH | 4-chloro-2-pyridyl |
| A-74 | NH | 5-chloro-2-pyridyl |
| A-75 | NH | 6-chloro-2-pyridyl |
| A-76 | NH | 1,3-benzodioxol-5-yl |
| A-77 | NH | 1-naphthyl |
| A-78 | NH | 2-naphthyl |
| A-79 | NH | 4,5-dihydro-1H-imidazol-2-yl |
| A-80 | NH | 2,3-dihydro-1,4-benzodioxin-6-yl |
| A-81 | NH | benzyl |
| A-82 | NH | C(O)CH₃ |
| A-83 | NH | C(O)C₂H₅ |
| A-84 | NH | C(O)CH(CH₃)₂ |
| A-85 | NH | C(O)-c-propyl |
| A-86 | NH | C(O)OCH₃ |
| A-87 | NH | C(O)OC₂H₅ |
| A-88 | NH | C(O)NHCH₃ |
| A-89 | NH | C(O)N(CH₃)₂ |
| A-90 | NH | C(O)NHCH₂CF₃ |
| A-91 | NH | C(O)NHC₆H₅ |
| A-92 | NH | C(S)CH₃ |
| A-93 | NH | C(S)C₂H₅ |
| A-94 | NH | C(S)CH(CH₃)₂ |
| A-95 | NH | C(S)-c-propyl |
| A-96 | NH | C(S)OCH₃ |
| A-97 | NH | C(S)OC₂H₅ |
| A-98 | NH | C(S)NHCH₃ |
| A-99 | NH | C(S)N(CH₃)₂ |
| A-100 | NH | C(S)NHCH₂CF₃ |
| A-101 | NH | C(S)NHC₆H₅ |
| A-102 | NH | SO₂—CH₃ |
| A-103 | NCH₃ | H |
| A-104 | NCH₃ | methyl |
| A-105 | NCH₃ | ethyl |
| A-106 | NCH₃ | isopropyl |
| A-107 | NCH₃ | CH₂CF₃ |
| A-108 | NCH₃ | (CH₂)₂CF₃ |
| A-109 | NCH₃ | CH₂-c-propyl |
| A-110 | NCH₃ | allyl |
| A-111 | NCH₃ | propargyl |
| A-112 | NCH₃ | phenyl |
| A-113 | NCH₃ | 2-fluorophenyl |
| A-114 | NCH₃ | 3-fluorophenyl |
| A-115 | NCH₃ | 4-fluorophenyl |
| A-116 | NCH₃ | 2,3-difluorophenyl |
| A-117 | NCH₃ | 2,4-difluorophenyl |
| A-118 | NCH₃ | 2,5-difluorophenyl |
| A-119 | NCH₃ | 2,6-difluorophenyl |
| A-120 | NCH₃ | 3,4-difluorophenyl |
| A-121 | NCH₃ | 3,5-difluorophenyl |
| A-122 | NCH₃ | 3,4,5-trifluorophenyl |
| A-123 | NCH₃ | 2,4,6-trifluorophenyl |
| A-124 | NCH₃ | 2,3,5,6-tetrafluorophenyl |
| A-125 | NCH₃ | 2-chlorophenyl |
| A-126 | NCH₃ | 3-chlorophenyl |
| A-127 | NCH₃ | 4-chlorophenyl |
| A-128 | NCH₃ | 3,4-dichlorophenyl |
| A-129 | NCH₃ | 3,5-dichlorophenyl |
| A-130 | NCH₃ | 3,4,5-trichlorophenyl |
| A-131 | NCH₃ | 2,4,6-trichlorophenyl |
| A-132 | NCH₃ | 3-chloro-4-fluorophenyl |
| A-133 | NCH₃ | 4-chloro-3-fluorophenyl |
| A-134 | NCH₃ | 2-cyanophenyl |
| A-135 | NCH₃ | 3-cyanophenyl |
| A-136 | NCH₃ | 4-cyanophenyl |
| A-137 | NCH₃ | 2-methylphenyl |
| A-138 | NCH₃ | 3-methylphenyl |
| A-139 | NCH₃ | 4-methylphenyl |
| A-140 | NCH₃ | 2,3-dimethylphenyl |
| A-141 | NCH₃ | 2,4-dimethylphenyl |
| A-142 | NCH₃ | 2,5-dimethylphenyl |
| A-143 | NCH₃ | 2,6-dimethylphenyl |
| A-144 | NCH₃ | 3,4-dimethylphenyl |
| A-145 | NCH₃ | 3,5-dimethylphenyl |
| A-146 | NCH₃ | 3,4,5-trimethylphenyl |
| A-147 | NCH₃ | 2,4,6-trimethylphenyl |
| A-148 | NCH₃ | 2-ethylphenyl |
| A-149 | NCH₃ | 3-ethylphenyl |
| A-150 | NCH₃ | 4-ethylphenyl |
| A-151 | NCH₃ | 2-(trifluoro)methylphenyl |
| A-152 | NCH₃ | 3-(trifluoro)methylphenyl |
| A-153 | NCH₃ | 4-(trifluoro)methylphenyl |
| A-154 | NCH₃ | 2-methoxyphenyl |
| A-155 | NCH₃ | 3-methoxyphenyl |
| A-156 | NCH₃ | 4-methoxyphenyl |
| A-157 | NCH₃ | 2-(trifluoromethoxy)phenyl |
| A-158 | NCH₃ | 3-(trifluoromethoxy)phenyl |
| A-159 | NCH₃ | 4-(trifluoromethoxy)phenyl |
| A-160 | NCH₃ | 2,6-difluoro-4-(trifluoromethyl)phenyl |
| A-161 | NCH₃ | 2,6-dichloro-4-(trifluoromethyl)phenyl |
| A-162 | NCH₃ | 2-pyridyl |
| A-163 | NCH₃ | 4-chloro-2-pyridyl |
| A-164 | NCH₃ | 5-chloro-2-pyridyl |
| A-165 | NCH₃ | 6-chloro-2-pyridyl |
| A-166 | NCH₃ | 1,3-benzodioxol-5-yl |
| A-167 | NCH₃ | 1-naphthyl |
| A-168 | NCH₃ | 2-naphthyl |
| A-169 | NCH₃ | 4,5-dihydro-1H-imidazol-2-yl |
| A-170 | NCH₃ | 2,3-dihydro-1,4-benzodioxin-6-yl |
| A-171 | NCH₃ | benzyl |
| A-172 | NCH₃ | C(O)CH₃ |
| A-173 | NCH₃ | C(O)C₂H₅ |
| A-174 | NCH₃ | C(O)CH(CH₃)₂ |
| A-175 | NCH₃ | C(O)-c-propyl |
| A-176 | NCH₃ | C(O)OCH₃ |
| A-177 | NCH₃ | C(O)OC₂H₅ |
| A-178 | NCH₃ | C(O)NHCH₃ |
| A-179 | NCH₃ | C(O)N(CH₃)₂ |
| A-180 | NCH₃ | C(O)NHCH₂CF₃ |
| A-181 | NCH₃ | C(O)NHC₆H₅ |
| A-182 | NCH₃ | C(S)CH₃ |
| A-183 | NCH₃ | C(S)C₂H₅ |
| A-184 | NCH₃ | C(S)CH(CH₃)₂ |
| A-185 | NCH₃ | C(S)-c-propyl |
| A-186 | NCH₃ | C(S)OCH₃ |
| A-187 | NCH₃ | C(S)OC₂H₅ |
| A-188 | NCH₃ | C(S)NHCH₃ |
| A-189 | NCH₃ | C(S)N(CH₃)₂ |
| A-190 | NCH₃ | C(S)NHCH₂CF₃ |
| A-191 | NCH₃ | C(S)NHC₆H₅ |
| A-192 | NCH₃ | SO₂—CH₃ |
| A-193 | NCH₂CH₃ | H |
| A-194 | NCH₂CH₃ | methyl |
| A-195 | NCH₂CH₃ | ethyl |
| A-196 | NCH₂CH₃ | isopropyl |
| A-197 | NCH₂CH₃ | CH₂CF₃ |
| A-198 | NCH₂CH₃ | (CH₂)₂CF₃ |
| A-199 | NCH₂CH₃ | CH₂-c-propyl |
| A-200 | NCH₂CH₃ | allyl |
| A-201 | NCH₂CH₃ | propargyl |
| A-202 | NCH₂CH₃ | phenyl |
| A-203 | NCH₂CH₃ | benzyl |
| A-204 | NCH₂CH₃ | C(O)CH₃ |
| A-205 | NCH₂CH₃ | C(O)C₂H₅ |
| A-206 | NCH₂CH₃ | C(O)CH(CH₃)₂ |
| A-207 | NCH₂CH₃ | C(O)-c-propyl |
| A-208 | NCH₂CH₃ | C(O)OCH₃ |
| A-209 | NCH₂CH₃ | C(O)OC₂H₅ |
| A-210 | NCH₂CH₃ | C(O)NHCH₃ |
| A-211 | NCH₂CH₃ | C(O)N(CH₃)₂ |
| A-212 | NCH₂CH₃ | C(O)NHCH₂CF₃ |
| A-213 | NCH₂CH₃ | C(O)NHC₆H₅ |
| A-214 | NCH₂CH₃ | C(S)CH₃ |
| A-215 | NCH₂CH₃ | C(S)C₂H₅ |
| A-216 | NCH₂CH₃ | C(S)CH(CH₃)₂ |
| A-217 | NCH₂CH₃ | C(S)-c-propyl |
| A-218 | NCH₂CH₃ | C(S)OCH₃ |
| A-219 | NCH₂CH₃ | C(S)OC₂H₅ |
| A-220 | NCH₂CH₃ | C(S)NHCH₃ |
| A-221 | NCH₂CH₃ | C(S)N(CH₃)₂ |
| A-222 | NCH₂CH₃ | C(S)NHCH₂CF₃ |
| A-223 | NCH₂CH₃ | C(S)NHC₆H₅ |
| A-224 | N(CH₂)₂CH₃ | H |
| A-225 | N(CH₂)₂CH₃ | methyl |
| A-226 | N(CH₂)₂CH₃ | ethyl |
| A-227 | N(CH₂)₂CH₃ | isopropyl |
| A-228 | N(CH₂)₂CH₃ | CH₂CF₃ |
| A-229 | N(CH₂)₂CH₃ | (CH₂)₂CF₃ |
| A-230 | N(CH₂)₂CH₃ | CH₂-c-propyl |
| A-231 | N(CH₂)₂CH₃ | allyl |
| A-232 | N(CH₂)₂CH₃ | propargyl |
| A-233 | N(CH₂)₂CH₃ | phenyl |
| A-234 | N(CH₂)₂CH₃ | benzyl |
| A-235 | N(CH₂)₂CH₃ | C(O)CH₃ |
| A-236 | N(CH₂)₂CH₃ | C(O)C₂H₅ |
| A-237 | N(CH₂)₂CH₃ | C(O)CH(CH₃)₂ |
| A-238 | N(CH₂)₂CH₃ | C(O)-c-propyl |
| A-239 | N(CH₂)₂CH₃ | C(O)OCH₃ |
| A-240 | N(CH₂)₂CH₃ | C(O)OC₂H₅ |
| A-241 | N(CH₂)₂CH₃ | C(O)NHCH₃ |
| A-242 | N(CH₂)₂CH₃ | C(O)N(CH₃)₂ |
| A-243 | N(CH₂)₂CH₃ | C(O)NHCH₂CF₃ |
| A-244 | N(CH₂)₂CH₃ | C(O)NHC₆H₅ |
| A-245 | N(CH₂)₂CH₃ | C(S)CH₃ |
| A-246 | N(CH₂)₂CH₃ | C(S)C₂H₅ |
| A-247 | N(CH₂)₂CH₃ | C(S)CH(CH₃)₂ |
| A-248 | N(CH₂)₂CH₃ | C(S)-c-propyl |
| A-249 | N(CH₂)₂CH₃ | C(S)OCH₃ |
| A-250 | N(CH₂)₂CH₃ | C(S)OC₂H₅ |
| A-251 | N(CH₂)₂CH₃ | C(S)NHCH₃ |
| A-252 | N(CH₂)₂CH₃ | C(S)N(CH₃)₂ |
| A-253 | N(CH₂)₂CH₃ | C(S)NHCH₂CF₃ |
| A-254 | N(CH₂)₂CH₃ | C(S)NHC₆H₅ |
| A-255 | N(CH₂)₃CH₃ | H |
| A-256 | N(CH₂)₃CH₃ | methyl |
| A-257 | N(CH₂)₃CH₃ | ethyl |
| A-258 | N(CH₂)₃CH₃ | isopropyl |
| A-259 | N(CH₂)₃CH₃ | CH₂CF₃ |
| A-260 | N(CH₂)₃CH₃ | (CH₂)₂CF₃ |
| A-261 | N(CH₂)₃CH₃ | CH₂-c-propyl |
| A-262 | N(CH₂)₃CH₃ | allyl |
| A-263 | N(CH₂)₃CH₃ | propargyl |
| A-264 | N(CH₂)₃CH₃ | phenyl |
| A-265 | N(CH₂)₃CH₃ | benzyl |
| A-266 | N(CH₂)₃CH₃ | C(O)CH₃ |
| A-267 | N(CH₂)₃CH₃ | C(O)C₂H₅ |
| A-268 | N(CH₂)₃CH₃ | C(O)CH(CH₃)₂ |
| A-269 | N(CH₂)₃CH₃ | C(O)-c-propyl |
| A-270 | N(CH₂)₃CH₃ | C(O)OCH₃ |
| A-271 | N(CH₂)₃CH₃ | C(O)OC₂H₅ |
| A-272 | N(CH₂)₃CH₃ | C(O)NHCH₃ |
| A-273 | N(CH₂)₃CH₃ | C(O)N(CH₃)₂ |
| A-274 | N(CH₂)₃CH₃ | C(O)NHCH₂CF₃ |
| A-275 | N(CH₂)₃CH₃ | C(O)NHC₆H₅ |
| A-276 | N(CH₂)₃CH₃ | C(S)CH₃ |
| A-277 | N(CH₂)₃CH₃ | C(S)C₂H₅ |
| A-278 | N(CH₂)₃CH₃ | C(S)CH(CH₃)₂ |
| A-279 | N(CH₂)₃CH₃ | C(S)-c-propyl |
| A-280 | N(CH₂)₃CH₃ | C(S)OCH₃ |
| A-281 | N(CH₂)₃CH₃ | C(S)OC₂H₅ |
| A-282 | N(CH₂)₃CH₃ | C(S)NHCH₃ |
| A-283 | N(CH₂)₃CH₃ | C(S)N(CH₃)₂ |
| A-284 | N(CH₂)₃CH₃ | C(S)NHCH₂CF₃ |
| A-285 | N(CH₂)₃CH₃ | C(S)NHC₆H₅ |
| A-286 | NC₆H₅ | H |
| A-287 | NC₆H₅ | methyl |
| A-288 | NC₆H₅ | ethyl |
| A-289 | NC₆H₅ | isopropyl |
| A-290 | NC₆H₅ | CH₂CF₃ |
| A-291 | NC₆H₅ | (CH₂)₂CF₃ |
| A-292 | NC₆H₅ | CH₂-c-propyl |
| A-293 | NC₆H₅ | allyl |
| A-294 | NC₆H₅ | propargyl |
| A-295 | NC₆H₅ | phenyl |
| A-296 | NC₆H₅ | benzyl |
| A-297 | NC₆H₅ | C(O)CH₃ |
| A-298 | NC₆H₅ | C(O)C₂H₅ |
| A-299 | NC₆H₅ | C(O)CH(CH₃)₂ |
| A-300 | NC₆H₅ | C(O)-c-propyl |
| A-301 | NC₆H₅ | C(O)OCH₃ |
| A-302 | NC₆H₅ | C(O)OC₂H₅ |
| A-303 | NC₆H₅ | C(O)NHCH₃ |
| A-304 | NC₆H₅ | C(O)N(CH₃)₂ |
| A-305 | NC₆H₅ | C(O)NHCH₂CF₃ |
| A-306 | NC₆H₅ | C(O)NHC₆H₅ |
| A-307 | NC₆H₅ | C(S)CH₃ |
| A-308 | NC₆H₅ | C(S)C₂H₅ |
| A-309 | NC₆H₅ | C(S)CH(CH₃)₂ |
| A-310 | NC₆H₅ | C(S)-c-propyl |
| A-311 | NC₆H₅ | C(S)OCH₃ |
| A-312 | NC₆H₅ | C(S)OC₂H₅ |
| A-313 | NC₆H₅ | C(S)NHCH₃ |
| A-314 | NC₆H₅ | C(S)N(CH₃)₂ |
| A-315 | NC₆H₅ | C(S)NHCH₂CF₃ |
| A-316 | NC₆H₅ | C(S)NHC₆H₅ |
| A-317 | — * | pyrrolidin-1-yl |
| A-318 | — | 2-chloropyrrolidin-1-yl |
| A-319 | — | 3-chloropyrrolidin-1-yl |
| A-320 | — | 2-oxopyrrolidin-1-yl |
| A-321 | — | pyrazolidin-1-yl |
| A-322 | — | 3-chloropyrazolidin-1-yl |
| A-323 | — | 4-chloropyrazolidin-1-yl |
| A-324 | — | 3-oxopyrazolidin-1-yl |
| A-325 | — | imidazolidin-1-yl |
| A-326 | — | 2-oxoimidazolidin-1-yl |
| A-327 | — | 2,4-dioxoimidazolidin-1-yl |
| A-328 | — | piperidin-1-yl |
| A-329 | — | 2-oxopiperidin-1-yl |
| A-330 | — | piperazin-1-yl |
| A-331 | — | 2-oxopiperazin-1-yl |
| A-332 | — | morpholin-4-yl |

c-propyl = cyclopropyl
C₆H₅ = phenyl
* dash (—) means that X = chemical bond

The compounds of the formula (I) can be prepared by standard methods of organic chemistry, e.g. by the methods described hereinafter or in the synthesis descriptions of the working examples. The substituents, variables and indices are as defined above for formula (I), if not otherwise specified.

Compounds of formula I wherein R² is different from halogen can be prepared by reaction of compounds of the formula 1 with heterocycles of the formula 2, where X is an appropriate leaving group (e.g. chloride, bromide, iodide) as depicted below in scheme 1 and as described, for example, by Roger et al., J. Org. Chem., 2009, 74 (3), pp 1179-1186 or Liegault et al., J. Org. Chem., 2009, 74 (5), pp 1826-1834. Reaction temperatures are typically between 50 and 150° C., and the reactions are typically performed using a catalyst (e.g. palladium(II) acetate, palladium(II) trifluoroacetate, tetrakis(triphenylphosphine) palladium(0)), with or without additives (e.g. tricyclohexylphosphine, tri(o-tolyl)phosphine), and a base (e.g. cesium carbonate, sodium carbonate, potassium carbonate, potassium acetate) in an appropriate solvent (e.g. dimethylformamide, dimethylacetamide).

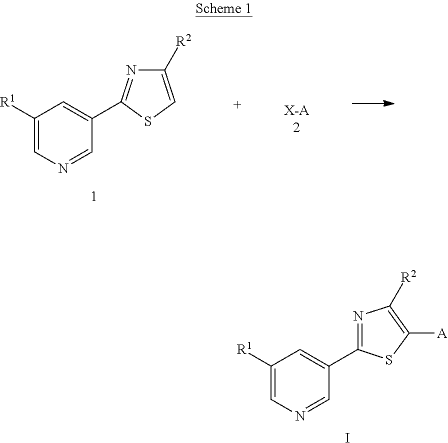

Alternatively, compound 1 can be reacted with a compound 3, as depicted in scheme 2, to compounds 4. In compounds 3 and 4, A′ corresponds to the heteroaromatic ring A, which is however devoid of the imine-derived group —C(Y)═N—X—R⁴. Instead, A′ carries an aldehyde or keto group —C(Y)═O. X is an appropriate leaving group (e.g. chloride, bromide, iodide). The reaction conditions are analogous to those for the reaction of scheme 1. Condensation of compounds 4 with amines of the formula 5 or ammonium salts thereof yields the compounds I. The reaction temperatures for the condensation reaction are typically between 0 and 120° C. The reactions can be performed with our without an additive (e.g. acetic acid, trifluoroacetic acid, potassium acetate, toluene-4-sulfonic acid, sodium acetate, molecular sieves) in an appropriate solvent (e.g. ethanol, methanol, toluene, water).

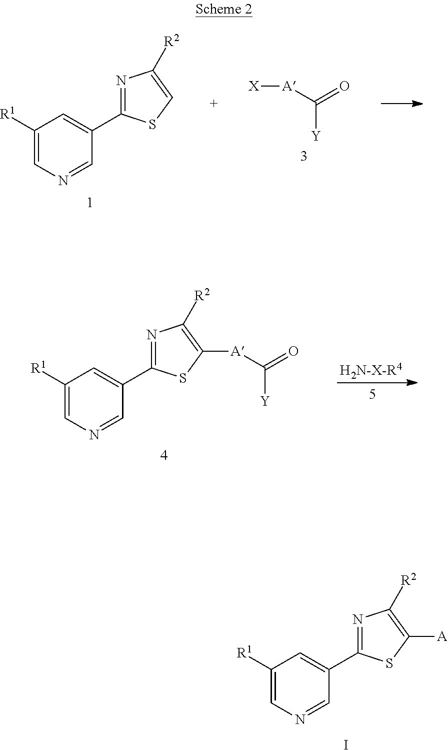

Compounds I wherein R² is a halogen atom can be prepared by reacting a compound I wherein R² is hydrogen with a halogenating agent, such as bromine, chlorine, fluorine, N-iodosuccinimide, N-bromosuccinimide, 1-chloromethyl-4-fluoro-1,4-diazoniabicyclo[2.2.2]octane bis(tetrafluoroborate or N-fluorobenzenesulfonimide. Reaction temperatures are typically between 0° C. and 150° C., and the reactions can be performed with or without an additive (e.g. benzoyl peroxide) and with or without a base (e.g. sodium sulfate) in an appropriate solvent (e.g. acetonitrile, dichloromethane, chloroform, tetrachloromethane).

Compounds of formula 1 can be prepared from the corresponding thio amide of the general formula 6 and compounds of the formula 7, where X is an appropriate leaving group (e.g. chloride, bromide), as shown in scheme 3 and as described by Bretschneider et al., US2011/212949 or in analogy to Begtrup et al., Acta Chemica Scandinavica, 1992, 46 (5), pp 372-383 or in analogy to Iwata et al., J. Org. Chem, 1992, 57, pp 3726-3727.

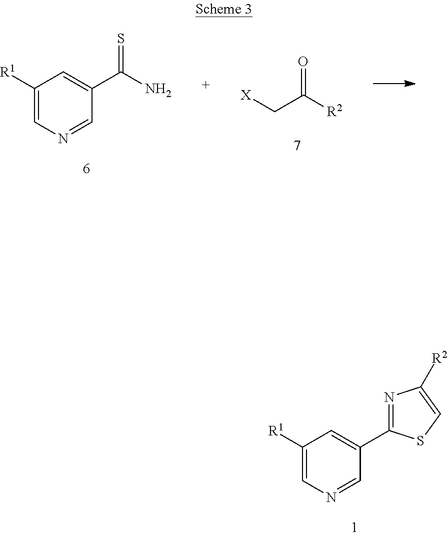

Compounds of formula 2 can be prepared by reacting the corresponding aldehyde or keto compound 3 with an amine 5 or ammonium salts thereof (Scheme 4). Here again, A′ corresponds to the heteroaromatic ring A, which is however devoid of the imine-derived group —C(Y)═N—X—R⁴ and carries instead an aldehyde or keto group —C(Y)═O. The reaction temperatures for the condensation are typically between 0 and 120° C. The reactions can be performed with our without an additive (e.g. acetic acid, trifluoroacetic acid, potassium acetate, toluene-4-sulfonic acid, sodium acetate, moleculare sieves) in an appropriate solvent (e.g. ethanol, methanol, toluene, water).

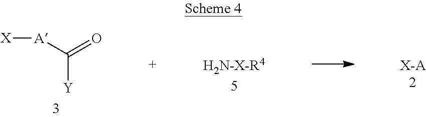

As a rule, the compounds of formula I including their stereoisomers, salts, and N-oxides, and their precursors in the synthesis process, can be prepared by the methods described above. If individual compounds can not be prepared via the above-described routes, they can be prepared by derivatization of other compounds I or the respective precursor or by customary modifications of the synthesis routes described. For example, in individual cases, certain compounds of formula I can advantageously be prepared from other compounds of formula I by derivatization, e.g. by ester hydrolysis, amidation, esterification, ether cleavage, olefination, reduction, oxidation and the like, or by customary modifications of the synthesis routes described.

The reaction mixtures are worked up in the customary manner, for example by mixing with water, separating the phases, and, if appropriate, purifying the crude products by chromatography, for example on alumina or on silica gel. Some of the intermediates and end products may be obtained in the form of colorless or pale brown viscous oils which are freed or purified from volatile components under reduced pressure and at moderately elevated temperature. If the intermediates and end products are obtained as solids, they may be purified by recrystallization or trituration.

Due to their excellent activity, the compounds of the present invention may be used for controlling invertebrate pests.

Accordingly, the present invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of the present invention or a composition as defined above.

Preferably, the method of the invention serves for protecting plant propagation material (such as seed) and the plant which grows therefrom from invertebrate pest attack or infestation and comprises treating the plant propagation material (such as seed) with a pesticidally effective amount of a compound of the present invention as defined above or with a pesticidally effective amount of an agricultural composition as defined above and below. The method of the invention is not limited to the protection of the “substrate”(plant, plant propagation materials, soil material etc.) which has been treated according to the invention, but also has a preventive effect, thus, for example, according protection to a plant which grows from a treated plant propagation materials (such as seed), the plant itself not having been treated.

In the sense of the present invention, “invertebrate pests” are preferably selected from arthropods and nematodes, more preferably from harmful insects, arachnids and nematodes, and even more preferably from insects, acarids and nematodes. In the sense of the present invention, “invertebrate pests” are most preferably insects.

The invention further provides an agricultural composition for combating invertebrate pests, which comprises such an amount of at least one compound according to the invention and at least one inert liquid and/or solid agronomically acceptable carrier that has a pesticidal action and, if desired, at least one surfactant.

Such a composition may comprise a single active compound of the present invention or a mixture of several active compounds of the present invention. The composition according to the present invention may comprise an individual isomer or mixtures of isomers or a salt as well as individual tautomers or mixtures of tautomers.

The compounds of the present invention, including their salts, stereoisomers and tautomers, are in particular suitable for efficiently controlling arthropodal pests such as arachnids, myriapedes and insects as well as nematodes. They are especially suitable for efficiently combating or controlling the following pests:

Insects from the order of the lepidopterans (Lepidoptera), for example Acronicta major, Adoxophyes orana, Aedia leucomelas, Agrotis spp., such as Agrotis fucosa, Agrotis segetum, Agrotis ipsilon, Alabama argillacea, Anticarsia gemmatalis, Anticarsia spp., Argyresthia conjugella, Autographa gamma, Barathra brassicae, Bucculatrix thurberiella, Bupalus piniarius, Cacoecia murinana, Cacoeciapodana, Capua reticulana, Carpocapsa pomonella, Cheimatobia brumata, Chilo spp. such as Chilo suppressalis, Choristoneura fumiferana, Choristoneura occidentalis, Cirphis unipuncta, Clysia ambiguella, Cnaphalocerus spp., Cydia pomonella, Dendrolimus pini, Diaphania nitidalis, Diatraea grandiosella, Earias insulana, Elasmopalpus lignosellus, Ephestia cautella, Ephestia kuehniella, Eupoecilia ambiguella, Euproctis chrysorrhoea, Euxoa spp., Evetria bouliana, Feltia spp. such as Feltia subterranea, Galleria mellonella, Grapholitha funebrana, Grapholitha molesta, Helicoverpa spp. such as Helicoverpa armigera, Helicoverpa zea, Heliothis spp. such as Heliothis armigera, Heliothis virescens, Heliothis zea; Hellula undalis, Hibernia defoliaria, Hofmannophila pseudospretella, Homona magnanima, Hyphantria cunea, Hyponomeuta padella, Hyponomeuta malinellus, Keiferia lycopersicella, Lambdina fiscellaria, Laphygma spp. such as Laphygma exigua, Leucoptera coffeella, Leucoptera scitella, Lithocolletis blancardella, Lithophane antennata, Lobesia botrana, Loxostege sticticalis, Lymantria spp. such as Lymantria dispar, Lymantria monacha, Lyonetia clerkella, Malacosoma neustria, Mamestra spp. such as Mamestra brassicae, Orgyia pseudotsugata, Oria spp., Ostrinia spp. such as Ostrinia nubilalis, Oulema oryzae, Panolis flammea, Pectinophora spp. such as Pectinophora gossypiella, Peridroma saucia, Phalera bucephala, Phthorimaea spp. such as Phthorimaea operculella, Phyllocnistis citrella, Pieris spp. such as Pieris brassicae, Pieris rapae, Plathypena scabra, Plutella maculipennis, Plutella xylostella, Prodenia spp., Pseudaletia spp., Pseudoplusia includens, Pyrausta nubilalis, Rhyacionia frustrana, Scrobipalpula absoluta, Sitotroga cerealella, Sparganothis pilleriana, Spodoptera spp. such as Spodoptera frugiperda, Spodoptera littoralis, Spodoptera litura, Thaumatopoea pityocampa, Thermesia gemmatalis, Tinea pellionella, Tineola bisselliella, Tortrix viridana, Trichoplusia spp. such as Trichoplusia ni, Tuta absoluta, and Zeiraphera canadensis;

beetles (Coleoptera), for example Acanthoscehdes obtectus, Adoretus spp., Agelastica alni, Agrilus sinuatus, Agriotes spp. such as Agriotes fuscicollis, Agriotes lineatus, Agriotes obscurus, Amphimallus solstitialis, Anisandrus dispar, Anobium punctatum Anomala rufocuprea, Anoplophora spp. such as Anoplophora glabripennis; Anthonomus spp. such as Anthonomus grandis, Anthonomus pomorum, Anthrenus spp., Aphthona euphoridae, Apogonia spp., Athous haemorrhoidalis, Atomaria spp. such as Atomaria linearis; Attagenus spp., Aulacophora femoralis, Blastophagus piniperda, Blitophaga undata, Bruchidius obtectus, Bruchus spp. such as Bruchus lentis, Bruchus pisorum, Bruchus rufimanus; Byctiscus betulae, Callosobruchus chinensis, Cassida nebulosa, Cerotoma trifurcata, Cetonia aurata, Ceuthorhynchus spp. such as Ceuthorrhynchus assimilis, Ceuthorrhynchus napi; Chaetocnema tibialis, Cleonus mendicus, Conoderus spp. such as Conoderus vespertinus; Cosmopolites spp., Costelytra zealandica, Crioceris asparagi, Cryptorhynchus lapathi, Ctenicera ssp. such as Ctenicera destructor; Curcullo spp., Dectes texanus, Dermestes spp., Diabrotica spp. such as Diabrotica 12-punctata Diabrotica speciosa, Diabrotica longicornis, Diabrotica semipunctata, Diabrotica virgifera; Epilachna spp. such as Epilachna varivestis, Epilachna vigintioctomaculata; Epitrix spp. such as Epitrix hirtipennis; Eutinobothrus brasiliensis, Faustinus cubae, Gibbium psylloides, Heteronychus arator, Hylamorpha elegans, Hylobius abletis, Hylotrupes bajulus, Hypera brunneipennis, Hypera postica, Hypothenemus spp., Ips typographus, Lachnosterna consanguinea, Lema bilineata, Lema melanopus, Leptinotarsa spp. such as Leptinotarsa decemlineata; Limonius californicus, Lissorhoptrus oryzophilus, Lissorhoptrus oryzophilus, Lixus spp., Lyctus spp. such as Lyctus bruneus, Melanotus communis, Meligethes spp. such as Meligethes aeneus; Melolontha hippocastani, Melolontha melolontha, Migdolus spp., Monochamus spp. such as Monochamus alternatus; Naupactus xanthographus, Niptus hololeucus, Oryctes rhinoceros, Oryzaephilus surinamensis, Otiorrhynchus sulcatus, Otiorrhynchus ovatus, Otiorrhynchus sulcatus, Oulema oryzae, Oxycetonia jucunda, Phaedon cochleariae, Phyllobius pyri, Phyllopertha horticola, Phyllophaga spp., Phyllotreta spp. such as Phyllotreta chrysocephala, Phyllotreta nemorum, Phyllotreta striolata; Phyllophaga spp., Phyllopertha horticola, Popillia japonica, Premnotrypes spp., Psylliodes chrysocephala, Ptinus spp., Rhizobius ventralis, Rhizopertha dominica, Sitona lineatus, Sitophilus spp. such as Sitophilus granaria, Sitophilus zeamais; Sphenophorus spp. such as Sphenophorus levis; Sternechus spp. such as Sternechus subsignatus; Symphyletes spp., Tenebrio molitor, Tribolium spp. such as Tribolium castaneum, Trogoderma spp., Tychius spp., Xylotrechus spp., and Zabrus spp. such as Zabrus tenebrioides,
flies, mosquitoes (Diptera), e.g. Aedes spp. such as Aedes aegypti, Aedes albopictus, Aedes vexans; Anastrepha ludens, Anopheles spp. such as Anopheles albimanus, Anopheles crucians, Anopheles freeborni, Anopheles gambiae, Anopheles leucosphyrus, Anopheles maculipennis, Anopheles minimus, Anopheles quadrimaculatus, Anopheles sinensis; Bibio hortulanus, Calliphora erythrocephala, Calliphora vicina, Cerafitis capitata, Ceratitis capitata, Chrysomyia spp. such as Chrysomya bezziana, Chrysomya hominivorax, Chrysomya macellaria; Chrysops atlanticus, Chrysops discalis, Chrysops silacea, Cochliomyia spp. such as Cochliomyia hominivorax; Contarinia spp. such as Contarinia sorghicola; Cordylobia anthropophaga, Culex spp. such as Culex nigripalpus, Culex pipiens, Culex quinquefasciatus, Culex tarsalis, Culex tritaeniorhynchus; Culicoides furens, Culiseta inornata, Culiseta melanura, Cuterebra spp., Dacus cucurbitae, Dacus oleae, Dasineura brassicae, Delia spp. such as Delia antique, Delia coarctata, Delia platura, Delia radicum; Dermatobia hominis, Drosophila spp., Fannia spp. such as Fannia canicularis; Gastraphilus spp. such as Gasterophilus intestinalis; Geomyza Tripunctata, Glossina fuscipes, Glossina morsitans, Glossina palpalis, Glossina tachinoides, Haematobia irritans, Haplodiplosis equestris, Hippelates spp., Hylemyia spp. such as Hylemyia platura, Hypoderma spp. such as Hypoderma lineata; Hyppobosca spp., Leptoconops torrens, Liriomyza spp. such as Liriomyza sativae, Liriomyza trifolii; Lucilia spp. such as Lucilia caprina, Lucilia cuprina, Lucilia sericata; Lycoria pectoralis, Mansonia titillanus, Mayetiola spp. such as Mayetiola destructor; Musca spp. such as Musca autumnalis, Musca domestica; Muscina stabulans, Oestrus spp. such as Oestrus ovis; Opomyza florum, Oscinella spp. such as Oscinella frit; Pegomya hysocyami, Phlebotomus argentipes, Phorbia spp. such as Phorbia antiqua, Phorbia brassicae, Phorbia coarctata, Prosimulium mixtum, Psila rosae, Psorophora columbiae, Psorophora discolor, Rhagoletis cerasi Rhagoletis pomonella, Sarcophaga spp. such as Sarcophaga haemorrhoidalis; Simulium vittatum, Stomoxys spp. such as Stomoxys calcitrans; Tabanus spp. such as Tabanus atratus, Tabanus bovinus, Tabanus lineola, Tabanus similis; Tannia spp., Tipula oleracea, Tipula paludosa, and Wohlfahrtia spp.,
thrips (Thysanoptera), e.g. Baliothrips biformis, Dichromothrips corbetti Dichromothrips ssp., Enneothrips flavens, Frankliniella spp. such as Frankliniella fusca, Frankliniella occidentalis, Frankliniella tritici; Heliothrips spp., Hercinothrips femoralis, Kakothrips spp., Rhipiphorothrips cruentatus, Scirtothrips spp. such as Scirtothrips citri; Taeniothrips cardamoni, Thrips spp. such as Thrips oryzae, Thrips palmi, Thrips tabaci;
termites (Isoptera), e.g. Calotermes flavicollis, Coptotermes formosanus, Heterotermes aureus, Heterotermes longiceps, Heterotermes tenuis, Leucotermes flavipes, Odontotermes spp., Reticulitermes spp. such as Reticulitermes speratus, Reticulitermes flavipes, Reticulitermes grassei, Reticulitermes lucifugus, Reticulitermes santonensis, Reticulitermes virginicus; Termes natalensis,
cockroaches (Blattaria-Blattodea), e.g. Acheta domesticus, Blatta orientalis, Blattella asahinae, Battella germanica, Gryllotapa spp., Leucophaea maderae, Locusta spp., Melanoplus spp., Periplaneta americana, Periplaneta australasiae, Periplaneta brunnea, Periplaneta fuligginosa, Periplaneta japonica,
bugs, aphids, leafhoppers, whiteflies, scale insects, cicadas (Hemiptera), e.g. Acrosternum spp. such as Acrosternum hilare; Acyrthosipon spp. such as Acyrthosiphon onobrychis, Acyrthosiphon pisum; Adelges laricis, Aeneolamia spp., Agonoscena spp., Aleurodes spp., Aleurolobus barodensis, Aleurothrixus spp., Amrasca spp., Anasa tristis, Antestiopsis spp., Anuraphis cardui, Aonidiella spp., Aphanostigma piri, Aphidula nasturtii Aphis spp. such as Aphis fabae, Aphis forbesi, Aphis gossypii, Aphis grossulariae, Aphis pomi, Aphis sambuci, Aphis schneideri, Aphis spiraecola, Arboridia apicalis, Arilus critatus, Aspidiella spp., Aspidiotus spp., Atanus spp., Aulacorthum solani, Bemisia spp. such as Bemisia argentifoli, Bemisia tabaci; Blissus spp. such as Blissus leucopterus; Brachycaudus cardui, Brachycaudus helichrysi, Brachycaudus persicae, Brachycaudus prunicola, Brachycolus spp., Brevicoryne brassicae, Calligypona marginata, Calocoris spp., Campylomma livida, Capitophorus horni Carneocephala fulgida, Cavelerius spp., Ceraplastes spp., Ceratovacuna lanigera, Cercopidae, Cerosipha gossypii, Chaetosiphon fragaefolii, Chionaspis tegalensis, Chlorita onukii, Chromaphis juglandicola, Chrysomphalus ficus, Cicadulina mbila, Cimex spp. such as Cimex hemipterus, Cimex lectularius; Coccomytilus halli, Coccus spp., Creontiades dilutus, Cryptomyzus ribis, Cryptomyzus ribis, Cyrtopeltis notatus, Dalbulus spp., Dasynus piperis, Dialeurades spp., Diaphorina spp., Diaspis spp., Dichelops furcatus, Diconocoris hewetti, Doralis spp., Dreyfusia nordmannianae, Dreyfusia piceae, Drosicha spp., Dysaphis spp. such as Dysaphis plantaginea, Dysaphis pyri, Dysaphis radicola; Dysaulacorthum pseudosolani, Dysdercus spp. such as Dysdercus cingulatus, Dysdercus intermedius; Dysmicoccus spp., Empoasca spp. such as Empoasca fabae, Empoasca solana; Eriosoma spp., Erythroneura spp., Eurygaster spp. such as Eurygaster integriceps; Euscelis bilobatus, Euschistus spp. such as Euschistuos heros, Euschistus impictiventris, Euschistus servus; Geococcus coffeae, Halyomorpha spp. such as Halyomorpha halys; Heliopeltis spp., Homalodisca coagulata, Horcias nobilellus, Hyalopterus pruni, Hyperomyzus lactucae, Icerya spp., Idiocerus spp., Idioscopus spp., Laodelphax striatellus, Lecanium spp., Lepidosaphes spp., Leptocorisa spp., Leptoglossus phyllopus, Lipaphis erysimi, Lygus spp. such as Lygus hesperus, Lygus lineolaris, Lygus pratensis; Macropes excavatus, Macrosiphum spp. such as Macrosiphum rosae, Macrosiphum avenae, Macrosiphum euphorbiae; Mahanarva fimbriolata, Megacopta cribraria, Megoura viciae, Melanaphis pyrarius, Melanaphis sacchar, Metcafiella spp., Metopolophium dirhodum, Miridae spp., Monellia costalis, Monelliopsis pecanis, Myzus spp. such as Myzus ascalonicus, Myzus cerasi, Myzus persicae, Myzus varians; Nasonovia ribis-nigri, Nephotettix spp. such as Nephotettix malayanus, Nephotettix nigropictus, Nephotettix parvus, Nephotettix virescens, Nezara spp. such as Nezara viridula; Nilaparvata lugens, Oebalus spp., Oncometopia spp., Orthezia praelonga, Parabemisia myricae, Paratrioza spp., Parlatoria spp., Pemphigus spp. such as Pemphigus bursarius; Pentomidae, Peregrinus maidis, Perkinsiella saccharicida, Phenacoccus spp., Phloeomyzus passerinii, Phorodon humuli, Phylloxera spp., Piesma quadrata, Piezodorus spp. such as Piezodorus guildinii, Pinnaspis aspidistrae, Planococcus spp., Protopulvinaria pyriformis, Psallus seriatus, Pseudacysta persea, Pseudaulacaspis pentagona, Pseudococcus spp. such as Pseudococcus comstocki; Psylla spp. such as Psylla mali, Psylla piri; Pteromalus spp., Pyrilla spp., Quadraspidiotus spp., Quesada gigas, Rastrococcus spp., Reduvius senilis, Rhodnius spp., Rhopalomyzus ascalonicus, Rhopalosiphum spp. such as Rhopalosiphum pseudobrassicas, Rhopalosiphum insertum, Rhopalosiphum maidis, Rhopalosiphum padi; Sagatodes spp., Sahlbergella singularis, Saissetia spp., Sappaphis mala, Sappaphis mali, Scaphoides titanus, Schizaphis graminum, Schizoneura lanuginosa, Scotinophora spp., Selenaspidus articulatus, Sitobion avenae, Sogata spp., Sogatella furcifera, Solubea insularis, Stephanitis nashi, Stictocephala festina, Tenalaphara malayensis, Thyanta spp. such as Thyanta perditor; Tibraca spp., Tinocallis caryaefoliae, Tomaspis spp., Toxoptera spp. such as Toxoptera aurantii; Trialeurodes spp. such as Trialeurodes vaporariorum; Triatoma spp., Trioza spp., Typhlocyba spp., Unaspis spp. such as Unaspis yanonensis; and Viteus vitifolii,
ants, bees, wasps, sawflies (Hymenoptera), e.g. Athalia rosae, Atta capiguara, Atta cephalotes, Atta cephalotes, Atta laevigata, Atta robusta, Atta sexdens, Atta texana, Bombus spp., Camponotus floridanus, Crematogaster spp., Dasymutilla occidentalis, Diprion spp., Dolichovespula maculata, Hoplocampa spp. such as Hoplocampa minuta, Hoplocampa testudinea; Lasius spp. such as Lasius niger, Linepithema humile, Monomorium pharaonis, Paravespula germanica, Paravespula pennsylvanica, Paravespula vulgaris, Pheidole megacephala, Pogonomyrmex barbatus, Pogonomyrmex californicus, Polistes rubiginosa, Solenopsis geminata, Solenopsis invicta, Solenopsis richter, Solenopsis xyloni, Vespa spp. such as Vespa crabro, and Vespula squamosa,
crickets, grasshoppers, locusts (Orthoptera), e.g. Acheta domestica, Calliptamus italicus, Chortoicetes terminifera, Dociostaurus maroccanus, Gryllotalpa africana, Gryllotalpa gryllotalpa, Hieroglyphus daganensis, Kraussaria angulifera, Locusta migratoria, Locustana pardalina, Melanoplus bivittatus, Melanoplus femurrubrum, Melanoplus mexicanus, Melanoplus sanguinipes, Melanoplus spretus, Nomadacris septemfasciata, Oedaleus senegalensis, Schistocerca americana, Schistocerca gregaria, Tachycines asynamorus, and Zonozerus variegatus,
arachnids (Arachnida), such as acari, e.g. of the families Argasidae, Ixodidae and Sarcoptidae, such as Amblyomma spp. (e.g. Amblyomma americanum, Amblyomma variegatum, Amblyomma maculatum), Argas spp. (e.g. Argas persicus), Boophilus spp. (e.g. Boophilus annulatus, Boophilus decoloratus, Boophilus microplus), Dermacentor silvarum, Dermacentor andersoni, Dermacentor variabilis, Hyalomma spp. (e.g. Hyalomma truncatum), Ixodes spp. (e.g. Ixodes ricinus, Ixodes rubicundus, Ixodes scapularis, Ixodes holocyclus, Ixodes pacificus), Ornithodorus spp. (e.g. Ornithodorus moubata, Ornithodorus hermsi, Ornithodorus turicata), Ornithonyssus bacoti, Otobius megnini, Dermanyssus gallinae, Psoroptes spp. (e.g. Psoroptes ovis), Rhipicephalus spp. (e.g. Rhipicephalus sanguineus, Rhipicephalus appendiculatus, Rhipicephalus evertsi), Rhizoglyphus spp., Sarcoptes spp. (e.g. Sarcoptes scabiei), and Eriophyidae spp. such as Acaria sheldoni, Aculops spp. (e.g. Aculops pelekassi) Aculus spp. (e.g. Aculus schlechtendali), Epitrimerus pyri, Phyllocoptruta oleivora and Eriophyes spp. (e.g. Eriophyes sheldoni); Tarsonemidae spp. such as Hemitarsonemus spp., Phytonemus pallidus and Polyphagotarsonemus latus, Stenotarsonemus spp.; Tenuipalpidae spp. such as Brevipalpus spp. (e.g. Brevipalpus phoenicis); Tetranychidae spp. such as Eotetranychus spp., Eutetranychus spp., Oligonychus spp., Tetranychus cinnabarinus, Tetranychus kanzawai, Tetranychus pacificus, Tetranychus telarius and Tetranychus urticae; Bryobia praetiosa, Panonychus spp. (e.g. Panonychus ulmi, Panonychus citri), Metatetranychus spp. and Oligonychus spp. (e.g. Oligonychus pratensis), Vasates Iycopersici; Araneida, e.g. Latrodectus mactans, and Loxosceles reclusa. And Acarus siro, Chorioptes spp., Scorpio maurus
fleas (Siphonaptera), e.g. Ceratophyllus spp., Ctenocephalides felis, Ctenocephalides canis, Xenopsylla cheopis, Pulex irritans, Tunga penetrans, and Nosopsyllus fasciatus,
silverfish, firebrat (Thysanura), e.g. Lepisma saccharina and Thermobia domestica,
centipedes (Chilopoda), e.g. Geophilus spp., Scutigera spp. such as Scutigera coleoptrata;
millipedes (Diplopoda), e.g. Blaniulus guttulatus, Narceus spp.,
Earwigs (Dermaptera), e.g. forficula auricularia,
lice (Phthiraptera), e.g. Damalinia spp., Pediculus spp. such as Pediculus humanus capitis, Pediculus humanus corporis; Pthirus pubis, Haematopinus spp. such as Haematopinus eurysternus, Haematopinus suis; Linognathus spp. such as Linognathus vituli; Bovicola bovis, Menopon gallinae, Menacanthus stramineus and Solenopotes capillatus, Trichodectes spp.,
springtails (Collembola), e.g. Onychiurus ssp. such as Onychiurus armatus,

They are also suitable for controlling nematodes: plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species, Heterodera avenae, Heterodera glycines, Heterodera schachtii, Heterodera trifolii, and other Heterodera species; Seed gall nematodes, Anguina species; Stem and foliar nematodes, Aphelenchoides species such as Aphelenchoides besseyi; Sting nematodes, Belonolaimus longicaudatus and other Belonolaimus species; Pine nematodes, Bursaphelenchus lignicolus Mamiya et Kiyohara, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus brachyurus, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species such as Tylenchulus semipenetrans; Dagger nematodes, Xiphinema species; and other plant parasitic nematode species.

Examples of further pest species which may be controlled by compounds of formula (I) include: from the class of the Bivalva, for example, Dreissena spp.; from the class of the Gastropoda, for example, Arion spp., Biomphalaria spp., Bulinus spp., Deroceras spp., Galba spp., Lymnaea spp., Oncomelania spp., Succinea spp., from the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medinensis, Echinococcus granulosus, Echinococcus multilocularis, Enterobius vermicularis, Faciola spp., Haemonchus spp. such as Haemonchus contortus; Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercora lis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichiura, Wuchereria bancrofti; from the order of the Isopoda, for example, Armadillidium vulgare, Oniscus asellus, Porcellio scaber; from the order of the Symphyla, for example, Scutigerella immaculate.

Further examples of pest species which may be controlled by compounds of formula (I) include: Anisoplia austriaca, Apamea spp., Austroasca viridigrisea, Baliothrips biformis, Caenorhabditis elegans, Cephus spp., Ceutorhynchus napi, Chaetocnema aridula, Chilo auricilius, Chilo indicus, Chilo polychrysus, Chortiocetes terminifera, Cnaphalocroci medinalis, Cnaphalocrosis spp., Colias eurytheme, Collops spp., Cornmitermes cumulans, Creontiades spp., Cyclocephala spp., Dalbulus maidis, Deraceras reticulatum, Diatrea saccharalis, Dichelops furcatus, Dicladispa armigera, Diloboderus spp. such as Diloboderus abderus; Edessa spp., Epinotia spp., Formicidae, Geocoris spp., Globitermes sulfureus, Gryllotalpidae, Halotydeus destructor, Hipnodes bicolor, Hydrellia philippina, Julus spp., Laodelphax spp., Leptocorsia acuta, Leptocorsia oratorius, Liogenys fuscus, Lucillia spp., Lyogenys fuscus, Mahanarva spp., Maladera matrida, Marasmia spp., Mastotermes spp., Mealybugs, Megascelis ssp, Metamasius hemipterus, Microtheca spp., Mocis latipes, Murgantia spp., Mythemina separata, Neocapritermes opacus, Neocapritermes parvus, Neomegalotomus spp., Neotermes spp., Nymphula depunctalis, Oebalus pugnax, Orseolia spp. such as Orseolia oryzae; Oxycaraenus hyalinipennis, Plusia spp., Pomacea canaliculata, Procornmitermes ssp, Procornitermes triacifer, Psylloides spp., Rachiplusia spp., Rhodopholus spp., Scaptocoris castanea, Scaptocoris spp., Scirpophaga spp. such as Scirpophaga incertulas, Scirpophaga innotata; Scotinophara spp. such as Scotinophara coarctata; Sesamia spp. such as Sesamia inferens, Sogaella frucifera, Solenapsis geminata, Spissistilus spp., Stalk borer, Stenchaetothrips biformis, Steneotarsonemus spinki, Sylepta derogata, Telehin licus, Trichostrongylus spp.

The compounds of the present invention, including their salts, stereoisomers and tautoems, are also suitable for controlling nematodes: plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

The compounds of the present invention, including their salts, stereoisomers and tautomers, are particularly useful for controlling insects, preferably sucking or piercing and chewing and biting insects such as insects from the genera Lepidoptera, Coleoptera and Hemiptera, in particular Lepidoptera, Coleoptera and true bugs.

The compounds of the present invention, including their salts, stereoisomers and tautomers, are moreover useful for controlling insects of the orders Thysanoptera, Diptera (especially flies, mosquitoes), Hymenoptera (especially ants) and Isoptera (especially termites.

The compounds of the present invention, including their salts, stereoisomers and tautomers, are particularly useful for controlling insects of the orders Lepidoptera and Coleoptera.

The invention also relates to agrochemical compositions comprising an auxiliary and at least one compound I according to the invention.

An agrochemical composition comprises a pesticidally effective amount of a compound I. The term “effective amount” denotes an amount of the composition or of the compounds I, which is sufficient for controlling harmful fungi on cultivated plants or in the protection of materials and which does not result in a substantial damage to the treated plants. Such an amount can vary in a broad range and is dependent on various factors, such as the species to be controlled, the treated cultivated plant or material, the climatic conditions and the specific compound I used.

The compounds I, their N-oxides and salts can be converted into customary types of agrochemical compositions, e. g. solutions, emulsions, suspensions, dusts, powders, pastes, granules, pressings, capsules, and mixtures thereof. Examples for composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further compositions types are defined in the “Catalogue of pesticide formulation types and international coding system”, Technical Monograph No. 2, 6^{th} Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters or monoglycerides. Examples of sugar-based surfactants are sorbitans, ethoxylated sorbitans, sucrose and glucose esters or alkylpolyglucosides. Examples of polymeric surfactants are home- or copolymers of vinylpyrrolidone, vinylalcohols, or vinylacetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethlene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the compound I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e.g. xanthan gum, carboxymethylcellulose), anorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo- and phthalocyanine colorants).

Suitable tackifiers or binders are polyvinylpyrrolidones, polyvinylacetates, polyvinyl alcohols, polyacrylates, biological or synthetic waxes, and cellulose ethers.

Examples for composition types and their preparation are:

### i) Water-Soluble Concentrates (SL, LS)

10-60 wt % of a compound I according to the invention and 5-15 wt % wetting agent (e.g. alcohol alkoxylates) are dissolved in water and/or in a water-soluble solvent (e.g. alcohols) ad 100 wt %. The active substance dissolves upon dilution with water.

### ii) Dispersible Concentrates (DC)

5-25 wt % of a compound I according to the invention and 1-10 wt % dispersant (e. g. polyvinylpyrrolidone) are dissolved in organic solvent (e.g. cyclohexanone) ad 100 wt %. Dilution with water gives a dispersion.

iii) Emulsifiable Concentrates (EC)

15-70 wt % of a compound I according to the invention and 5-10 wt % emulsifiers (e.g. calcium dodecylbenzenesulfonate and castor oil ethoxylate) are dissolved in water-insoluble organic solvent (e.g. aromatic hydrocarbon) ad 100 wt %. Dilution with water gives an emulsion.

### iv) Emulsions (EW, EO, ES)

5-40 wt % of a compound I according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into water ad 100 wt % by means of an emulsifying machine and made into a homogeneous emulsion. Dilution with water gives an emulsion.

### v) Suspensions (SC, OD, FS)

In an agitated ball mill, 20-60 wt % of a compound I according to the invention are comminuted with addition of 2-10 wt % dispersants and wetting agents (e.g. sodium lignosulfonate and alcohol ethoxylate), 0.1-2 wt % thickener (e.g. xanthan gum) and water ad 100 wt % to give a fine active substance suspension. Dilution with water gives a stable suspension of the active substance. For FS type composition up to 40 wt % binder (e.g. polyvinylalcohol) is added.

### vi) Water-Dispersible Granules and Water-Soluble Granules (WG, SG)

50-80 wt % of a compound I according to the invention are ground finely with addition of dispersants and wetting agents (e.g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.

vii) Water-Dispersible Powders and Water-Soluble Powders (WP, SP, WS)

50-80 wt % of a compound I according to the invention are ground in a rotor-stator mill with addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the active substance.

viii) Gel (GW, GF)

In an agitated ball mill, 5-25 wt % of a compound I according to the invention are comminuted with addition of 3-10 wt % dispersants (e.g. sodium lignosulfonate), 1-5 wt % thickener (e.g. carboxymethylcellulose) and water ad 100 wt % to give a fine suspension of the active substance. Dilution with water gives a stable suspension of the active substance.

### iv) Microemulsion (ME)

5-20 wt % of a compound I according to the invention are added to 5-30 wt % organic solvent blend (e.g. fatty acid dimethylamide and cyclohexanone), 10-25 wt % surfactant blend (e.g. alkohol ethoxylate and arylphenol ethoxylate), and water ad 100%. This mixture is stirred for 1 h to produce spontaneously a thermodynamically stable microemulsion.

### iv) Microcapsules (CS)

An oil phase comprising 5-50 wt % of a compound I according to the invention, 0-40 wt % water insoluble organic solvent (e.g. aromatic hydrocarbon), 2-15 wt % acrylic monomers (e.g. methylmethacrylate, methacrylic acid and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of a compound I according to the invention, 0-40 wt % water insoluble organic solvent (e.g. aromatic hydrocarbon), and an isocyanate monomer (e.g. diphenylmethene-4,4′-diisocyanatae) are dispersed into an aqueous solution of a protective colloid (e.g. polyvinyl alcohol). The addition of a polyamine (e.g. hexamethylenediamine) results in the formation of a polyurea microcapsules. The monomers amount to 1-10 wt %. The wt % relate to the total CS composition.

### ix) Dustable Powders (DP, DS)

1-10 wt % of a compound I according to the invention are ground finely and mixed intimately with solid carrier (e.g. finely divided kaolin) ad 100 wt %.

### x) Granules (GR, FG)

0.5-30 wt % of a compound I according to the invention is ground finely and associated with solid carrier (e.g. silicate) ad 100 wt %. Granulation is achieved by extrusion, spray-drying or the fluidized bed.

### xi) Ultra-Low Volume Liquids (UL)

1-50 wt % of a compound I according to the invention are dissolved in organic solvent (e.g. aromatic hydrocarbon) ad 100 wt %.

The compositions types i) to xi) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, and in particular between 0.5 and 75%, by weight of active substance. The active substances are employed in a purity of from 90% to 100%, preferably from 95% to 100% (according to NMR spectrum).

Solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds. The compositions in question give, after two-to-tenfold dilution, active substance concentrations of from 0.01 to 60% by weight, preferably from 0.1 to 40% by weight, in the ready-to-use preparations. Application can be carried out before or during sowing. Methods for applying compound I and compositions thereof, respectively, on to plant propagation material, especially seeds include dressing, coating, pelleting, dusting, soaking and in-furrow application methods of the propagation material. Preferably, compound I or the compositions thereof, respectively, are applied on to the plant propagation material by a method such that germination is not induced, e. g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials such as seeds, e. g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seeds) are generally required. When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Various types of oils, wetters, adjuvants, fertilizer, or micronutrients, and further pesticides (e.g. herbicides, insecticides, fungicides, growth regulators, safeners) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank and further auxiliaries may be added, if appropriate. In a further embodiment, either individual components of the composition according to the invention or partially premixed components, e. g. components comprising compounds I and/or active substances from the groups M) or F) (see below), may be mixed by the user in a spray tank and further auxiliaries and additives may be added, if appropriate.

In a further embodiment, either individual components of the composition according to the invention or partially premixed components, e. g. components comprising compounds I and/or active substances from the groups M) or F) (see below), can be applied jointly (e.g. after tank mix) or consecutively.

The following list M of pesticides, grouped according to the Mode of Action Classification of the Insecticide Resistance Action Committee (IRAC), and together with which the compounds according to the present invention can be used and with which potential synergistic effects might be produced, is intended to illustrate the possible combinations, but not to impose any limitation:

M.1 Acetylcholine esterase (AChE) inhibitors from the class of
M.1A carbamates, for example aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb and triazamate; or from the class of
M.1B organophosphates, for example acephate, azamethiphos, azinphosmethyl, azinphosmethyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O- (methoxyaminothio-phosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon and vamidothion;
M.2. GABA-gated chloride channel antagonists such as:
M.2A cyclodiene organochlorine compounds, as for example endosulfan or chlordane; or
M.2B fiproles (phenylpyrazoles), as for example ethiprole, fipronil, flufiprole, pyrafluprole and pyriprole;
M.3 Sodium channel modulators from the class of
M.3A pyrethroids, for example acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zetacypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, meperfluthrin, metofluthrin, momfluorothrin, permethrin, phenothrin, prallethrin, profluthrin, pyrethrin (pyrethrum), resmethrin, silafluofen, tefluthrin, tetramethylfluthrin, tetramethrin, tralomethrin and transfluthrin; or
M.3B sodium channel modulators such as DDT or methoxychlor;
M.4 Nicotinic acetylcholine receptor agonists (nAChR) from the class of
M.4A neonicotinoids, for example acteamiprid, chlothianidin, dinotefuran, imidacloprid, nitenpyram, thiacloprid and thiamethoxam; or the compounds
M.4A.1: 1-[(6-chloro-3-pyridinyl)methyl]-2,3,5,6,7,8-hexahydro-9-nitro-(5S,8R)-5,8-Epoxy-1 H-imidazo[1,2-a]azepine; or
M.4A.2: 1-[(6-chloro-3-pyridyl)methyl]-2-nitro-1-[(E)-pentylideneamino]guanidine; or
M4.A.3: 1-[(6-chloro-3-pyridyl)methyl]-7-methyl-8-nitro-5-propoxy-3,5,6,7-tetrahydro-2H-imidazo[1,2-a]pyridine;
or
M.4B nicotine.
M.5 Nicotinic acetylcholine receptor allosteric activators from the class of spinosyns, for example spinosad or spinetoram;
M.6 Chloride channel activators from the class of avermectins and milbemycins, for example abamectin, emamectin benzoate, ivermectin, lepimectin or milbemectin;
M.7 Juvenile hormone mimics, such as
M.7A juvenile hormone analogues as hydroprene, kinoprene and methoprene; or others as
M.7B fenoxycarb, or
M.7C pyriproxyfen;
M.8 miscellaneous non-specific (multi-site) inhibitors, for example
M.8A alkyl halides as methyl bromide and other alkyl halides, or
M.8B chloropicrin, or
M.8C sulfuryl fluoride, or
M.8D borax, or
M.8E tartar emetic;
M.9 Selective homopteran feeding blockers, for example
M.9B pymetrozine, or
M.9C flonicamid;
M.10 Mite growth inhibitors, for example
M.10A clofentezine, hexythiazox and diflovidazin, or
M.10B etoxazole;
M.11 Microbial disruptors of insect midgut membranes, for example bacillus thuringiensis or bacillus sphaericus and the insecticidal proteins they produce such as bacillus thuringiensis subsp. israelensis, bacillus sphaericus, bacillus thuringiensis subsp. aizawai, bacillus thuringiensis subsp. kurstaki and bacillus thuringiensis subsp. tenebrionis, or the Bt crop proteins: Cry1Ab, Cry1Ac, Cry1Fa, Cry2Ab, mCry3A, Cry3Ab, Cry3Bb and Cry34/35Ab1;
M.12 Inhibitors of mitochondrial ATP synthase, for example
M.12A diafenthiuron, or
M.12B organotin miticides such as azocyclotin, cyhexatin or fenbutatin oxide, or M.12C propargite, or
M.12D tetradifon;
M.13 Uncouplers of oxidative phosphorylation via disruption of the proton gradient, for example chlorfenapyr, DNOC or sulfluramid;
M.14 Nicotinic acetylcholine receptor (nAChR) channel blockers, for example nereistoxin analogues as bensultap, cartap hydrochloride, thiocyclam or thiosultap sodium;
M.15 Inhibitors of the chitin biosynthesis type 0, such as benzoylureas as for example bistrifluron, chlorfluazuron, diflubenzuron, flucycloxuron, flufenoxuron, hexaflumuron, lufenuron, novaluron, noviflumuron, teflubenzuron or triflumuron;
M.16 Inhibitors of the chitin biosynthesis type 1, as for example buprofezin;
M.17 Moulting disruptors, Dipteran, as for example cyromazine;
M.18 Ecdyson receptor agonists such as diacylhydrazines, for example methoxyfenozide, tebufenozide, halofenozide, fufenozide or chromafenozide;
M.19 Octopamin receptor agonists, as for example amitraz;
M.20 Mitochondrial complex III electron transport inhibitors, for example
M.20A hydramethylnon, or
M.20B acequinocyl, or
M.20C fluacrypyrim;
M.21 Mitochondrial complex I electron transport inhibitors, for example
M.21A METI acaricides and insecticides such as fenazaquin, fenpyroximate, pyrimidifen, pyridaben, tebufenpyrad or tolfenpyrad, or
M.21B rotenone;
M.22 Voltage-dependent sodium channel blockers, for example
M.22A indoxacarb, or
M.22B metaflumizone, or
M.22C 1-[(E)-[2-(4-cyanophenyl)-1-[3-(trifluoromethyl)phenyl]ethylidene]amino]-3-[4-(difluoromethoxy)phenyl]urea;
M.23 Inhibitors of the of acetyl CoA carboxylase, such as Tetronic and Tetramic acid derivatives, for example spirodiclofen, spiromesifen or spirotetramat;
M.24 Mitochondrial complex IV electron transport inhibitors, for example
M.24A phosphine such as aluminium phosphide, calcium phosphide, phosphine or zinc phosphide, or
M.24B cyanide.
M.25 Mitochondrial complex II electron transport inhibitors, such as beta-ketonitrile derivatives, for example cyenopyrafen or cyflumetofen;
M.28 Ryanodine receptor-modulators from the class of diamides, as for example flubendiamide, chloranthraniliprole (Rynaxypyr®), cyanthraniliprole (Cyazypyr®), or
the phthalamide compounds
M.28.1: (R)-3-Chlor-N1-{2-methyl-4-[1,2,2,2-tetrafluor-1-(trifluormethyl)ethyl]phenyl}-N2-(1-methyl-2-methylsulfonylethyl)phthalamid and
M.28.2: (S)-3-Chlor-N1-{2-methyl-4-[1,2,2,2-tetrafluor-1-(trifluormethyl)ethyl]phenyl}-N2-(1-methyl-2-methylsulfonylethyl)phthalamid, or the compound
M.28.3: 3-bromo-N-{2-bromo-4-chloro-6-[(1-cyclopropylethyl)carbamoyl]phenyl}-1-(3-chlorpyridin-2-yl)-1H-pyrazole-5-carboxamide (proposed ISO name: cyclaniliprole), or the compound
M.28.4: methyl-2-[3,5-dibromo-2-({[3-bromo-1-(3-chlorpyridin-2-yl)-1H-pyrazol-5-yl]carbonyl}amino)benzoyl]-1,2-dimethylhydrazinecarboxylate; or a compound selected from M.28.5a) to M.28.5 l):M.28.5a)N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide;
M.28.5b)N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide;
M.28.5c)N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide;
M.28.5d)N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide;
M.28.5e)N-[4,6-dichloro-2-[(diethyl-lambda-4-sulfanylidene)carbamoyl]-phenyl]-2-(3-chloro-2-pyridyl)-5-(difluoromethyl)pyrazole-3-carboxamide;
M.28.5f)N-[4,6-dibromo-2-[(di-2-propyl-lambda-4-sulfanylidene)carbamoyl]-phenyl]-2-(3-chloro-2-pyridyl)-5-(trifluoromethyl)pyrazole-3-carboxamide;
M.28.5g)N-[4-chloro-2-[(di-2-propyl-lambda-4-sulfanylidene)carbamoyl]-6-cyano-phenyl]-2-(3-chloro-2-pyridyl)-5-(trifluoromethyl)pyrazole-3-carboxamide;
M.28.5h)N-[4,6-dibromo-2-[(diethyl-lambda-4-sulfanylidene)carbamoyl]-phenyl]-2-(3-chloro-2-pyridyl)-5-(trifluoromethyl)pyrazole-3-carboxamide;
M.28.5i)N-[2-(5-amino-1,3,4-thiadiazol-2-yl)-4-chloro-6-methyl-phenyl]-5-bromo-2-(3-chloro-2-pyridyl)pyrazole-3-carboxamide;
M.28.5j) 5-chloro-2-(3-chloro-2-pyridyl)-N-[2,4-dichloro-6-[(1-cyano-1-methyl-ethyl)carbamoyl]phenyl]pyrazole-3-carboxamide;
M.28.5k) 5-bromo-N-[2,4-dichloro-6-(methylcarbamoyl)phenyl]-2-(3,5-dichloro-2-pyridyl)pyrazole-3-carboxamide;
M.28.5l) N-[2-(tert-butylcarbamoyl)-4-chloro-6-methyl-phenyl]-2-(3-chloro-2-pyridyl)-5-(fluoromethoxy)pyrazole-3-carboxamide; or a compound selected from
M.28.6 N2-(1-cyano-1-methyl-ethyl)-N1-(2,4-dimethylphenyl)-3-iodo-phthalamide; or
M.28.7 3-chloro-N2-(1-cyano-1-methyl-ethyl)-N1-(2,4-dimethylphenyl)phthalamide;
M.UN.X insecticidal active compounds of unknown or uncertain mode of action, as for example afidopyropen, azadirachtin, amidoflumet, benzoximate, bifenazate, bromopropylate, chinomethionat, cryolite, dicofol, flufenerim, flometoquin, fluensulfone, flupyradifurone, piperonyl butoxide, pyridalyl, pyrifluquinazon, sulfoxaflor, pyflubumide, or the compounds
M.UN.X.1: 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide, or the compound
M.UN.X.2: 4-[5-[3-chloro-5-(trifluoromethyl)phenyl]-5-(trifluoromethyl)-4H-isoxazol-3-yl]-N-[2-oxo-2-(2,2,2-trifluoroethylamino)ethyl]naphthalene-1-carboxamide, or the compound,
M.UN.X.3: 11-(4-chloro-2,6-dimethylphenyl)-12-hydroxy-1,4-dioxa-9-azadispiro[4.2.4.2]-tetradec-11-en-10-one, or the compound
M.UN.X.4: 3-(4′-fluoro-2,4-dimethylbiphenyl-3-yl)-4-hydroxy-8-oxa-1-azaspiro[4.5]dec-3-en-2-one, or the compound
M.UN.X.5: 1-[2-fluoro-4-methyl-5-[(2,2,2-trifluoroethyl)sulfinyl]phenyl]-3-(trifluoromethyl)-1H-1,2,4-triazole-5-amine, or actives on basis of bacillus firmus (Votivo, I-1582);
M.UN.X.6; a compound selected from the group of
M.UN.X.6a) (E/Z)-N-[1-[(6-chloro-3-pyridyl)methyl]-2-pyridylidene]-2,2,2-trifluoroacetamide;
M.UN.X.6b) (E/Z)-N-[1-[(6-chloro-5-fluoro-3-pyridyl)methyl]-2-pyridylidene]-2,2,2-trifluoroacetamide;
M.UN.X.6c) (E/Z)-2,2,2-trifluoro-N-[1-[(6-fluoro-3-pyridyl)methyl]-2-pyridylidene]acetamide;
M.UN.X.6d) (E/Z)-N-[1-[(6-bromo-3-pyridyl)methyl]-2-pyridylidene]-2,2,2-trifluoroacetamide;
M.UN.X.6e) (E/Z)-N-[1-[1-(6-chloro-3-pyridyl)ethyl]-2-pyridylidene]-2,2,2-trifluoroacetamide;
M.UN.X.6f) (E/Z)-N-[1-[(6-chloro-3-pyridyl)methyl]-2-pyridylidene]-2,2-difluoroacetamide;
M.UN.X. 6g) (E/Z)-2-chloro-N-[1-[(6-chloro-3-pyridyl)methyl]-2-pyridylidene]-2,2-difluoroacetamide;
M.UN.X.6h) (E/Z)-N-[1-[(2-chloropyrimidin-5-yl)methyl]-2-pyridylidene]-2,2,2-trifluoroacetamide and
M.UN.X.6i) (E/Z)-N-[1-[(6-chloro-3-pyridyl)methyl]-2-pyridylidene]-2,2,3,3,3-pentafluoropropanamide); or of the compounds
M.UN.X.7: 3-[3-chloro-5-(trifluoromethyl)phenyl]-4-oxo-1-(pyrimidin-5-ylmethyl)pyrido[1,2-a]pyrimidin-1-ium-2-olate; or
M.UN.X.8: 8-chloro-N-[2-chloro-5-methoxyphenyl)sulfonyl]-6-trifluoromethyl)imidazo[1,2-a]pyridine-2-carboxamide; or
M.UN.X.9: 4-[5-(3,5-dichlorophenyl)-5-(trifluoromethyl)-4H-isoxazol-3-yl]-2-methyl-N-(1-oxothietan-3-yl)benzamide; or
M.UN.X. 10: 5-[3-[2,6-dichloro-4-(3,3-dichloroallyloxyl)phenoxy]propoxy]-1H-pyrazole.

The commercially available compounds of the group M listed above may be found in The Pesticide Manual, 15th Edition, C. D. S. Tomlin, British Crop Protection Council (2011) among other publications.

The quinoline derivative flometoquin is shown in WO2006/013896. The aminofuranone compounds flupyradifurone is known from WO 2007/115644. The sulfoximine compound sulfoxaflor is known from WO2007/149134. The pyrethroid momfluorothrin is known from U.S. Pat. No. 6,908,945. The pyrazole acaricide pyflubumide is known from WO2007/020986. The isoxazoline compounds have been described likewise M.UN.X.1 in WO2005/085216, M.UN.X2. in WO2009/002809 and in WO2011/149749 and the isoxazoline M.UN.X.9 in WO2013/050317. The pyripyropene derivative afidopyropen has been described in WO 2006/129714. The spiroketal-substituted cyclic ketoenol derivative M.UN.X.3 is known from WO2006/089633 and the biphenyl-substituted spirocyclic ketoenol derivative M.UN.X.4 from WO2008/067911. Finally triazoylphenylsulfide like M.UN.X.5 have been described in WO2006/043635 and biological control agents on basis of bacillus firmus in WO2009/124707. The neonicotinoids 4A.1 is known from WO20120/069266 and WO2011/06946, the M.4.A.2 from WO2013/003977, the M4.A.3. from WO2010/069266.

The Metaflumizone analogue M.22C is described in CN 10171577. The phthalamides M.28.1 and M.28.2 are both known from WO 2007/101540. The anthranilamide M.28.3 has been described in WO2005/077934. The hydrazide compound M.28.4 has been described in WO 2007/043677. The anthranilamides M.28.5a) to M.28.5h) can be prepared as described in WO 2007/006670, WO2013/024009 and WO2013/024010, the anthranilamide M.28.5i) is described in WO2011/085575, the M.28.5j) in WO2008/134969, the M.28.5k) in US2011/046186 and the M.28.5l) in WO2012/034403. The diamide compounds M.28.6 and M.28.7 can be found in CN102613183.

The compounds M.UN.X.6a) to M.UN.X.6i) listed in M.UN.X.6 have been described in WO2012/029672. The mesoionic antagonist compound M.UN.X.7 was described in WO2012/092115, the nematicide M.UN.X.8 in WO2013/055584 and the Pyridalyl-type analogue M.UN.X.10 in WO2010/060379.

Preferred additional pesticidally active ingredients are those selected from the IRAC group 1, the Acetylcholinesterase (AChE) inhibitors, herein from the group 1A (Carbamates) Thiodicarb, Methomyl and Carbaryl, and from the group 1B(Organophosphates), especially Acephate, Chlorpyriphos and Dimethoate, from the group 2B, the fiproles, here especially ethiprole and fipronil, from the group 3, the pyrethroids, here especially lambda-cyhalothrin, alpha-cypermethrin or deltametrin, and from the group 4A, the neonicotinoids, here especially acetamiprid, clothianidin, dinotefuran, imidacloprid, nitenpyram, thiacloprid or thiomethoxam.

Especially combinations of compounds of the invention with fiproles, neonicotinoids or pyrethroids may possibly exhibit synergistic control of stinkbugs (according to the Colby formula), in particular Euschistus, e.g. Euschistus heros.

The following list F of active substances, in conjunction with which the compounds according to the invention can be used, is intended to illustrate the possible combinations but does not limit them:

### F.I) Respiration Inhibitors

F.I-1) Inhibitors of complex III at Qo site
strobilurins: azoxystrobin, coumethoxystrobin, coumoxystrobin, dimoxystrobin, enestroburin, fluoxastrobin, kresoxim-methyl, metominostrobin, orysastrobin, picoxystrobin, pyraclostrobin, pyrametostrobin, pyraoxystrobin, pyribencarb, triclopyricarb/chlorodincarb, trifloxystrobin, 2-[2-(2,5-dimethyl-phenoxymethyl)-phenyl]-3-methoxy-acrylic acid methyl ester and 2 (2-(3-(2,6-dichlorophenyl)-1-methyl-allylideneaminooxymethyl)phenyl)-2-methoxyimino-N methyl-acetamide;
oxazolidinediones and imidazolinones: famoxadone, fenamidone;
F.I-2) Inhibitors of complex II (e.g. carboxamides):
carboxanilides: benodanil, benzovindiflupyr, bixafen, boscalid, carboxin, fenfuram, fenhexamid, fluopyram, flutolanil, furametpyr, isopyrazam, isotianil, mepronil, oxycarboxin, penflufen, penthiopyrad, sedaxane, tecloftalam, thifluzamide, tiadinil, 2-amino-4 methyl-thiazole-5-carboxanilide, N-(3′,4′,5′ trifluorobiphenyl-2 yl)-3-difluoromethyl-1-methyl-1H-pyrazole-4 carboxamide (fluxapyroxad), N-(4′-trifluoromethylthiobiphenyl-2-yl)-3 difluoromethyl-1-methyl-1H pyrazole-4-carboxamide, N-(2-(1,3,3-trimethyl-butyl)phenyl)-1,3-dimethyl-5 fluoro-1H-pyrazole-4 carboxamide, 3-(difluoromethyl)-1-methyl-N-(1,1,3-trimethylindan-4-yl)pyrazole-4-carboxamide, 3-(trifluoromethyl)-1-methyl-N-(1,1,3-trimethylindan-4-yl)pyrazole-4-carboxamide, 1,3-dimethyl-N-(1,1,3-trimethylindan-4-yl)pyrazole-4-carboxamide, 3-(trifluoromethyl)-1,5-dimethyl-N-(1,1,3-trimethylindan-4-yl)pyrazole-4-carboxamide, 3-(difluoromethyl)-1,5-dimethyl-N-(1,1,3-trimethylindan-4-yl)pyrazole-4-carboxamide, 1,3,5-trimethyl-N-(1,1,3-trimethylindan-4-yl)pyrazole-4-carboxamide, 3-(difluoromethyl)-1-methyl-N-(1,1,3-trimethylindan-4-yl)pyrazole-4-carboxamide, 3-(trifluoromethyl)-1-methyl-N-(1,1,3-trimethylindan-4-yl)pyrazole-4-carboxamide, 1,3-dimethyl-N-(1,1,3-trimethylindan-4-yl)pyrazole-4-carboxamide, 3-(trifluoromethyl)-1,5-dimethyl-N-(1,1,3-trimethylindan-4-yl)pyrazole-4-carboxamide, 3-(difluoromethyl)-1,5-dimethyl-N-(1,1,3-trimethylindan-4-yl)pyrazole-4-carboxamide, 1,3,5-trimethyl-N-(1,1,3-trimethylindan-4-yl)pyrazole-4-carboxamide;
F.I-3) Inhibitors of complex III at Qi site: cyazofamid, amisulbrom, [(3S,6S,7R,8R)-8-benzyl-3-[(3-acetoxy-4-methoxy-pyridine-2-carbonyl)amino]-6-methyl-4,9-dioxo-1,5-dioxonan-7-yl]2-methylpropanoate, [(3S,6S,7R,8R)-8-benzyl-3-[[3-(acetoxymethoxy)-4-methoxy-pyridine-2-carbonyl]amino]-6-methyl-4,9-dioxo-1,5-dioxonan-7-yl]2-methylpropanoate, [(3S,6S,7R,8R)-8-benzyl-3-[(3-isobutoxycarbonyloxy-4-methoxy-pyridine-2-carbonyl)amino]-6-methyl-4,9-dioxo-1,5-dioxonan-7-yl]2-methylpropanoate, [(3S,6S,7R,8R)-8-benzyl-3-[[3-(1,3-benzodioxol-5-ylmethoxy)-4-methoxy-pyridine-2-carbonyl]amino]-6-methyl-4,9-dioxo-1,5-dioxonan-7-yl]2-methylpropanoate, 3S,6S,7R,8R)-3-[[(3-hydroxy-4-methoxy-2-pyridinyl)carbonyl]amino]-6-methyl-4,9-dioxo-8-(phenylmethyl)-1,5-dioxonan-7-yl 2-methylpropanoate;
F.I-4) Other respiration inhibitors (complex I, uncouplers) diflumetorim; (5,8-difluoroquinazolin-4-yl)-{2-[2-fluoro-4-(4-trifluoromethylpyridin-2-yloxy)-phenyl]-ethyl}-amine; tecnazen; ametoctradin; silthiofam;
nitrophenyl derivates: binapacryl, dinobuton, dinocap, ferimzone; fluazinam, nitrthalisopropyl,
and including organometal compounds: fentin salts, such as fentin-acetate, fentin chloride or fentin hydroxide;
F.II) Sterol biosynthesis inhibitors (SBI fungicides)
F.II-1) C14 demethylase inhibitors (DMI fungicides, e.g. triazoles, imidazoles) triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, paclobutrazole, penconazole, propiconazole, prothioconazole, simeconazole, tebuconazole, tetraconazole, triadimefon, triadimenol, triticonazole, uniconazole, 1-[rel-(2 S3R)-3-(2-chlorophenyl)-2-(2,4-difluorophenyl)-oxiranylmethyl]-5-thiocyanato-1H[1,2,4]triazole, 2-[re/(2S;3R)-3-(2-chlorophenyl)-2-(2,4-difluorophenyl)-oxiranylmethyl]-2H-[1,2,4]triazole-3-thiol;
imidazoles: imazalil, pefurazoate, oxpoconazole, prochloraz, triflumizole;
pyrimidines, pyridines and piperazines: fenarimol, nuarimol, pyrifenox, triforine, 1-[rel-(2S,3R)-3-(2-chlorophenyl)-2-(2,4-difluorophenyl)-oxiranylmethyl]-5-thiocyanato-1H[1,2,4]triazole, 2-[rel-(2S;3R)-3-(2-chlorophenyl)-2-(2,4-difluorophenyl)-oxiranylmethyl]-2H-[1,2,4]triazole-3-thiol;
F.II-2) Delta14-reductase inhibitors (Amines, e.g. morpholines, piperidines) morpholines: aldimorph, dodemorph, dodemorph-acetate, fenpropimorph, tridemorph; piperidines: fenpropidin, piperalin;
spiroketalamines: spiroxamine;
F.II-3) Inhibitors of 3-keto reductase: hydroxyanilides: fenhexamid;
F.III) Nucleic acid synthesis inhibitors
F.III-1) RNA, DNA synthesis phenylamides or acyl amino acid fungicides: benalaxyl, benalaxyl-M, kiralaxyl, metalaxyl, metalaxyl-M (mefenoxam), ofurace, oxadixyl;
isoxazoles and iosothiazolones: hymexazole, octhilinone;
F.III-2) DNA topisomerase inhibitors: oxolinic acid;
F.III-3) Nucleotide metabolism (e.g. adenosin-deaminase) hydroxy (2-amino)-pyrimidines: bupirimate;
F.IV) Inhibitors of cell division and or cytoskeleton
F.IV-1) Tubulin inhibitors: benzimidazoles and thiophanates: benomyl, carbendazim, fuberidazole, thiabendazole, thiophanate-methyl; triazolopyrimidines: 5-chloro-7 (4-methylpiperidin-1-yl)-6-(2,4,6-trifluorophenyl)[1,2,4]triazolo[1,5 a]pyrimidine
F.IV-2) Other cell division inhibitors benzamides and phenyl acetamides: diethofencarb, ethaboxam, pencycuron, fluopicolide, zoxamide;
F.IV-3) Actin inhibitors: benzophenones: metrafenone, pyriofenone;
F.V) Inhibitors of amino acid and protein synthesis
F.V-1) Methionine synthesis inhibitors (anilino-pyrimidines) anilino-pyrimidines: cyprodinil, mepanipyrim, nitrapyrin, pyrimethanil;
F.V-2) Protein synthesis inhibitors (anilino-pyrimidines) antibiotics: blasticidin-S, kasugamycin, kasugamycin hydrochloride-hydrate, mildiomycin, streptomycin, oxytetracyclin, polyoxine, validamycin A;
F.VI) Signal transduction inhibitors
F.VI-1) MAP/Histidine kinase inhibitors (e.g. anilino-pyrimidines) dicarboximides: fluoroimid, iprodione, procymidone, vinclozolin;
phenylpyrroles: fenpiclonil, fludioxonil;
F.VI-2) G protein inhibitors: quinolines: quinoxyfen;
F.VII) Lipid and membrane synthesis inhibitors
F.VII-1) Phospholipid biosynthesis inhibitors
organophosphorus compounds: edifenphos, iprobenfos, pyrazophos;
dithiolanes: isoprothiolane;
F.VII-2) Lipid peroxidation: aromatic hydrocarbons: dicloran, quintozene, tecnazene, tolclofos-methyl, biphenyl, chloroneb, etridiazole;
F.VII-3) Carboxyl acid amides (CAA fungicides) cinnamic or mandelic acid amides: dimethomorph, flumorph, mandiproamid, pyrimorph; valinamide carbamates: benthiavalicarb, iprovalicarb, pyribencarb, valifenalate and N-(1-(1-(4-cyano-phenyl)ethanesulfonyl)-but-2-yl) carbamic acid-(4-fluorophenyl) ester;
F.VII-4) Compounds affecting cell membrane permeability and fatty acids: 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone,
carbamates: propamocarb, propamocarb-hydrochlorid,
F.VII-5) fatty acid amide hydrolase inhibitors: 1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone;
F.VIII) Inhibitors with Multi Site Action
F.VIII-1) Inorganic active substances: Bordeaux mixture, copper acetate, copper hydroxide, copper oxychloride, basic copper sulfate, sulfur;
F.VIII-2) Thio- and dithiocarbamates: ferbam, mancozeb, maneb, metam, methasulphocarb, metiram, propineb, thiram, zineb, ziram;
F.VIII-3) Organochlorine compounds (e.g. phthalimides, sulfamides, chloronitriles): anilazine, chlorothalonil, captafol, captan, folpet, dichlofluanid, dichlorophen, flusulfamide, hexachlorobenzene, pentachlorphenole and its salts, phthalide, tolylfluanid, N-(4-chloro-2-nitro-phenyl)-N-ethyl-4-methyl-benzenesulfonamide;
F.VIII-4) Guanidines and other: guanidine, dodine, dodine free base, guazatine, guazatine-acetate, iminoctadine, iminoctadine-triacetate, iminoctadine-tris(albesilate), 2,6-dimethyl-1H,5H-[1,4]dithiino[2,3-c:5,6-c′]dipyrrole-1,3,5,7(2H,6H)-tetraone;
F.VIII-5) Ahtraquinones: dithianon;
F.IX) Cell wall synthesis inhibitors
F.IX-1) Inhibitors of glucan synthesis: validamycin, polyoxin B;
F.IX-2) Melanin synthesis inhibitors: pyroquilon, tricyclazole, carpropamide, dicyclomet, fenoxanil;
F.X) Plant defense inducers
F.X-1) Salicylic acid pathway: acibenzolar-S-methyl;
F.X-2) Others: probenazole, isotianil, tiadinil, prohexadione-calcium;
phosphonates: fosetyl, fosetyl-aluminum, phosphorous acid and its salts;
F.XI) Unknown mode of action:
bronopol, chinomethionat, cyflufenamid, cymoxanil, dazomet, debacarb, diclomezine, difenzoquat, difenzoquat-methylsulfate, diphenylamin, fenpyrazamine, flumetover, flusulfamide, flutianil, methasulfocarb, nitrapyrin, nitrothal-isopropyl, oxathiapiprolin, oxin-copper, proquinazid, tebufloquin, tecloftalam, triazoxide, 2-butoxy-6-iodo-3-propylchromen-4-one, N-(cyclopropylmethoxyimino-(6-difluoro-methoxy-2,3-difluorophenyl)-methyl)-2-phenyl acetamide, N′-(4-(4-chloro-3-trifluoromethyl-phenoxy)-2,5-dimethyl-phenyl)-N-ethyl-N methyl formamidine, N′ (4-(4-fluoro-3-trifluoromethyl-phenoxy)-2,5-dimethyl-phenyl)-N-ethyl-N-methyl formamidine, N′-(2-methyl-5-trifluoromethyl-4-(3-trimethylsilanyl-propoxy)-phenyl)-N-ethyl-N-methyl formamidine, N′-(5-difluoromethyl-2 methyl-4-(3-trimethylsilanyl-propoxy)-phenyl)-N-ethyl-N-methyl formamidine, 2-{1-[2-(5-methyl-3-trifluoromethyl-pyrazole-1-yl)-acetyl]-piperidin-4-yl}-thiazole-4-carboxylic acid methyl-(1,2,3,4-tetrahydro-naphthalen-1-yl)-amide, 2-{1-[2-(5-methyl-3-trifluoromethyl-pyrazole-1-yl)-acetyl]-piperidin-4-yl}-thiazole-4-carboxylic acid methyl-(R)-1,2,3,4-tetrahydro-naphthalen-1-yl-amide, methoxy-acetic acid 6-tert-butyl-8-fluoro-2,3-dimethyl-quinolin-4-yl ester and N-Methyl-2-{1-[(5-methyl-3-trifluoromethyl-1H-pyrazol-1-yl)-acetyl]-piperidin-4-yl}-N-[(1R)-1,2,3,4-tetrahydronaphthalen-1-yl]-4-thiazolecarboxamide, 3-[5-(4-chloro-phenyl)-2,3-dimethyl-isoxazolidin-3 yl]-pyridine, pyrisoxazole, 5-amino-2-isopropyl-3-oxo-4-ortho-tolyl-2,3-dihydro-pyrazole-1 carbothioic acid S-allyl ester, N-(6-methoxy-pyridin-3-yl) cyclopropanecarboxylic acid amide, 5-chloro-1 (4,6-dimethoxy-pyrimidin-2-yl)-2-methyl-1H-benzoimidazole, 2-(4-chloro-phenyl)-N-[4-(3,4-dimethoxy-phenyl)-isoxazol-5-yl]-2-prop-2-ynyloxy-acetamide;
F.XII) Growth regulators:
abscisic acid, amidochlor, ancymidol, 6-benzylaminopurine, brassinolide, butralin, chlormequat (chlormequat chloride), choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat (mepiquat chloride), naphthaleneacetic acid, N 6 benzyladenine, paclobutrazol, prohexadione (prohexadione-calcium), prohydrojasmon, thidiazuron, triapenthenol, tributyl phosphorotrithioate, 2,3,5 tri iodobenzoic acid, trinexapac-ethyl and uniconazole;
F.XIII) Biological control agents
Ampelomyces quisqualis (e.g. AQ 10® from Intrachem Bio GmbH & Co. KG, Germany), Aspergillus flavus (e.g. AFLAGUARD® from Syngenta, CH), Aureobasidium pullulans (e.g. BOTECTOR® from bio-ferm GmbH, Germany), Bacillus pumilus (e.g. NRRL Accession No. B-30087 in SONATA® and BALLAD® Plus from AgraQuest Inc., USA), Bacillus subtilis (e.g. isolate NRRL-Nr. B-21661 in RHAPSODY®, SERENADE® MAX and SERENADE® ASO from AgraQuest Inc., USA), Bacillus subtilis var. amyloliquefaciens FZB24 (e.g. TAEGRO® from Novozyme Biologicals, Inc., USA), Candida oleophila I-82 (e.g. ASPIRE® from Ecogen Inc., USA), Candida saitoana (e.g. BIOCURE® (in mixture with lysozyme) and BIOCOAT® from Micro Flo Company, USA (BASF SE) and Arysta), Chitosan (e.g. ARMOUR-ZEN from BotriZen Ltd., NZ), Clonostachys rosea f. catenulata, also named Gliocladium catenulatum (e.g. isolate J1446: PRESTOP® from Verdera, Finland), Coniothyrium minitans (e.g. CONTANS® from Prophyta, Germany), Cryphonectria parasitica (e.g. Endothia parasitica from CNICM, France), Cryptococcus albidus (e.g. YIELD PLUS® from Anchor Bio-Technologies, South Africa), Fusarium oxysporum (e.g. BIOFOX® from S.I.A.P.A., Italy, FUSACLEAN® from Natural Plant Protection, France), Metschnikowia fructicola (e.g. SHEMER® from Agrogreen, Israel), Microdochium dimerum (e.g. ANTIBOT® from Agrauxine, France), Phlebiopsis gigantea (e.g. ROTSOP® from Verdera, Finland), Pseudozyma flocculosa (e.g. SPORODEX® from Plant Products Co. Ltd., Canada), Pythium oligandrum DV74 (e.g. POLYVERSUM® from Remeslo SSRO, Biopreparaty, Czech Rep.), Reynoutria sachlinensis (e.g. REGALIA® from Marrone Biolnnovations, USA), Talaromyces flavus V117b (e.g. PROTUS® from Prophyta, Germany), Trichoderma asperellum SKT-1 (e.g. ECO-HOPE® from Kumiai Chemical Industry Co., Ltd., Japan), T. atroviride LC52 (e.g. SENTINEL® from Agrimm Technologies Ltd, NZ), T. harzianum T-22 (e.g. PLANTSHIELD® der Firma BioWorks Inc., USA), T. harzianum TH 35 (e.g. ROOT PRO® from Mycontrol Ltd., Israel), T. harzianum T-39 (e.g. TRICHODEX® and TRICHODERMA 2000® from Mycontrol Ltd., Israel and Makhteshim Ltd., Israel), T. harzianum and T. viride (e.g. TRICHOPEL from Agrimm Technologies Ltd, NZ), T. harzianum ICC012 and T. viride ICC080 (e.g. REMEDIER® WP from Isagro Ricerca, Italy), T. polysporum and T. harzianum (e.g. BINAB® from BINAB Bio-Innovation AB, Sweden), T. stromaticum (e.g. TRICOVAB® from C.E.P.L.A.C., Brazil), T. virens GL-21 (e.g. SOILGARD® from Certis LLC, USA), T. viride (e.g. TRIECO® from Ecosense Labs. (India) Pvt. Ltd., Indien, BIOCURE® F from T. Stanes & Co. Ltd., Indien), T. viride TV1 (e.g. T. viride TV1 from Agribiotec srl, Italy), Ulocladium oudemansii HRU3 (e.g. BOTRY-ZEN® from Botry-Zen Ltd, NZ).

The commercially available compounds II of the group F listed above may be found in The Pesticide Manual, 15th Edition, C. D. S. Tomlin, British Crop Protection Council (2011) among other publications. Their preparation and their activity against harmful fungi is known (cf.: http://www.alanwood.net/pesticides/); these substances are commercially available. The compounds described by IUPAC nomenclature, their preparation and their fungicidal activity are also known (cf. Can. J. Plant Sci. 48(6), 587-94, 1968; EP A 141 317; EP-A 152 031; EP-A 226 917; EP A 243 970; EP A 256 503; EPA 428 941; EP-A 532 022; EP-A 1 028 125; EP-A 1 035 122; EP A 1 201 648; EP A 1 122 244, JP 2002316902; DE 19650197; DE 10021412; DE 102005009458; U.S. Pat. No. 3,296,272; U.S. Pat. No. 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 11/028657).

The invertebrate pest (also referred to as “animal pest”), i.e. the insects, arachnids and nematodes, the plant, soil or water in which the plant is growing or may grow can be contacted with the compounds of the present invention or composition(s) comprising them by any application method known in the art. As such, “contacting” includes both direct contact (applying the compounds/compositions directly on the invertebrate pest or plant—typically to the foliage, stem or roots of the plant) and indirect contact (applying the compounds/compositions to the locus of the invertebrate pest or plant).

The compounds of the present invention or the pesticidal compositions comprising them may be used to protect growing plants and crops from attack or infestation by animal pests, especially insects, acaridae or arachnids by contacting the plant/crop with a pesticidally effective amount of compounds of the present invention. The term “crop” refers both to growing and harvested crops.

The compounds of the present invention and the compositions comprising them are particularly important in the control of a multitude of insects on various cultivated plants, such as cereal, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

The compounds of the present invention are employed as such or in form of compositions by treating the insects or the plants, plant propagation materials, such as seeds, soil, surfaces, materials or rooms to be protected from insecticidal attack with an insecticidally effective amount of the active compounds. The application can be carried out both before and after the infection of the plants, plant propagation materials, such as seeds, soil, surfaces, materials or rooms by the insects.

Moreover, invertebrate pests may be controlled by contacting the target pest, its food supply, habitat, breeding ground or its locus with a pesticidally effective amount of compounds of the present invention. As such, the application may be carried out before or after the infection of the locus, growing crops, or harvested crops by the pest.

The compounds of the present invention can also be applied preventively to places at which occurrence of the pests is expected.

The compounds of the present invention may be also used to protect growing plants from attack or infestation by pests by contacting the plant with a pesticidally effective amount of compounds of the present invention. As such, “contacting” includes both direct contact (applying the compounds/compositions directly on the pest and/or plant—typically to the foliage, stem or roots of the plant) and indirect contact (applying the compounds/compositions to the locus of the pest and/or plant).

“Locus” means a habitat, breeding ground, plant, seed, soil, area, material or environment in which a pest or parasite is growing or may grow.

In general, “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95 weight %, preferably from 0.1 to 45 weight %, and more preferably from 1 to 25 weight % of at least one repellent and/or insecticide.

For use in treating crop plants, the rate of application of the active ingredients of this invention may be in the range of 0.1 g to 4000 g per hectare, desirably from 5 g to 500 g per hectare, more desirably from 5 g to 200 g per hectare.

The compounds of the present invention are effective through both contact (via soil, glass, wall, bed net, carpet, plant parts or animal parts), and ingestion (bait, or plant part).

The compounds of the present invention may also be applied against non-crop insect pests, such as ants, termites, wasps, flies, mosquitoes, crickets, or cockroaches. For use against said non-crop pests, compounds of the present invention are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickiness, moisture retention or aging characteristics.

The bait employed in the composition is a product, which is sufficiently attractive to incite insects such as ants, termites, wasps, flies, mosquitoes, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

For use in bait compositions, the typical content of active ingredient is from 0.001 weight % to 15 weight %, desirably from 0.001 weight % to 5% weight % of active ingredient.

Formulations of compounds of the present invention as aerosols (e.g in spray cans), oil sprays or pump sprays are highly suitable for the non-professional user for controlling pests such as flies, fleas, ticks, mosquitoes or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N-methylpyrrolidone, dimethyl sulfoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

For use in spray compositions, the content of active ingredient is from 0.001 to 80 weights %, preferably from 0.01 to 50 weight % and most preferably from 0.01 to 15 weight %.

The compounds of the present invention and its respective compositions can also be used in mosquito and fumigating coils, smoke cartridges, vaporizer plates or long-term vaporizers and also in moth papers, moth pads or other heat-independent vaporizer systems.

Methods to control infectious diseases transmitted by insects (e.g. malaria, dengue and yellow fever, lymphatic filariasis, and leishmaniasis) with compounds of the present invention and its respective compositions also comprise treating surfaces of huts and houses, air spraying and impregnation of curtains, tents, clothing items, bed nets, tsetse-fly trap or the like. Insecticidal compositions for application to fibers, fabric, knit-goods, nonwovens, netting material or foils and tarpaulins preferably comprise a mixture including the insecticide, optionally a repellent and at least one binder. Suitable repellents for example are N,N-Diethyl-meta-toluamide (DEET), N,N-diethylphenylacetamide (DEPA), 1-(3-cyclohexan-1-yl-carbonyl)-2-methylpiperine, (2-hydroxymethylcyclohexyl) acetic acid lactone, 2-ethyl-1,3-hexandiol, indalone, Methyl-neodecanamide (MNDA), a pyrethroid not used for insect control such as {(+/−)-3-allyl-2-methyl-4-oxocyclopent-2-(+)-enyl-(+)-trans-chrysanthemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and di-ethylenically unsaturated hydrocarbons carbons, such as styrene, and aliphatic diens, such as butadiene.

The impregnation of curtains and bednets is done in general by dipping the textile material into emulsions or dispersions of the insecticide or spraying them onto the nets.

The compounds of the present invention and their compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of the present invention are applied not only to the surrounding soil surface or into the under-floor soil in order to protect wooden materials but it can also be applied to lumbered articles such as surfaces of the under-floor concrete, alcove posts, beams, plywoods, furniture, etc., wooden articles such as particle boards, half boards, etc. and vinyl articles such as coated electric wires, vinyl sheets, heat insulating material such as styrene foams, etc.

In case of application against ants doing harm to crops or human beings, the ant controller of the present invention is applied to the crops or the surrounding soil, or is directly applied to the nest of ants or the like.

The compounds of the present invention are also suitable for the treatment of plant propagation material, especially seeds, in order to protect them from insect pest, in particular from soil-living insect pests and the resulting plant's roots and shoots against soil pests and foliar insects.

The compounds of the present invention are particularly useful for the protection of the seed from soil pests and the resulting plant's roots and shoots against soil pests and foliar insects. The protection of the resulting plant's roots and shoots is preferred.

More preferred is the protection of resulting plant's shoots from piercing and sucking insects, wherein the protection from aphids is most preferred.

The present invention therefore comprises a method for the protection of seeds from insects, in particular from soil insects and of the seedlings' roots and shoots from insects, in particular from soil and foliar insects, said method comprising contacting the seeds before sowing and/or after pregermination with a compound of the present invention, including a salt thereof. Particularly preferred is a method, wherein the plant's roots and shoots are protected, more preferably a method, wherein the plants shoots are protected form piercing and sucking insects, most preferably a method, wherein the plants shoots are protected from aphids.

The term seed embraces seeds and plant propagules of all kinds including but not limited to true seeds, seed pieces, suckers, corms, bulbs, fruit, tubers, grains, cuttings, cut shoots and the like and means in a preferred embodiment true seeds.

The term seed treatment comprises all suitable seed treatment techniques known in the art, such as seed dressing, seed coating, seed dusting, seed soaking and seed pelleting.

The present invention also comprises seeds coated with or containing the active compound.

The term “coated with and/or containing” generally signifies that the active ingredient is for the most part on the surface of the propagation product at the time of application, although a greater or lesser part of the ingredient may penetrate into the propagation product, depending on the method of application. When the said propagation product is (re)planted, it may absorb the active ingredient.

Suitable seed is seed of cereals, root crops, oil crops, vegetables, spices, ornamentals, for example seed of durum and other wheat, barley, oats, rye, maize (fodder maize and sugar maize/sweet and field corn), soybeans, oil crops, crucifers, cotton, sunflowers, bananas, rice, oilseed rape, turnip rape, sugarbeet, fodder beet, eggplants, potatoes, grass, lawn, turf, fodder grass, tomatoes, leeks, pumpkin/squash, cabbage, iceberg lettuce, pepper, cucumbers, melons, Brassica species, melons, beans, peas, garlic, onions, carrots, tuberous plants such as potatoes, sugar cane, tobacco, grapes, petunias, geranium/pelargoniums, pansies and impatiens.

In addition, the active compound may also be used for the treatment seeds from plants, which tolerate the action of herbicides or fungicides or insecticides owing to breeding, including genetic engineering methods.

For example, the active compound can be employed in treatment of seeds from plants, which are resistant to herbicides from the group consisting of the sulfonylureas, imidazolinones, glufosinate-ammonium or glyphosate-isopropylammonium and analogous active substances (see for example, EP-A 242 236, EP-A 242 246) (WO 92/00377) (EP-A 257 993, U.S. Pat. No. 5,013,659) or in transgenic crop plants, for example cotton, with the capability of producing Bacillus thuringiensis toxins (Bt toxins) which make the plants resistant to certain pests (EP-A 142 924, EP-A 193 259),

Furthermore, the active compound can be used also for the treatment of seeds from plants, which have modified characteristics in comparison with existing plants consist, which can be generated for example by traditional breeding methods and/or the generation of mutants, or by recombinant procedures). For example, a number of cases have been described of recombinant modifications of crop plants for the purpose of modifying the starch synthesized in the plants (e.g. WO 92/11376, WO 92/14827, WO 91/19806) or of transgenic crop plants having a modified fatty acid composition (WO 91/13972).

The seed treatment application of the active compound is carried out by spraying or by dusting the seeds before sowing of the plants and before emergence of the plants.

Compositions which are especially useful for seed treatment are e.g.:

A Soluble concentrates (SL, LS)

### D Emulsions (EW, EO, ES)

### E Suspensions (SC, OD, FS)

F Water-dispersible granules and water-soluble granules (WG, SG)
G Water-dispersible powders and water-soluble powders (WP, SP, WS)

### H Gel-Formulations (GF)

I Dustable powders (DP, DS)

Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter.

In a preferred embodiment a FS formulation is used for seed treatment. Typically, a FS formulation may comprise 1-800 g/l of active ingredient, 1-200 g/I Surfactant, 0 to 200 g/I antifreezing agent, 0 to 400 g/l of binder, 0 to 200 g/l of a pigment and up to 1 liter of a solvent, preferably water.

Especially preferred FS formulations of compounds of the present invention for seed treatment usually comprise from 0.1 to 80% by weight (1 to 800 g/I) of the active ingredient, from 0.1 to 20% by weight (1 to 200 g/I) of at least one surfactant, e.g. 0.05 to 5% by weight of a wetter and from 0.5 to 15% by weight of a dispersing agent, up to 20% by weight, e.g. from 5 to 20% of an anti-freeze agent, from 0 to 15% by weight, e.g. 1 to 15% by weight of a pigment and/or a dye, from 0 to 40% by weight, e.g. 1 to 40% by weight of a binder (sticker/adhesion agent), optionally up to 5% by weight, e.g. from 0.1 to 5% by weight of a thickener, optionally from 0.1 to 2% of an anti-foam agent, and optionally a preservative such as a biocide, antioxidant or the like, e.g. in an amount from 0.01 to 1% by weight and a filler/vehicle up to 100% by weight.

Seed Treatment formulations may additionally also comprise binders and optionally colorants.

Binders can be added to improve the adhesion of the active materials on the seeds after treatment. Suitable binders are homo- and copolymers from alkylene oxides like ethylene oxide or propylene oxide, polyvinylacetate, polyvinylalcohols, polyvinylpyrrolidones, and copolymers thereof, ethylene-vinyl acetate copolymers, acrylic homo- and copolymers, polyethyleneamines, polyethyleneamides and polyethyleneimines, polysaccharides like celluloses, tylose and starch, polyolefin homo- and copolymers like olefin/maleic anhydride copolymers, polyurethanes, polyesters, polystyrene homo and copolymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

### Examples of a Gelling Agent is Carrageen (Satiagel®)

In the treatment of seed, the application rates of the compounds of the present invention are generally from 0.01 g to 10 kg per 100 kg of seed, preferably from 0.05 g to 5 kg per 100 kg of seed, more preferably from 0.1 g to 1000 g per 100 kg of seed and in particular from 0.1 g to 200 g per 100 kg of seed.

The invention therefore also relates to seed comprising a compound of the present invention, including an agriculturally useful salt of it, as defined herein. The amount of the compound of the present invention, including an agriculturally useful salt thereof will in general vary from 0.01 g to 10 kg per 100 kg of seed, preferably from 0.05 g to 5 kg per 100 kg of seed, in particular from 0.1 g to 1000 g per 100 kg of seed. For specific crops such as lettuce the rate can be higher.

Methods which can be employed for treating the seed are, in principle, all suitable seed treatment and especially seed dressing techniques known in the art, such as seed coating (e.g. seed pelleting), seed dusting and seed imbibition (e.g. seed soaking).

Here, “seed treatment” refers to all methods that bring seeds and the compounds of the present invention into contact with each other, and “seed dressing” to methods of seed treatment which provide the seeds with an amount of the compounds of the present invention, i.e. which generate a seed comprising a compound of the present invention. In principle, the treatment can be applied to the seed at any time from the harvest of the seed to the sowing of the seed. The seed can be treated immediately before, or during, the planting of the seed, for example using the “planter's box” method. However, the treatment may also be carried out several weeks or months, for example up to 12 months, before planting the seed, for example in the form of a seed dressing treatment, without a substantially reduced efficacy being observed.

Expediently, the treatment is applied to unsown seed. As used herein, the term “unsown seed” is meant to include seed at any period from the harvest of the seed to the sowing of the seed in the ground for the purpose of germination and growth of the plant.

Specifically, a procedure is followed in the treatment in which the seed is mixed, in a suitable device, for example a mixing device for solid or solid/liquid mixing partners, with the desired amount of seed treatment formulations, either as such or after previous dilution with water, until the composition is distributed uniformly on the seed. If appropriate, this is followed by a drying step.

The compounds of the present invention, including their stereoisomers, veterinarily acceptable salts or N-oxides, are in particular also suitable for being used for combating parasites in and on animals.

An object of the present invention is therefore also to provide new methods to control parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions comprising a parasiticidally effective amount of compounds of the present invention, including their stereoisomers, veterinarily acceptable salts or N-oxides, and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of the present invention, including its stereoisomers, veterinarily acceptable salts or N-oxides, or a composition comprising it.

The invention also provides a process for the preparation of a composition for treating, controlling, preventing or protecting animals against infestation or infection by parasites which comprises a parasiticidally effective amount of a compound of the present invention, including its stereoisomers, veterinarily acceptable salts or N-oxides, or a composition comprising it.

Activity of compounds against agricultural pests does not suggest their suitability for control of endo- and ectoparasites in and on animals which requires, for example, low, non-emetic dosages in the case of oral application, metabolic compatibility with the animal, low toxicity, and a safe handling.

Surprisingly it has now been found that compounds of formula (I) and their stereoisomers, veterinarily acceptable salts, tautomers and N-oxides, are suitable for combating endo- and ectoparasites in and on animals.

The compounds of the present invention, especially compounds of formula (I) and their stereoisomers, veterinarily acceptable salts, tautomers and N-oxides, and compositions comprising them are preferably used for controlling and preventing infestations of and infections in animals including warm-blooded animals (including humans) and fish.

They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels. Compounds of the present invention, including their stereoisomers, veterinarily acceptable salts or N-oxides, and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of the present invention, including their stereoisomers, veterinarily acceptable salts or N-oxides, and compositions comprising them are suitable for systemic and/or non-systemic control of ecto- and/or endoparasites. They are active against all or some stages of development.

The compounds of the present invention are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus, cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,
flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,
lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.
ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus bacoti and Dermanyssus gallinae,
Actinedida (Prostigmata) und Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp,
Bugs (Heteropterida): Cimex lectularius, Cimex hemipterus, Reduvius senilis, Triatoma spp., Rhodnius ssp., Panstrongylus ssp. and Arilus critatus, Anoplurida, e.g. Haematopinus spp., Linognathus spp., Pediculus spp., Phtirus spp., and Solenopotes spp,
Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp,

### Roundworms Nematoda:

Wipeworms and Trichinosis (Trichosyringida), e.g. Trichinellidae (Trichinella spp.), (Trichuridae) Trichuris spp., Capillaria spp,
Rhabditida, e.g. Rhabditis spp, Strongyloides spp., Helicephalobus spp, Strongylida, e.g. Strongylus spp., Ancylostoma spp., Necator americanus, Bunostomum spp. (Hookworm), Trichostrongylus spp., Haemonchus contortus., Ostertagia spp., Cooperia spp., Nematodirus spp., Dictyocaulus spp., Cyathostoma spp., Oesophagostomum spp., Stephanurus dentatus, Ollulanus spp., Chabertia spp., Stephanurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale,
Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi,
Camallanida, e.g. Dracunculus medinensis (guinea worm)
Spirurida, e.g. Thelazia spp. Wuchereria spp., Brugia spp., Onchocerca spp., Dirofilari spp.a, Dipetalonema spp., Setaria spp., Elaeophora spp., Spirocerca lupi, and Habronema spp.,
Thorny headed worms (Acanthocephala), e.g. Acanthocephalus spp., Macracanthorhynchus hirudinaceus and Oncicola spp,

### Planarians (Plathelminthes):

Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp, Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The present invention relates to the therapeutic and the non-therapeutic use of compounds of the present invention and compositions comprising them for controlling and/or combating parasites in and/or on animals. The compounds of the present invention and compositions comprising them may be used to protect the animals from attack or infestation by parasites by contacting them with a parasiticidally effective amount of compounds of the present invention and compositions containing them.

The compounds of the present invention and compositions comprising them can be effective through both contact (via soil, glass, wall, bed net, carpet, blankets or animal parts) and ingestion (e.g. baits). As such, “contacting” includes both direct contact (applying the pesticidal mixtures/compositions containing the compounds of the present invention directly on the parasite, which may include an indirect contact at its locus-P, and optionally also administrating the pesticidal mixtures/composition directly on the animal to be protected) and indirect contact (applying the compounds/compositions to the locus of the parasite). The contact of the parasite through application to its locus is an example of a non-therapeutic use of compounds of the present invention. “Locus-P” as used above means the habitat, food supply, breeding ground, area, material or environment in which a parasite is growing or may grow outside of the animal.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions of the present invention. A parasiticidally effective amount of the compositions will also vary according to the prevailing conditions such as desired parasiticidal effect and duration, target species, mode of application, and the like.

The compounds of the present invention can also be applied preventively to places at which occurrence of the pests or parasites are expected.

Administration can be carried out both prophylactically and therapeutically.

Administration of the active compounds is carried out directly or in the form of suitable preparations, orally, topically/dermally or parenterally.

### EXAMPLES

The present invention is now illustrated in further details by the following examples, without imposing any limitation thereto.

### A. Preparation Examples

Compounds can be characterized e.g. by coupled High Performance Liquid Chromatography/mass spectrometry (HPLC/MS), by ¹H-NMR and/or by their melting points.

Analytical HPLC: Phenomenex Kinetex 1.7 μm XB-C18 100A; 50×2.1 mm

Elution: A: acetonitrile+0.1% trifluoroacetic acid (TFA)/water+0.1% trifluoroacetic acid (TFA) in a ratio of from 5:95 to 95:5 in 1.5 minutes at 50° C.

RT or r.t.=HPLC retention time; m/z of the [M+H]+, [M+Na]+ or [M+K]+ peaks.

¹H-NMR, respectively ¹³C-NMR: The signals are characterized by chemical shift (ppm, δ [delta]) vs. tetramethylsilane, respectively CDCl₃ for ¹³C-NMR, by their multiplicity and by their integral (relative number of hydrogen atoms given). The following abbreviations are used to characterize the multiplicity of the signals: m=multiplett, q=quartett, t=triplett, d=doublet and s=singulett.

Abbreviations used are: h for hour(s), min for minute(s), r.t./room temperature for 20-25° C., THF for tetrahydrofuran, OAc for acetate.

### C.1 Compound Examples 1

Compound examples 1-1 to 1-18 correspond to compounds of formula C.1:

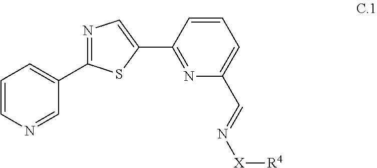

wherein X and R⁴ of each synthesized compound is defined in one row of table C.1 below.

Compounds 1-1 to 1-9 were synthesized in analogy to Synthesis Example S.1, and compounds 1-10 to 1-18 were synthesized in analogy to Synthesis Example S.2.

TABLE C.1

| | | | | HPLC-MS: Rₜ |
|---|---|---|---|---|
| | | | | (min) and |
| Ex. | X | R⁴ | ¹H NMR | [M + H]⁺ |
| 1-1 | O | CH₃ | d-DMSO, 360 MHz, δ 9.24-9.19 (m, 1H), | 0.911 min, |
| | | | 8.74-8.67 (m, 2H), 8.42-8.35 (m, 1H), 8.17 | [M + H]⁺ = |
| | | | (s, 1H), 8.11 (d, 1H, J = 3.9 Hz), 7.97 (t, 1H, | 297 |
| | | | J = 7.8 Hz), 7.74 (d, 1H, J = 3.9 Hz), 7.60-7.54 | |
| | | | (m, 1H), 3.98 (s, 3H) | |
| 1-2 | O | C₂H₅ | THF-d₈, 400 MHz, δ 9.23 (s, 1H), 8.62 (d, | 0.996 min, |
| | | | 1H, J = 2.4 Hz), 8.45 (s, 1H), 8.32 (d, 1H, | [M + H]⁺ = |
| | | | J = 4.0 Hz), 8.12 (s, 1H), 7.87 (d, 1H, | 311 |
| | | | J = 3.5 Hz), 7.80 (q, 2H, J = 8.1 Hz), 7.46-7.39 | |
| | | | (m, 1H), 4.25 (q, 2H, J = 7.0 Hz), 0.89 (t, | |
| | | | 3H, J = 6.2 Hz) | |
| 1-3 | O | CH(CH₃)₂ | THF-d₈, 500 MHz, δ 9.22 (s, 1H), 8.64-8.58 | 1.117 min, |
| | | | (m, 1H), 8.44 (s, 1H), 8.32 (d, 1H, | [M + H]⁺ = |
| | | | J = 3.4 Hz), 8.09 (s, 1H), 7.88-7.84 (m, 1H), | 325 |
| | | | 7.84-7.74 (m, 2H), 7.44-7.38 (m, 1H), | |
| | | | 4.53-4.41 (m, 1H), 1.31 (d, 6H, J = 3.3 Hz) | |
| 1-4 | O | CH₂—^{c}C₃H₅ | THF-d₈, 500 MHz, δ 9.22 (s, 1H), 8.65-8.59 | 1.108 min, |
| | | | (m, 1H), 8.44 (s, 1H), 8.32 (d, 1H, | [M + H]⁺ = |
| | | | J = 3.9 Hz), 8.13 (s, 1H), 7.88-7.84 (m, 1H), | 337 |
| | | | 7.84-7.74 (m, 2H), 7.45-7.36 (m, 1H), 4.02 | |
| | | | (d, 2H, J = 3.3 Hz), 1.26 (m, 1H), 0.55 (d, | |
| | | | 2H, J = 3.4 Hz), 0.38-0.29 (m, 2H) | |
| 1-5 | O | allyl | THF-d₈, 500 MHz, δ 9.21 (s, 1H), 8.62-8.56 | 1.058 min, |
| | | | (m, 1H), 8.45 (m, 1H), 8.32 (d, 1H, | [M + H]⁺ = |
| | | | J = 4.6 Hz), 8.16 (s, 1H), 7.87 (d, 1H, | 323 |
| | | | J = 3.7 Hz), 7.84-7.72 (m, 2H), 7.44-7.35 | |
| | | | (m, 1H), 6.10-5.98 (m, 1H), 5.33 (d, 1H, | |
| | | | J = 8.6 Hz), 5.20 (d, 1H, J = 5.0 Hz), 4.70 (d, | |
| | | | 2H, J = 2.6 Hz) | |
| 1-6 | O | propargyl | THF- d₈, 500 MHz, δ 9.22 (s, 1H), 8.61 (d, | 0.947 min, |
| | | | 1H, J = 4.5 Hz), 8.48-8.43 (m, 1H), 8.35- | [M + H]⁺ = |
| | | | 8.29 (m, 1H), 8.19-8.15 (m, 1H), 7.94-7.87 | 321.1 |
| | | | (m, 1H), 7.87-7.78 (m, 2H), 7.44-7.38 (m, | |
| | | | 1H), 4.80 (s, 2H), 2.96-2.92 (m, 1H) | |
| 1-7 | O | benzyl | d-DMSO, 500 MHz, δ 9.20 (s, 1H), 8.70 (s, | 1.148 min, |
| | | | 2H), 8.37 (dt, 1H, J = 8.4 Hz, 2.3 Hz), 8.25 | [M + H]⁺ = |
| | | | (s, 1H), 8.11 (d, 1H, J = 3.8 Hz), 7.96 (t, 1H, | 373 |
| | | | J = 8.0 Hz), 7.73 (d, 1H, J = 3.8 Hz), 7.58-7.54 | |
| | | | (m, 1H), 7.45 (d, 2H, J = 3.8 Hz), 7.40 (t, | |
| | | | 2H, J = 7.5 Hz), 7.38-7.32 (m, 1H), 5.27 (s, | |
| | | | 2H) | |
| 1-8 | NCH₃ | CH₃ | THF-d₈, 500 MHz, δ 9.21 (s, 1H), 8.64-8.55 | 0.850 min, |
| | | | (m, 1H), 8.38 (s, 1H), 8.30 (d, 1H, | [M + H]⁺ = |
| | | | J = 4.4 Hz), 7.71 (d, 1H, J = 3.5 Hz), 7.68-7.58 | 310 |
| | | | (m, 1H), 7.43-7.36 (m, 1H), 7.25 (s, 1H), | |
| | | | 3.05 (s, 6H) | |
| 1-9 | NH | phenyl | THF-d₈, 500 MHz, δ 9.80 (s, 1H), 9.22 (s, | 1.096 min, |
| | | | 1H), 8.63-8.57 (m, 1H), 8.45-8.40 (m, 1H), | [M + H]⁺ = |
| | | | 8.35-8.29 (m, 1H), 7.93 (d, 1H, J = 3.5 Hz), | 358 |
| | | | 7.81 (s, 1H), 7.79-7.69 (m, 2H), 7.44-7.39 | |
| | | | (m, 1H), 7.20 (t, 2H, J = 6.9 Hz), 7.13 (d, | |
| | | | 2H, J = 4.0 Hz), 6.79 (t, 1H, J = 7.5 Hz) | |
| 1-10 | O | phenyl | d-DMSO, 400 MHz, δ 9.25-9.20 (m, 1H), | 1.185 min, |
| | | | 8.76 (s, 1H), 8.71 (d, 1H, J = 2.7 Hz), 8.64 | [M + H]⁺ = |
| | | | (s, 1H), 8.40 (d, 1H, J = 3.7 Hz), 8.22 (d, 1H, | 359 |
| | | | J = 3.7 Hz), 8.07 (t, 1H, J = 7.5 Hz), 7.98 (d, | |
| | | | 1H, J = 3.7 Hz), 7.58 (q, 1H, J = 4.8 Hz), 7.42 | |
| | | | (t, 2H, J = 7.5 Hz), 7.32 (d, 2H, J = 4.3 Hz), | |
| | | | 7.12 (t, 1H, J = 7.5 Hz) | |
| 1-11 | O | H | d-DMSO, 400 MHz, δ 9.21 (s, 1H), 8.71 (t, | 0.751 min, |
| | | | 2H, J = 5.5 Hz), 8.39 (d, 1H, J = 4.5 Hz), 8.11- | [M + H]⁺ = |
| | | | 8.07 (m, 2H), 7.96 (t, 1H, J = 7.9 Hz), 7.75 | 283 |
| | | | (d, 1H, J = 3.7 Hz), 7.61-7.55 (m, 1H) | |
| 1-12 | O | CH₂CF₃ | d-DMSO, 400 MHz, δ 9.23-9.18 (m, 1H), | 1.072 min, |
| | | | 8.74-8.68 (m, 2H), 8.42-8.35 (m, 2H), 8.18 | [M + H]⁺ = |
| | | | (d, 1H, J = 4.0 Hz), 8.02 (t, 1H, J = 8.0 Hz), | 365 |
| | | | 7.75 (d, 1H, J = 4.0 Hz), 7.61-7.54 (m, 1H), | |
| | | | 4.89 (q, 2H, J = 9.6 Hz) | |
| 1-13 | NH | benzyl | d-DMSO, 400 MHz, δ 9.22-9.18 (m, 1H), | 1.055 min, |
| | | | 8.68 (d, 1H, J = 2.2 Hz), 8.62 (s, 1H), 8.48- | [M + H]⁺ = |
| | | | 8.43 (m, 1H), 8.37 (d, 1H, J = 4.0 Hz), 7.88- | 372 |
| | | | 7.77 (m, 2H), 7.66 (d, 1H, J = 3.5 Hz), 7.56 | |
| | | | (q, 1H, J = 4.9 Hz), 7.49 (s, 1H), 7.37 (d, 4H, | |
| | | | J = 2.2 Hz), 7.31-7.25 (m, 1H), 4.49-4.44 | |
| | | | (m, 2H) | |
| 1-14 | NH | COCH₃ | d-DMSO, 360 MHz, δ 11.64 and 11.56 (s, | 0.745 min, |
| | | | 1H), 9.24-9.19 (m, 1H), 8.72-8.67 (m, 2H), | [M + H]⁺ = |
| | | | 8.39 (dt, 1H, J = 8.2 Hz 2.0 Hz), 8.20 and | 324 |
| | | | 8.02 (s, 1H), 8.11-8.05 (m, 1H), 8.00-7.95 | |
| | | | (m, 1H), 7.90-7.84 (m, 1H), 7.60-7.54 (m, | |
| | | | 1H), 2.25 and 2.01 (s, 3H) | |
| 1-15 | NH | CO- | d-DMSO, 360 MHz, δ 12.12 (s, 1H), 9.30- | 0.880 min, |
| | | phenyl | 9.21 (m, 1H), 8.77-8.69 (m, 2H), 8.54 (s, | [M + H]⁺ = |
| | | | 1H), 8.44-8.38 (m, 1H), 8.17-7.91 (m, 5H), | 386 |
| | | | 7.71-7.53 (m, 4H) | |
| 1-16 | NH | CO— | d-DMSO, 400 MHz, δ 10.75 (s, 1H), 9.24- | 0.751 min, |
| | | NHCH₃ | 9.19 (m, 1H), 8.71-8.64 (m, 2H), 8.43-8.36 | [M + H]⁺ = |
| | | | (m, 1H), 8.15 (d, 1H, J = 3.9) 8.03 (d, 1H, | 339 |
| | | | J = 3.9 Hz), 7.95 (t, 1H, J = 7.8 Hz), 7.87 (s, | |
| | | | 1H), 7.60-7.52 (m, 1H), 7.23-7.15 (m, 1H), | |
| | | | 2.73 (d, 3H, J = 2.1 Hz) | |
| 1-17 | NH | CO—NH— | d-DMSO, 400 MHz, δ 11.07 (s, 1H), 9.24- | 0.878 min, |
| | | CH₂CF₃ | 9.19 (m, 1H), 8.73-8.67 (m, 2H), 8.40 (d, | [M + H]⁺ = |
| | | | 1H, J = 4.1 Hz), 8.20 (d, 1H, J = 3.7 Hz), 8.07 | 407 |
| | | | (d, 1H, J = 4.1 Hz), 7.98 (t, 1H, J = 7.7 Hz), | |
| | | | 7.93 (s, 1H), 7.88 (t, 1H, J = 6.5 Hz), 7.60- | |
| | | | 7.54 (m, 1H), 4.00-3.88 (m, 2H) | |
| 1-18 | NH | CO—NH— | d-DMSO, 360 MHz, δ 10.72 (s, 1H), 9.25- | 0.905 min, |
| | | CH₂—^{c}C₃H₅ | 9.20 (m, 1H), 8.74-8.67 (m, 2H), 8.40 (dt, | [M + H]⁺ = |
| | | | 1H, J = 8.1 Hz 1.9 Hz), 8.15 (d, 1H, | 379 |
| | | | J = 3.9 Hz), 8.04 (d, 1H, J = 3.9 Hz), 7.95 (t, | |
| | | | 1H, J = 7.7 Hz), 7.89 (s, 1H), 7.61-7.54 (m, | |
| | | | 1H), 7.30 (t, 1H, J = 6.0 Hz), 3.06 (t, 2H, | |
| | | | J = 6.5 Hz), 1.08-1.03 (m, 1H), 0.46-0.38 | |
| | | | (m, 2H), 0.28-0.20 (m, 2H) | |

^{c}C₃H₅ = cyclopropyl

### C.2 Compound Examples 2

Compound example 2-1 corresponds to compounds of formula C.2:

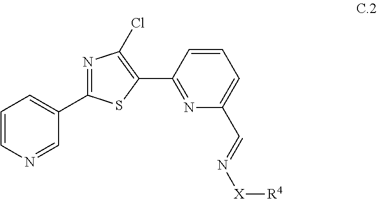

wherein X and R⁴ of each synthesized compound is defined in one row of table C.2 below.

TABLE C.2

| | | | | HPLC-MS: Rₜ |
|---|---|---|---|---|
| | | | | (min) and |
| Ex. | X | R⁴ | ¹H NMR | [M + H]⁺ |
| 2-1 | O | CH₃ | THF- d₈, 500 MHz, δ 9.20 (s, 1H), | 1.113 min, |
| | | | 8.69-8.62 (m, 1H), 8.31 (d, 2H, J = | [M + H]⁺ = |
| | | | 4.0 Hz), 8.09 (s, 1H), 7.91 (t, 1H, J = | 331 |
| | | | 8.0 Hz), 7.84 (d, 1h, J = 4.0 Hz), 7.47- | |
| | | | 7.41 (m, 1H), 3.99 (s, 3H) | |

### C.3 Compound Examples 3

Compound examples 3-1 to 3-65 correspond to compounds of formula C.3:

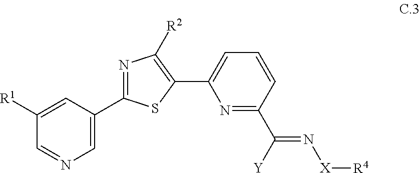

wherein R¹, R², Y and X—R₄ of each synthesized compound is defined in one row of table C.3 below.

TABLE C.3

| | | | | | MS- | MS- |
|---|---|---|---|---|---|---|
| | | | | | Retention | Mass |
| | | | | | Time | Charge |
| Ex. | R¹ | R² | Y | X—R⁴ | [min] | Ratio |
| 3-1 | H | H | CH₃ | 2-pyridylamino | 0.800 | 373.2 |
| 3-2 | H | H | CH₃ | OCH₃ | 1.049 | 311.5 |
| 3-3 | H | H | CH₃ | NHC₆H₅ | 1.169 | 372.2 |
| 3-4 | H | H | H | 4-chloroanilino | 1.177 | 392.1 |
| 3-5 | H | H | H | 3,5-difluoroanilino | 1.159 | 394.2 |
| 3-6 | H | H | H | 1,3-benzodioxol-5- | 1.087 | 402.2 |
| | | | | ylamino | | |
| 3-7 | H | H | H | 4-(trifluoromethoxy) | 1.222 | 442.1 |
| | | | | anilino | | |
| 3-8 | H | H | H | 1-naphthylamino | 1.247 | 408.1 |
| 3-9 | H | H | H | 3-(trifluoromethyl) | 1.238 | 426.2 |
| | | | | anilino | | |
| 3-10 | H | H | H | 3,5-dichloroanilino | 1.295 | 427.9 |
| 3-11 | H | H | H | morpholino | 0.879 | 352.2 |
| 3-12 | H | H | H | pyrrolidin-1-yl | 0.939 | 336.2 |
| 3-13 | H | H | H | N-butylanilino | 1.347 | 414.4 |
| 3-14 | H | H | H | 2-fluoroanilino | 1.120 | 376.0 |
| 3-15 | H | H | H | 3-fluoroanilino | 1.110 | 376.0 |
| 3-16 | H | H | H | 4-cyanoanilino | 1.082 | 383.1 |
| 3-17 | H | H | H | 3,5-dimethylanilino | 1.201 | 386.1 |
| 3-18 | H | H | H | 2-methoxyanilino | 1.126 | 387.9 |
| 3-19 | H | H | H | 4-methoxyanilino | 1.052 | 388.1 |
| 3-20 | H | H | H | 4-(trifluoromethyl) | 1.207 | 426.1 |
| | | | | anilino | | |
| 3-21 | H | H | H | 3-chloroanilino | 1.174 | 392.0 |
| 3-22 | H | H | H | 2-ethylanilino | 1.243 | 386.2 |
| 3-23 | H | H | H | 2,3,5,6-tetrafluoroanilino | 1.152 | 430.1 |
| 3-24 | H | H | H | 4,5-dihydro-1H- | 0.711 | 350.1 |
| | | | | imidazol-2-ylamino | | |
| 3-25 | H | H | H | 2,4,6-trimethylanilino | 1.239 | 400.2 |
| 3-26 | H | H | H | 2,3-dihydro-1,4- | 1.073 | 416.2 |
| | | | | benzodioxin-6-ylamino | | |
| 3-27 | H | H | H | N-methylanilino | 1.160 | 372.1 |
| 3-28 | H | H | H | 2-pyridylamino | 0.685 | 359.1 |
| 3-29 | H | H | H | 4-chloropyrazol-1-yl | 1.101 | 367.1 |
| 3-30 | H | H | H | (6-chloro-2- | 1.104 | 393.1 |
| | | | | pyridyl)amino | | |
| 3-31 | H | H | H | (5-chloro-2- | 0.999 | 393.1 |
| | | | | pyridyl)amino | | |
| 3-32 | H | H | H | 2,6-dichloro-4- | 1.339 | 494.1 |
| | | | | (trifluoromethyl) | | |
| | | | | anilino | | |
| 3-33 | H | H | H | 4-fluoroanilino | 1.136 | 376.1 |
| 3-34 | H | H | H | 4-methylanilino | 1.148 | 372.2 |
| 3-35 | H | H | H | N-benzylanilino | 1.342 | 448.2 |
| 3-36 | H | H | H | 3-chloro-4-fluoro- | 1.181 | 410.1 |
| | | | | anilino | | |
| 3-37 | H | H | CH₃ | 4-(trifluoromethoxy) | 1.286 | 456.2 |
| | | | | anilino | | |
| 3-38 | H | H | CH₃ | NHC(O)CH₃ | 0.818 | 338.1 |
| 3-39 | H | H | CH₃ | O(CH₂)₂OCH₂CH₃ | 1.065 | 369.1 |
| 3-40 | H | H | CH₃ | 3,5-difluoroanilino | 1.245 | 408.1 |
| 3-41 | H | H | CH₃ | OⁱPr | 1.238 | 339.1 |
| 3-42 | H | H | CH₃ | 4-(trifluoromethyl) | 1.272 | 440.2 |
| | | | | anilino | | |
| 3-43 | F | H | H | OCH₃ | 1.203 | 315.1 |
| 3-44 | F | H | H | NHC₆H₅ | 1.318 | 376.1 |
| 3-45 | F | H | H | N(CH₃)₂ | 1.126 | 328.1 |
| 3-46 | F | CI | H | OCH₃ | 1.353 | 349.1 |
| 3-47 | H | H | CF₃ | OCH₃ | 1.050 | 365.1 |
| 3-48 | H | H | CF₃ | NHC₆H₅ | 1.356 | 426.1 |
| 3-49 | H | H | CF₃ | NHCH₂Ph | 1.306 | 440.1 |
| 3-50 | H | CI | H | N(CH₃)₂ | 1.102 | 344.1 |
| 3-51 | H | H | H | O(CH₂)₂OCH₂CH₃ | 0.984 | 354.9 |
| 3-52 | H | H | H | 2,4-dioxoimidazolidin- | 0.694 | 365.5 |
| | | | | 1-yl | | |
| 3-53 | H | H | H | 1-piperidyl | 1.064 | 350.6 |
| 3-54 | H | CI | H | NHC₆H₅ | 1.286 | 391.7 |
| 3-55 | H | CF₃ | H | N(CH₃)₂ | 1.116 | 377.8 |
| 3-56 | H | CF₃ | H | OCH₃ | 1.145 | 365.5 |
| 3-57 | H | H | H | 2-oxopyrrolidin-1-yl | 0.747 | 349.8 |
| 3-58 | H | H | H | 2-oxo-1-piperidyl | 0.778 | 363.8 |
| 3-59 | H | H | H | methanesulfonamido | 0.761 | 360.5 |
| 3-60 | H | CH₂OH | H | OCH₃ | 0.825 | 327.5 |
| 3-61 | H | CHF₂ | H | OCH₃ | 1.041 | 346.8 |
| 3-62 | H | CH₂F | H | OCH₃ | 0.994 | 329.5 |
| 3-63 | H | CHF₂ | H | N(CH₃)₂ | 1.048 | 360.6 |
| 3-64 | H | CH₂F | H | N(CH₃)₂ | 0.947 | 342.6 |
| 3-65 | H | CH₂OH | H | N(CH₃)₂ | 0.745 | 340.6 |

### C.4 Compound Examples 4

Compound examples 4-1 to 4-4 correspond to compounds of formula C.4:

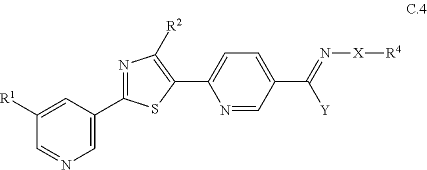

wherein R¹, R², Y and X—R⁴ of each synthesized compound is defined in one row of table C.4 below.

TABLE C.4

| | | | | | MS- | MS- |
|---|---|---|---|---|---|---|
| | | | | | Retention | Mass |
| | | | | | Time | Charge |
| Ex. | R¹ | R² | Y | X—R⁴ | [min] | Ratio |
| 4-1 | H | H | H | NHC₆H₅ | 1.052 | 358.1 |
| 4-2 | H | H | H | N(CH₃)₂ | 0.92 | 310.1 |
| 4-3 | H | H | H | OMe | 0.903 | 297.1 |
| 4-4 | H | H | H | NHCH₂C₆H₅ | 1.009 | 372.2 |

### C.5 Compound Examples 5

Compound examples 5-1 to 5-5 correspond to compounds of formula C.5:

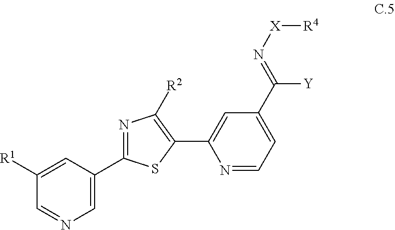

wherein R¹, R², Y and X—R⁴ of each synthesized compound is defined in one row of table C.5 below.

TABLE C.5

| | | | | | MS- | MS-Mass |
|---|---|---|---|---|---|---|
| | | | | | Retention | Charge |
| Ex. | R¹ | R² | Y | X—R⁴ | Time [min] | Ratio |
| 5-1 | H | H | H | OCH₃ | 0.885 | 297.1 |
| 5-2 | H | H | H | NHC₆H₅ | 0.979 | 358.1 |
| 5-3 | H | H | H | OCH₂CCH | 0.917 | 321.1 |
| 5-4 | H | H | H | NHCH₂C₆H₅ | 0.966 | 372.2 |
| 5-5 | H | H | H | N(CH₃)₂ | 0.742 | 310.2 |

### C.6 Compound Examples 6

Compound examples 6-1 to 6-4 correspond to compounds of formula C.6:

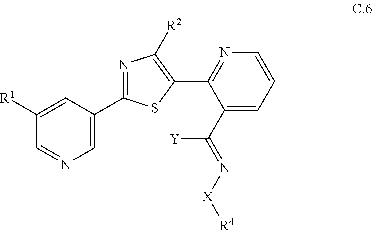

wherein R¹, R², Y and X—R⁴ of each synthesized compound is defined in one row of table C.6 below.

TABLE C.6

| | | | | | MS- | MS-Mass |
|---|---|---|---|---|---|---|
| | | | | | Retention | Charge |
| Ex. | R¹ | R² | Y | X—R⁴ | Time [min] | Ratio |
| 6-1 | H | H | H | NHC₆H₅ | 1.037 | 358.2 |
| 6-2 | H | H | H | OCH₃ | 0.843 | 297.2 |
| 6-3 | H | H | H | N(CH₃)₂ | 0.821 | 310.2 |
| 6-4 | H | H | H | NHC(O)CH₃ | 0.687 | 324.1 |

### SYNTHESIS EXAMPLES

### Synthesis of the Precursors

### 1. Synthesis of the Precursors of Formula P.1

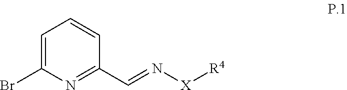

### 1.1 Synthesis of 6-bromopyridine-2-carbaldehyde-O-methyloxime (compound P.1-1 of formula P.1 wherein X═O and R⁴=methyl)

A mixture of 6-bromopyridine-2-carbaldehyde (2.00 g, 10.8 mmol) and O-methylhydroxylamine hydrochloride (1.08 g, 12.9 mmol) in acetic acid (2 mL) and ethanol (20 mL) was heated at 70° C. for 2 h. The reaction mixture was concentrated under reduced pressure. The residue dissolved in ethyl acetate and extracted with a saturated aqueous solution of sodium bicarbonate. The organic phase was dried over magnesium sulfate and concentrated under reduced pressure to give P.1-1 in sufficient purity for the next step (1.91 g, 83%).

The following precursors were synthesized analogously:

| Precursor | X | R⁴ |
|---|---|---|
| P.1-2 | O | C₂H₅ |
| P.1-3 | O | CH(CH₃)₂ |
| P.1-4 | O | CH₂—^{c}C₃H₅ |
| P.1-5 | O | allyl |
| P.1-6 | O | propargyl |
| P.1-7 | O | benzyl |
| P.1-8 | NCH₃ | CH₃ |
| P.1-9 | NH | phenyl |

### 1.2 Synthesis of Compound P.2

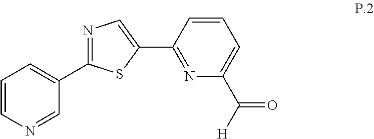 [SMILES: O=Cc1cccc(-c2cnc(-c3cccnc3)s2)n1]

A mixture of 3-(thiazol-2-yl)-pyridine (406 mg, 2.50 mmol), 6-bromopyridine-2-carbaldehyde (511 mg, 2.75 mmol), palladium acetate (56 mg, 0.25 mmol), tricyclohexylphosphonium tetrafluoroborate (184 mg, 0.50 mmol), pivalic acid (51 mg, 0.50 mmol), and potassium carbonate (518 mg, 3.75 mmol) in dimethylacetamide (7 mL) were heated at 100° C. for 16 h. The reaction mixture was diluted with dichloromethane and extracted with H₂O. The organic phase was dried over magnesium sulfate and concentrated under reduced pressure. The residue was purified by flash chromatography eluting with a dichloromethane/methanol gradient to afford the compound P.2 (551 mg, 83%).

### 2. Synthesis of Compounds I

### Synthesis Example S1

### 6-(2-Pyridin-3-yl-thiazol-5-yl)-pyridine-2-carbaldehyde-O-methyloxime (Compound example 1-1; compound of formula C.1, wherein X is O and R⁴ is CH₃)

A mixture of 3-(thiazol-2-yl)-pyridine (406 mg, 2.50 mmol), pyridine bromide P.1-1 (591 mg, 2.75 mmol), palladium acetate (56 mg, 0.25 mmol), tricyclohexylphosphonium tetrafluoroborate (184 mg, 0.50 mmol), pivalic acid (51 mg, 0.50 mmol), and potassium carbonate (518 mg, 3.75 mmol) in dimethylacetamide (7 mL) were heated at 100° C. for 16 h. The reaction mixture was diluted with dichloromethane and extracted with H₂O. The organic phase was dried over magnesium sulfate and concentrated under reduced pressure. The residue was purified by flash chromatography eluting with a dichloromethane/methanol gradient to afford the title compound (478 mg, 65%).

### Synthesis Example S2

### 6-(2-Pyridin-3-yl-thiazol-5-yl)-pyridine-2-carbaldehyde-O-phenyloxime (Compound example 1-10; compound of formula C.1, wherein X is O and R⁴ is phenyl)

A mixture of aldehyde P.2 (348 mg, 1.30 mmol) and O-phenylhydroxylamine hydrochloride (227 mg, 1.56 mmol) in acetic acid (0.4 mL) and ethanol (4.0 mL) was heated at 70° C. for 2 h. The reaction mixture was concentrated under reduced pressure. The residue dissolved in ethyl acetate and extracted with a saturated solution of sodium bicarbonate. The organic phase was dried over magnesium sulfate and concentrated under reduced pressure. The residue was triturated with diisopropyl ether and tetrahydrofuran to give the title compound (298 mg, 64%).

### Synthesis Example S.3

### 6-(4-Cloro-2-pyridin-3-yl-thiazol-5-yl)-pyridine-2-carbaldehyde-O-methyloxime (Compound example 2-1; compound of formula C.2, wherein X is O and R⁴ is CH₃)

A mixture of compound 1-1 (241 mg, 0.81 mmol) and N-chlorosuccinimide (543 mg, 4.07 mmol) in acetonitrile (5 mL) was heated at 60° C. for 16 h. The reaction mixture was cooled to room temperature diluted with dichloromethane and extracted with aqueous sodium thiosulfate solution. The organic phase was dried over magnesium sulfate and concentrated under reduced pressure. The residue was purified by flash chromatography eluting with a dichloromethane/methanol gradient to afford the title compound (43 mg, 16%).

### B. Biological Examples

The activity of the compounds of formula I of the present invention can be demonstrated and evaluated by the following biological test.

### General Conditions

If not otherwise specified, most test solutions are to be prepared as follows: The active compound is dissolved at the desired concentration in a mixture of 1:1 (vol:vol) distilled water:acteon. The test solutions are prepared at the day of use (and, if not otherwise specified, in general at concentrations wt/vol).

### B.1 Vetch Aphid (Megoura viciae)

For evaluating control of vetch aphid (Megoura viciae) through contact or systemic means the test unit consisted of 24-well-microtiter plates containing broad bean leaf disks.

The compounds were formulated using a solution containing 75% v/v water and 25% v/v DMSO. Different concentrations of formulated compounds were sprayed onto the leaf disks at 2.5 μl, using a custom built micro atomizer, at two replications.

After application, the leaf disks were air-dried and 5 to 8 adult aphids were placed on the leaf disks inside the microtiter plate wells. The aphids were then allowed to suck on the treated leaf disks and were incubated at about 23±1° C. and about 50±5% relative humidity for 5 days. Aphid mortality and fecundity were then visually assessed.

In this test, the compound 1-1, 1-2, 1-3, 1-4, 1-5, 1-6, 1-7, 1-8, 1-9, 1-10, 1-11, 1-12, 1-13 1-14, 1-15, 1-18, 2-1, 3-2, 3-3, 3-4, 3-5, 3-6, 3-7, 3-8, 3-9, 3-10, 3-11, 3-15, 3-16, 3-17, 3-18, 3-19, 3-20, 3-21, 3-22, 3-23, 3-25, 3-26, 3-27, 3-28, 3-32, 3-33, 3-34, 3-35, 3-36, 3-37, 3-39, 3-40, 3-41, 3-43, 3-44, 3-45, 3-46, 3-47, 3-48, 3-50, 3-51, 3-53, 3-54, 3-55, 3-56, 3-58, 3-58, 4-1, 4-2, 4-3, 4-4 and 6-2 at 800 ppm showed a mortality of at least 75% in comparison with untreated controls.

### B.2 Green Peach Aphid (Myzus persicae)

For evaluating control of green peach aphid (Myzus persicae) through systemic means the test unit consisted of 96-well-microtiter plates containing liquid artificial diet under an artificial membrane.

The compounds were formulated using a solution containing 75% v/v water and 25% v/v DMSO. Different concentrations of formulated compounds were pipetted into the aphid diet, using a custom built pipetter, at two replications.

After application, 5 to 8 adult aphids were placed on the artificial membrane inside the microtiter plate wells. The aphids were then allowed to suck on the treated aphid diet and were incubated at about 23±1° C. and about 50±5% relative humidity for 3 days. Aphid mortality and fecundity was then visually assessed.

In this test, the compound 1-1, 1-2, 1-8, 1-9, 1-15, 1-12, 1-18, 2-1, 3-2, 3-3, 3-4, 3-5, 3-6, 3-7, 3-9, 3-11, 3-12, 3-14, 3-15, 3-18, 3-23, 3-26, 3-28, 3-31, 3-33, 3-34, 3-35, 3-36, 3-37, 3-38, 3-39, 3-43, 3-44, 3-45, 3-46, 3-47, 3-48, 3-50, 3-51, 3-53, 3-54, 3-57, 3-58, 3-59, 5-1 and 6-2 at 800 ppm showed a mortality of at least 75% in comparison with untreated controls.

### B.3. Cotton Aphid (Aphis gossypii)

The active compounds were formulated in cyclohexanone as a 10′ 000 ppm solution supplied in 1.3 ml ABgene® tubes. These tubes were inserted into an automated electrostatic sprayer equipped with an atomizing nozzle and they served as stock solutions for which lower dilutions were made in 1:1 (vol:vol) water:aceton. A nonionic surfactant (Kinetic®) was included in the solution at a volume of 0.01% (v/v).

Cotton plants at the cotyledon stage were infested with aphids prior to treatment by placing a heavily infested leaf from the main aphid colony on top of each cotyledon. Aphids were allowed to transfer overnight to accomplish an infestation of 80-100 aphids per plant and the host leaf was removed. The infested plants were then sprayed by an automated electrostatic plant sprayer equipped with an atomizing spray nozzle. The plants were dried in the sprayer fume hood, removed from the sprayer, and then maintained in a growth room under fluorescent lighting in a 24-hr photoperiod at 25° C. and 20-40% relative humidity. Aphid mortality on the treated plants, relative to mortality on untreated control plants, was determined after 5 days.

In this test, the compound 1-1, 1-2, 1-3, 1-4, 1-5, 1-6, 1-7, 1-8, 1-9, 1-12, 1-13, 1-17, 2-1, 3-2, 3-3, 3-4, 3-5, 3-6, 3-7, 3-10, 3-11, 3-12, 3-14, 3-15, 3-19, 3-20, 3-35, 3-36, 3-41, 3-42, 3-43, 3-44, 3-45 and 4-2 at 300 ppm showed at least 75% mortality in comparison with untreated controls.

### B.4 Cowpea Aphid (Aphis craccivora)

The active compound was dissolved at the desired concentration in a mixture of 1:1 (vol:vol) distilled water:acetone. The test solution was prepared at the day of use. Potted cowpea plants colonized with approximately 100 to 150 aphids of various stages were sprayed after the pest population had been recorded. Population reduction was assessed after 24, 72, and 120 hours.

In this test, the compound 1-1, 1-2, 1-3, 1-4, 1-5, 1-6, 1-7, 1-8, 1-9, 1-10, 1-11, 1-12, 1-13, 1-14, 1-15, 1-16, 1-17, 1-18, 1-22, 2-1, 3-2, 3-3, 3-4, 3-5, 3-6, 3-7, 3-8, 3-9, 3-10, 3-11, 3-12, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38, 3-39, 3-40, 3-41, 3-42, 3-43, 3-44, 3-45, 3-46, 3-47, 3-48, 3-49, 3-50, 3-51, 3-52, 3-53, 3-54, 3-56, 3-57, 3-58, 4-1, 4-2, 4-3, 4-4, 5-1, 6-2 and 6-3 at 300 ppm showed a mortality of at least 75% in comparison with untreated controls.

### B.5 Silverleaf Whitefly (Bemisia Argentifolii)

The active compounds were formulated in cyclohexanone as a 10,000 ppm solution supplied in 1.3 ml ABgene® tubes. These tubes were inserted into an automated electrostatic sprayer equipped with an atomizing nozzle and they served as stock solutions for which lower dilutions were made in 1:1 (vol:vol) water:aceton. A nonionic surfactant (Kinetic®) was included in the solution at a volume of 0.01% (v/v).

Cotton plants at the cotyledon stage (one plant per pot) were sprayed by an automated electrostatic plant sprayer equipped with an atomizing spray nozzle. The plants were dried in the sprayer fume hood and then removed from the sprayer. Each pot was placed into a plastic cup and 10 to 12 whitefly adults (approximately 3 to 5 days old) were introduced. The insects were collected using an aspirator and 0.6 cm, nontoxic Tygon® tubing (R-3603) connected to a barrier pipette tip. The tip, containing the collected insects, was then gently inserted into the soil containing the treated plant, allowing insects to crawl out of the tip to reach the foliage for feeding. Cups were covered with a reusable screened lid (150-micron mesh polyester screen PeCap from Tetko, Inc.). Test plants were maintained in a growth room at 25° C. and 20-40% relative humidity for 3 days, avoiding direct exposure to fluorescent light (24 hour photoperiod) to prevent trapping of heat inside the cup. Mortality was assessed 3 days after treatment, compared to untreated control plants.

In this test, the compound 1-1, 1-2, 1-3, 1-4, 1-5, 1-6, 1-7, 1-8, 1-9, 2-1, 3-2, 3-4, 3-5, 3-6, 3-7, 3-8, 3-10, 3-11, 3-14, 3-15, 3-19, 3-35, 3-56, 3-41, 3-42, 3-43, 3-44, 3-45 and 4-2 at 300 ppm showed a mortality of at least 75% in comparison with untreated controls.

1-49. (canceled)

50: A substituted 2-(pyridin-3-yl)-5-hetaryl-thiazole compound of the general formula I 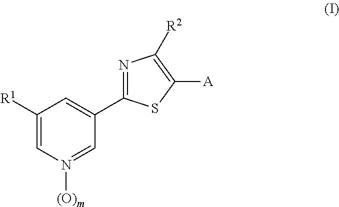
wherein
R¹ is selected from the group consisting of hydrogen, cyano and halogen;
R² is selected from the group consisting of hydrogen, halogen, cyano, C₁-C₆-alkyl and C₃-C₆-cycloalkyl, where the two last-mentioned radicals may be partially or fully halogenated and/or may be substituted by one or more radicals R³;
A is a heteroaromatic radical selected from the radicals of formulae A-1 to A-29: 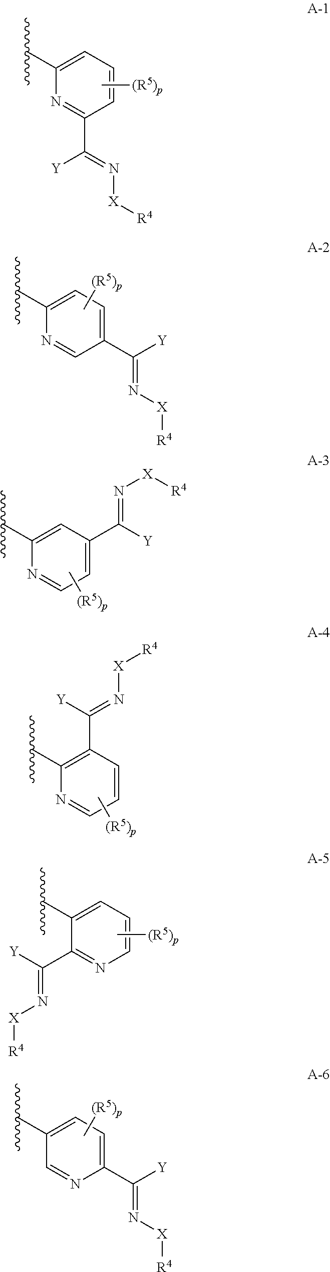 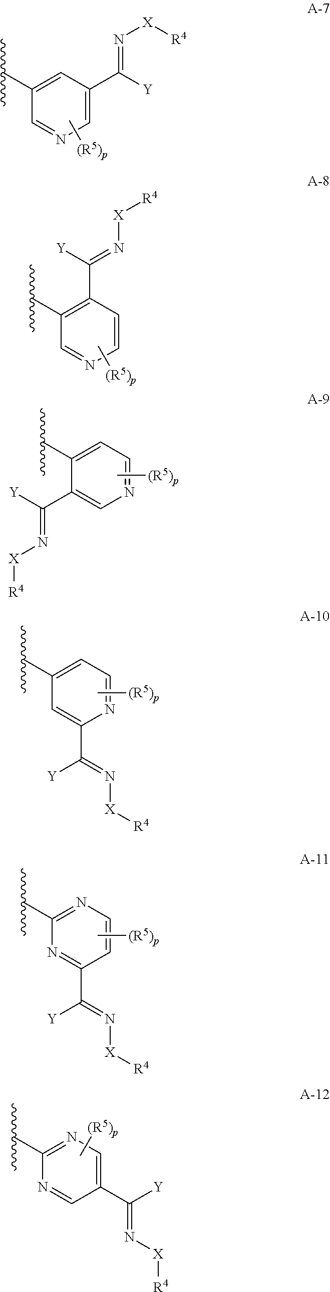 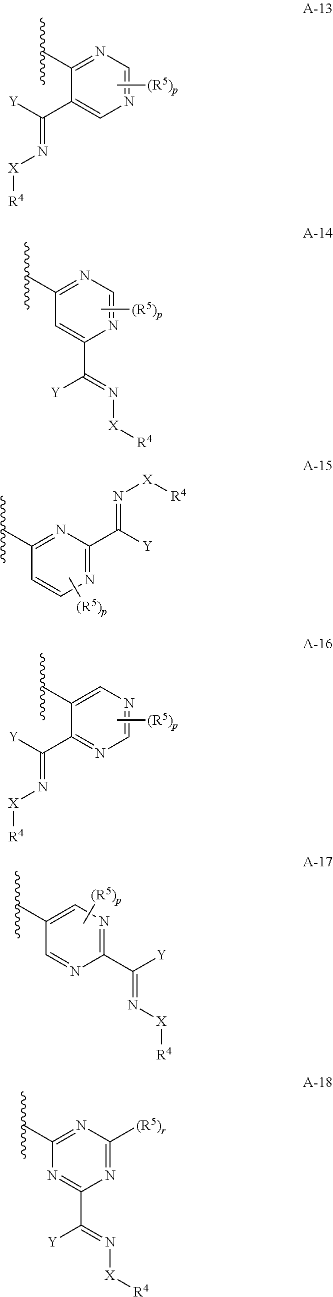 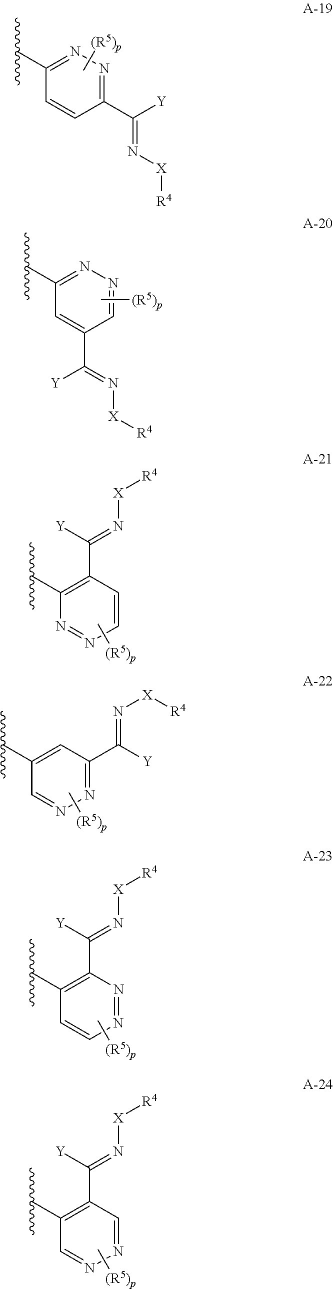 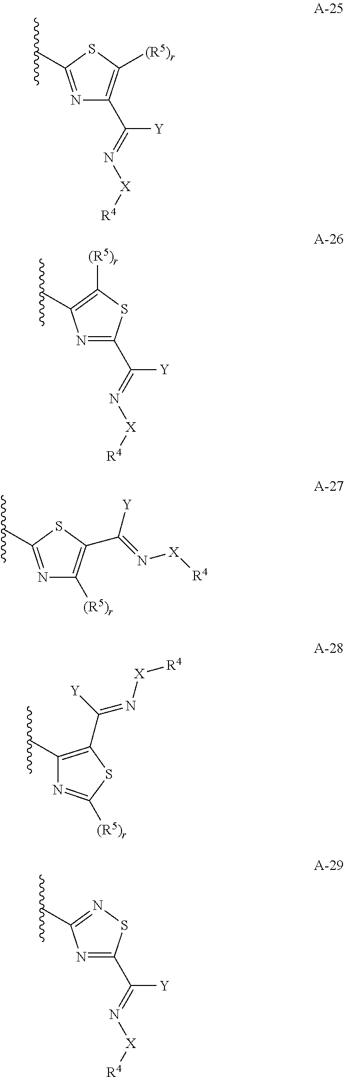
wherein
the zigzag line denotes the bond to the thiazole ring;
X is O, N—R^{4a}, S(O)ₙ or a chemical bond;
Y is selected from hydrogen, C₁-C₆-alkyl, C₃-C₆-cycloalkyl, where the two last-mentioned radicals may be partially or fully halogenated and/or may be substituted by one or more radicals R⁷;
C(═O)NR^{9a}R^{9b} and C(═NR^{9a})R⁷;
R³ is each independently from one another selected from the group consisting of cyano, —SCN, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, C₃-C₈-cycloalkyl, C₃-C₈-halocycloalkyl, Si(R¹¹)₂R¹², OR¹⁶, OSO₂R¹⁶, S(O)ₙR¹⁶, S(O)ₙNR^{17a}R^{17b}, NR^{17a}R^{17b}, C(═O)NR^{17a}R^{17b}, C(═S)NR^{17a}R^{17b}, C(═O)OR¹⁶,
phenyl, optionally substituted with 1, 2, 3, 4 or 5 substituents R¹⁵; and
a 3-, 4-, 5-, or 6-membered saturated, partly unsaturated or maximally unsaturated heterocyclic ring comprising 1, 2 or 3 heteroatoms or heteroatom groups selected from N, O, S, NO, SO and SO₂ as ring members, wherein the heterocyclic ring is optionally substituted with one or more substituents R¹⁵; and
R³ as a substituent on a cycloalkyl ring is additionally selected from C₁-C₆-alkyl, C₁-C₆-haloalkyl, C₂-C₆-alkenyl, C₂-C₆-haloalkenyl, C₂-C₆-alkynyl and C₂-C₆-haloalkynyl;
R⁴ is selected from the group consisting of hydrogen, cyano, C₁-C₁₀-alkyl, C₃-C₈-cycloalkyl, C₂-C₁₀-alkenyl, C₂-C₁₀-alkynyl, wherein the four last-mentioned radicals may be partially or fully halogenated and/or may be substituted with one or more radicals R⁷;
OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
phenyl, optionally substituted with 1, 2, 3, 4 or 5 substituents R¹⁰; and
a 3-, 4-, 5-, 6- or 7-membered saturated, partly unsaturated or maximally unsaturated heterocyclic ring comprising 1, 2, 3 or 4 heteroatoms or heteroatom groups selected from N, O, S, NO, SO and SO₂ as ring members, where the heterocyclic ring is optionally substituted with one or more substituents R¹⁰;
R^{4a} is selected from the group consisting of hydrogen, C₁-C₆-alkyl, C₁-C₆-haloalkyl, C₃-C₈-cycloalkyl, C₃-C₈-halocycloalkyl, C₁-C₆-alkoxy, C₁-C₆-haloalkoxy, phenyl and benzyl, where the phenyl ring in the two last-mentioned radicals may carry 1, 2, 3, 4 or 5 substituents selected from halogen, C₁-C₄-alkyl, C₁-C₄-haloalkyl, C₁-C₄-alkoxy and C₁-C₄-haloalkoxy;
R⁵ is each independently from one another selected from the group consisting of halogen, cyano, azido, nitro, SCN, SF₅, C₁-C₁₀-alkyl, C₃-C₈-cycloalkyl, C₂-C₁₀-alkenyl, C₂-C₁₀-alkynyl, wherein the four last-mentioned radicals may be partially or fully halogenated and/or may be substituted with one or more radicals R⁷;
OR¹⁶, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
phenyl, optionally substituted with 1, 2, 3, 4, or 5 substituents R⁶;
and
a 3-, 4-, 5-, 6- or 7-membered saturated, partly unsaturated or maximally unsaturated heterocyclic ring comprising 1, 2, 3 or 4 heteroatoms or heteroatom groups selected from N, O, S, NO, SO and SO₂ as ring members, where the heterocyclic ring is optionally substituted with one or more substituents R⁶; or two R⁵, together with the ring atoms they are bonded to, form a 3-, 4-, 5-, 6-, 7- or 8-membered saturated, partially unsaturated or maximally unsaturated carbocyclic or heterocyclic ring, where the heterocyclic ring contains 1 or 2 heteroatoms or heteroatom groups selected from N, O, S, NO, SO and SO₂ as ring members, where the carbocyclic or heterocyclic ring may contain 1 or 2 groups selected from C═O, C═S and C═NR^{17a} as ring members; and where the carbocyclic or heterocyclic ring may be substituted by one or more radicals selected from the group consisting of halogen, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₁-C₆-alkylthio, C₁-C₆-haloalkylthio, phenyl which may be substituted by 1, 2, 3, 4 or 5 radicals R¹⁵, and a 3-, 4-, 5-, 6- or 7-membered saturated, partly unsaturated or maximally unsaturated heterocyclic ring containing 1, 2 or 3 heteroatoms or heteroatom groups selected from N, O, S, NO, SO and SO₂, as ring members, where the heterocyclic ring may be substituted by one or more radicals R¹⁵;
R⁶ is each independently from one another selected from the group consisting of halogen, cyano, nitro, SCN, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may be partially or fully halogenated and/or may be substituted with one or more radicals R⁷;
OR⁸, NR^{9a}R^{9b}, S(O)ₙR⁸, S(O)ₙNR^{9a}R^{9b}, C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, C(═NR^{9a})R⁷, C(═NR^{9a})NR^{9a}R^{9b}, Si(R¹¹)₂R¹²;
phenyl, optionally substituted with 1, 2, 3, 4, or 5 substituents R¹⁰; and
a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more substituents selected independently from one another from halogen, cyano, NO₂, C₁-C₆-alkyl, C₁-C₆-haloalkyl, C₁-C₆-alkoxy and C₁-C₆-haloalkoxy;
or
two R⁶ present together on one carbon atom of a partly saturated heterocyclic ring may form together a group ═O, ═CR¹³R¹⁴; ═S, ═NR^{17a}, ═NOR¹⁶ or ═NNR^{17a};
or
two R⁶ bound on adjacent carbon atoms may together form a bridge selected from CH₂CH₂CH₂CH₂, CH═CH—CH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR^{17a}, CH₂CH═N, CH═CH—NR^{17a}, OCH═N and SCH═N, thus forming together with the carbon atoms to which they are bonded to a 5-membered or 6-membered saturated, partly unsaturated or aromatic carbocyclic or heterocyclic ring, wherein the ring may optionally be substituted with one or two substituents selected from ═O, OH, CH₃, OCH₃, halogen, cyano, halomethyl and halomethoxy;
R⁷ is each independently from one another selected from the group consisting of cyano, azido, nitro, —SCN, SF₅, C₃-C₈-cycloalkyl, C₃-C₈-halocycloalkyl, Si(R¹¹)₂R¹², OR¹⁶, OSO₂R¹⁶, S(O)ₙR¹⁶, S(O)ₙNR^{17a}R^{17b}, NR^{17a}R^{17b}, C(═O)NR^{17a}R^{17b}, C(═S)NR^{17a}R^{17b}, C(═O)OR¹⁶,
phenyl, optionally substituted with 1, 2, 3, 4 or 5 substituents R¹⁵, and a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more substituents R¹⁵,
or
two R⁷ present on the same carbon atom of an alkyl, alkenyl, alkynyl or cycloalkyl group may together form a group ═O, ═CR¹³R¹⁴; ═S, ═NR^{17a}, ═NOR¹⁶ or ═NNR^{17a};
or
two R⁷, together with the carbon atom(s) of an alkyl, alkenyl, alkynyl or cycloalkyl group to which they are bonded, may form a 3-, 4-, 5-, 6-, 7- or 8-membered saturated or partly unsaturated carbocyclic or heterocyclic ring, where the heterocyclic ring comprises 1, 2, 3 or 4 heteroatoms or heteroatom groups selected from N, O, S, NO, SO and SO₂ as ring members, and where the carbocyclic or heterocyclic ring is optionally substituted with one or more substituents R¹⁵; and
R⁷ as a substituent on a cycloalkyl ring is additionally selected from the group consisting of C₁-C₆-alkyl, C₁-C₆-haloalkyl, C₂-C₆-alkenyl, C₂-C₆-haloalkenyl, C₂-C₆-alkynyl and C₂-C₆-haloalkynyl, where the aliphatic moieties in these six radicals may be substituted by one or more radicals R¹⁹; and
R⁷ in the groups —C(═NR^{9a})R⁷, —C(═O)R⁷ and —C(═S)R⁷ is additionally selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl and C₂-C₆-haloalkynyl, where the aliphatic moieties in the six last-mentioned radicals may be substituted by one or more radicals R¹⁹;
R⁸ is each independently from one another selected from the group consisting of hydrogen, cyano, C₁-C₆-alkyl, C₁-C₆-haloalkyl, C₃-C₈-cycloalkyl, C₃-C₈-cycloalkyl-C₁-C₄-alkyl-, C₃-C₈-halocycloalkyl, C₂-C₆-alkenyl, C₂-C₆-haloalkenyl, C₂-C₆-alkynyl, C₂-C₆-haloalkynyl, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, —Si(R¹¹)₂R¹², S(O)ₙR¹⁶, S(O)ₙNR^{17a}R^{17b}, NR^{17a}R^{17b}, —N═CR¹³R¹⁴, —C(═O)R¹⁸, C(═O)NR^{17a}R^{17b}, C(═S)NR^{17a}R^{17b}, C(═O)OR¹⁶,
phenyl, optionally substituted with 1, 2, 3, 4 or 5 substituents R¹⁵; and
a 3-, 4-, 5-, 6- or 7-membered saturated, partly unsaturated or maximally unsaturated heterocyclic ring comprising 1, 2 or 3 heteroatoms selected from N, O, S, NO, SO and SO₂ as ring members, where the heterocyclic ring is optionally substituted with one or more substituents R¹⁵;
R^{9a}, R^{9b} are each independently from one another selected from the group consisting of hydrogen, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl, C₁-C₆-alkoxy, C₁-C₆-haloalkoxy, C₁-C₆-alkylthio, C₁-C₆-haloalkylthio, S(O)ₙR¹⁶, —S(O)ₙNR^{17a}R^{17b}, C(═O)R¹⁸, C(═O)OR¹⁶, C(═O)NR^{17a}R^{17b}, C(═S)R¹⁸, C(═S)SR¹⁶, C(═S)NR^{17a}R^{17b}, C(═NR^{17a})R¹⁸;
phenyl, optionally substituted with 1, 2, 3, 4 or 5 substituents R¹⁵; and
a 3-, 4-, 5-, 6- or 7-membered saturated, partly unsaturated or maximally unsaturated heterocyclic ring comprising 1, 2, 3 or 4 heteroatoms or heteroatom groups selected from N, O, S, NO, SO and SO₂ as ring members, where the heterocyclic ring is optionally substituted with one or more substituents R¹⁵;
or,
R^{9a} and R^{9b}, together with the nitrogen atom they are bonded to, form a 3-, 4-, 5-, 6-, 7- or 8-membered saturated, partly unsaturated or maximally unsaturated ring, wherein the heterocyclic ring may additionally contain one or two heteroatoms or heteroatom groups selected from N, O, S, NO, SO and SO₂ as ring members, wherein the heterocyclic ring may optionally be substituted with one or more radicals selected from halogen, cyano, C₁-C₆-alkyl, C₁-C₆-haloalkyl, C₃-C₈-cycloalkyl, C₃-C₈-halocycloalkyl, C₂-C₆-alkenyl, C₂-C₆-haloalkenyl, C₂-C₆-alkynyl, C₂-C₆ haloalkynyl, C₁-C₆-alkoxy, C₁-C₆-haloalkoxy, C₁-C₆-alkylthio, C₁-C₆-haloalkylthio, phenyl, optionally substituted with 1, 2, 3, 4 or 5 substituents R¹⁵; and a 3-, 4-, 5-, -6, or 7-membered saturated, partly unsaturated or maximally unsaturated heterocyclic ring comprising 1, 2 or 3 heteroatoms selected from N, O, S, NO, SO and SO₂ as ring members, where the heterocyclic ring is optionally substituted with one or more substituents R¹⁵;
or
R^{9a} and R^{9b} together form a═CR¹³R¹⁴, ═NR^{17a} or ═NOR¹⁶ radical;
R¹⁰ is each independently from one another selected from the group consisting of halogen, cyano, azido, nitro, SCN, SF₅, C₁-C₁₀-alkyl, C₃-C₈-cycloalkyl, C₂-C₁₀-alkenyl, C₂-C₁₀-alkynyl, wherein the four last-mentioned aliphatic and cycloaliphatic radicals may partially or fully halogenated and/or may be substituted with one or more radicals R¹⁹;
Si(R¹¹)₂R¹², OR¹⁶, OS(O)ₙR¹⁶, —S(O)ₙR¹⁶, S(O)ₙNR^{17a}R^{17b}, NR^{17a}R^{17b}, C(═O)R¹⁸, C(═O)OR¹⁶, —C(═NR^{17a})R¹⁸, C(═O)NR^{17a}R^{17b}, C(═S)NR^{17a}R^{17b},
phenyl, optionally substituted with 1, 2, 3, 4 or 5 radicals selected from halogen, cyano, nitro, C₁-C₆-alkyl, C₁-C₆-haloalkyl, C₁-C₆-alkoxy and C₁-C₆-haloalkoxy;
and
a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more substituents selected independently from one another from halogen, cyano, NO₂, C₁-C₆-alkyl, C₁-C₆-haloalkyl, C₁-C₆-alkoxy and C₁-C₆-haloalkoxy;
or
two R¹⁰ present together on the same carbon atom of a heterocyclic may together form a group ═O, ═CR¹³R¹⁴; ═S, ═NR^{17a}, ═NOR¹⁶ or ═NNR^{17a};
or,
two R¹⁰ on adjacent carbon atoms may together form a bridge selected from CH₂CH₂CH₂CH₂, CH═CH—CH═CH, N═CH—CH═CH, CH═N—CH═CH, N═CH—N═CH, OCH₂CH₂CH₂, OCH═CHCH₂, CH₂OCH₂CH₂, OCH₂CH₂O, OCH₂OCH₂, CH₂CH₂CH₂, CH═CHCH₂, CH₂CH₂O, CH═CHO, CH₂OCH₂, CH₂C(═O)O, C(═O)OCH₂, O(CH₂)O, SCH₂CH₂CH₂, SCH═CHCH₂, CH₂SCH₂CH₂, SCH₂CH₂S, SCH₂SCH₂, CH₂CH₂S, CH═CHS, CH₂SCH₂, CH₂C(═S)S, C(═S)SCH₂, S(CH₂)S, CH₂CH₂NR^{17a}, CH₂CH═N, CH═CH—NR^{17a} and OCH═N, SCH═N, thus forming together with the carbon atoms to which they are bonded a 5-membered or 6-membered partly unsaturated or aromatic carbocyclic or heterocyclic ring, wherein the ring may be substituted with one or two substituents selected from ═O, OH, CH₃, OCH₃, halogen, cyano, halomethyl and halomethoxy;
R¹¹, R¹² are each independently from one another selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy, C₁-C₆-haloalkoxy, C₁-C₆-alkoxy-C₁-C₄-alkyl, C₁-C₆-haloalkoxy-C₁-C₄-alkyl and
phenyl, optionally substituted with 1, 2, 3, 4 or 5 substituents R¹⁵;
R¹³, R¹⁴ are each independently from one another selected from the group consisting of hydrogen, C₁-C₄-alkyl, C₃-C₆-cycloalkyl, C₁-C₄-alkoxy-C₁-C₄-alkyl, phenyl and benzyl;
R¹⁵ is each independently from one another selected from the group consisting of halogen, cyano, nitro, OH, SH, SCN, SF₅, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl,
C₁-C₆-alkyl, C₂-C₆-alkenyl, C₂-C₆-alkynyl, C₃-C₈-cycloalkyl, wherein the four last-mentioned aliphatic and cycloaliphatic radicals may be unsubstituted, partially or fully halogenated and/or may carry 1 or 2 radicals selected from hydroxyl and C₁-C₄-alkoxy;
phenyl, benzyl, pyridyl and phenoxy, wherein the four last-mentioned radicals may be unsubstituted or may carry 1, 2 or 3 substituents selected from halogen, C₁-C₆-alkyl, C₁-C₆-haloalkyl, C₁-C₆-alkoxy, C₁-C₆-haloalkoxy, (C₁-C₆-alkoxy)carbonyl, (C₁-C₆-alkyl)amino and di-(C₁-C₆-alkyl)amino;
or
two R¹⁵ present on the same carbon atom of a heterocyclic ring may together form a group ═O, ═CH(C₁-C₄), ═C(C₁-C₄-alkyl)C₁-C₄-alkyl, ═N(C₁-C₆-alkyl) or ═NO(C₁-C₆-alkyl);
R¹⁶ is each independently from one another selected from the group consisting of hydrogen, C₁-C₆-alkyl, C₂-C₆-alkenyl, C₂-C₆-alkynyl, C₃-C₈-cycloalkyl, wherein the four last-mentioned radicals may be unsubstituted, partially or fully halogenated and/or may carry 1 or 2 radicals selected from hydroxyl and C₁-C₄-alkoxy,
phenyl, benzyl, pyridyl and phenoxy, wherein the four last-mentioned radicals may be unsubstituted or carry 1, 2 or 3 substituents selected from halogen, C₁-C₆-alkyl, C₁-C₆-haloalkyl, C₁-C₆-alkoxy, C₁-C₆-haloalkoxy and (C₁-C₆-alkoxy)carbonyl;
R^{17a}, R^{17b} are each independently from one another selected from the group consisting of hydrogen, cyano, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl,
C₁-C₆-alkyl, C₂-C₆-alkenyl, C₂-C₆-alkynyl, C₃-C₈-cycloalkyl, wherein the four last-mentioned aliphatic and cycloaliphatic radicals may be unsubstituted, partially or fully halogenated and/or may carry 1 or 2 radicals selected from hydroxyl and C₁-C₄-alkoxy; C₃-C₈-cycloalkyl-C₁-C₄-alkyl,
phenyl, benzyl, pyridyl and phenoxy, wherein the four last-mentioned radicals may be unsubstituted or carry 1, 2 or 3 substituents selected from halogen, C₁-C₆-alkyl, C₁-C₆-haloalkyl, C₁-C₆-alkoxy, C₁-C₆-haloalkoxy and (C₁-C₆-alkoxy)carbonyl,
or
R^{17a} and R^{17b}, together with the nitrogen atom they are bonded to, form a 3-, 4-, 5-, 6- or 7-membered saturated, partly unsaturated or maximally unsaturated ring, wherein the heterocyclic ring may additionally contain one or two heteroatoms or heteroatom groups selected from N, O, S, NO, SO and SO₂ as ring members, wherein the heterocyclic ring may be substituted with one or more substituents selected from halogen, cyano, C₁-C₄-alkyl, C₁-C₄-haloalkyl, C₁-C₄-alkoxy and C₁-C₄-haloalkoxy;
R¹⁸ is each independently from one another selected from the group consisting hydrogen, halogen, OH, SH, SCN, SF₅, C₁-C₆-alkyl, C₂-C₆-alkenyl, C₂-C₆-alkynyl, C₃-C₈-cycloalkyl, wherein the four last-mentioned aliphatic and cycloaliphatic radicals may be unsubstituted, partially or fully halogenated and/or may carry 1 or 2 radicals selected from hydroxyl and C₁-C₄-alkoxy;
C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl,
phenyl, benzyl, pyridyl and phenoxy, wherein the four last-mentioned radicals may be unsubstituted or may carry 1, 2 or 3 substituents selected from halogen, C₁-C₆-alkyl, C₁-C₆-haloalkyl, C₁-C₆-alkoxy, C₁-C₆-haloalkoxy, (C₁-C₆-alkoxy)carbonyl, (C₁-C₆-alkyl)amino and di-(C₁-C₆-alkyl)amino,
R¹⁹ is each independently from one another selected from the group consisting of cyano, nitro, OH, SH, SCN, SF₅, C₁-C₆-alkoxy, C₁-C₆-haloalkoxy, C₁-C₆-alkylthio, C₁-C₆-haloalkylthio, C₁-C₆-alkylsulfinyl, C₁-C₆-haloalkylsulfinyl, C₁-C₆-alkylsulfonyl, C₁-C₆-haloalkylsulfonyl, trimethylsilyl, triethylsilyl, tert-butyldimethylsilyl,
C₃-C₈-cycloalkyl, wherein the last-mentioned radical may be unsubstituted, partially or fully halogenated and/or may carry 1 or 2 radicals selected from hydroxyl and C₁-C₄-alkoxy;
phenyl, benzyl, pyridyl and phenoxy, wherein the four last-mentioned radicals may be unsubstituted or may carry 1, 2 or 3 substituents selected from halogen, C₁-C₆-alkyl, C₁-C₆-haloalkyl, C₁-C₆-alkoxy, C₁-C₆-haloalkoxy, (C₁-C₆-alkoxy)carbonyl, (C₁-C₆-alkyl)amino and di-(C₁-C₆-alkyl)amino,
or
two R¹⁹ present on the same carbon atom may together form a group ═O, ═CH(C₁-C₄), ═C(C₁-C₄-alkyl)C₁-C₄-alkyl, ═N(C₁-C₆-alkyl) or ═NO(C₁-C₆-alkyl);
and
R¹⁹ as a substituent on a cycloalkyl ring is additionally selected from the group consisting of C₁-C₆-alkyl, C₁-C₆-haloalkyl, C₂-C₆-alkenyl, C₂-C₆-haloalkenyl, C₂-C₆-alkynyl and C₂-C₆-haloalkynyl;
m is 0 or 1;
n is 0, 1 or 2;
p is 0, 1 or 2; and
r is 0 or 1;
and/or an enantiomer, diastereomer, E/Z-isomer or agriculturally or veterinarily acceptable salts thereof.

51: The compound as claimed in claim 50, wherein R¹ is selected from hydrogen and halogen.

52: The compound as claimed in claim 50, wherein R² is selected from hydrogen, cyano, C₁-C₆-alkyl, C₃-C₆-cycloalkyl and C₃-C₆-halocycloalkyl, where the three last-mentioned radicals may be substituted by one or more radicals R³;
where R³ is as defined in claim 50.

53: The compound as claimed in claim 52, where R² is selected from hydrogen and C₁-C₆-alkyl which may be substituted by one or more radicals R³.

54: The compound as claimed in claim 50, wherein R² is selected from hydrogen, halogen and C₁-C₄-alkyl.

55: The compound as claimed in claim 50, wherein R² is selected from C₁-C₄-haloalkyl.

56: The compound as claimed in claim 50, wherein A is selected from the radicals of formulae A-1 to A-10.

57: The compound as claimed in claim 54, wherein A is selected from the radicals of formulae A-1 to A-4.

58: The compound as claimed in claim 50, wherein X is selected from O, NR^{4a} and a chemical bond, where R^{4a} is as defined in claim 50.

59: The compound as claimed in claim 50, wherein X is selected from O and NR^{4a}, where R^{4a} is as defined in claim 50.

60: The compound as claimed in claim 50, wherein Y is selected from hydrogen, C₁-C₄-alkyl, C₁-C₄-haloalkyl, C₃-C₆-cycloalkyl and C₃-C₆-halocycloalkyl.

61: The compound as claimed in claim 50, wherein R^{4a} is selected from hydrogen, C₁-C₄-alkyl, C₁-C₄-haloalkyl, C₃-C₆-cycloalkyl, C₃-C₆-halocycloalkyl and phenyl which may carry 1, 2 or 3 substituents selected from halogen, C₁-C₄-alkyl, C₁-C₄-haloalkyl, C₁-C₄-alkoxy and C₁-C₄-haloalkoxy.

62: The compound as claimed in claim 50, wherein R⁴ is selected from hydrogen, C₁-C₁₀-alkyl, C₃-C₈-cycloalkyl, C₂-C₁₀-alkenyl, C₂-C₁₀-alkynyl, wherein the four last-mentioned radicals may be partially or fully halogenated and/or may be substituted with one or more radicals R⁷; C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, S(O)ₙR⁸, phenyl, optionally substituted with 1, 2, 3, 4 or 5 substituents R¹⁰; and a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more substituents R¹⁰.

63: The compound as claimed in claim 50, wherein R⁴ is selected from hydrogen, C₁-C₁₀-alkyl, C₃-C₈-cycloalkyl, C₂-C₁₀-alkenyl, C₂-C₁₀-alkynyl, wherein the four last-mentioned radicals may be partially or fully halogenated and/or may be substituted with one or more radicals R⁷; C(═O)R⁷, C(═O)NR^{9a}R^{9b}, C(═O)OR⁸, C(═S)R⁷, C(═S)NR^{9a}R^{9b}, C(═S)OR⁸, C(═S)SR⁸, phenyl, optionally substituted with 1, 2, 3 or 4, especially 1, 2 or 3 substituents R¹⁰; and a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more substituents R¹⁰.

64: The compound as claimed in claim 63, wherein R⁴ is selected from hydrogen, C₁-C₆-alkyl, C₁-C₆-haloalkyl, C₁-C₆-alkyl substituted by one radical R⁷, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl, C(═O)R⁷, C(═O)NR^{9a}R^{9b}, C(═O)OR⁸, phenyl, optionally substituted with 1, 2 or 3 substituents selected from halogen, cyano, C₁-C₄-alkyl, C₁-C₄-haloalkyl, C₁-C₄-alkoxy and C₁-C₄-haloalkoxy; and a 5- or 6-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with 1 or 2 substituents selected from halogen, cyano, C₁-C₄-alkyl, C₁-C₄-haloalkyl, C₁-C₄-alkoxy and C₁-C₄-haloalkoxy.

65: The compound as claimed in claim 64, wherein R⁴ is selected from hydrogen, C₁-C₆-alkyl, C₁-C₆-haloalkyl, C₁-C₆-alkyl substituted by one radical R⁷, C₃-C₆-cycloalkyl, C₃-C₆-halocycloalkyl, C₂-C₆-alkenyl, C₂-C₆-alkynyl, C(═O)R⁷, C(═O)NR^{9a}R^{9b}, C(═O)OR⁸ and phenyl, optionally substituted with 1, 2 or 3 substituents selected from halogen.

66: The compound as claimed in claim 63, wherein R⁷ as a substituent on an alkyl, cycloalkyl, alkenyl or alkynyl group is selected from cyano, C₃-C₆-cycloalkyl, C₃-C₆-halocycloalkyl, C₁-C₆-alkoxy, C₁-C₆-haloalkoxy, C₁-C₆-alkylthio, C₁-C₆-haloalkylthio, NR^{17a}R^{17b}, C(═O)NR^{17a}R^{17b}, C(═S)NR^{17a}R^{17b}, C(═O)OR¹⁶, phenyl, optionally substituted with 1, 2 or 3 substituents selected from halogen, cyano, C₁-C₄-alkyl, C₁-C₄-haloalkyl, C₁-C₄-alkoxy and C₁-C₄-haloalkoxy; and a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more substituents selected from halogen, cyano, C₁-C₄-alkyl, C₁-C₄-haloalkyl, C₁-C₄-alkoxy and C₁-C₄-haloalkoxy.

67: The compound as claimed in claim 66, wherein R⁷ as a substituent on an alkyl, cycloalkyl, alkenyl or alkynyl group is selected from C₃-C₆-cycloalkyl, C₃-C₆-halocycloalkyl and phenyl, optionally substituted with 1, 2 or 3 substituents selected from halogen, C₁-C₄-alkyl, C₁-C₄-haloalkyl, C₁-C₄-alkoxy and C₁-C₄-haloalkoxy.

68: The compound as claimed in claim 63, wherein R⁷ as a substituent in a C(═O)R⁷ or C(═S)R⁷ group is selected from C₁-C₄-alkyl, C₁-C₄-haloalkyl, C₃-C₆-cycloalkyl, C₃-C₆-cycloalkyl-C₁-C₄-alkyl, C₃-C₆-halocycloalkyl, C₁-C₆-alkoxy, C₁-C₆-haloalkoxy, C₁-C₆-alkylthio, C₁-C₆-haloalkylthio, NR^{17a}R^{17b}, phenyl, benzyl, where the phenyl ring in the two last-mentioned radicals is optionally substituted with 1, 2 or 3 substituents selected from halogen, cyano, C₁-C₄-alkyl, C₁-C₄-haloalkyl, C₁-C₄-alkoxy and C₁-C₄-haloalkoxy; and a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more substituents selected from halogen, cyano, C₁-C₄-alkyl, C₁-C₄-haloalkyl, C₁-C₄-alkoxy and C₁-C₄-haloalkoxy.

69: The compound as claimed in claim 68, wherein R⁷ as a substituent in a C(═O)R⁷ or C(═S)R⁷ group is selected from C₁-C₄-alkyl, C₁-C₄-haloalkyl, C₃-C₆-cycloalkyl, C₃-C₆-cycloalkyl-C₁-C₄-alkyl, C₃-C₆-halocycloalkyl, C₁-C₆-alkoxy, C₁-C₆-haloalkoxy, NR^{17a}R^{17b}, phenyl and benzyl, where the phenyl ring in the two last-mentioned radicals is optionally substituted with 1, 2 or 3 substituents selected from halogen, C₁-C₄-alkyl, C₁-C₄-haloalkyl, C₁-C₄-alkoxy and C₁-C₄-haloalkoxy.

70: The compound as claimed in claim 63, wherein R⁸ is selected from hydrogen, C₁-C₄-alkyl, C₁-C₄-haloalkyl, C₃-C₆-cycloalkyl, C₃-C₆-cycloalkyl-C₁-C₄-alkyl, C₃-C₆-halocycloalkyl, phenyl, benzyl, where the phenyl ring in the two last-mentioned radicals is optionally substituted with 1, 2 or 3 substituents selected from halogen, cyano, C₁-C₄-alkyl, C₁-C₄-haloalkyl, C₁-C₄-alkoxy and C₁-C₄-haloalkoxy; and a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more substituents selected from halogen, cyano, C₁-C₄-alkyl, C₁-C₄-haloalkyl, C₁-C₄-alkoxy and C₁-C₄-haloalkoxy.

71: The compound as claimed in claim 63, wherein R^{9a}, R^{9b}, R^{17a} and R^{17b}, independently of each other, are selected from hydrogen, C₁-C₄-alkyl, C₁-C₄-haloalkyl, C₃-C₆-cycloalkyl, C₃-C₆-cycloalkyl-C₁-C₄-alkyl, C₃-C₆-halocycloalkyl, phenyl, benzyl, where the phenyl ring in the two last-mentioned radicals is optionally substituted with 1, 2 or 3 substituents selected from halogen, cyano, C₁-C₄-alkyl, C₁-C₄-haloalkyl, C₁-C₄-alkoxy and C₁-C₄-haloalkoxy; and a 3-, 4-, 5-, 6- or 7-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more substituents selected from halogen, cyano, C₁-C₄-alkyl, C₁-C₄-haloalkyl, C₁-C₄-alkoxy and C₁-C₄-haloalkoxy.

72: The compound as claimed in claim 62, wherein R⁴ is selected from hydrogen, C₁-C₆-alkyl, C₁-C₆-haloalkyl, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl, C₁-C₆-alkyl carrying one radical R⁷; C(═O)R⁷, C(═O)NR^{9a}R^{9b}, S(O)ₙR⁸, phenyl, optionally substituted with 1, 2, 3, 4 or 5 substituents R¹⁰; and a 5- or 6-membered saturated, partly unsaturated or maximally unsaturated heterocyclic ring comprising 1, 2 or 3 heteroatoms or heteroatom groups selected from N, O, S, NO, SO and SO₂ as ring members, where the heterocyclic ring is optionally substituted with one or more substituents R¹⁰;
where R⁷, R⁸, R^{9a}, R^{9b}, R¹⁰ and n are as defined in claim 50.

73: The compound as claimed in claim 72, wherein
R⁷ as a substituent on C₁-C₆-alkyl is selected from C₃-C₆-cycloalkyl, C₃-C₆-halocycloalkyl, OR¹⁶ and phenyl, optionally substituted with 1, 2 or 3 substituents selected from halogen, cyano, C₁-C₄-alkyl, C₁-C₄-haloalkyl, C₁-C₄-alkoxy and C₁-C₄-haloalkoxy; and
R⁷ as a substituent in a C(═O)R⁷ group is selected from C₁-C₄-alkyl, C₁-C₄-haloalkyl, NR^{17a}R^{17b} and phenyl which is optionally substituted with 1, 2 or 3 substituents selected from halogen, cyano, C₁-C₄-alkyl, C₁-C₄-haloalkyl, C₁-C₄-alkoxy and C₁-C₄-haloalkoxy;
R⁸ is selected from C₁-C₆-alkyl, C₁-C₆-haloalkyl and phenyl which is optionally substituted with 1, 2 or 3 substituents selected from halogen, cyano, C₁-C₄-alkyl, C₁-C₄-haloalkyl, C₁-C₄-alkoxy and C₁-C₄-haloalkoxy;
R^{9a} is selected from hydrogen and C₁-C₆-alkyl;
R^{9b} is selected from hydrogen, C₁-C₆-alkyl and C₁-C₆-haloalkyl;
R¹⁰ is selected from halogen, cyano, C₁-C₆-alkyl, C₁-C₆-haloalkyl, C₁-C₆-alkoxy, and C₁-C₆-haloalkoxy,
or
two R¹⁰ present together on the same carbon atom of a heterocyclic may together form a group ═O;
or,
two R¹⁰ on adjacent carbon atoms may together form a bridge selected from CH₂CH₂CH₂CH₂, CH═CH—CH═CH, OCH₂CH₂CH₂, CH₂OCH₂CH₂, OCH₂CH₂O, OCH₂OCH₂, CH₂CH₂CH₂, CH═CHCH₂, CH₂CH₂O, CH₂OCH₂, and O(CH₂)O;
thus forming together with the carbon atoms to which they are bonded a 5-membered or 6-membered carbocyclic or heterocyclic ring; and
n is 2.

74: The compound as claimed in claim 73, wherein
R^{17a} is selected from hydrogen and C₁-C₆-alkyl; and
R^{17a} is selected from hydrogen, C₁-C₆-alkyl, C₁-C₆-haloalkyl, C₃-C₆-cycloalkyl, C₃-C₆-halocycloalkyl, C₃-C₆-cycloalkyl-C₁-C₄-alkyl, phenyl and benzyl.

75: The compound as claimed in claim 50, wherein R⁵ is selected from halogen, cyano, C₁-C₆-alkyl, C₁-C₆-haloalkyl, C₃-C₆-cycloalkyl, C₃-C₆-halocycloalkyl, C₂-C₆-alkenyl, C₂-C₆-haloalkenyl, C₂-C₆-alkynyl, C₂-C₆-haloalkynyl, C₁-C₄-alkoxy, C₁-C₄-haloalkoxy, C₁-C₄-alkylthio, C₁-C₄-haloalkylthio, phenyl, optionally substituted with 1, 2 or 3 substituents R⁶; and a 3-, 4-, 5-, 6- or 7-membered saturated, partly unsaturated or maximally unsaturated heterocyclic ring comprising 1, 2, 3 or 4 heteroatoms or heteroatom groups selected from N, O, S, NO, SO and SO₂ as ring members, where the heterocyclic ring is optionally substituted with one or more substituents R⁶.

76: The compound as claimed in claim 75, wherein R⁵ is selected from halogen, C₁-C₆-alkyl, C₁-C₆-haloalkyl, C₁-C₄-alkoxy, C₁-C₄-haloalkoxy, C₁-C₄-alkylthio, C₁-C₄-haloalkylthio, phenyl, optionally substituted with 1, 2 or 3 substituents R⁶; and a 3-, 4-, 5-, 6- or 7-membered saturated, partly unsaturated or maximally unsaturated heterocyclic ring comprising 1, 2, 3 or 4 heteroatoms or heteroatom groups selected from N, O, S, NO, SO and SO₂ as ring members, where the heterocyclic ring is optionally substituted with one or more substituents R⁶.

77: The compound as claimed in claim 50, wherein the 3-, 4-, 5-, 6- or 7-membered saturated, partly unsaturated or maximally unsaturated heterocyclic ring R⁵ is selected from rings of formulae B-1 to B-135: 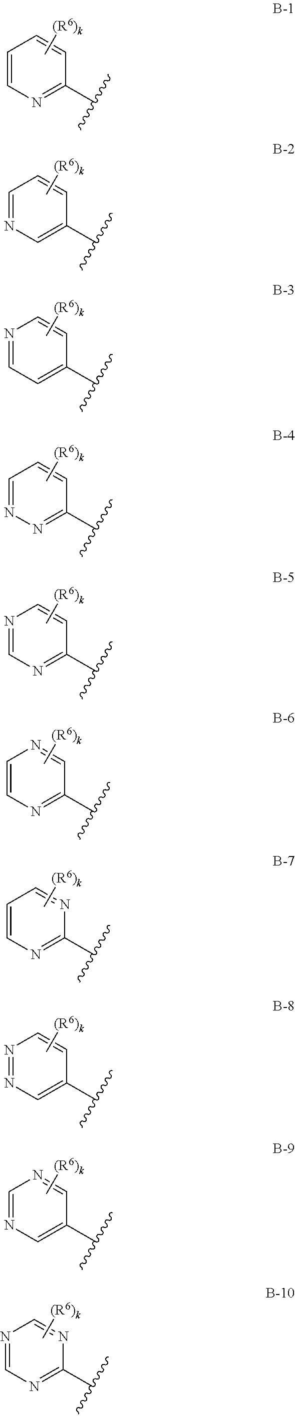 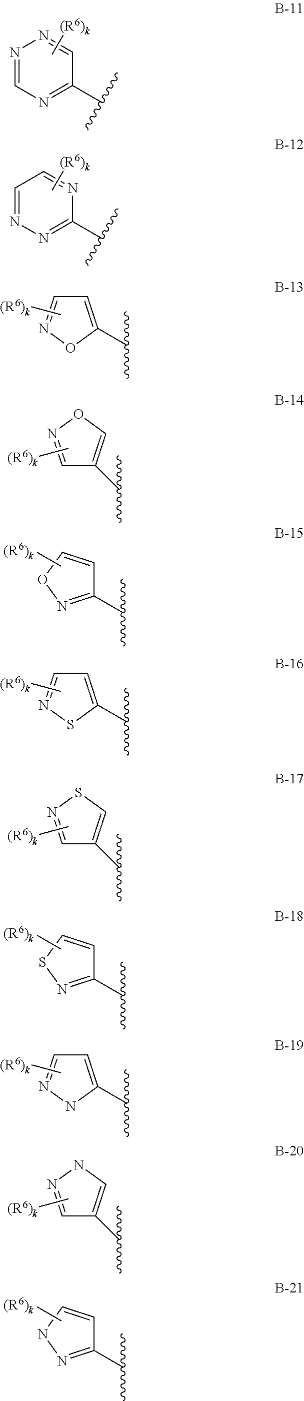 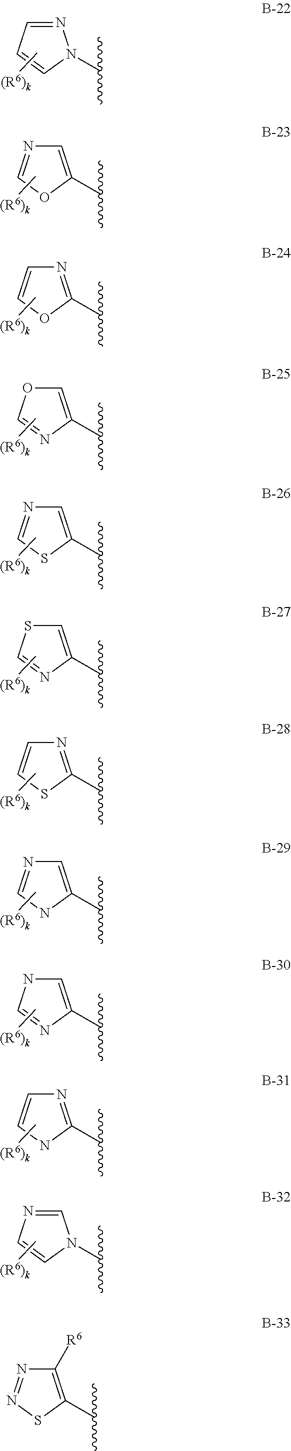 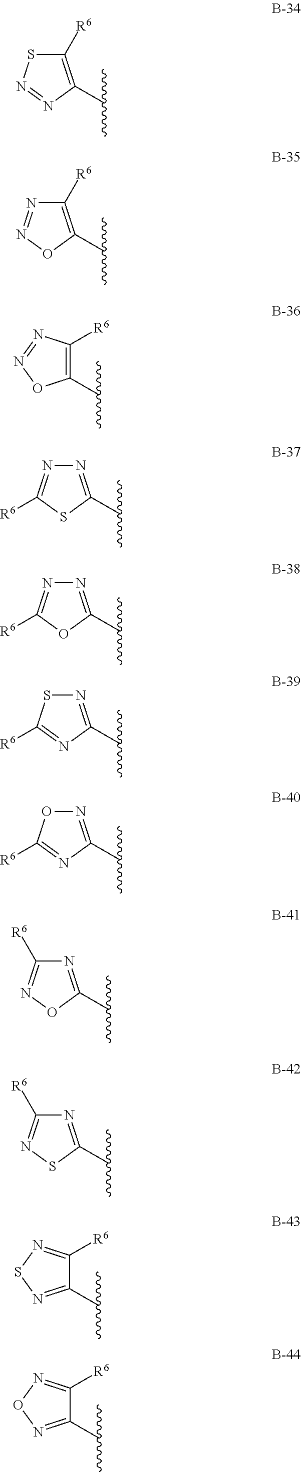 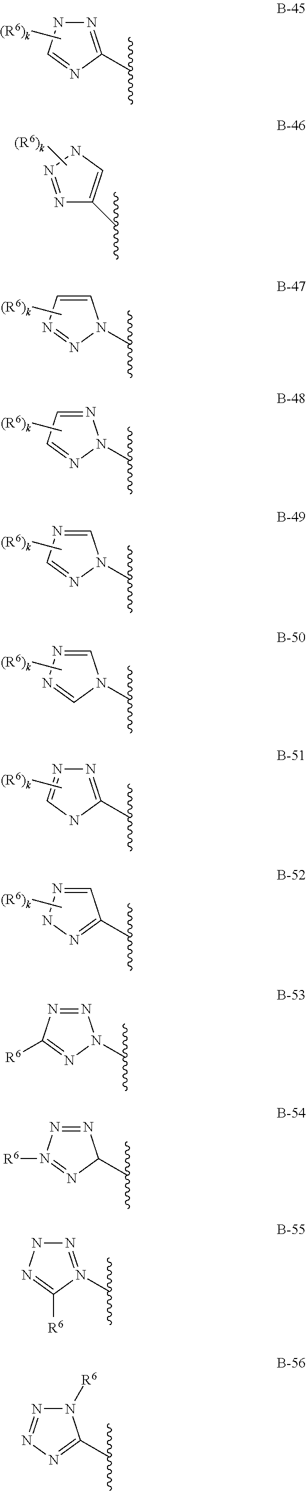 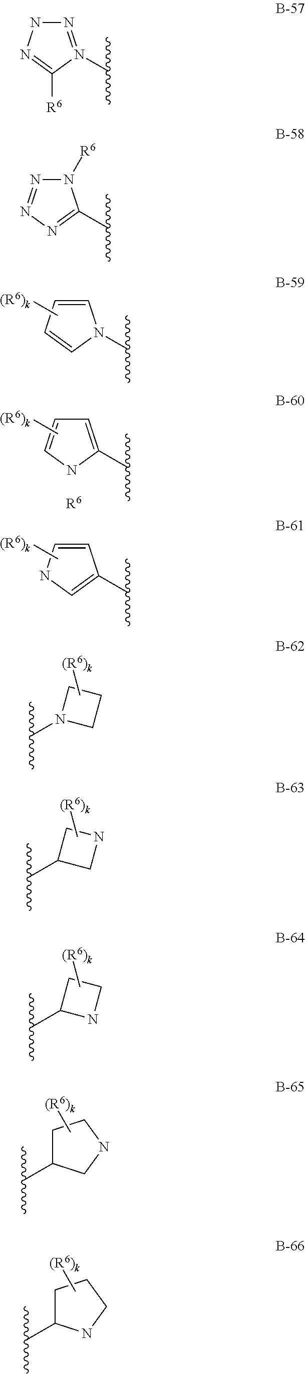 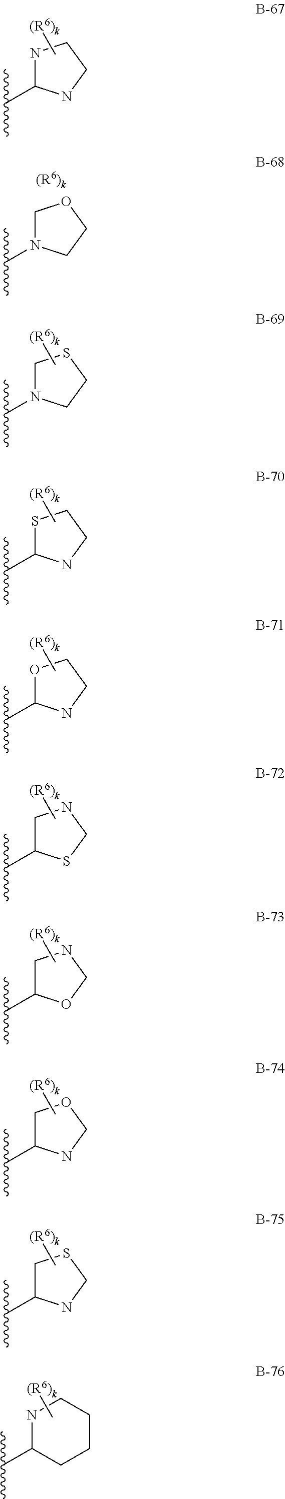 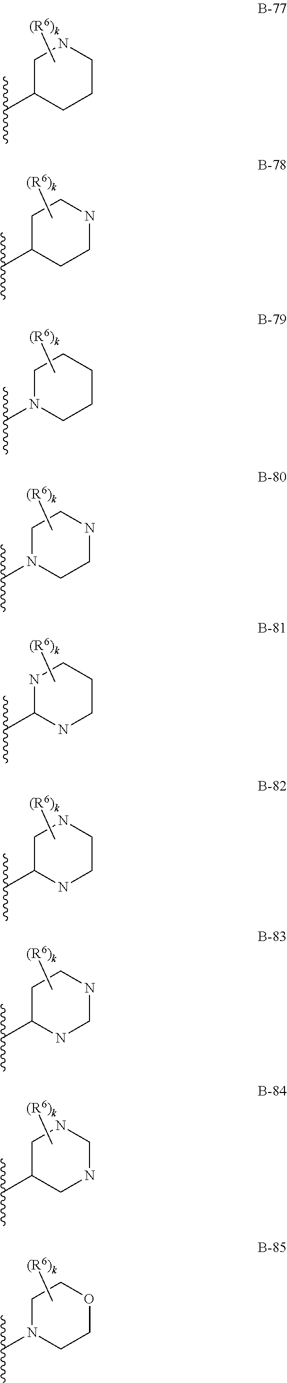 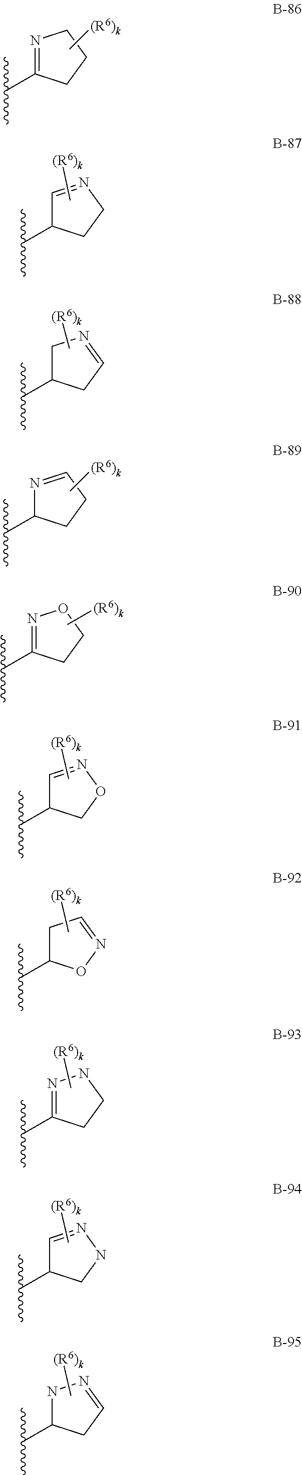 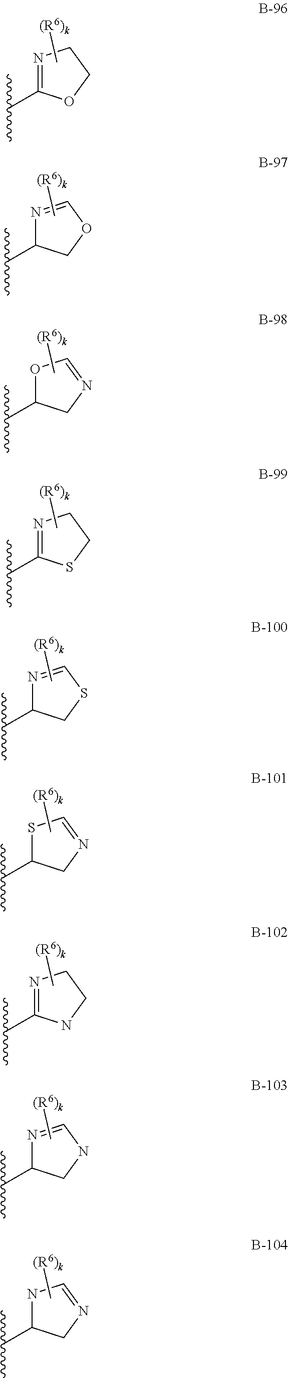 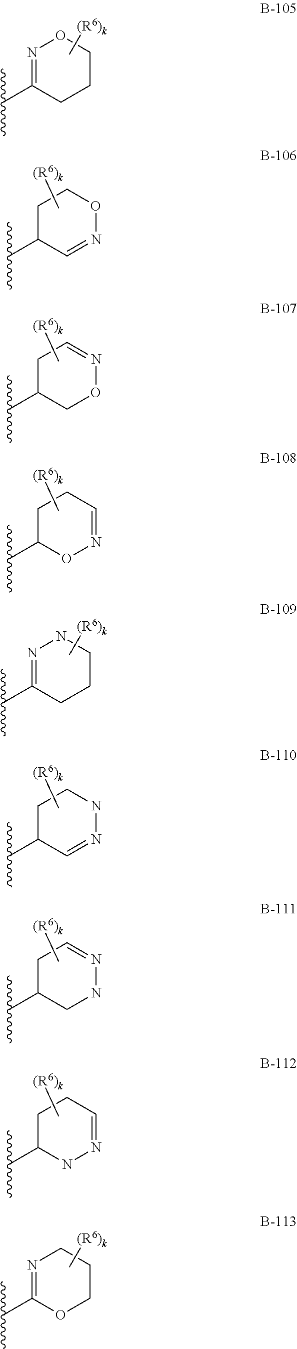 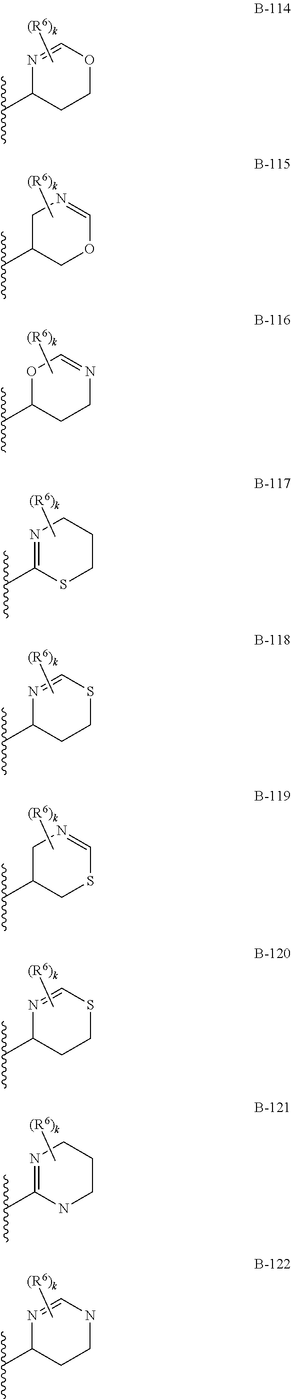 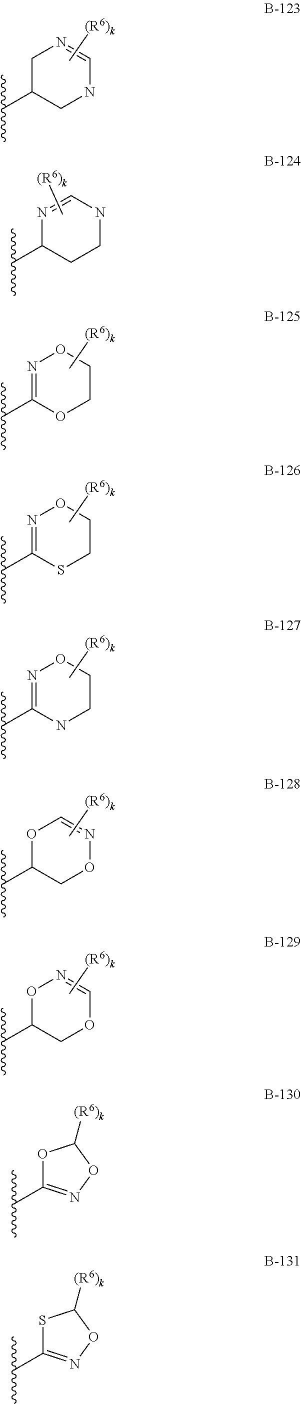 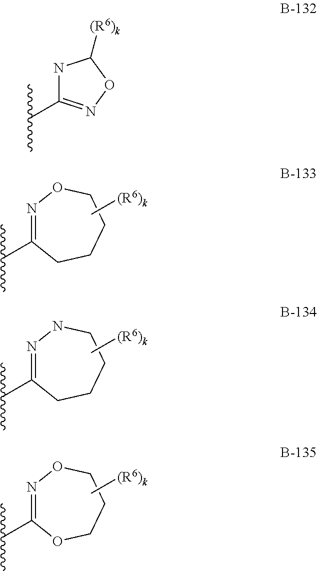
wherein the zigzag line denotes the bond to the remainder of the molecule and k is 0, 1, 2, 3, 4, 5 or 6;
and wherein the 3-, 4-, 5-, 6- or 7-membered saturated, partly unsaturated or maximally unsaturated heterocyclic ring R⁵ is selected from rings of formulae B-1 to B-32.

78: The compound as claimed in claim 50, wherein each R⁶ is independently selected from the group consisting of halogen, cyano, nitro, C₁-C₆-alkyl, C₃-C₈-cycloalkyl, wherein the two last-mentioned aliphatic and cycloaliphatic radicals may be partially or fully halogenated and/or may be substituted with one or more radicals R⁷; OR⁸, NR^{9a}R^{9b}, S(O)ₙR⁸, S(O)ₙNR^{9a}R^{9b}, C(═O)R⁷, C(═O)NR^{9a}R^{9b} and C(═O)OR⁸, or
two R⁶ present together on one carbon atom of a partly saturated heterocyclic ring may form together a group ═O, ═CR¹³R¹⁴; ═S, ═NR^{17a}, ═NOR¹⁶ or ═NNR^{17a}.

79: The compound as claimed in claim 77, wherein k is 0 or 1.

80: The compound as claimed in claim 50, wherein p is 0 or 1.

81: The compound as claimed in claim 50, wherein r is 0.

82: The compound as claimed in claim 50, of formula I.1 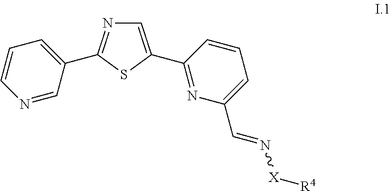
wherein X and R⁴ are as defined in claim 50.

83: The compounds as claimed in claim 82, wherein
X is O and R⁴ is methyl; or
X is O and R⁴ is ethyl; or
X is O and R⁴ is isopropyl; or
X is O and R⁴ is cyclopropylmethyl; or
X is O and R⁴ is allyl; or
X is O and R⁴ is propargyl; or
X is O and R⁴ is benzyl; or
X is O and R⁴ is phenyl; or
X is O and R⁴ is hydrogen; or
X is O and R⁴ is 2,2,2-trifluoroethyl; or
X is O and R⁴ is 3,3,3-trifluoropropyl; or
X is NCH₃ and R⁴ is CH³; or
X is NH and R⁴ is phenyl; or
X is NH and R⁴ is benzyl; or
X is NH and R⁴ is C(O)—CH₃; or
X is NH and R⁴ is C(O)-phenyl; or
X is NH and R⁴ is C(O)—NH—CH₃; or
X is NH and R⁴ is C(O)—NH—CH₂CF₃; or
X is NH and R⁴ is C(O)—NH—CH₂-cyclopropyl.

84: The compound as claimed in claim 50, of formula I.2 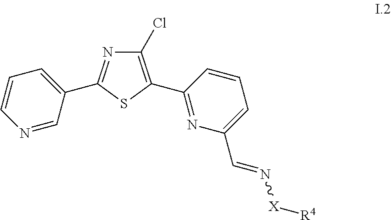
wherein X and R⁴ are as defined in claim 50.

85: The compound as claimed in claim 84, wherein
X is O and R⁴ is methyl.

86: The compound as claimed in claim 50, of formula I.3 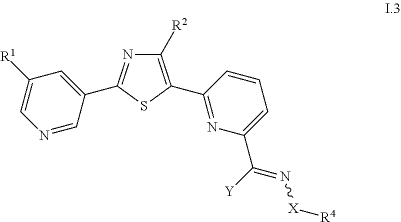
wherein
R¹ is H, R² is H, Y is CH₃ and —X—R⁴ is 2-pyridylamino;
R¹ is H, R² is H, Y is CH₃ and —X—R⁴ is OCH₃;
R¹ is H, R² is H, Y is CH₃ and —X—R⁴ is NHC₆H₅;
R¹ is H, R² is H, Y is H and —X—R⁴ is 4-chloroanilino;
R¹ is H, R² is H, Y is H and —X—R⁴ is 3,5-difluoroanilino;
R¹ is H, R² is H, Y is H and —X—R⁴ is 1,3-benzodioxol-5-ylamino;
R¹ is H, R² is H, Y is H and —X—R⁴ is 4-(trifluoromethoxy)anilino;
R¹ is H, R² is H, Y is H and —X—R⁴ is 1-naphthylamino;
R¹ is H, R² is H, Y is H and —X—R⁴ is 3-(trifluoromethyl)anilino;
R¹ is H, R² is H, Y is H and —X—R⁴ is 3,5-dichloroanilino;
R¹ is H, R² is H, Y is H and —X—R⁴ is morpholino;
R¹ is H, R² is H, Y is H and —X—R⁴ is pyrrolidin-1-yl;
R¹ is H, R² is H, Y is H and —X—R⁴ is N-butylanilino;
R¹ is H, R² is H, Y is H and —X—R⁴ is 2-fluoroanilino;
R¹ is H, R² is H, Y is H and —X—R⁴ is 3-fluoroanilino;
R¹ is H, R² is H, Y is H and —X—R⁴ is 4-cyanoanilino;
R¹ is H, R² is H, Y is H and —X—R⁴ is 3,5-dimethylanilino;
R¹ is H, R² is H, Y is H and —X—R⁴ is 2-methoxyanilino;
R¹ is H, R² is H, Y is H and —X—R⁴ is 4-methoxyanilino;
R¹ is H, R² is H, Y is H and —X—R⁴ is 4-(trifluoromethyl)anilino;
R¹ is H, R² is H, Y is H and —X—R⁴ is 3-chloroanilino;
R¹ is H, R² is H, Y is H and —X—R⁴ is 2-ethylanilino;
R¹ is H, R² is H, Y is H and —X—R⁴ is 2,3,5,6-tetrafluoroanilino;
R¹ is H, R² is H, Y is H and —X—R⁴ is 4,5-dihydro-1H-imidazol-2-ylamino;
R¹ is H, R² is H, Y is H and —X—R⁴ is 2,4,6-trimethylanilino;
R¹ is H, R² is H, Y is H and —X—R⁴ is 2,3-dihydro-1,4-benzodioxin-6-ylamino;
R¹ is H, R² is H, Y is H and —X—R⁴ is N-methylanilino;
R¹ is H, R² is H, Y is H and —X—R⁴ is 2-pyridylamino;
R¹ is H, R² is H, Y is H and —X—R⁴ is 4-chloropyrazol-1-yl;
R¹ is H, R² is H, Y is H and —X—R⁴ is (6-chloro-2-pyridyl)amino;
R¹ is H, R² is H, Y is H and —X—R⁴ is (5-chloro-2-pyridyl)amino;
R¹ is H, R² is H, Y is H and —X—R⁴ is 2,6-dichloro-4-(trifluoromethyl)anilino;
R¹ is H, R² is H, Y is H and —X—R⁴ is 4-fluoroanilino;
R¹ is H, R² is H, Y is H and —X—R⁴ is 4-methylanilino;
R¹ is H, R² is H, Y is H and —X—R⁴ is N-benzylanilino;
R¹ is H, R² is H, Y is H and —X—R⁴ is 3-chloro-4-fluoro-anilino;
R¹ is H, R² is H, Y is CH₃ and —X—R⁴ is 4-(trifluoromethoxy)anilino;
R¹ is H, R² is H, Y is CH₃ and —X—R⁴ is NHC(O)CH₃;
R¹ is H, R² is H, Y is CH₃ and —X—R⁴ is O(CH₂)₂OCH₂CH₃
R¹ is H, R² is H, Y is CH₃ and —X—R⁴ is 3,5-difluoroanilino;
R¹ is H, R² is H, Y is CH₃ and —X—R⁴ is isopropoxy;
R¹ is H, R² is H, Y is CH₃ and —X—R⁴ is 4-(trifluoromethyl)anilino;
R¹ is F, R² is H, Y is H and —X—R⁴ is OCH₃;
R¹ is F, R² is H, Y is H and —X—R⁴ is NHC₆H₅;
R¹ is F, R² is H, Y is H and —X—R⁴ is N(CH₃)₂;
R¹ is F, R² is Cl, Y is H and —X—R⁴ is OCH₃;
R¹ is H, R² is H, Y is CF₃ and —X—R⁴ is OCH₃;
R¹ is H, R² is H, Y is CF₃ and —X—R⁴ is NHC₆H₅;
R¹ is H, R² is H, Y is CF₃ and —X—R⁴ is NHCH₂—C₆H₅;
R¹ is H, R² is Cl, Y is H and —X—R⁴ is N(CH₃)₂;
R¹ is H, R² is H, Y is H and —X—R⁴ is O(CH₂)₂OCH₂CH₃;
R¹ is H, R² is H, Y is H and —X—R⁴ is 2,4-dioxoimidazolidin-1-yl;
R¹ is H, R² is H, Y is H and —X—R⁴ is 1-piperidyl;
R¹ is H, R² is Cl, Y is H and —X—R⁴ is NHC₆H₅;
R¹ is H, R² is CF₃, Y is H and —X—R⁴ is N(CH₃)₂;
R¹ is H, R² is CF₃, Y is H and —X—R⁴ is OCH₃;
R¹ is H, R² is H, Y is H and —X—R⁴ is 2-oxopyrrolidin-1-yl;
R¹ is H, R² is H, Y is H and —X—R⁴ is 2-oxo-1-piperidyl;
R¹ is H, R² is H, Y is H and —X—R⁴ is methanesulfonamido;
R¹ is H, R² is CH₂OH, Y is H and —X—R⁴ is OCH₃;
R¹ is H, R² is CHF₂, Y is H and —X—R⁴ is OCH₃;
R¹ is H, R² is CH₂F, Y is H and —X—R⁴ is OCH₃;
R¹ is H, R² is CHF₂, Y is H and —X—R⁴ is N(CH₃)₂;
R¹ is H, R² is CH₂F, Y is H and —X—R⁴ is N(CH₃)₂; or
R¹ is H, R² is CH₂OH, Y is H and —X—R⁴ is N(CH₃)₂.

87: The compound as claimed in claim 50, of formula I.4 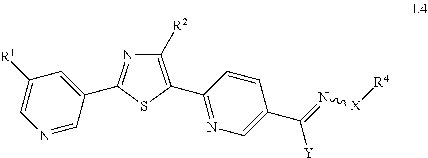
wherein
R¹ is H, R² is H, Y is H and —X—R⁴ is NHC₆H₅;
R¹ is H, R² is H, Y is H and —X—R⁴ is N(CH₃)₂;
R¹ is H, R² is H, Y is H and —X—R⁴ is OCH₃; or
R¹ is H, R² is H, Y is H and —X—R⁴ is NH—CH₂—C₆H₅.

88: The compound as claimed in claim 50, of formula I.5 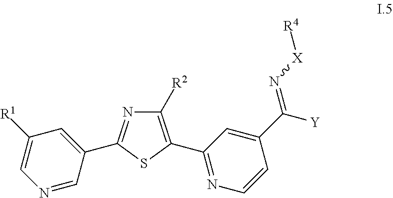
wherein
R¹ is H, R² is H, Y is H and —X—R⁴ is OCH₃;
R¹ is H, R² is H, Y is H and —X—R⁴ is NHC₆H₅;
R¹ is H, R² is H, Y is H and —X—R⁴ is OCH₂CCH;
R¹ is H, R² is H, Y is H and —X—R⁴ is NH—CH₂—C₆H₅; or
R¹ is H, R² is H, Y is H and —X—R⁴ is N(CH₃)₂.

89: The compound as claimed in claim 50, of formula I.6 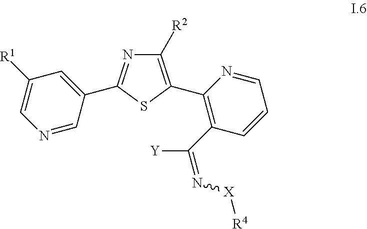
wherein
R¹ is H, R² is H, Y is H and —X—R⁴ is NHC₆H₅;
R¹ is H, R² is H, Y is H and —X—R⁴ is OCH₃;
R¹ is H, R² is H, Y is H and —X—R⁴ is N(CH₃)₂; or
R¹ is H, R² is H, Y is H and —X—R⁴ is NHC(O)CH₃.

90: An agricultural composition comprising at least one compound of the formula I, as defined in claim 50, a stereoisomer thereof and/or at least one agriculturally acceptable salt thereof, and at least one inert liquid and/or solid agriculturally acceptable carrier.

91: A veterinary composition comprising at least one compound of the formula I, as defined in claim 50, a stereoisomer thereof and/or at least one veterinarily acceptable salt thereof, and at least one inert liquid and/or solid veterinarily acceptable carrier.

92: A method for controlling invertebrate pests which method comprises treating the pests, their food supply, their habitat or their breeding ground or a plant, plant propagation material, soil, area, material or environment in which the pests are growing or may grow, or the materials, plants, plant propagation material, soils, surfaces or spaces to be protected from invertebrate pest attack or infestation with a pesticidally effective amount of at least one imine compound of the formula I as defined in claim 50, a stereoisomer thereof and/or at least one agriculturally acceptable salt thereof.

93: The method as claimed in claim 45, for protecting plants from attack or infestation by invertebrate pests, which method comprises treating the plants with a pesticidally effective amount of at least one compound of the formula I as defined in claim 50, a stereoisomer thereof and/or at least one agriculturally acceptable salt thereof.

94: The method as claimed in claim 45, for protecting plant propagation material and/or the plants which grow therefrom from attack or infestation by invertebrate pests, which method comprises treating the plant propagation material with a pesticidally effective amount of at least one compound of the formula I as defined in claim 50, a stereoisomer thereof and/or at least one agriculturally acceptable salt thereof.

95: Plant propagation material, comprising at least one compound of the formula I as defined in claim 50, a stereoisomer thereof and/or at least one agriculturally acceptable salt thereof.

96: A method for treating or protecting an animal from infestation or infection by invertebrate pests which comprises bringing the animal in contact with a pesticidally effective amount of at least one compound of the formula I as defined in claim 50, a stereoisomer thereof and/or at least one veterinarily acceptable salt thereof.
